SEQUENCE LISTING (866 sequences)

SEQ ID NO: 1 - DNA, 14000 bp, Homo sapiens
  source 1..14000: mol_type = genomic DNA
ggctcaagcctgtaatctgtaatctcagcattttgggaggctgaggcagcaggatcacttgaggccaggagcttgagaccagactaggcaacatattaagacaaaaaaatacaaaatatagctgggagtggtggcatgtgtctgtattcccagctactcaggaggctgaggtgggagggtctgaggctgcagtacaaggtggcaccactgtaatccagcttgggcggcagaggaaggctctgtctcaaaaacaaagaaacaaacaaaaatgggccacgcgtggtggctcacgcctgtaatcccagcagtctgggagattgaggtgggtggatcacttgaggtcaggagttcgagaccagcctggtcaacatggcggaaccctgtctctactaaaatacaaaaattagctgggcatcatggcacatgcttgtaatcccagctatccgggaggctgagacaggagaattgcttgaacccaggaggcagaggttgcagtgagccaagatcgtgccactgcactccagcttgagtgttggagtgagactctgtctcaaacaaatgaaaagaaacaaacaaaaaagaaatgagatactgatacctgctatgtgataagctttgaaaacattatgctaagtgaaagaagccagttgcagatcacatattataaaatttcatttatatggaatatccaaatctatagaggtagaaagtagatcagtggttgcctagggttagggagggtgactgctaaaggacataaggctgccttctgtggtgataaaaatggtcttaaactgattgtaatgatgctgcataactctcttaagacactaaaaaccattgagttgtacactttaatttttttgtgttggcgggggtggatggagtctcgctctgttgtccaggctggaatgcagtggcacgatctcggtttactgcaacctctgcatcccgggttcaagcgattctcctgcctcggcctcctgagtagggaattacagacctcgttatcgtggcaccttacccttctgatgttaaaaaaaaaaaaaaaaagagcgagagagagagagagaaacatttgtgaagtaggttgttgagtctcagcactattgaccttttgggcaggatacttctttgttgtgggggattgttctgtgtgtcgtgtgatgtttagtgggattgctggcccttacctaccagatgccagtgtccctccaccctgagttgtgacaacccagattgtctccagacactcctaaatgtccctggccggcaaaattgccgctgctcaagaatcacggctttgacgattagactttgtgatatttgtttcagtctgtttaggttttttttcttctacctgtatttttttctggttctgggtggttgtaattagtaggttattgatcgattcacctaacatttcatgaaagtttcatgtgtgtgtgtgtttcaatagaagcataaactatactccctagtctcaagatacacaggaaggaaaataagcacaaatgtgtcaccagggcacagactagtactaggtcctcagcaggccaggtgtcttatccgctgtctgggtctgctctagctccaggcttagaacccctgccacacgactccacagctcggttggcaccctttccctcctccgacttctgctgcctcgagcttggttagccatccccctgcccctgcctcatcctcagctccagttccttgctcaggctgcagcagtctccatcccctgtgcagacactgccgttcctccacggcccagtatcaggctttccctgggcctctcctctctcctggcccatctcccatcatccatctctgcctggcccaggccctttggcaccaagcaggctgactcttgtcactggctaatctgttctgtggtacattttctctcctcaccctcccatatcaattcctcgaaggcagggccgatctggagactaggaagccacttctctttcgacagcccccaccacagcccagcccgtgccaggcacccagcagctcctgaagcccactggcattgaacatggcattcaatccctgccaagcctgcccttcccatctggtttcccagggctcttcccaacacctcctcctccacctgccagttaaaatcttcccagactcagctcaaggagatgctcctaaggtggaatgaaatctcttcttccccacctggagacaatctacttcctctccctacacctggcaactggcgcacaaccttgtatcttaaattagattcagcctgagactgtctcccaccaatccctgctccctgtcctgctgagcaccttgaggaaagggctttggggctgtttatctttgtcctggaaaccatccttcaactcactctggggcctgcctagcatgtcaaccgagtttggagaatagggcagaatagggcaggacaggacaggacaagacagggcaggataggataggagcgagccagctcagtagctcacatttgtaatcccagcgccttggggggctgcggtaggagaatcgctttgggagcaggagttgcaggccgcagtgagctatgatcagcttgggcgactgagcgagaccctgtctctaaaacaaacacacaagtccgggcgcggtggctcatgcctgtaatcttagcactttgggaggccgaggtgggcggatcacgaggtcaagaaatcgagaccatcctggccaacatggtgaaaccccgtctctactaaaaatacaaaaattagctgggcgtggtggtgcgcgcctgtagtcccagctactcgggaggctgaggcaggagaatcgcttgaacccgggaggcagaggttgcagtgagccgagatcgtgccactgcactccagcctggcgacagagtgagactccgtctcagaacaaacaaacaaaaggatagaaaggcgagcacaaatattcccaattcataacactccctcgcactgtcaatgccccagacacgcgctatcatctctagcaaactcccccaggcgcctgcaggatgggttaaggaaggcgacgagcaccagctgccctgctgaggctgtcccgacgtcacatgattctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggtaggagagcggccgcgcagacctctcgcctgctcctgcccaggggcccgccagggccatgtgagcttgaggttcccctggagtctcagccggagacaacagaagaaccgcttactgaaactccttgggggttctgatacactagggggagttttatgggaaagaggaagcagtaattgcagtgacgccccgttagaaggggctttctacctccccagcattcccccaaagcagggaccacaccattcttgacccagctccacccctgtcggtaggtgctggcttcttcccctctcctggtggtggtgggtggttcccgcggcggcctggagccggaggggcgcgcgaccctgggctgggagctccgagggcctgggaacgagacctgagaccttggcttctcgaaggtagtagggacttgggagtggtgactgaacctggtctggctcctccttacttcctcttgttgcgggtgggacgagctagcttccgcctctcccagccactttttcctgctcatttgcagctaggttggctccccttttgggaatttcctctccccttggcactcggagttggggggtgccacctagtggaagataacggagctagggtcttgaagaggctgctgtcccctctggctgttttggcggtgtagggtggcatgagagactgcgactcgcctcctcatccctgtttctgtatgcgagtgcttgtattcagtagaagcatacactatactccctcaatttagggtaaacaggaggggccacatgcacaggtaattcaccagggagccgaacactcctgtgcagacagactccccttcccagcaagccatggcagcggacagcctgctgagaacacccaggaagcaggcggtgccagctgcaggtgctttgcctgggagctgtggggctgaggagagggtccactgtccaggaccagtgaacttcatccttatctgtccaggaggtggcctcttggggatgctgagttaggggaggggcacttgaggaaagccaggtggagcagagaggatgtgagtgactgggtgggtgagatttcctgcccctccccccgcagtggtatccacacctagactcgtggggtaactgaggcacagacagagagcaacttctcaggccctcacagttggcaattctaggattaggacccaagtgcgattttcaggcagtccctgtaccctgtttctgttgtacctgttgcaccattcccaggcactgcccatcgtgccactagtgatatgaacccaggtccaatacgctctggggccatcaaagcctgacgtcaccatgacctgatgtgtgacgtgttataggtgtcccttggtatcttcacggaactggttccaggaccccaaaatctgtgggtgctcaagcccctgagataaaatggtgtaatatttgcatataacctatacatactttaaatcatttctagattacttatacctaatacaatggaaatgacatgtcggctgggcgtggtggctcatgcctgtaatcccaccactttgggaggccgtggcaggtggatcacctgaggtctggagtttgagaccagcctgaccaacatggtgaaacccccatctctactaaaaatacaaaaattagccaggtgtggtagcgcacacctataatcccacctacttgggaggctgaggcaggagaattgcttgaacctgggaggcggagttcgcagtaagctgagatcgcgccactgtactacagcctgggtgacagagcaggactccatctcaaaaaaaaaagagaaaaagaaaaagaaatgccatgtaaatagttgtgatcctgaattgtttagggaataataagaaagaactatctgtagatgttcagtatagatgcacccatcgtaagcctaactacattgtataactcagcaacgatgtaacattttcaggggtttttttgttttgttttttgagacagaatctcagtctcactctgtcacccaggctggagtatgttggcgtgatctctgctcactgcaacctccacctcctgggctcaagcgattctcctgcctcagcctcttgagtagctgggattgcaggtgtgcgctaccacgcatggctaatttttgtatttttaatagagatggggttttaccacgttggtcaggctggtcttgaactcctgaccttgggatccgcccacctgggcctcccaaagtgctgggattacaggcgttagccaccgcgcccaatatattttgatccctggttggatatggagggctgactgtacttaacatctctaagcttcagtttcctcctttaaaataaaggtgtggctgggtgtggtggttcaagcctgtaatcccagcacttagggaggctgaggtgggtggatcagctgaggtcaggagttcaagaccagcctgaccaatatggtgaaaccccctctctgctaaaaatacaaaaattagccaggcgtggtggcgagcgcctgtagtcccagctacttgcttgaacttgggaggcagaggttgcagtgagctgagatcgtgccactgaactcgagcatgggcaacagagcaagactgtctcaaaaaaaaaaaaaaaaagggggtgagcagacgtggtggcacgctcccacagtcccagctacttagtaggaggccaaggttggaggattgcttgatcccaggagtctgagtccagcctgggcaacatggcaatacctcatctctaaaaataaaataaaagtaaaggtattaattactactttggatggttgttgcaaagaaatatatataaaataatggagagtcttgtaactggctcccaagaggctcaacagacattactgtttttgcttcttcattatgagttacctctctggccaccccactgaactagctgggctagctgagcctgggagaagagttgtttaggaagtgagaggctgctctccacagagactcaaggctcagttcctcctggtgactcagatgggcagcccagtgggcacacgtggtctctctccacatgtggctgagtttcacttccagaatagatggagaggcaagggcagggtttagcatgcttgaggaatctcagagggccctggtggtgtgggggaccctcagaacacaggtgtctcaagggctgacccagcttctgtgtccttttctctgggtgaggaggggacattcatgggcagatggtgacctctggggaaggcagcccagactccactggccaccatatttcctttttcacaactttctcacccctgtggtttcccatgtcatcatgtggccgcttcccgcaaggccttagcggggtgcaggtatgaacatagtgtcaggcaaggaggcatctggaggggaaccctggcttttcctggggggactccctccctgcaccctagccctgtcctctcccatggctactgatgccttcccctcaccccagaggtggcccacatctgcacagatcagacccacaaaaatcacgtcttcctgactctcataagcctgcccagtgaggcccaggcattaggccatgtgctggggactcagacccacacatatacgcatgtcagcattcatgcttacaggtccgcacatgctggggcaagtgtcacacacggggcgctgtaggaagctgactctcagcccctgcagatttctgcctgcctggacagggaggtgttgagaaggctcaggcagtcctgggccaggaccttggcctggggctagggtactgagtgaccctagaatcaagggtggcgtgggcttaagcagttgccagacgttccttggtactttgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggtgagtgcccctcagcctaggcaagagctggcagcctgggttttcccaaagggtcatcttggattggccagaggaggacgccaggcacaagtctgtggtttatcattttccctgtctttctaggacaaatggcccacaacactgagcaggcatctgggtggcccctgccaggttgatgcccactgctctgccggccactcctgcatctttaccgtctcagggacttccagttgctgccccttcccagaggtgagcgtgccatcagcccagtggaggggcttaggtctgcatttatgcttttcctgcactctaccacctgcagataaaagggccctgccaatgcaggtttctctgtgttccacaggccgtggcatgcggggatggccatcactgctgcccacggggcttccactgcagtgcagacgggcgatcctgcttccaaagatcaggtgcagctggggtgtgggtgcagggcaggcagacgggcagcatgtggagtctggaacccaggagcccagctggcgggggcagccctgattcctgcccttgtgccctcattcatgtggcatctgtactaagcaacagccctgctgtggacagaggggcagcactggggataggagggtgcgggagaaagtgcaagactccaggtccaggcgttgtgggggtggggagaggtcgagctgggccggtctaataccaacccatggtcagtgggtgccccttccccatgccatcttgctgagggagggactggattgtgaggagggtgagttaggcctgcctaggagatcactgagccttagtgtcaccctcaaaccccagtagctgggcttgcaggccctggtgccaccagctccttgtgtgatgggggagtcaccttccctgagtgggctggtagtatcctgggtcatcttgtccacaggtaacaactccgtgggtgccatccagtgccctgatagtcagttcgaatgcccggacttctccacgtgctgtgttatggtcgatggctcctgggggtgctgccccatgccccaggtacaaatctgggggagatgggggtatgtggagggaagtgggggcagagttgggggccaggggcagggggtgaagacggagtcaggaccattttttctcaggcttcctgctgtgaagacagggtgcactgctgtccgcacggtgccttctgcgacctggttcacacccgctgcatcacacccacgggcacccaccccctggcaaagaagctccctgcccagaggactaacagggcaggtgaggaggtgggagagcatcaggccaggggctggggcggggcctcattgactccaagtgtaggaaaaagtttcctccatcctggctgcccctcacgtttgctcctcttccagtggccttgtccagctcggtcatgtgtccggacgcacggtcccggtgccctgatggttctacctgctgtgagctgcccagtgggaagtatggctgctgcccaatgcccaacgtgagtgaggggctggagccagcttggctgtgtgcccccagccacctggccctgacacgcaccttacaggggctctgtggcatggggctggctggctgcttgctgggagcctggctgatgcagggttcatgctaccccctagtgggggattggggcagtgccagccatcagcctggctgctccctgtgtgctactgagcctggaagtgacaaagacccacccctgtccccactcaggccacctgctgctccgatcacctgcactgctgcccccaagacactgtgtgtgacctgatccagagtaagtgcctctccaaggagaacgctaccacggacctcctcactaagctgcctgcgcacacaggtaccagaggcagggtgcagatacaggggtggggccccctttcctcccttttaggcctggccttaggatcactgcaaggtggtgtaagcggtaccctccatcttcaacacctggttccagctgtggagccggcaaagggttgatacccctgagggtccccagtgccacttctgacctgtcctctctgcttccctcacagtgggggatgtgaaatgtgacatggaggtgagctgcccagatggctatacctgctgccgtctacagtcgggggcctggggctgctgcccttttacccaggtacccaggggtggcgggtgggtgggctgagcacagtgtggcaggcagccgggccccagtgcccacctgcccttcttcatctgccctaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggtatatgggaggggacagcatcttggcctgggcaggtgggtggccaagctcctattgctttctgccctccgcatagcccataggtgatacccagctctgacagattcgtccccagctggaggtgctgtaagcaggagaggcgggctggagtaggtaggggctcggcactgcgccccacatagtggctacctacaacgccctttcctgcccaccccccaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgtgagtgcctccctgcctgcccctggataggggagctaagcccagtgaggggacaggaacataatgccattctgtgctcccttccccgccaggctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccaggtcagtgccaacccccatcctggggctgggtatggccagggaccaggtcccacctcgtccaaccctctcgcccccctctgaccatccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcttaacagtgtctgatttgccgccctgcctgccctccccagggccccagaacaggggttcacgtccactgccaacactcccctcccctaccccacagaaagacatacacagccttaacctcaccagttttatatttgctgctgcccacatcggctgtatcacattaccccctagtccccagagctgtcccagccaccagccctgtgacatcgtagcccagagatgggctagtcagcaagcagcaccccctcccctcccaggggtccacaaagaacgccccctcccttcccagccctcacactagcagctgaggctgggtcacccctcctgctttcccacaatagagctttctatgtacagccacgtctacacaggcactgcttccccccagccctcctccccggcacctccccgtgggggtctggaccccccctcctccccggcttggaggcagacacagggtcccttgcaagacacgacccagcaccaaccacggaacagctccaaggcccctgggcccctctccggcctggggctgggagctacgcgcgagggcccccgcgggcccccggggcgcgcaccctgggtgcgggcccgcgcgggaggggcggtgccaggccctgcgcgggcgctacttgacgttgaacaccatcagcggccgctcctcccgccgctgccacgggctcttcttgtctcccgcctcgtcgcccacctccgcccccagctcgtcctccgggctggtgagcgagtcgcgccgcagctcgctggcgctgctgcgggagctgtccccgcggctgcggccccgcccccggggtaccgtggccgcccggccctcgggcgccgccacctcgtccagcagcggcgtcagcgagttgtcctccggagagcagaggcccgtgcggctctcgctgctgctgggctccgtgtcctcgctgagcgccaggcgcgggggcgcgggcggcggcggcggcgcggcaggcggggcgcgctcccgctcctccctcgcgggccggcggcgcgcgggccgcgcctcgtggacatctacgcccgagtccaggctggcgtcggtgctgcgtgccccgccgcccgcctgcaggtcgatgtaagagtggcgcacttgcgagttggagcgcccgtcgagggacacgaaccaggcgcgcgggtgcggcttcacgcccagttccagcagcttcttctcggtcagggcctgcagctccccgttgagctgcgccatggtggactcgttgaatagcacagggatggtgactgagccactgacgggcgccgcgcgcccggccccccagccctcgccgccgcccccgccgccctcgccccccgggcccgagtggcccggcatctgcgggcgctgggggtcgggctggggaaaagcgcgcgccgggccgggtgccgtgccctccggcggggccggctcgtcgcccacgccggcggcgcccgcctcgccgccgaggcgcacgtagtgcgcggggatcaccagggtgggcatgacgttgcggtagacgttgtccttgaggtggtcgatggagccgcagaagatgagctgccccgcctggcccagcgacggcggccgcgccagctggtccaccgactgcgacagcaggaagtcgggggtcttgccctcggccgcccccttgtggcccaggtagtggtcgaagggcggcggcggcgagggcggctcgtgcaggaaggccgcagcccccgagggcccccgccggtgctcctctaggccgggctccagcccgccggggccctcggccgagcgagcgcccttgagcccggcgctctcgcccccgcgggcacccgaaggctcggcggccgggcggctggcagagcgcggcttggtgcggaagaagtcatcccgggaggaggccaagtcccgggagctggagaaggccgagtggagggggcctggaggcggagcctcggggtcccccgacggggcgggttccaggggtcccccacagatgaggtggagctgggacatcgaggtggcctggtctcgtttgttaccgtcagagggccccgagagctgcagcttgcggtgctgttgcctcggcttcaggcagcgcctcctggaagggagggagcagaaggggccgctcaggaaagggtgagggccaaagtaggccccgaacccccttagatgcaagacaagagacgaatatagtggaagaggtggggagaatgcctggggcgctcaggagagatggcggggagccgccgtgtccgtttggcgatcccctcagggtcaactctgcactcctctgcatgtaggatgtgctgcgagaaccagggatgagcatctaagctgtctatgggtgtgtccggcctggagtcctctgggagagagagctgggctggtaggggcagtggcagatctctctggaggggccgggctgcaagtccttaagccctgcctcccctcccacctccccgctgtcttcaaccaccagcaccagcccttcaggtcttagcatgtttgcaggaagtcttttcctgtctgccccttgaggaccagaagtggattttatgcatctttggggccccagggcccggtacaagtgcaggggagggtgagggaagccccgtgctcaccggcagtagtagatgagcagacacagcaggataagcaccagcagggccagggctgccaggatggtgagcaagaagatggtgtggtaggtgccgatgtcctggatgcccgacgtgatggtgaccagccctgtgccaggcgagggcttagcgttcaggtgtagacacccttcctgcctttcagaagtctgctctcccagctcccctcctccgcggcctctacgcccactgccccactactcttaccagccgtgggggaggccatggcggccacccagtaccccagctg

SEQ ID NO: 2 - RNA, 2356 nt, Homo sapiens
  source 1..2356: mol_type = mRNA
attctccaatcacatgatccctagaaatggggtgtggggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcttaaaaaaaaaaaa

SEQ ID NO: 3 - DNA, 19 bp, synthetic construct
  source 1..19: mol_type = other DNA
aggactaacagggcagtgg

SEQ ID NO: 4 - DNA, 18 bp, synthetic construct
  source 1..18: mol_type = other DNA
cagcagccatacttccca

SEQ ID NO: 5 - DNA, 23 bp, synthetic construct
  source 1..23: mol_type = other DNA
ttgtccagctcggtcatgtgtcc

SEQ ID NO: 6-9 - skipped (4 entries)

SEQ ID NO: 10 - DNA, 18 bp, synthetic construct
  source 1..18: mol_type = other DNA
ttagtttaatcacgctcg

SEQ ID NO: 11 - skipped

SEQ ID NO: 12 - DNA, 18 bp, synthetic construct
  source 1..18: mol_type = other DNA
atcatgtgattggagaat

SEQ ID NO: 13 - DNA, 18 bp, synthetic construct
  source 1..18: mol_type = other DNA
tcctccctgcttcctctc

SEQ ID NO: 14 - DNA, 18 bp, synthetic construct
  source 1..18: mol_type = other DNA
cagcctggaatgctgtgt

SEQ ID NO: 15 - DNA, 18 bp, synthetic construct
  source 1..18: mol_type = other DNA
acccgctcccattggcta

SEQ ID NO: 16 - DNA, 18 bp, synthetic construct
  source 1..18: mol_type = other DNA
acccgcctacctcagttt

SEQ ID NO: 17 - DNA, 18 bp, synthetic construct
  source 1..18: mol_type = other DNA
accgggtagcgctcagac

SEQ ID NO: 18 - DNA, 18 bp, synthetic construct
  source 1..18: mol_type = other DNA
ggcagcagcaaccgggta

SEQ ID NO: 19 - DNA, 18 bp, synthetic construct
  source 1..18: mol_type = other DNA
gcggtccttgggcagcag

SEQ ID NO: 20 - DNA, 18 bp, synthetic construct
  source 1..18: mol_type = other DNA
gtccgactccgcggtcct

SEQ ID NO: 21 - DNA, 18 bp, synthetic construct
  source 1..18: mol_type = other DNA
gtctgcctgcgtccgact

SEQ ID NO: 22 - DNA, 18 bp, synthetic construct
  source 1..18: mol_type = other DNA
ggtccacatggtctgcct

SEQ ID NO: 23 - DNA, 18 bp, synthetic construct
  source 1..18: mol_type = other DNA
acccagctcaccagggtc

SEQ ID NO: 24 - DNA, 18 bp, synthetic construct
  source 1..18: mol_type = other DNA
actgaccatctgggcacc

SEQ ID NO: 25 - DNA, 18 bp, synthetic construct
  source 1..18: mol_type = other DNA
gcagctgtagctggctcc

SEQ ID NO: 26 - DNA, 18 bp, synthetic construct
  source 1..18: mol_type = other DNA
cccagatgcctgctcagt

SEQ ID NO: 27 - DNA, 18 bp, synthetic construct
  source 1..18: mol_type = other DNA
agatgcaggagtggccgg

SEQ ID NO: 28 - DNA, 18 bp, synthetic construct
  source 1..18: mol_type = other DNA
tgccacggcctctgggaa

SEQ ID NO: 29 - DNA, 18 bp, synthetic construct
  source 1..18: mol_type = other DNA
tctgcactgcagtggaag

SEQ ID NO: 30 - DNA, 18 bp, synthetic construct
  source 1..18: mol_type = other DNA
ggatggcacccacggagt

SEQ ID NO: 31 - DNA, 18 bp, synthetic construct
  source 1..18: mol_type = other DNA
aacacagcacgtggagaa

SEQ ID NO: 32 - DNA, 18 bp, synthetic construct
  source 1..18: mol_type = other DNA
cagcaggaagcctggggc

SEQ ID NO: 33 - DNA, 18 bp, synthetic construct
  source 1..18: mol_type = other DNA
gaaccaggtcgcagaagg

SEQ ID NO: 34 - DNA, 18 bp, synthetic construct
  source 1..18: mol_type = other DNA
gagcttctttgccagggg

SEQ ID NO: 35 - DNA, 18 bp, synthetic construct
  source 1..18: mol_type = other DNA
cacatgaccgagctggac

SEQ ID NO: 36 - DNA, 18 bp, synthetic construct
  source 1..18: mol_type = other DNA
gcagctcacagcaggtag

SEQ ID NO: 37 - DNA, 18 bp, synthetic construct
  source 1..18: mol_type = other DNA
ggagcagcaggtggcgtt

SEQ ID NO: 38 - DNA, 18 bp, synthetic construct
  source 1..18: mol_type = other DNA
ctctggatcaggtcacac

SEQ ID NO: 39 - DNA, 18 bp, synthetic construct
  source 1..18: mol_type = other DNA
gcagcttagtgaggaggt

SEQ ID NO: 40 - DNA, 18 bp, synthetic construct
  source 1..18: mol_type = other DNA
tgggcagctcacctccat

SEQ ID NO: 41 - DNA, 18 bp, synthetic construct
  source 1..18: mol_type = other DNA
gggcagcagccccaggcc

SEQ ID NO: 42 - DNA, 18 bp, synthetic construct
  source 1..18: mol_type = other DNA
ccgcgggacagcagtgta

SEQ ID NO: 43 - DNA, 18 bp, synthetic construct
  source 1..18: mol_type = other DNA
ctggtggggcccctgttc

SEQ ID NO: 44 - DNA, 18 bp, synthetic construct
  source 1..18: mol_type = other DNA
tgtgggtctggcaggctg

SEQ ID NO: 45 - DNA, 18 bp, synthetic construct
  source 1..18: mol_type = other DNA
aggagggacagctgctga

SEQ ID NO: 46 - DNA, 18 bp, synthetic construct
  source 1..18: mol_type = other DNA
gattggacagcagcccca

SEQ ID NO: 47 - DNA, 18 bp, synthetic construct
  source 1..18: mol_type = other DNA
tagccctgggggcagcag

SEQ ID NO: 48 - DNA, 18 bp, synthetic construct
  source 1..18: mol_type = other DNA
cagccacgatctcgcttc

SEQ ID NO: 49 - DNA, 18 bp, synthetic construct
  source 1..18: mol_type = other DNA
gtctctggggtgggataa

SEQ ID NO: 50 - DNA, 18 bp, synthetic construct
  source 1..18: mol_type = other DNA
gggcagcaggtctgcccc

SEQ ID NO: 51 - DNA, 18 bp, synthetic construct
  source 1..18: mol_type = other DNA
agcacacagcatggggca

SEQ ID NO: 52 - DNA, 18 bp, synthetic construct
  source 1..18: mol_type = other DNA
cttcacgttgcaggtgta

SEQ ID NO: 53 - DNA, 18 bp, synthetic construct
  source 1..18: mol_type = other DNA
aggaaggtggcaggctgg

SEQ ID NO: 54 - DNA, 18 bp, synthetic construct
  source 1..18: mol_type = other DNA
gtccttccccacactcca

SEQ ID NO: 55 - DNA, 18 bp, synthetic construct
  source 1..18: mol_type = other DNA
ccagccctgtcggttgtc

SEQ ID NO: 56 - DNA, 18 bp, synthetic construct
  source 1..18: mol_type = other DNA
cagtggcgccgatcagca

SEQ ID NO: 57 - DNA, 18 bp, synthetic construct
  source 1..18: mol_type = other DNA
tgcgcaaacacttggtac

SEQ ID NO: 58 - DNA, 18 bp, synthetic construct
  source 1..18: mol_type = other DNA
ctgtctcaaggctgggtc

SEQ ID NO: 59 - DNA, 18 bp, synthetic construct
  source 1..18: mol_type = other DNA
tactgtccctcacagcag

SEQ ID NO: 60 - DNA, 18 bp, synthetic construct
  source 1..18: mol_type = other DNA
gagtcttcagtactgtcc

SEQ ID NO: 61 - DNA, 18 bp, synthetic construct
  source 1..18: mol_type = other DNA
gagggctgcagagtcttc

SEQ ID NO: 62 - DNA, 18 bp, synthetic construct
  source 1..18: mol_type = other DNA
gtggggtcccgagggctg

SEQ ID NO: 63 - DNA, 18 bp, synthetic construct
  source 1..18: mol_type = other DNA
caccctccgagtggggtc

SEQ ID NO: 64 - DNA, 18 bp, synthetic construct
  source 1..18: mol_type = other DNA
gagcagagggcaccctcc

SEQ ID NO: 65 - DNA, 18 bp, synthetic construct
  source 1..18: mol_type = other DNA
agggaggcctgagcagag

SEQ ID NO: 66 - DNA, 18 bp, synthetic construct
  source 1..18: mol_type = other DNA
gggaggtgctagggaggc

SEQ ID NO: 67 - DNA, 18 bp, synthetic construct
  source 1..18: mol_type = other DNA
tttggttagggggaggtg

SEQ ID NO: 68 - DNA, 18 bp, synthetic construct
  source 1..18: mol_type = other DNA
ccagggagaatttggtta

SEQ ID NO: 69 - DNA, 18 bp, synthetic construct
  source 1..18: mol_type = other DNA
agaatggggtccagggag

SEQ ID NO: 70 - DNA, 18 bp, synthetic construct
  source 1..18: mol_type = other DNA
tggggagctcagaatggg

SEQ ID NO: 71 - DNA, 18 bp, synthetic construct
  source 1..18: mol_type = other DNA
cccatggtgatggggagc

SEQ ID NO: 72 - DNA, 18 bp, synthetic construct
  source 1..18: mol_type = other DNA
ggccccacctcccatggt

SEQ ID NO: 73 - DNA, 18 bp, synthetic construct
  source 1..18: mol_type = other DNA
cttagattgaggccccac

SEQ ID NO: 74 - DNA, 18 bp, synthetic construct
  source 1..18: mol_type = other DNA
cagggaaggccttagatt

SEQ ID NO: 75 - DNA, 18 bp, synthetic construct
  source 1..18: mol_type = other DNA
ccccttctgacagggaag

SEQ ID NO: 76 - DNA, 18 bp, synthetic construct
  source 1..18: mol_type = other DNA
ttgccacaacccccttct

SEQ ID NO: 77 - DNA, 18 bp, synthetic construct
  source 1..18: mol_type = other DNA
aatgtggcttttgccaca

SEQ ID NO: 78 - DNA, 18 bp, synthetic construct
  source 1..18: mol_type = other DNA
ggcagcttgtaatgtggc

SEQ ID NO: 79 - DNA, 18 bp, synthetic construct
  source 1..18: mol_type = other DNA
gggaggggatggcagctt

SEQ ID NO: 80 - DNA, 18 bp, synthetic construct
  source 1..18: mol_type = other DNA
cactgaaacggggagggg

SEQ ID NO: 81 - DNA, 18 bp, synthetic construct
  source 1..18: mol_type = other DNA
ccacagggtccactgaaa

SEQ ID NO: 82 - DNA, 18 bp, synthetic construct
  source 1..18: mol_type = other DNA
aagcacctggccacaggg

SEQ ID NO: 83 - DNA, 18 bp, synthetic construct
  source 1..18: mol_type = other DNA
ggatagggaaaagcacct

SEQ ID NO: 84 - DNA, 18 bp, synthetic construct
  source 1..18: mol_type = other DNA
acacccctgtggataggg

SEQ ID NO: 85 - DNA, 18 bp, synthetic construct
  source 1..18: mol_type = other DNA
acacacacaaacacccct

SEQ ID NO: 86 - DNA, 18 bp, synthetic construct
  source 1..18: mol_type = other DNA
gcacacgcgcacacacac

SEQ ID NO: 87 - DNA, 18 bp, synthetic construct
  source 1..18: mol_type = other DNA
ttattgaaacgcacacgc

SEQ ID NO: 88 - DNA, 18 bp, synthetic construct
  source 1..18: mol_type = other DNA
tgtacaaactttattgaa

SEQ ID NO: 89 - DNA, 18 bp, synthetic construct
  source 1..18: mol_type = other DNA
tttaagaaagtgtacaaa

SEQ ID NO: 90 - DNA, 18 bp, synthetic construct
  source 1..18: mol_type = other DNA
agggatcatgtgattgga

SEQ ID NO: 91 - DNA, 18 bp, synthetic construct
  source 1..18: mol_type = other DNA
actctcctccctgcttcc

SEQ ID NO: 92 - DNA, 18 bp, synthetic construct
  source 1..18: mol_type = other DNA
gggccagcctggaatgct

SEQ ID NO: 93 - DNA, 18 bp, synthetic construct
  source 1..18: mol_type = other DNA
ggctacccgctcccattg

SEQ ID NO: 94 - DNA, 18 bp, synthetic construct
  source 1..18: mol_type = other DNA
gatgacccgcctacctca

SEQ ID NO: 95 - DNA, 18 bp, synthetic construct
  source 1..18: mol_type = other DNA
aaccgggtagcgctcaga

SEQ ID NO: 96 - DNA, 18 bp, synthetic construct
  source 1..18: mol_type = other DNA
gggcagcagcaaccgggt

SEQ ID NO: 97 - DNA, 18 bp, synthetic construct
  source 1..18: mol_type = other DNA
cgcggtccttgggcagca

SEQ ID NO: 98 - DNA, 18 bp, synthetic construct
  source 1..18: mol_type = other DNA
cgtccgactccgcggtcc

SEQ ID NO: 99 - DNA, 18 bp, synthetic construct
  source 1..18: mol_type = other DNA
ggtctgcctgcgtccgac

SEQ ID NO: 100 - DNA, 18 bp, synthetic construct
  source 1..18: mol_type = other DNA
gggtccacatggtctgcc

SEQ ID NO: 101 - DNA, 18 bp, synthetic construct
  source 1..18: mol_type = other DNA
aggccacccagctcacca

SEQ ID NO: 102 - DNA, 18 bp, synthetic construct
  source 1..18: mol_type = other DNA
gcagaactgaccatctgg

SEQ ID NO: 103 - DNA, 18 bp, synthetic construct
  source 1..18: mol_type = other DNA
cggcagcagctgtagctg

SEQ ID NO: 104 - DNA, 18 bp, synthetic construct
  source 1..18: mol_type = other DNA
ggccacccagatgcctgc

SEQ ID NO: 105 - DNA, 18 bp, synthetic construct
  source 1..18: mol_type = other DNA
ggtaaagatgcaggagtg

SEQ ID NO: 106 - DNA, 18 bp, synthetic construct
  source 1..18: mol_type = other DNA
ccgcatgccacggcctct

SEQ ID NO: 107 - DNA, 18 bp, synthetic construct
  source 1..18: mol_type = other DNA
gcccgtctgcactgcagt

SEQ ID NO: 108 - DNA, 18 bp, synthetic construct
  source 1..18: mol_type = other DNA
gcactggatggcacccac

SEQ ID NO: 109 - DNA, 18 bp, synthetic construct
  source 1..18: mol_type = other DNA
accataacacagcacgtg

SEQ ID NO: 110 - DNA, 18 bp, synthetic construct
  source 1..18: mol_type = other DNA
cttcacagcaggaagcct

SEQ ID NO: 111 - DNA, 18 bp, synthetic construct
  source 1..18: mol_type = other DNA
ggtgtgaaccaggtcgca

SEQ ID NO: 112 - DNA, 18 bp, synthetic construct
  source 1..18: mol_type = other DNA
gcagggagcttctttgcc

SEQ ID NO: 113 - DNA, 18 bp, synthetic construct
  source 1..18: mol_type = other DNA
ccggacacatgaccgagc

SEQ ID NO: 114 - DNA, 18 bp, synthetic construct
  source 1..18: mol_type = other DNA
actgggcagctcacagca

SEQ ID NO: 115 - DNA, 18 bp, synthetic construct
  source 1..18: mol_type = other DNA
tgatcggagcagcaggtg

SEQ ID NO: 116 - DNA, 18 bp, synthetic construct
  source 1..18: mol_type = other DNA
acttactctggatcaggt

SEQ ID NO: 117 - DNA, 18 bp, synthetic construct
  source 1..18: mol_type = other DNA
cgcaggcagcttagtgag

SEQ ID NO: 118 - DNA, 18 bp, synthetic construct
  source 1..18: mol_type = other DNA
ccatctgggcagctcacc

SEQ ID NO: 119 - DNA, 18 bp, synthetic construct
  source 1..18: mol_type = other DNA
taaaagggcagcagcccc

SEQ ID NO: 120 - DNA, 18 bp, synthetic construct
  source 1..18: mol_type = other DNA
aaaccccgcgggacagca

SEQ ID NO: 121 - DNA, 18 bp, synthetic construct
  source 1..18: mol_type = other DNA
ggcacctggtggggcccc

SEQ ID NO: 122 - DNA, 18 bp, synthetic construct
  source 1..18: mol_type = other DNA
aggcttgtgggtctggca

SEQ ID NO: 123 - DNA, 18 bp, synthetic construct
  source 1..18: mol_type = other DNA
atcggaggagggacagct

SEQ ID NO: 124 - DNA, 18 bp, synthetic construct
  source 1..18: mol_type = other DNA
tctgggattggacagcag

SEQ ID NO: 125 - DNA, 18 bp, synthetic construct
  source 1..18: mol_type = other DNA
acgtgtagccctgggggc

SEQ ID NO: 126 - DNA, 18 bp, synthetic construct
  source 1..18: mol_type = other DNA
cagtccagccacgatctc

SEQ ID NO: 127 - DNA, 18 bp, synthetic construct
  source 1..18: mol_type = other DNA
ccgatgtctctggggtgg

SEQ ID NO: 128 - DNA, 18 bp, synthetic construct
  source 1..18: mol_type = other DNA
ggctcgggcagcaggtct

SEQ ID NO: 129 - DNA, 18 bp, synthetic construct
  source 1..18: mol_type = other DNA
ctcgcagcacacagcatg

SEQ ID NO: 130 - DNA, 18 bp, synthetic construct
  source 1..18: mol_type = other DNA
cgagccttcacgttgcag

SEQ ID NO: 131 - DNA, 18 bp, synthetic construct
  source 1..18: mol_type = other DNA
gggccaggaaggtggcag

SEQ ID NO: 132 - DNA, 18 bp, synthetic construct
  source 1..18: mol_type = other DNA
gaagtgtccttccccaca

SEQ ID NO: 133 - DNA, 18 bp, synthetic construct
  source 1..18: mol_type = other DNA
caggcccagccctgtcgg

SEQ ID NO: 134 - DNA, 18 bp, synthetic construct
  source 1..18: mol_type = other DNA
gacagcagtggcgccgat

SEQ ID NO: 135 - DNA, 18 bp, synthetic construct
  source 1..18: mol_type = other DNA
ctccctgcgcaaacactt

SEQ ID NO: 136 - DNA, 18 bp, synthetic construct
  source 1..18: mol_type = other DNA
agcagctgtctcaaggct

SEQ ID NO: 137 - DNA, 18 bp, synthetic construct
  source 1..18: mol_type = other DNA
gtactgtccctcacagca

SEQ ID NO: 138 - DNA, 18 bp, synthetic construct
  source 1..18: mol_type = other DNA
agagtcttcagtactgtc

SEQ ID NO: 139 - DNA, 18 bp, synthetic construct
  source 1..18: mol_type = other DNA
cgagggctgcagagtctt

SEQ ID NO: 140 - DNA, 18 bp, synthetic construct
  source 1..18: mol_type = other DNA
agtggggtcccgagggct

SEQ ID NO: 141 - DNA, 18 bp, synthetic construct
  source 1..18: mol_type = other DNA
gcaccctccgagtggggt

SEQ ID NO: 142 - DNA, 18 bp, synthetic construct
  source 1..18: mol_type = other DNA
tgagcagagggcaccctc

SEQ ID NO: 143 - DNA, 18 bp, synthetic construct
  source 1..18: mol_type = other DNA
tagggaggcctgagcaga

SEQ ID NO: 144 - DNA, 18 bp, synthetic construct
  source 1..18: mol_type = other DNA
ggggaggtgctagggagg

SEQ ID NO: 145 - DNA, 18 bp, synthetic construct
  source 1..18: mol_type = other DNA
atttggttagggggaggt

SEQ ID NO: 146 - DNA, 18 bp, synthetic construct
  source 1..18: mol_type = other DNA
tccagggagaatttggtt

SEQ ID NO: 147 - DNA, 18 bp, synthetic construct
  source 1..18: mol_type = other DNA
cagaatggggtccaggga

SEQ ID NO: 148 - DNA, 18 bp, synthetic construct
  source 1..18: mol_type = other DNA
atggggagctcagaatgg

SEQ ID NO: 149 - DNA, 18 bp, synthetic construct
  source 1..18: mol_type = other DNA
tcccatggtgatggggag

SEQ ID NO: 150 - DNA, 18 bp, synthetic construct
  source 1..18: mol_type = other DNA
aggccccacctcccatgg

SEQ ID NO: 151 - DNA, 18 bp, synthetic construct
  source 1..18: mol_type = other DNA
ccttagattgaggcccca

SEQ ID NO: 152 - DNA, 18 bp, synthetic construct
  source 1..18: mol_type = other DNA
acagggaaggccttagat

SEQ ID NO: 153 - DNA, 18 bp, synthetic construct
  source 1..18: mol_type = other DNA
cccccttctgacagggaa

SEQ ID NO: 154 - DNA, 18 bp, synthetic construct
  source 1..18: mol_type = other DNA
tttgccacaacccccttc

SEQ ID NO: 155 - DNA, 18 bp, synthetic construct
  source 1..18: mol_type = other DNA
taatgtggcttttgccac

SEQ ID NO: 156 - DNA, 18 bp, synthetic construct
  source 1..18: mol_type = other DNA
tggcagcttgtaatgtgg

SEQ ID NO: 157 - DNA, 18 bp, synthetic construct
  source 1..18: mol_type = other DNA
ggggaggggatggcagct

SEQ ID NO: 158 - DNA, 18 bp, synthetic construct
  source 1..18: mol_type = other DNA
ccactgaaacggggaggg

SEQ ID NO: 159 - DNA, 18 bp, synthetic construct
  source 1..18: mol_type = other DNA
gccacagggtccactgaa

SEQ ID NO: 160 - DNA, 18 bp, synthetic construct
  source 1..18: mol_type = other DNA
aaagcacctggccacagg

SEQ ID NO: 161 - DNA, 18 bp, synthetic construct
  source 1..18: mol_type = other DNA
tggatagggaaaagcacc

SEQ ID NO: 162 - DNA, 18 bp, synthetic construct
  source 1..18: mol_type = other DNA
aacacccctgtggatagg

SEQ ID NO: 163 - DNA, 18 bp, synthetic construct
  source 1..18: mol_type = other DNA
cacacacacaaacacccc

SEQ ID NO: 164 - DNA, 18 bp, synthetic construct
  source 1..18: mol_type = other DNA
cgcacacgcgcacacaca

SEQ ID NO: 165 - DNA, 18 bp, synthetic construct
  source 1..18: mol_type = other DNA
tttattgaaacgcacacg

SEQ ID NO: 166 - DNA, 18 bp, synthetic construct
  source 1..18: mol_type = other DNA
gtgtacaaactttattga

SEQ ID NO: 167 - DNA, 18 bp, synthetic construct
  source 1..18: mol_type = other DNA
ttttaagaaagtgtacaa

SEQ ID NO: 168 - DNA, 18 bp, synthetic construct
  source 1..18: mol_type = other DNA
ttctagggatcatgtgat

SEQ ID NO: 169 - DNA, 18 bp, synthetic construct
  source 1..18: mol_type = other DNA
aatcactctcctccctgc

SEQ ID NO: 170 - DNA, 18 bp, synthetic construct
  source 1..18: mol_type = other DNA
ggtggggccagcctggaa

SEQ ID NO: 171 - DNA, 18 bp, synthetic construct
  source 1..18: mol_type = other DNA
tcagggctacccgctccc

SEQ ID NO: 172 - DNA, 18 bp, synthetic construct
  source 1..18: mol_type = other DNA
gcgcgatgacccgcctac

SEQ ID NO: 173 - DNA, 18 bp, synthetic construct
  source 1..18: mol_type = other DNA
caaccgggtagcgctcag

SEQ ID NO: 174 - DNA, 18 bp, synthetic construct
  source 1..18: mol_type = other DNA
tgggcagcagcaaccggg

SEQ ID NO: 175 - DNA, 18 bp, synthetic construct
  source 1..18: mol_type = other DNA
ccgcggtccttgggcagc

SEQ ID NO: 176 - DNA, 18 bp, synthetic construct
  source 1..18: mol_type = other DNA
gcgtccgactccgcggtc

SEQ ID NO: 177 - DNA, 18 bp, synthetic construct
  source 1..18: mol_type = other DNA
tggtctgcctgcgtccga

SEQ ID NO: 178 - DNA, 18 bp, synthetic construct
  source 1..18: mol_type = other DNA
agggtccacatggtctgc

SEQ ID NO: 179 - DNA, 18 bp, synthetic construct
  source 1..18: mol_type = other DNA
tgttaaggccacccagct

SEQ ID NO: 180 - DNA, 18 bp, synthetic construct
  source 1..18: mol_type = other DNA
acagggcagaactgacca

SEQ ID NO: 181 - DNA, 18 bp, synthetic construct
  source 1..18: mol_type = other DNA
ggggacggcagcagctgt

SEQ ID NO: 182 - DNA, 18 bp, synthetic construct
  source 1..18: mol_type = other DNA
gcaggggccacccagatg

SEQ ID NO: 183 - DNA, 18 bp, synthetic construct
  source 1..18: mol_type = other DNA
gagacggtaaagatgcag

SEQ ID NO: 184 - DNA, 18 bp, synthetic construct
  source 1..18: mol_type = other DNA
catccccgcatgccacgg

SEQ ID NO: 185 - DNA, 18 bp, synthetic construct
  source 1..18: mol_type = other DNA
ggatcgcccgtctgcact

SEQ ID NO: 186 - DNA, 18 bp, synthetic construct
  source 1..18: mol_type = other DNA
tcagggcactggatggca

SEQ ID NO: 187 - DNA, 18 bp, synthetic construct
  source 1..18: mol_type = other DNA
catcgaccataacacagc

SEQ ID NO: 188 - DNA, 18 bp, synthetic construct
  source 1..18: mol_type = other DNA
cctgtcttcacagcagga

SEQ ID NO: 189 - DNA, 18 bp, synthetic construct
  source 1..18: mol_type = other DNA
cagcgggtgtgaaccagg

SEQ ID NO: 190 - DNA, 18 bp, synthetic construct
  source 1..18: mol_type = other DNA
tctgggcagggagcttct

SEQ ID NO: 191 - DNA, 18 bp, synthetic construct
  source 1..18: mol_type = other DNA
tgcgtccggacacatgac

SEQ ID NO: 192 - DNA, 18 bp, synthetic construct
  source 1..18: mol_type = other DNA
ttcccactgggcagctca

SEQ ID NO: 193 - DNA, 18 bp, synthetic construct
  source 1..18: mol_type = other DNA
gcaggtgatcggagcagc

SEQ ID NO: 194 - DNA, 18 bp, synthetic construct
  source 1..18: mol_type = other DNA
gaggcacttactctggat

SEQ ID NO: 195 - DNA, 18 bp, synthetic construct
  source 1..18: mol_type = other DNA
gtgtgcgcaggcagctta

SEQ ID NO: 196 - DNA, 18 bp, synthetic construct
  source 1..18: mol_type = other DNA
tatagccatctgggcagc

SEQ ID NO: 197 - DNA, 18 bp, synthetic construct
  source 1..18: mol_type = other DNA
ctgggtaaaagggcagca

SEQ ID NO: 198 - DNA, 18 bp, synthetic construct
  source 1..18: mol_type = other DNA
cacgtaaaccccgcggga

SEQ ID NO: 199 - DNA, 18 bp, synthetic construct
  source 1..18: mol_type = other DNA
tccagggcacctggtggg

SEQ ID NO: 200 - DNA, 18 bp, synthetic construct
  source 1..18: mol_type = other DNA
cttcaaggcttgtgggtc

SEQ ID NO: 201 - DNA, 18 bp, synthetic construct
  source 1..18: mol_type = other DNA
caggtatcggaggaggga

SEQ ID NO: 202 - DNA, 18 bp, synthetic construct
  source 1..18: mol_type = other DNA
cagcctctgggattggac

SEQ ID NO: 203 - DNA, 18 bp, synthetic construct
  source 1..18: mol_type = other DNA
tacacacgtgtagccctg

SEQ ID NO: 204 - DNA, 18 bp, synthetic construct
  source 1..18: mol_type = other DNA
ttctccagtccagccacg

SEQ ID NO: 205 - DNA, 18 bp, synthetic construct
  source 1..18: mol_type = other DNA
cacagccgatgtctctgg

SEQ ID NO: 206 - DNA, 18 bp, synthetic construct
  source 1..18: mol_type = other DNA
acccaggctcgggcagca

SEQ ID NO: 207 - DNA, 18 bp, synthetic construct
  source 1..18: mol_type = other DNA
cgatcctcgcagcacaca

SEQ ID NO: 208 - DNA, 18 bp, synthetic construct
  source 1..18: mol_type = other DNA
aggatcgagccttcacgt

SEQ ID NO: 209 - DNA, 18 bp, synthetic construct
  source 1..18: mol_type = other DNA
gctacgggccaggaaggt

SEQ ID NO: 210 - DNA, 18 bp, synthetic construct
  source 1..18: mol_type = other DNA
tggcagaagtgtccttcc

SEQ ID NO: 211 - DNA, 18 bp, synthetic construct
  source 1..18: mol_type = other DNA
gacagcaggcccagccct

SEQ ID NO: 212 - DNA, 18 bp, synthetic construct
  source 1..18: mol_type = other DNA
agcaggacagcagtggcg

SEQ ID NO: 213 - DNA, 18 bp, synthetic construct
  source 1..18: mol_type = other DNA
ggggcctccctgcgcaaa

SEQ ID NO: 214 - DNA, 18 bp, synthetic construct
  source 1..18: mol_type = other DNA
ctcacagcagctgtctca

SEQ ID NO: 215 - DNA, 18 bp, synthetic construct
  source 1..18: mol_type = other DNA
agtactgtccctcacagc

SEQ ID NO: 216 - DNA, 18 bp, synthetic construct
  source 1..18: mol_type = other DNA
cagagtcttcagtactgt

SEQ ID NO: 217 - DNA, 18 bp, synthetic construct
  source 1..18: mol_type = other DNA
ccgagggctgcagagtct

SEQ ID NO: 218 - DNA, 18 bp, synthetic construct
  source 1..18: mol_type = other DNA
gagtggggtcccgagggc

SEQ ID NO: 219 - DNA, 18 bp, synthetic construct
  source 1..18: mol_type = other DNA
ggcaccctccgagtgggg

SEQ ID NO: 220 - DNA, 18 bp, synthetic construct
  source 1..18: mol_type = other DNA
ctgagcagagggcaccct

SEQ ID NO: 221 - DNA, 18 bp, synthetic construct
  source 1..18: mol_type = other DNA
ctagggaggcctgagcag

SEQ ID NO: 222 - DNA, 18 bp, synthetic construct
  source 1..18: mol_type = other DNA
gggggaggtgctagggag

SEQ ID NO: 223 - DNA, 18 bp, synthetic construct
  source 1..18: mol_type = other DNA
aatttggttagggggagg

SEQ ID NO: 224 - DNA, 18 bp, synthetic construct
  source 1..18: mol_type = other DNA
gtccagggagaatttggt

SEQ ID NO: 225 - DNA, 18 bp, synthetic construct
  source 1..18: mol_type = other DNA
tcagaatggggtccaggg

SEQ ID NO: 226 - DNA, 18 bp, synthetic construct
  source 1..18: mol_type = other DNA
gatggggagctcagaatg

SEQ ID NO: 227 - DNA, 18 bp, synthetic construct
  source 1..18: mol_type = other DNA
ctcccatggtgatgggga

SEQ ID NO: 228 - DNA, 18 bp, synthetic construct
  source 1..18: mol_type = other DNA
gaggccccacctcccatg

SEQ ID NO: 229 - DNA, 18 bp, synthetic construct
  source 1..18: mol_type = other DNA
gccttagattgaggcccc

SEQ ID NO: 230 - DNA, 18 bp, synthetic construct
  source 1..18: mol_type = other DNA
gacagggaaggccttaga

SEQ ID NO: 231 - DNA, 18 bp, synthetic construct
  source 1..18: mol_type = other DNA
acccccttctgacaggga

SEQ ID NO: 232 - DNA, 18 bp, synthetic construct
  source 1..18: mol_type = other DNA
ttttgccacaaccccctt

SEQ ID NO: 233 - DNA, 18 bp, synthetic construct
  source 1..18: mol_type = other DNA
gtaatgtggcttttgcca

SEQ ID NO: 234 - DNA, 18 bp, synthetic construct
  source 1..18: mol_type = other DNA
atggcagcttgtaatgtg

SEQ ID NO: 235 - DNA, 18 bp, synthetic construct
  source 1..18: mol_type = other DNA
cggggaggggatggcagc

SEQ ID NO: 236 - DNA, 18 bp, synthetic construct
  source 1..18: mol_type = other DNA
tccactgaaacggggagg

SEQ ID NO: 237 - DNA, 18 bp, synthetic construct
  source 1..18: mol_type = other DNA
ggccacagggtccactga

SEQ ID NO: 238 - DNA, 18 bp, synthetic construct
  source 1..18: mol_type = other DNA
aaaagcacctggccacag

SEQ ID NO: 239 - DNA, 18 bp, synthetic construct
  source 1..18: mol_type = other DNA
gtggatagggaaaagcac

SEQ ID NO: 240 - DNA, 18 bp, synthetic construct
  source 1..18: mol_type = other DNA
aaacacccctgtggatag

SEQ ID NO: 241 - DNA, 18 bp, synthetic construct
  source 1..18: mol_type = other DNA
gcacacacacaaacaccc

SEQ ID NO: 242 - DNA, 18 bp, synthetic construct
  source 1..18: mol_type = other DNA
acgcacacgcgcacacac

SEQ ID NO: 243 - DNA, 18 bp, synthetic construct
  source 1..18: mol_type = other DNA
ctttattgaaacgcacac

SEQ ID NO: 244 - DNA, 18 bp, synthetic construct
  source 1..18: mol_type = other DNA
agtgtacaaactttattg

SEQ ID NO: 245 - DNA, 18 bp, synthetic construct
  source 1..18: mol_type = other DNA
tttttaagaaagtgtaca

SEQ ID NO: 246 - DNA, 18 bp, synthetic construct
  source 1..18: mol_type = other DNA
gtagggacagcaggccca

SEQ ID NO: 247 - DNA, 18 bp, synthetic construct
  source 1..18: mol_type = other DNA
ccatttctagggatcatg

SEQ ID NO: 248 - DNA, 18 bp, synthetic construct
  source 1..18: mol_type = other DNA
ctcaaatcactctcctcc

SEQ ID NO: 249 - DNA, 18 bp, synthetic construct
  source 1..18: mol_type = other DNA
tagaggtggggccagcct

SEQ ID NO: 250 - DNA, 18 bp, synthetic construct
  source 1..18: mol_type = other DNA
gggatcagggctacccgc

SEQ ID NO: 251 - DNA, 18 bp, synthetic construct
  source 1..18: mol_type = other DNA
cccagcgcgatgacccgc

SEQ ID NO: 252 - DNA, 18 bp, synthetic construct
  source 1..18: mol_type = other DNA
gcaaccgggtagcgctca

SEQ ID NO: 253 - DNA, 18 bp, synthetic construct
  source 1..18: mol_type = other DNA
ttgggcagcagcaaccgg

SEQ ID NO: 254 - DNA, 18 bp, synthetic construct
  source 1..18: mol_type = other DNA
tccgcggtccttgggcag

SEQ ID NO: 255 - DNA, 18 bp, synthetic construct
  source 1..18: mol_type = other DNA
tgcgtccgactccgcggt

SEQ ID NO: 256 - DNA, 18 bp, synthetic construct
  source 1..18: mol_type = other DNA
atggtctgcctgcgtccg

SEQ ID NO: 257 - DNA, 18 bp, synthetic construct
  source 1..18: mol_type = other DNA
cagggtccacatggtctg

SEQ ID NO: 258 - DNA, 18 bp, synthetic construct
  source 1..18: mol_type = other DNA
cctgctgttaaggccacc

SEQ ID NO: 259 - DNA, 18 bp, synthetic construct
  source 1..18: mol_type = other DNA
aggccacagggcagaact

SEQ ID NO: 260 - DNA, 18 bp, synthetic construct
  source 1..18: mol_type = other DNA
cagaaggggacggcagca

SEQ ID NO: 261 - DNA, 18 bp, synthetic construct
  source 1..18: mol_type = other DNA
acctggcaggggccaccc

SEQ ID NO: 262 - DNA, 18 bp, synthetic construct
  source 1..18: mol_type = other DNA
tccctgagacggtaaaga

SEQ ID NO: 263 - DNA, 18 bp, synthetic construct
  source 1..18: mol_type = other DNA
atggccatccccgcatgc

SEQ ID NO: 264 - DNA, 18 bp, synthetic construct
  source 1..18: mol_type = other DNA
aagcaggatcgcccgtct

SEQ ID NO: 265 - DNA, 18 bp, synthetic construct
  source 1..18: mol_type = other DNA
gactatcagggcactgga

SEQ ID NO: 266 - DNA, 18 bp, synthetic construct
  source 1..18: mol_type = other DNA
ggagccatcgaccataac

SEQ ID NO: 267 - DNA, 18 bp, synthetic construct
  source 1..18: mol_type = other DNA
tgcaccctgtcttcacag

SEQ ID NO: 268 - DNA, 18 bp, synthetic construct
  source 1..18: mol_type = other DNA
tgatgcagcgggtgtgaa

SEQ ID NO: 269 - DNA, 18 bp, synthetic construct
  source 1..18: mol_type = other DNA
agtcctctgggcagggag

SEQ ID NO: 270 - DNA, 18 bp, synthetic construct
  source 1..18: mol_type = other DNA
gaccgtgcgtccggacac

SEQ ID NO: 271 - DNA, 18 bp, synthetic construct
  source 1..18: mol_type = other DNA
catacttcccactgggca

SEQ ID NO: 272 - DNA, 18 bp, synthetic construct
  source 1..18: mol_type = other DNA
gcagtgcaggtgatcgga

SEQ ID NO: 273 - DNA, 18 bp, synthetic construct
  source 1..18: mol_type = other DNA
ttggagaggcacttactc

SEQ ID NO: 274 - DNA, 18 bp, synthetic construct
  source 1..18: mol_type = other DNA
ccactgtgtgcgcaggca

SEQ ID NO: 275 - DNA, 18 bp, synthetic construct
  source 1..18: mol_type = other DNA
gcaggtatagccatctgg

SEQ ID NO: 276 - DNA, 18 bp, synthetic construct
  source 1..18: mol_type = other DNA
acagcctgggtaaaaggg

SEQ ID NO: 277 - DNA, 18 bp, synthetic construct
  source 1..18: mol_type = other DNA
tgtcacacgtaaaccccg

SEQ ID NO: 278 - DNA, 18 bp, synthetic construct
  source 1..18: mol_type = other DNA
ctccatccagggcacctg

SEQ ID NO: 279 - DNA, 18 bp, synthetic construct
  source 1..18: mol_type = other DNA
tctctcttcaaggcttgt

SEQ ID NO: 280 - DNA, 18 bp, synthetic construct
  source 1..18: mol_type = other DNA
ggcagcaggtatcggagg

SEQ ID NO: 281 - DNA, 18 bp, synthetic construct
  source 1..18: mol_type = other DNA
gcagacagcctctgggat

SEQ ID NO: 282 - DNA, 18 bp, synthetic construct
  source 1..18: mol_type = other DNA
tcagctacacacgtgtag

SEQ ID NO: 283 - DNA, 18 bp, synthetic construct
  source 1..18: mol_type = other DNA
gcatcttctccagtccag

SEQ ID NO: 284 - DNA, 18 bp, synthetic construct
  source 1..18: mol_type = other DNA
ctggtcacagccgatgtc

SEQ ID NO: 285 - DNA, 18 bp, synthetic construct
  source 1..18: mol_type = other DNA
ctcccacccaggctcggg

SEQ ID NO: 286 - DNA, 18 bp, synthetic construct
  source 1..18: mol_type = other DNA
gctggcgatcctcgcagc

SEQ ID NO: 287 - DNA, 18 bp, synthetic construct
  source 1..18: mol_type = other DNA
ctcgcaggatcgagcctt

SEQ ID NO: 288 - DNA, 18 bp, synthetic construct
  source 1..18: mol_type = other DNA
tgagggctacgggccagg

SEQ ID NO: 289 - DNA, 18 bp, synthetic construct
  source 1..18: mol_type = other DNA
tatcatggcagaagtgtc

SEQ ID NO: 290 - DNA, 18 bp, synthetic construct
  source 1..18: mol_type = other DNA
aagccagcaggacagcag

SEQ ID NO: 291 - DNA, 18 bp, synthetic construct
  source 1..18: mol_type = other DNA
agcgcggggcctccctgc

SEQ ID NO: 292 - DNA, 18 bp, synthetic construct
  source 1..18: mol_type = other DNA
cctcacagcagctgtctc

SEQ ID NO: 293 - DNA, 18 bp, synthetic construct
  source 1..18: mol_type = other DNA
cagtactgtccctcacag

SEQ ID NO: 294 - DNA, 18 bp, synthetic construct
  source 1..18: mol_type = other DNA
gcagagtcttcagtactg

SEQ ID NO: 295 - DNA, 18 bp, synthetic construct
  source 1..18: mol_type = other DNA
cccgagggctgcagagtc

SEQ ID NO: 296 - DNA, 18 bp, synthetic construct
  source 1..18: mol_type = other DNA
cgagtggggtcccgaggg

SEQ ID NO: 297 - DNA, 18 bp, synthetic construct
  source 1..18: mol_type = other DNA
gggcaccctccgagtggg

SEQ ID NO: 298 - DNA, 18 bp, synthetic construct
  source 1..18: mol_type = other DNA
cctgagcagagggcaccc

SEQ ID NO: 299 - DNA, 18 bp, synthetic construct
  source 1..18: mol_type = other DNA
gctagggaggcctgagca

SEQ ID NO: 300 - DNA, 18 bp, synthetic construct
  source 1..18: mol_type = other DNA
agggggaggtgctaggga

SEQ ID NO: 301 - DNA, 18 bp, synthetic construct
  source 1..18: mol_type = other DNA
gaatttggttagggggag

SEQ ID NO: 302 - DNA, 18 bp, synthetic construct
  source 1..18: mol_type = other DNA
ggtccagggagaatttgg

SEQ ID NO: 303 - DNA, 18 bp, synthetic construct
  source 1..18: mol_type = other DNA
ctcagaatggggtccagg

SEQ ID NO: 304 - DNA, 18 bp, synthetic construct
  source 1..18: mol_type = other DNA
tgatggggagctcagaat

SEQ ID NO: 305 - DNA, 18 bp, synthetic construct
  source 1..18: mol_type = other DNA
cctcccatggtgatgggg

SEQ ID NO: 306 - DNA, 18 bp, synthetic construct
  source 1..18: mol_type = other DNA
tgaggccccacctcccat

SEQ ID NO: 307 - DNA, 18 bp, synthetic construct
  source 1..18: mol_type = other DNA
ggccttagattgaggccc

SEQ ID NO: 308 - DNA, 18 bp, synthetic construct
  source 1..18: mol_type = other DNA
tgacagggaaggccttag

SEQ ID NO: 309 - DNA, 18 bp, synthetic construct
  source 1..18: mol_type = other DNA
aacccccttctgacaggg

SEQ ID NO: 310 - DNA, 18 bp, synthetic construct
  source 1..18: mol_type = other DNA
cttttgccacaaccccct

SEQ ID NO: 311 - DNA, 18 bp, synthetic construct
  source 1..18: mol_type = other DNA
tgtaatgtggcttttgcc

SEQ ID NO: 312 - DNA, 18 bp, synthetic construct
  source 1..18: mol_type = other DNA
gatggcagcttgtaatgt

SEQ ID NO: 313 - DNA, 18 bp, synthetic construct
  source 1..18: mol_type = other DNA
acggggaggggatggcag

SEQ ID NO: 314 - DNA, 18 bp, synthetic construct
  source 1..18: mol_type = other DNA
gtccactgaaacggggag

SEQ ID NO: 315 - DNA, 18 bp, synthetic construct
  source 1..18: mol_type = other DNA
tggccacagggtccactg

SEQ ID NO: 316 - DNA, 18 bp, synthetic construct
  source 1..18: mol_type = other DNA
gaaaagcacctggccaca

SEQ ID NO: 317 - DNA, 18 bp, synthetic construct
  source 1..18: mol_type = other DNA
tgtggatagggaaaagca

SEQ ID NO: 318 - DNA, 18 bp, synthetic construct
  source 1..18: mol_type = other DNA
caaacacccctgtggata

SEQ ID NO: 319 - DNA, 18 bp, synthetic construct
  source 1..18: mol_type = other DNA
cgcacacacacaaacacc

SEQ ID NO: 320 - DNA, 18 bp, synthetic construct
  source 1..18: mol_type = other DNA
aacgcacacgcgcacaca

SEQ ID NO: 321 - DNA, 18 bp, synthetic construct
  source 1..18: mol_type = other DNA
actttattgaaacgcaca

SEQ ID NO: 322 - DNA, 18 bp, synthetic construct
  source 1..18: mol_type = other DNA
aagtgtacaaactttatt

SEQ ID NO: 323 - DNA, 18 bp, synthetic construct
  source 1..18: mol_type = other DNA
ttttttaagaaagtgtac

SEQ ID NO: 324 - DNA, 18 bp, synthetic construct
  source 1..18: mol_type = other DNA
caccccatttctagggat

SEQ ID NO: 325 - DNA, 18 bp, synthetic construct
  source 1..18: mol_type = other DNA
tctactcaaatcactctc

SEQ ID NO: 326 - DNA, 18 bp, synthetic construct
  source 1..18: mol_type = other DNA
aatatagaggtggggcca

SEQ ID NO: 327 - DNA, 18 bp, synthetic construct
  source 1..18: mol_type = other DNA
gccagggatcagggctac

SEQ ID NO: 328 - DNA, 18 bp, synthetic construct
  source 1..18: mol_type = other DNA
agaccccagcgcgatgac

SEQ ID NO: 329 - DNA, 18 bp, synthetic construct
  source 1..18: mol_type = other DNA
agcaaccgggtagcgctc

SEQ ID NO: 330 - DNA, 18 bp, synthetic construct
  source 1..18: mol_type = other DNA
cttgggcagcagcaaccg

SEQ ID NO: 331 - DNA, 18 bp, synthetic construct
  source 1..18: mol_type = other DNA
ctccgcggtccttgggca

SEQ ID NO: 332 - DNA, 18 bp, synthetic construct
  source 1..18: mol_type = other DNA
ctgcgtccgactccgcgg

SEQ ID NO: 333 - DNA, 18 bp, synthetic construct
  source 1..18: mol_type = other DNA
catggtctgcctgcgtcc

SEQ ID NO: 334 - DNA, 18 bp, synthetic construct
  source 1..18: mol_type = other DNA
ccagggtccacatggtct

SEQ ID NO: 335 - DNA, 18 bp, synthetic construct
  source 1..18: mol_type = other DNA
ccagccctgctgttaagg

SEQ ID NO: 336 - DNA, 18 bp, synthetic construct
  source 1..18: mol_type = other DNA
gcagcaggccacagggca

SEQ ID NO: 337 - DNA, 18 bp, synthetic construct
  source 1..18: mol_type = other DNA
ttgtccagaaggggacgg

SEQ ID NO: 338 - DNA, 18 bp, synthetic construct
  source 1..18: mol_type = other DNA
catcaacctggcaggggc

SEQ ID NO: 339 - DNA, 18 bp, synthetic construct
  source 1..18: mol_type = other DNA
ggaagtccctgagacggt

SEQ ID NO: 340 - DNA, 18 bp, synthetic construct
  source 1..18: mol_type = other DNA
cagtgatggccatccccg

SEQ ID NO: 341 - DNA, 18 bp, synthetic construct
  source 1..18: mol_type = other DNA
tttggaagcaggatcgcc

SEQ ID NO: 342 - DNA, 18 bp, synthetic construct
  source 1..18: mol_type = other DNA
gaactgactatcagggca

SEQ ID NO: 343 - DNA, 18 bp, synthetic construct
  source 1..18: mol_type = other DNA
ccccaggagccatcgacc

SEQ ID NO: 344 - DNA, 18 bp, synthetic construct
  source 1..18: mol_type = other DNA
agcagtgcaccctgtctt

SEQ ID NO: 345 - DNA, 18 bp, synthetic construct
  source 1..18: mol_type = other DNA
gggtgtgatgcagcgggt

SEQ ID NO: 346 - DNA, 18 bp, synthetic construct
  source 1..18: mol_type = other DNA
ctgttagtcctctgggca

SEQ ID NO: 347 - DNA, 18 bp, synthetic construct
  source 1..18: mol_type = other DNA
accgggaccgtgcgtccg

SEQ ID NO: 348 - DNA, 18 bp, synthetic construct
  source 1..18: mol_type = other DNA
gcagccatacttcccact

SEQ ID NO: 349 - DNA, 18 bp, synthetic construct
  source 1..18: mol_type = other DNA
gggcagcagtgcaggtga

SEQ ID NO: 350 - DNA, 18 bp, synthetic construct
  source 1..18: mol_type = other DNA
tctccttggagaggcact

SEQ ID NO: 351 - DNA, 18 bp, synthetic construct
  source 1..18: mol_type = other DNA
atcccccactgtgtgcgc

SEQ ID NO: 352 - DNA, 18 bp, synthetic construct
  source 1..18: mol_type = other DNA
cggcagcaggtatagcca

SEQ ID NO: 353 - DNA, 18 bp, synthetic construct
  source 1..18: mol_type = other DNA
agcacacagcctgggtaa

SEQ ID NO: 354 - DNA, 18 bp, synthetic construct
  source 1..18: mol_type = other DNA
ctgcgtgtcacacgtaaa

SEQ ID NO: 355 - DNA, 18 bp, synthetic construct
  source 1..18: mol_type = other DNA
gccttctccatccagggc

SEQ ID NO: 356 - DNA, 18 bp, synthetic construct
  source 1..18: mol_type = other DNA
ggacatctctcttcaagg

SEQ ID NO: 357 - DNA, 18 bp, synthetic construct
  source 1..18: mol_type = other DNA
gagttggcagcaggtatc

SEQ ID NO: 358 - DNA, 18 bp, synthetic construct
  source 1..18: mol_type = other DNA
gagcagcagacagcctct

SEQ ID NO: 359 - DNA, 18 bp, synthetic construct
  source 1..18: mol_type = other DNA
gcccctcagctacacacg

SEQ ID NO: 360 - DNA, 18 bp, synthetic construct
  source 1..18: mol_type = other DNA
ggcaggcatcttctccag

SEQ ID NO: 361 - DNA, 18 bp, synthetic construct
  source 1..18: mol_type = other DNA
gtgtgctggtcacagccg

SEQ ID NO: 362 - DNA, 18 bp, synthetic construct
  source 1..18: mol_type = other DNA
cccagctcccacccaggc

SEQ ID NO: 363 - DNA, 18 bp, synthetic construct
  source 1..18: mol_type = other DNA
gcagtgctggcgatcctc

SEQ ID NO: 364 - DNA, 18 bp, synthetic construct
  source 1..18: mol_type = other DNA
tccttctcgcaggatcga

SEQ ID NO: 365 - DNA, 18 bp, synthetic construct
  source 1..18: mol_type = other DNA
ccacgtgagggctacggg

SEQ ID NO: 366 - DNA, 18 bp, synthetic construct
  source 1..18: mol_type = other DNA
ctggttatcatggcagaa

SEQ ID NO: 367 - DNA, 18 bp, synthetic construct
  source 1..18: mol_type = other DNA
tggcggtagggacagcag

SEQ ID NO: 368 - DNA, 18 bp, synthetic construct
  source 1..18: mol_type = other DNA
agcggaagccagcaggac

SEQ ID NO: 369 - DNA, 18 bp, synthetic construct
  source 1..18: mol_type = other DNA
gtcccagcgcggggcctc

SEQ ID NO: 370 - DNA, 18 bp, synthetic construct
  source 1..18: mol_type = other DNA
ccctcacagcagctgtct

SEQ ID NO: 371 - DNA, 18 bp, synthetic construct
  source 1..18: mol_type = other DNA
tcagtactgtccctcaca

SEQ ID NO: 372 - DNA, 18 bp, synthetic construct
  source 1..18: mol_type = other DNA
tgcagagtcttcagtact

SEQ ID NO: 373 - DNA, 18 bp, synthetic construct
  source 1..18: mol_type = other DNA
tcccgagggctgcagagt

SEQ ID NO: 374 - DNA, 18 bp, synthetic construct
  source 1..18: mol_type = other DNA
ccgagtggggtcccgagg

SEQ ID NO: 375 - DNA, 18 bp, synthetic construct
  source 1..18: mol_type = other DNA
agggcaccctccgagtgg

SEQ ID NO: 376 - DNA, 18 bp, synthetic construct
  source 1..18: mol_type = other DNA
gcctgagcagagggcacc

SEQ ID NO: 377 - DNA, 18 bp, synthetic construct
  source 1..18: mol_type = other DNA
tgctagggaggcctgagc

SEQ ID NO: 378 - DNA, 18 bp, synthetic construct
  source 1..18: mol_type = other DNA
tagggggaggtgctaggg

SEQ ID NO: 379 - DNA, 18 bp, synthetic construct
  source 1..18: mol_type = other DNA
agaatttggttaggggga

SEQ ID NO: 380 - DNA, 18 bp, synthetic construct
  source 1..18: mol_type = other DNA
gggtccagggagaatttg

SEQ ID NO: 381 - DNA, 18 bp, synthetic construct
  source 1..18: mol_type = other DNA
gctcagaatggggtccag

SEQ ID NO: 382 - DNA, 18 bp, synthetic construct
  source 1..18: mol_type = other DNA
gtgatggggagctcagaa

SEQ ID NO: 383 - DNA, 18 bp, synthetic construct
  source 1..18: mol_type = other DNA
acctcccatggtgatggg

SEQ ID NO: 384 - DNA, 18 bp, synthetic construct
  source 1..18: mol_type = other DNA
ttgaggccccacctccca

SEQ ID NO: 385 - DNA, 18 bp, synthetic construct
  source 1..18: mol_type = other DNA
aggccttagattgaggcc

SEQ ID NO: 386 - DNA, 18 bp, synthetic construct
  source 1..18: mol_type = other DNA
ctgacagggaaggcctta

SEQ ID NO: 387 - DNA, 18 bp, synthetic construct
  source 1..18: mol_type = other DNA
caacccccttctgacagg

SEQ ID NO: 388 - DNA, 18 bp, synthetic construct
  source 1..18: mol_type = other DNA
gcttttgccacaaccccc

SEQ ID NO: 389 - DNA, 18 bp, synthetic construct
  source 1..18: mol_type = other DNA
ttgtaatgtggcttttgc

SEQ ID NO: 390 - DNA, 18 bp, synthetic construct
  source 1..18: mol_type = other DNA
ggatggcagcttgtaatg

SEQ ID NO: 391 - DNA, 18 bp, synthetic construct
  source 1..18: mol_type = other DNA
aacggggaggggatggca

SEQ ID NO: 392 - DNA, 18 bp, synthetic construct
  source 1..18: mol_type = other DNA
ggtccactgaaacgggga

SEQ ID NO: 393 - DNA, 18 bp, synthetic construct
  source 1..18: mol_type = other DNA
ctggccacagggtccact

SEQ ID NO: 394 - DNA, 18 bp, synthetic construct
  source 1..18: mol_type = other DNA
ggaaaagcacctggccac

SEQ ID NO: 395 - DNA, 18 bp, synthetic construct
  source 1..18: mol_type = other DNA
ctgtggatagggaaaagc

SEQ ID NO: 396 - DNA, 18 bp, synthetic construct
  source 1..18: mol_type = other DNA
acaaacacccctgtggat

SEQ ID NO: 397 - DNA, 18 bp, synthetic construct
  source 1..18: mol_type = other DNA
gcgcacacacacaaacac

SEQ ID NO: 398 - DNA, 18 bp, synthetic construct
  source 1..18: mol_type = other DNA
aaacgcacacgcgcacac

SEQ ID NO: 399 - DNA, 18 bp, synthetic construct
  source 1..18: mol_type = other DNA
aactttattgaaacgcac

SEQ ID NO: 400 - DNA, 18 bp, synthetic construct
  source 1..18: mol_type = other DNA
aaagtgtacaaactttat

SEQ ID NO: 401 - DNA, 18 bp, synthetic construct
  source 1..18: mol_type = other DNA
tttttttaagaaagtgta

SEQ ID NO: 402 - DNA, 18 bp, synthetic construct
  source 1..18: mol_type = other DNA
caggtctggttatcatgg

SEQ ID NO: 403 - DNA, 18 bp, synthetic construct
  source 1..18: mol_type = other DNA
cccacaccccatttctag

SEQ ID NO: 404 - DNA, 18 bp, synthetic construct
  source 1..18: mol_type = other DNA
cttttctactcaaatcac

SEQ ID NO: 405 - DNA, 18 bp, synthetic construct
  source 1..18: mol_type = other DNA
tatcaatatagaggtggg

SEQ ID NO: 406 - DNA, 18 bp, synthetic construct
  source 1..18: mol_type = other DNA
attggccagggatcaggg

SEQ ID NO: 407 - DNA, 18 bp, synthetic construct
  source 1..18: mol_type = other DNA
ctacagaccccagcgcga

SEQ ID NO: 408 - DNA, 18 bp, synthetic construct
  source 1..18: mol_type = other DNA
cagcaaccgggtagcgct

SEQ ID NO: 409 - DNA, 18 bp, synthetic construct
  source 1..18: mol_type = other DNA
ccttgggcagcagcaacc

SEQ ID NO: 410 - DNA, 18 bp, synthetic construct
  source 1..18: mol_type = other DNA
actccgcggtccttgggc

SEQ ID NO: 411 - DNA, 18 bp, synthetic construct
  source 1..18: mol_type = other DNA
cctgcgtccgactccgcg

SEQ ID NO: 412 - DNA, 18 bp, synthetic construct
  source 1..18: mol_type = other DNA
acatggtctgcctgcgtc

SEQ ID NO: 413 - DNA, 18 bp, synthetic construct
  source 1..18: mol_type = other DNA
accagggtccacatggtc

SEQ ID NO: 414 - DNA, 18 bp, synthetic construct
  source 1..18: mol_type = other DNA
agccaccagccctgctgt

SEQ ID NO: 415 - DNA, 18 bp, synthetic construct
  source 1..18: mol_type = other DNA
tccaggcagcaggccaca

SEQ ID NO: 416 - DNA, 18 bp, synthetic construct
  source 1..18: mol_type = other DNA
gccatttgtccagaaggg

SEQ ID NO: 417 - DNA, 18 bp, synthetic construct
  source 1..18: mol_type = other DNA
gtgggcatcaacctggca

SEQ ID NO: 418 - DNA, 18 bp, synthetic construct
  source 1..18: mol_type = other DNA
caactggaagtccctgag

SEQ ID NO: 419 - DNA, 18 bp, synthetic construct
  source 1..18: mol_type = other DNA
ggcagcagtgatggccat

SEQ ID NO: 420 - DNA, 18 bp, synthetic construct
  source 1..18: mol_type = other DNA
tgatctttggaagcagga

SEQ ID NO: 421 - DNA, 18 bp, synthetic construct
  source 1..18: mol_type = other DNA
cattcgaactgactatca

SEQ ID NO: 422 - DNA, 18 bp, synthetic construct
  source 1..18: mol_type = other DNA
agcacccccaggagccat

SEQ ID NO: 423 - DNA, 18 bp, synthetic construct
  source 1..18: mol_type = other DNA
cggacagcagtgcaccct

SEQ ID NO: 424 - DNA, 18 bp, synthetic construct
  source 1..18: mol_type = other DNA
cccgtgggtgtgatgcag

SEQ ID NO: 425 - DNA, 18 bp, synthetic construct
  source 1..18: mol_type = other DNA
ctgccctgttagtcctct

SEQ ID NO: 426 - DNA, 18 bp, synthetic construct
  source 1..18: mol_type = other DNA
agggcaccgggaccgtgc

SEQ ID NO: 427 - DNA, 18 bp, synthetic construct
  source 1..18: mol_type = other DNA
gggcagcagccatacttc

SEQ ID NO: 428 - DNA, 18 bp, synthetic construct
  source 1..18: mol_type = other DNA
cttgggggcagcagtgca

SEQ ID NO: 429 - DNA, 18 bp, synthetic construct
  source 1..18: mol_type = other DNA
agcgttctccttggagag

SEQ ID NO: 430 - DNA, 18 bp, synthetic construct
  source 1..18: mol_type = other DNA
ttcacatcccccactgtg

SEQ ID NO: 431 - DNA, 18 bp, synthetic construct
  source 1..18: mol_type = other DNA
gtagacggcagcaggtat

SEQ ID NO: 432 - DNA, 18 bp, synthetic construct
  source 1..18: mol_type = other DNA
ctcacagcacacagcctg

SEQ ID NO: 433 - DNA, 18 bp, synthetic construct
  source 1..18: mol_type = other DNA
cccttctgcgtgtcacac

SEQ ID NO: 434 - DNA, 18 bp, synthetic construct
  source 1..18: mol_type = other DNA
ctggggccttctccatcc

SEQ ID NO: 435 - DNA, 18 bp, synthetic construct
  source 1..18: mol_type = other DNA
acaggggacatctctctt

SEQ ID NO: 436 - DNA, 18 bp, synthetic construct
  source 1..18: mol_type = other DNA
gacgtgagttggcagcag

SEQ ID NO: 437 - DNA, 18 bp, synthetic construct
  source 1..18: mol_type = other DNA
ggtccgagcagcagacag

SEQ ID NO: 438 - DNA, 18 bp, synthetic construct
  source 1..18: mol_type = other DNA
acactgcccctcagctac

SEQ ID NO: 439 - DNA, 18 bp, synthetic construct
  source 1..18: mol_type = other DNA
cggcgggcaggcatcttc

SEQ ID NO: 440 - DNA, 18 bp, synthetic construct
  source 1..18: mol_type = other DNA
agctggtgtgctggtcac

SEQ ID NO: 441 - DNA, 18 bp, synthetic construct
  source 1..18: mol_type = other DNA
gcaggcccagctcccacc

SEQ ID NO: 442 - DNA, 18 bp, synthetic construct
  source 1..18: mol_type = other DNA
gggcagcagtgctggcga

SEQ ID NO: 443 - DNA, 18 bp, synthetic construct
  source 1..18: mol_type = other DNA
ccacttccttctcgcagg

SEQ ID NO: 444 - DNA, 18 bp, synthetic construct
  source 1..18: mol_type = other DNA
cacacccacgtgagggct

SEQ ID NO: 445 - DNA, 18 bp, synthetic construct
  source 1..18: mol_type = other DNA
cgccctggcggtagggac

SEQ ID NO: 446 - DNA, 18 bp, synthetic construct
  source 1..18: mol_type = other DNA
tgcgcagcggaagccagc

SEQ ID NO: 447 - DNA, 18 bp, synthetic construct
  source 1..18: mol_type = other DNA
ggggcgtcccagcgcggg

SEQ ID NO: 448 - DNA, 18 bp, synthetic construct
  source 1..18: mol_type = other DNA
tccctcacagcagctgtc

SEQ ID NO: 449 - DNA, 18 bp, synthetic construct
  source 1..18: mol_type = other DNA
ttcagtactgtccctcac

SEQ ID NO: 450 - DNA, 18 bp, synthetic construct
  source 1..18: mol_type = other DNA
ctgcagagtcttcagtac

SEQ ID NO: 451 - DNA, 18 bp, synthetic construct
  source 1..18: mol_type = other DNA
gtcccgagggctgcagag

SEQ ID NO: 452 - DNA, 18 bp, synthetic construct
  source 1..18: mol_type = other DNA
tccgagtggggtcccgag

SEQ ID NO: 453 - DNA, 18 bp, synthetic construct
  source 1..18: mol_type = other DNA
gagggcaccctccgagtg

SEQ ID NO: 454 - DNA, 18 bp, synthetic construct
  source 1..18: mol_type = other DNA
ggcctgagcagagggcac

SEQ ID NO: 455 - DNA, 18 bp, synthetic construct
  source 1..18: mol_type = other DNA
gtgctagggaggcctgag

SEQ ID NO: 456 - DNA, 18 bp, synthetic construct
  source 1..18: mol_type = other DNA
ttagggggaggtgctagg

SEQ ID NO: 457 - DNA, 18 bp, synthetic construct
  source 1..18: mol_type = other DNA
gagaatttggttaggggg

SEQ ID NO: 458 - DNA, 18 bp, synthetic construct
  source 1..18: mol_type = other DNA
ggggtccagggagaattt

SEQ ID NO: 459 - DNA, 18 bp, synthetic construct
  source 1..18: mol_type = other DNA
agctcagaatggggtcca

SEQ ID NO: 460 - DNA, 18 bp, synthetic construct
  source 1..18: mol_type = other DNA
ggtgatggggagctcaga

SEQ ID NO: 461 - DNA, 18 bp, synthetic construct
  source 1..18: mol_type = other DNA
cacctcccatggtgatgg

SEQ ID NO: 462 - DNA, 18 bp, synthetic construct
  source 1..18: mol_type = other DNA
attgaggccccacctccc

SEQ ID NO: 463 - DNA, 18 bp, synthetic construct
  source 1..18: mol_type = other DNA
aaggccttagattgaggc

SEQ ID NO: 464 - DNA, 18 bp, synthetic construct
  source 1..18: mol_type = other DNA
tctgacagggaaggcctt

SEQ ID NO: 465 - DNA, 18 bp, synthetic construct
  source 1..18: mol_type = other DNA
acaacccccttctgacag

SEQ ID NO: 466 - DNA, 18 bp, synthetic construct
  source 1..18: mol_type = other DNA
ggcttttgccacaacccc

SEQ ID NO: 467 - DNA, 18 bp, synthetic construct
  source 1..18: mol_type = other DNA
cttgtaatgtggcttttg

SEQ ID NO: 468 - DNA, 18 bp, synthetic construct
  source 1..18: mol_type = other DNA
gggatggcagcttgtaat

SEQ ID NO: 469 - DNA, 18 bp, synthetic construct
  source 1..18: mol_type = other DNA
aaacggggaggggatggc

SEQ ID NO: 470 - DNA, 18 bp, synthetic construct
  source 1..18: mol_type = other DNA
gggtccactgaaacgggg

SEQ ID NO: 471 - DNA, 18 bp, synthetic construct
  source 1..18: mol_type = other DNA
cctggccacagggtccac

SEQ ID NO: 472 - DNA, 18 bp, synthetic construct
  source 1..18: mol_type = other DNA
gggaaaagcacctggcca

SEQ ID NO: 473 - DNA, 18 bp, synthetic construct
  source 1..18: mol_type = other DNA
cctgtggatagggaaaag

SEQ ID NO: 474 - DNA, 18 bp, synthetic construct
  source 1..18: mol_type = other DNA
cacaaacacccctgtgga

SEQ ID NO: 475 - DNA, 18 bp, synthetic construct
  source 1..18: mol_type = other DNA
cgcgcacacacacaaaca

SEQ ID NO: 476 - DNA, 18 bp, synthetic construct
  source 1..18: mol_type = other DNA
gaaacgcacacgcgcaca

SEQ ID NO: 477 - DNA, 18 bp, synthetic construct
  source 1..18: mol_type = other DNA
aaactttattgaaacgca

SEQ ID NO: 478 - DNA, 18 bp, synthetic construct
  source 1..18: mol_type = other DNA
gaaagtgtacaaacttta

SEQ ID NO: 479 - DNA, 18 bp, synthetic construct
  source 1..18: mol_type = other DNA
ttttttttaagaaagtgt

SEQ ID NO: 480 - DNA, 18 bp, synthetic construct
  source 1..18: mol_type = other DNA
tcgccccacaccccattt

SEQ ID NO: 481 - DNA, 18 bp, synthetic construct
  source 1..18: mol_type = other DNA
gtttcttttctactcaaa

SEQ ID NO: 482 - DNA, 18 bp, synthetic construct
  source 1..18: mol_type = other DNA
tacttatcaatatagagg

SEQ ID NO: 483 - DNA, 18 bp, synthetic construct
  source 1..18: mol_type = other DNA
ttccattggccagggatc

SEQ ID NO: 484 - DNA, 18 bp, synthetic construct
  source 1..18: mol_type = other DNA
cagactacagaccccagc

SEQ ID NO: 485 - DNA, 18 bp, synthetic construct
  source 1..18: mol_type = other DNA
gcagcaaccgggtagcgc

SEQ ID NO: 486 - DNA, 18 bp, synthetic construct
  source 1..18: mol_type = other DNA
tccttgggcagcagcaac

SEQ ID NO: 487 - DNA, 18 bp, synthetic construct
  source 1..18: mol_type = other DNA
gactccgcggtccttggg

SEQ ID NO: 488 - DNA, 18 bp, synthetic construct
  source 1..18: mol_type = other DNA
gcctgcgtccgactccgc

SEQ ID NO: 489 - DNA, 18 bp, synthetic construct
  source 1..18: mol_type = other DNA
cacatggtctgcctgcgt

SEQ ID NO: 490 - DNA, 18 bp, synthetic construct
  source 1..18: mol_type = other DNA
caccagggtccacatggt

SEQ ID NO: 491 - DNA, 18 bp, synthetic construct
  source 1..18: mol_type = other DNA
gttccagccaccagccct

SEQ ID NO: 492 - DNA, 18 bp, synthetic construct
  source 1..18: mol_type = other DNA
cggggtccaggcagcagg

SEQ ID NO: 493 - DNA, 18 bp, synthetic construct
  source 1..18: mol_type = other DNA
tgtgggccatttgtccag

SEQ ID NO: 494 - DNA, 18 bp, synthetic construct
  source 1..18: mol_type = other DNA
gagcagtgggcatcaacc

SEQ ID NO: 495 - DNA, 18 bp, synthetic construct
  source 1..18: mol_type = other DNA
ggcagcaactggaagtcc

SEQ ID NO: 496 - DNA, 18 bp, synthetic construct
  source 1..18: mol_type = other DNA
ccgtgggcagcagtgatg

SEQ ID NO: 497 - DNA, 18 bp, synthetic construct
  source 1..18: mol_type = other DNA
ttacctgatctttggaag

SEQ ID NO: 498 - DNA, 18 bp, synthetic construct
  source 1..18: mol_type = other DNA
ccgggcattcgaactgac

SEQ ID NO: 499 - DNA, 18 bp, synthetic construct
  source 1..18: mol_type = other DNA
ggggcagcacccccagga

SEQ ID NO: 500 - DNA, 18 bp, synthetic construct
  source 1..18: mol_type = other DNA
ccgtgcggacagcagtgc

SEQ ID NO: 501 - DNA, 18 bp, synthetic construct
  source 1..18: mol_type = other DNA
gggtgcccgtgggtgtga

SEQ ID NO: 502 - DNA, 18 bp, synthetic construct
  source 1..18: mol_type = other DNA
ggccactgccctgttagt

SEQ ID NO: 503 - DNA, 18 bp, synthetic construct
  source 1..18: mol_type = other DNA
ccatcagggcaccgggac

SEQ ID NO: 504 - DNA, 18 bp, synthetic construct
  source 1..18: mol_type = other DNA
gcattgggcagcagccat

SEQ ID NO: 505 - DNA, 18 bp, synthetic construct
  source 1..18: mol_type = other DNA
agtgtcttgggggcagca

SEQ ID NO: 506 - DNA, 18 bp, synthetic construct
  source 1..18: mol_type = other DNA
gtggtagcgttctccttg

SEQ ID NO: 507 - DNA, 18 bp, synthetic construct
  source 1..18: mol_type = other DNA
cacatttcacatccccca

SEQ ID NO: 508 - DNA, 18 bp, synthetic construct
  source 1..18: mol_type = other DNA
cgactgtagacggcagca

SEQ ID NO: 509 - DNA, 18 bp, synthetic construct
  source 1..18: mol_type = other DNA
tggtcctcacagcacaca

SEQ ID NO: 510 - DNA, 18 bp, synthetic construct
  source 1..18: mol_type = other DNA
aggtacccttctgcgtgt

SEQ ID NO: 511 - DNA, 18 bp, synthetic construct
  source 1..18: mol_type = other DNA
gtgagctggggccttctc

SEQ ID NO: 512 - DNA, 18 bp, synthetic construct
  source 1..18: mol_type = other DNA
ttatcacaggggacatct

SEQ ID NO: 513 - DNA, 18 bp, synthetic construct
  source 1..18: mol_type = other DNA
ccccagacgtgagttggc

SEQ ID NO: 514 - DNA, 18 bp, synthetic construct
  source 1..18: mol_type = other DNA
ctggtggtccgagcagca

SEQ ID NO: 515 - DNA, 18 bp, synthetic construct
  source 1..18: mol_type = other DNA
cgctgacactgcccctca

SEQ ID NO: 516 - DNA, 18 bp, synthetic construct
  source 1..18: mol_type = other DNA
aagcccggcgggcaggca

SEQ ID NO: 517 - DNA, 18 bp, synthetic construct
  source 1..18: mol_type = other DNA
cgggcagctggtgtgctg

SEQ ID NO: 518 - DNA, 18 bp, synthetic construct
  source 1..18: mol_type = other DNA
tggcagcaggcccagctc

SEQ ID NO: 519 - DNA, 18 bp, synthetic construct
  source 1..18: mol_type = other DNA
cagccgggcagcagtgct

SEQ ID NO: 520 - DNA, 18 bp, synthetic construct
  source 1..18: mol_type = other DNA
agagaccacttccttctc

SEQ ID NO: 521 - DNA, 18 bp, synthetic construct
  source 1..18: mol_type = other DNA
tccttcacacccacgtga

SEQ ID NO: 522 - DNA, 18 bp, synthetic construct
  source 1..18: mol_type = other DNA
ggcagcaggtctggttat

SEQ ID NO: 523 - DNA, 18 bp, synthetic construct
  source 1..18: mol_type = other DNA
acagacgccctggcggta

SEQ ID NO: 524 - DNA, 18 bp, synthetic construct
  source 1..18: mol_type = other DNA
ctggctgcgcagcggaag

SEQ ID NO: 525 - DNA, 18 bp, synthetic construct
  source 1..18: mol_type = other DNA
tcaaaggggcgtcccagc

SEQ ID NO: 526 - DNA, 18 bp, synthetic construct
  source 1..18: mol_type = other DNA
gtccctcacagcagctgt

SEQ ID NO: 527 - DNA, 18 bp, synthetic construct
  source 1..18: mol_type = other DNA
cttcagtactgtccctca

SEQ ID NO: 528 - DNA, 18 bp, synthetic construct
  source 1..18: mol_type = other DNA
gctgcagagtcttcagta

SEQ ID NO: 529 - DNA, 18 bp, synthetic construct
  source 1..18: mol_type = other DNA
ggtcccgagggctgcaga

SEQ ID NO: 530 - DNA, 18 bp, synthetic construct
  source 1..18: mol_type = other DNA
ctccgagtggggtcccga

SEQ ID NO: 531 - DNA, 18 bp, synthetic construct
  source 1..18: mol_type = other DNA
agagggcaccctccgagt

SEQ ID NO: 532 - DNA, 18 bp, synthetic construct
  source 1..18: mol_type = other DNA
aggcctgagcagagggca

SEQ ID NO: 533 - DNA, 18 bp, synthetic construct
  source 1..18: mol_type = other DNA
ggtgctagggaggcctga

SEQ ID NO: 534 - DNA, 18 bp, synthetic construct
  source 1..18: mol_type = other DNA
gttagggggaggtgctag

SEQ ID NO: 535 - DNA, 18 bp, synthetic construct
  source 1..18: mol_type = other DNA
ggagaatttggttagggg

SEQ ID NO: 536 - DNA, 18 bp, synthetic construct
  source 1..18: mol_type = other DNA
tggggtccagggagaatt

SEQ ID NO: 537 - DNA, 18 bp, synthetic construct
  source 1..18: mol_type = other DNA
gagctcagaatggggtcc

SEQ ID NO: 538 - DNA, 18 bp, synthetic construct
  source 1..18: mol_type = other DNA
tggtgatggggagctcag

SEQ ID NO: 539 - DNA, 18 bp, synthetic construct
  source 1..18: mol_type = other DNA
ccacctcccatggtgatg

SEQ ID NO: 540 - DNA, 18 bp, synthetic construct
  source 1..18: mol_type = other DNA
gattgaggccccacctcc

SEQ ID NO: 541 - DNA, 18 bp, synthetic construct
  source 1..18: mol_type = other DNA
gaaggccttagattgagg

SEQ ID NO: 542 - DNA, 18 bp, synthetic construct
  source 1..18: mol_type = other DNA
ttctgacagggaaggcct

SEQ ID NO: 543 - DNA, 18 bp, synthetic construct
  source 1..18: mol_type = other DNA
cacaacccccttctgaca

SEQ ID NO: 544 - DNA, 18 bp, synthetic construct
  source 1..18: mol_type = other DNA
tggcttttgccacaaccc

SEQ ID NO: 545 - DNA, 18 bp, synthetic construct
  source 1..18: mol_type = other DNA
gcttgtaatgtggctttt

SEQ ID NO: 546 - DNA, 18 bp, synthetic construct
  source 1..18: mol_type = other DNA
ggggatggcagcttgtaa

SEQ ID NO: 547 - DNA, 18 bp, synthetic construct
  source 1..18: mol_type = other DNA
gaaacggggaggggatgg

SEQ ID NO: 548 - DNA, 18 bp, synthetic construct
  source 1..18: mol_type = other DNA
agggtccactgaaacggg

SEQ ID NO: 549 - DNA, 18 bp, synthetic construct
  source 1..18: mol_type = other DNA
acctggccacagggtcca

SEQ ID NO: 550 - DNA, 18 bp, synthetic construct
  source 1..18: mol_type = other DNA
agggaaaagcacctggcc

SEQ ID NO: 551 - DNA, 18 bp, synthetic construct
  source 1..18: mol_type = other DNA
ccctgtggatagggaaaa

SEQ ID NO: 552 - DNA, 18 bp, synthetic construct
  source 1..18: mol_type = other DNA
acacaaacacccctgtgg

SEQ ID NO: 553 - DNA, 18 bp, synthetic construct
  source 1..18: mol_type = other DNA
acgcgcacacacacaaac

SEQ ID NO: 554 - DNA, 18 bp, synthetic construct
  source 1..18: mol_type = other DNA
tgaaacgcacacgcgcac

SEQ ID NO: 555 - DNA, 18 bp, synthetic construct
  source 1..18: mol_type = other DNA
caaactttattgaaacgc

SEQ ID NO: 556 - DNA, 18 bp, synthetic construct
  source 1..18: mol_type = other DNA
agaaagtgtacaaacttt

SEQ ID NO: 557 - DNA, 18 bp, synthetic construct
  source 1..18: mol_type = other DNA
tttttttttaagaaagtg

SEQ ID NO: 558 - DNA, 18 bp, synthetic construct
  source 1..18: mol_type = other DNA
cctctcgccccacacccc

SEQ ID NO: 559 - DNA, 18 bp, synthetic construct
  source 1..18: mol_type = other DNA
ctgtgtttcttttctact

SEQ ID NO: 560 - DNA, 18 bp, synthetic construct
  source 1..18: mol_type = other DNA
tggctacttatcaatata

SEQ ID NO: 561 - DNA, 18 bp, synthetic construct
  source 1..18: mol_type = other DNA
cagtttccattggccagg

SEQ ID NO: 562 - DNA, 18 bp, synthetic construct
  source 1..18: mol_type = other DNA
cgctcagactacagaccc

SEQ ID NO: 563 - DNA, 18 bp, synthetic construct
  source 1..18: mol_type = other DNA
agcagcaaccgggtagcg

SEQ ID NO: 564 - DNA, 18 bp, synthetic construct
  source 1..18: mol_type = other DNA
gtccttgggcagcagcaa

SEQ ID NO: 565 - DNA, 18 bp, synthetic construct
  source 1..18: mol_type = other DNA
cgactccgcggtccttgg

SEQ ID NO: 566 - DNA, 18 bp, synthetic construct
  source 1..18: mol_type = other DNA
tgcctgcgtccgactccg

SEQ ID NO: 567 - DNA, 18 bp, synthetic construct
  source 1..18: mol_type = other DNA
ccacatggtctgcctgcg

SEQ ID NO: 568 - DNA, 18 bp, synthetic construct
  source 1..18: mol_type = other DNA
tcaccagggtccacatgg

SEQ ID NO: 569 - DNA, 18 bp, synthetic construct
  source 1..18: mol_type = other DNA
accgcgttccagccacca

SEQ ID NO: 570 - DNA, 18 bp, synthetic construct
  source 1..18: mol_type = other DNA
tcctccggggtccaggca

SEQ ID NO: 571 - DNA, 18 bp, synthetic construct
  source 1..18: mol_type = other DNA
agtgttgtgggccatttg

SEQ ID NO: 572 - DNA, 18 bp, synthetic construct
  source 1..18: mol_type = other DNA
cggcagagcagtgggcat

SEQ ID NO: 573 - DNA, 18 bp, synthetic construct
  source 1..18: mol_type = other DNA
gaaggggcagcaactgga

SEQ ID NO: 574 - DNA, 18 bp, synthetic construct
  source 1..18: mol_type = other DNA
aagccccgtgggcagcag

SEQ ID NO: 575 - DNA, 18 bp, synthetic construct
  source 1..18: mol_type = other DNA
agttgttacctgatcttt

SEQ ID NO: 576 - DNA, 18 bp, synthetic construct
  source 1..18: mol_type = other DNA
gaagtccgggcattcgaa

SEQ ID NO: 577 - DNA, 18 bp, synthetic construct
  source 1..18: mol_type = other DNA
ggcatggggcagcacccc

SEQ ID NO: 578 - DNA, 18 bp, synthetic construct
  source 1..18: mol_type = other DNA
aggcaccgtgcggacagc

SEQ ID NO: 579 - DNA, 18 bp, synthetic construct
  source 1..18: mol_type = other DNA
ggggtgggtgcccgtggg

SEQ ID NO: 580 - DNA, 18 bp, synthetic construct
  source 1..18: mol_type = other DNA
gacaaggccactgccctg

SEQ ID NO: 581 - DNA, 18 bp, synthetic construct
  source 1..18: mol_type = other DNA
tagaaccatcagggcacc

SEQ ID NO: 582 - DNA, 18 bp, synthetic construct
  source 1..18: mol_type = other DNA
gttgggcattgggcagca

SEQ ID NO: 583 - DNA, 18 bp, synthetic construct
  source 1..18: mol_type = other DNA
cacacagtgtcttggggg

SEQ ID NO: 584 - DNA, 18 bp, synthetic construct
  source 1..18: mol_type = other DNA
ggtccgtggtagcgttct

SEQ ID NO: 585 - DNA, 18 bp, synthetic construct
  source 1..18: mol_type = other DNA
catgtcacatttcacatc

SEQ ID NO: 586 - DNA, 18 bp, synthetic construct
  source 1..18: mol_type = other DNA
gcccccgactgtagacgg

SEQ ID NO: 587 - DNA, 18 bp, synthetic construct
  source 1..18: mol_type = other DNA
gtatgtggtcctcacagc

SEQ ID NO: 588 - DNA, 18 bp, synthetic construct
  source 1..18: mol_type = other DNA
ttcacaggtacccttctg

SEQ ID NO: 589 - DNA, 18 bp, synthetic construct
  source 1..18: mol_type = other DNA
ctgaggtgagctggggcc

SEQ ID NO: 590 - DNA, 18 bp, synthetic construct
  source 1..18: mol_type = other DNA
tgacattatcacagggga

SEQ ID NO: 591 - DNA, 18 bp, synthetic construct
  source 1..18: mol_type = other DNA
ccactccccagacgtgag

SEQ ID NO: 592 - DNA, 18 bp, synthetic construct
  source 1..18: mol_type = other DNA
cagtgctggtggtccgag

SEQ ID NO: 593 - DNA, 18 bp, synthetic construct
  source 1..18: mol_type = other DNA
ttcctcgctgacactgcc

SEQ ID NO: 594 - DNA, 18 bp, synthetic construct
  source 1..18: mol_type = other DNA
taaggaagcccggcgggc

SEQ ID NO: 595 - DNA, 18 bp, synthetic construct
  source 1..18: mol_type = other DNA
cccaccgggcagctggtg

SEQ ID NO: 596 - DNA, 18 bp, synthetic construct
  source 1..18: mol_type = other DNA
gcaactggcagcaggccc

SEQ ID NO: 597 - DNA, 18 bp, synthetic construct
  source 1..18: mol_type = other DNA
gtagccagccgggcagca

SEQ ID NO: 598 - DNA, 18 bp, synthetic construct
  source 1..18: mol_type = other DNA
tgggcagagaccacttcc

SEQ ID NO: 599 - DNA, 18 bp, synthetic construct
  source 1..18: mol_type = other DNA
ccacgtccttcacaccca

SEQ ID NO: 600 - DNA, 18 bp, synthetic construct
  source 1..18: mol_type = other DNA
gtctcggcagcaggtctg

SEQ ID NO: 601 - DNA, 18 bp, synthetic construct
  source 1..18: mol_type = other DNA
gcacaacagacgccctgg

SEQ ID NO: 602 - DNA, 18 bp, synthetic construct
  source 1..18: mol_type = other DNA
tacccctggctgcgcagc

SEQ ID NO: 603 - DNA, 18 bp, synthetic construct
  source 1..18: mol_type = other DNA
gtccctcaaaggggcgtc

SEQ ID NO: 604 - DNA, 18 bp, synthetic construct
  source 1..18: mol_type = other DNA
tgtccctcacagcagctg

SEQ ID NO: 605 - DNA, 18 bp, synthetic construct
  source 1..18: mol_type = other DNA
tcttcagtactgtccctc

SEQ ID NO: 606 - DNA, 18 bp, synthetic construct
  source 1..18: mol_type = other DNA
ggctgcagagtcttcagt

SEQ ID NO: 607 - DNA, 18 bp, synthetic construct
  source 1..18: mol_type = other DNA
gggtcccgagggctgcag

SEQ ID NO: 608 - DNA, 18 bp, synthetic construct
  source 1..18: mol_type = other DNA
cctccgagtggggtcccg

SEQ ID NO: 609 - DNA, 18 bp, synthetic construct
  source 1..18: mol_type = other DNA
cagagggcaccctccgag

SEQ ID NO: 610 - DNA, 18 bp, synthetic construct
  source 1..18: mol_type = other DNA
gaggcctgagcagagggc

SEQ ID NO: 611 - DNA, 18 bp, synthetic construct
  source 1..18: mol_type = other DNA
aggtgctagggaggcctg

SEQ ID NO: 612 - DNA, 18 bp, synthetic construct
  source 1..18: mol_type = other DNA
ggttagggggaggtgcta

SEQ ID NO: 613 - DNA, 18 bp, synthetic construct
  source 1..18: mol_type = other DNA
gggagaatttggttaggg

SEQ ID NO: 614 - DNA, 18 bp, synthetic construct
  source 1..18: mol_type = other DNA
atggggtccagggagaat

SEQ ID NO: 615 - DNA, 18 bp, synthetic construct
  source 1..18: mol_type = other DNA
ggagctcagaatggggtc

SEQ ID NO: 616 - DNA, 18 bp, synthetic construct
  source 1..18: mol_type = other DNA
atggtgatggggagctca

SEQ ID NO: 617 - DNA, 18 bp, synthetic construct
  source 1..18: mol_type = other DNA
cccacctcccatggtgat

SEQ ID NO: 618 - DNA, 18 bp, synthetic construct
  source 1..18: mol_type = other DNA
agattgaggccccacctc

SEQ ID NO: 619 - DNA, 18 bp, synthetic construct
  source 1..18: mol_type = other DNA
ggaaggccttagattgag

SEQ ID NO: 620 - DNA, 18 bp, synthetic construct
  source 1..18: mol_type = other DNA
cttctgacagggaaggcc

SEQ ID NO: 621 - DNA, 18 bp, synthetic construct
  source 1..18: mol_type = other DNA
ccacaacccccttctgac

SEQ ID NO: 622 - DNA, 18 bp, synthetic construct
  source 1..18: mol_type = other DNA
gtggcttttgccacaacc

SEQ ID NO: 623 - DNA, 18 bp, synthetic construct
  source 1..18: mol_type = other DNA
agcttgtaatgtggcttt

SEQ ID NO: 624 - DNA, 18 bp, synthetic construct
  source 1..18: mol_type = other DNA
aggggatggcagcttgta

SEQ ID NO: 625 - DNA, 18 bp, synthetic construct
  source 1..18: mol_type = other DNA
tgaaacggggaggggatg

SEQ ID NO: 626 - DNA, 18 bp, synthetic construct
  source 1..18: mol_type = other DNA
cagggtccactgaaacgg

SEQ ID NO: 627 - DNA, 18 bp, synthetic construct
  source 1..18: mol_type = other DNA
cacctggccacagggtcc

SEQ ID NO: 628 - DNA, 18 bp, synthetic construct
  source 1..18: mol_type = other DNA
tagggaaaagcacctggc

SEQ ID NO: 629 - DNA, 18 bp, synthetic construct
  source 1..18: mol_type = other DNA
cccctgtggatagggaaa

SEQ ID NO: 630 - DNA, 18 bp, synthetic construct
  source 1..18: mol_type = other DNA
cacacaaacacccctgtg

SEQ ID NO: 631 - DNA, 18 bp, synthetic construct
  source 1..18: mol_type = other DNA
cacgcgcacacacacaaa

SEQ ID NO: 632 - DNA, 18 bp, synthetic construct
  source 1..18: mol_type = other DNA
ttgaaacgcacacgcgca

SEQ ID NO: 633 - DNA, 18 bp, synthetic construct
  source 1..18: mol_type = other DNA
acaaactttattgaaacg

SEQ ID NO: 634 - DNA, 18 bp, synthetic construct
  source 1..18: mol_type = other DNA
aagaaagtgtacaaactt

SEQ ID NO: 635 - DNA, 18 bp, synthetic construct
  source 1..18: mol_type = other DNA
ttttttttttaagaaagt

SEQ ID NO: 636 - DNA, 18 bp, synthetic construct
  source 1..18: mol_type = other DNA
gcttcctctcgccccaca

SEQ ID NO: 637 - DNA, 18 bp, synthetic construct
  source 1..18: mol_type = other DNA
aatgctgtgtttcttttc

SEQ ID NO: 638 - DNA, 18 bp, synthetic construct
  source 1..18: mol_type = other DNA
ccattggctacttatcaa

SEQ ID NO: 639 - DNA, 18 bp, synthetic construct
  source 1..18: mol_type = other DNA
acctcagtttccattggc

SEQ ID NO: 640 - DNA, 18 bp, synthetic construct
  source 1..18: mol_type = other DNA
gtagcgctcagactacag

SEQ ID NO: 641 - DNA, 18 bp, synthetic construct
  source 1..18: mol_type = other DNA
cagcagcaaccgggtagc

SEQ ID NO: 642 - DNA, 18 bp, synthetic construct
  source 1..18: mol_type = other DNA
ggtccttgggcagcagca

SEQ ID NO: 643 - DNA, 18 bp, synthetic construct
  source 1..18: mol_type = other DNA
ccgactccgcggtccttg

SEQ ID NO: 644 - DNA, 18 bp, synthetic construct
  source 1..18: mol_type = other DNA
ctgcctgcgtccgactcc

SEQ ID NO: 645 - DNA, 18 bp, synthetic construct
  source 1..18: mol_type = other DNA
tccacatggtctgcctgc

SEQ ID NO: 646 - DNA, 18 bp, synthetic construct
  source 1..18: mol_type = other DNA
ctcaccagggtccacatg

SEQ ID NO: 647 - DNA, 18 bp, synthetic construct
  source 1..18: mol_type = other DNA
tgggcaccgcgttccagc

SEQ ID NO: 648 - DNA, 18 bp, synthetic construct
  source 1..18: mol_type = other DNA
ctggctcctccggggtcc

SEQ ID NO: 649 - DNA, 18 bp, synthetic construct
  source 1..18: mol_type = other DNA
tgctcagtgttgtgggcc

SEQ ID NO: 650 - DNA, 18 bp, synthetic construct
  source 1..18: mol_type = other DNA
gtggccggcagagcagtg

SEQ ID NO: 651 - DNA, 18 bp, synthetic construct
  source 1..18: mol_type = other DNA
tctgggaaggggcagcaa

SEQ ID NO: 652 - DNA, 18 bp, synthetic construct
  source 1..18: mol_type = other DNA
agtggaagccccgtgggc

SEQ ID NO: 653 - DNA, 18 bp, synthetic construct
  source 1..18: mol_type = other DNA
cacggagttgttacctga

SEQ ID NO: 654 - DNA, 18 bp, synthetic construct
  source 1..18: mol_type = other DNA
gtggagaagtccgggcat

SEQ ID NO: 655 - DNA, 18 bp, synthetic construct
  source 1..18: mol_type = other DNA
cctggggcatggggcagc

SEQ ID NO: 656 - DNA, 18 bp, synthetic construct
  source 1..18: mol_type = other DNA
gcagaaggcaccgtgcgg

SEQ ID NO: 657 - DNA, 18 bp, synthetic construct
  source 1..18: mol_type = other DNA
gccagggggtgggtgccc

SEQ ID NO: 658 - DNA, 18 bp, synthetic construct
  source 1..18: mol_type = other DNA
agctggacaaggccactg

SEQ ID NO: 659 - DNA, 18 bp, synthetic construct
  source 1..18: mol_type = other DNA
gcaggtagaaccatcagg

SEQ ID NO: 660 - DNA, 18 bp, synthetic construct
  source 1..18: mol_type = other DNA
gtggcgttgggcattggg

SEQ ID NO: 661 - DNA, 18 bp, synthetic construct
  source 1..18: mol_type = other DNA
ggtcacacacagtgtctt

SEQ ID NO: 662 - DNA, 18 bp, synthetic construct
  source 1..18: mol_type = other DNA
gaggaggtccgtggtagc

SEQ ID NO: 663 - DNA, 18 bp, synthetic construct
  source 1..18: mol_type = other DNA
acctccatgtcacatttc

SEQ ID NO: 664 - DNA, 18 bp, synthetic construct
  source 1..18: mol_type = other DNA
cccaggcccccgactgta

SEQ ID NO: 665 - DNA, 18 bp, synthetic construct
  source 1..18: mol_type = other DNA
gcagtgtatgtggtcctc

SEQ ID NO: 666 - DNA, 18 bp, synthetic construct
  source 1..18: mol_type = other DNA
ccctgttcacaggtaccc

SEQ ID NO: 667 - DNA, 18 bp, synthetic construct
  source 1..18: mol_type = other DNA
gcaggctgaggtgagctg

SEQ ID NO: 668 - DNA, 18 bp, synthetic construct
  source 1..18: mol_type = other DNA
gctgctgacattatcaca

SEQ ID NO: 669 - DNA, 18 bp, synthetic construct
  source 1..18: mol_type = other DNA
cagccccactccccagac

SEQ ID NO: 670 - DNA, 18 bp, synthetic construct
  source 1..18: mol_type = other DNA
ggcagcagtgctggtggt

SEQ ID NO: 671 - DNA, 18 bp, synthetic construct
  source 1..18: mol_type = other DNA
ctcgcttcctcgctgaca

SEQ ID NO: 672 - DNA, 18 bp, synthetic construct
  source 1..18: mol_type = other DNA
tgggataaggaagcccgg

SEQ ID NO: 673 - DNA, 18 bp, synthetic construct
  source 1..18: mol_type = other DNA
tctgccccaccgggcagc

SEQ ID NO: 674 - DNA, 18 bp, synthetic construct
  source 1..18: mol_type = other DNA
atggggcaactggcagca

SEQ ID NO: 675 - DNA, 18 bp, synthetic construct
  source 1..18: mol_type = other DNA
caggtgtagccagccggg

SEQ ID NO: 676 - DNA, 18 bp, synthetic construct
  source 1..18: mol_type = other DNA
caggctgggcagagacca

SEQ ID NO: 677 - DNA, 18 bp, synthetic construct
  source 1..18: mol_type = other DNA
acactccacgtccttcac

SEQ ID NO: 678 - DNA, 18 bp, synthetic construct
  source 1..18: mol_type = other DNA
cggttgtctcggcagcag

SEQ ID NO: 679 - DNA, 18 bp, synthetic construct
  source 1..18: mol_type = other DNA
gatcagcacaacagacgc

SEQ ID NO: 680 - DNA, 18 bp, synthetic construct
  source 1..18: mol_type = other DNA
cttggtacccctggctgc

SEQ ID NO: 681 - DNA, 18 bp, synthetic construct
  source 1..18: mol_type = other DNA
gctgggtccctcaaaggg

SEQ ID NO: 682 - DNA, 18 bp, synthetic construct
  source 1..18: mol_type = other DNA
ctgtccctcacagcagct

SEQ ID NO: 683 - DNA, 18 bp, synthetic construct
  source 1..18: mol_type = other DNA
gtcttcagtactgtccct

SEQ ID NO: 684 - DNA, 18 bp, synthetic construct
  source 1..18: mol_type = other DNA
gggctgcagagtcttcag

SEQ ID NO: 685 - DNA, 18 bp, synthetic construct
  source 1..18: mol_type = other DNA
ggggtcccgagggctgca

SEQ ID NO: 686 - DNA, 18 bp, synthetic construct
  source 1..18: mol_type = other DNA
ccctccgagtggggtccc

SEQ ID NO: 687 - DNA, 18 bp, synthetic construct
  source 1..18: mol_type = other DNA
gcagagggcaccctccga

SEQ ID NO: 688 - DNA, 18 bp, synthetic construct
  source 1..18: mol_type = other DNA
ggaggcctgagcagaggg

SEQ ID NO: 689 - DNA, 18 bp, synthetic construct
  source 1..18: mol_type = other DNA
gaggtgctagggaggcct

SEQ ID NO: 690 - DNA, 18 bp, synthetic construct
  source 1..18: mol_type = other DNA
tggttagggggaggtgct

SEQ ID NO: 691 - DNA, 18 bp, synthetic construct
  source 1..18: mol_type = other DNA
agggagaatttggttagg

SEQ ID NO: 692 - DNA, 18 bp, synthetic construct
  source 1..18: mol_type = other DNA
aatggggtccagggagaa

SEQ ID NO: 693 - DNA, 18 bp, synthetic construct
  source 1..18: mol_type = other DNA
gggagctcagaatggggt

SEQ ID NO: 694 - DNA, 18 bp, synthetic construct
  source 1..18: mol_type = other DNA
catggtgatggggagctc

SEQ ID NO: 695 - DNA, 18 bp, synthetic construct
  source 1..18: mol_type = other DNA
ccccacctcccatggtga

SEQ ID NO: 696 - DNA, 18 bp, synthetic construct
  source 1..18: mol_type = other DNA
tagattgaggccccacct

SEQ ID NO: 697 - DNA, 18 bp, synthetic construct
  source 1..18: mol_type = other DNA
gggaaggccttagattga

SEQ ID NO: 698 - DNA, 18 bp, synthetic construct
  source 1..18: mol_type = other DNA
ccttctgacagggaaggc

SEQ ID NO: 699 - DNA, 18 bp, synthetic construct
  source 1..18: mol_type = other DNA
gccacaacccccttctga

SEQ ID NO: 700 - DNA, 18 bp, synthetic construct
  source 1..18: mol_type = other DNA
tgtggcttttgccacaac

SEQ ID NO: 701 - DNA, 18 bp, synthetic construct
  source 1..18: mol_type = other DNA
cagcttgtaatgtggctt

SEQ ID NO: 702 - DNA, 18 bp, synthetic construct
  source 1..18: mol_type = other DNA
gaggggatggcagcttgt

SEQ ID NO: 703 - DNA, 18 bp, synthetic construct
  source 1..18: mol_type = other DNA
ctgaaacggggaggggat

SEQ ID NO: 704 - DNA, 18 bp, synthetic construct
  source 1..18: mol_type = other DNA
acagggtccactgaaacg

SEQ ID NO: 705 - DNA, 18 bp, synthetic construct
  source 1..18: mol_type = other DNA
gcacctggccacagggtc

SEQ ID NO: 706 - DNA, 18 bp, synthetic construct
  source 1..18: mol_type = other DNA
atagggaaaagcacctgg

SEQ ID NO: 707 - DNA, 18 bp, synthetic construct
  source 1..18: mol_type = other DNA
acccctgtggatagggaa

SEQ ID NO: 708 - DNA, 18 bp, synthetic construct
  source 1..18: mol_type = other DNA
acacacaaacacccctgt

SEQ ID NO: 709 - DNA, 18 bp, synthetic construct
  source 1..18: mol_type = other DNA
acacgcgcacacacacaa

SEQ ID NO: 710 - DNA, 18 bp, synthetic construct
  source 1..18: mol_type = other DNA
attgaaacgcacacgcgc

SEQ ID NO: 711 - DNA, 18 bp, synthetic construct
  source 1..18: mol_type = other DNA
tacaaactttattgaaac

SEQ ID NO: 712 - DNA, 18 bp, synthetic construct
  source 1..18: mol_type = other DNA
taagaaagtgtacaaact

SEQ ID NO: 713 - DNA, 18 bp, synthetic construct
  source 1..18: mol_type = other DNA
tttttttttttaagaaag

SEQ ID NO: 714 - DNA, 18 bp, synthetic construct
  source 1..18: mol_type = other DNA
ccctgcttcctctcgccc

SEQ ID NO: 715 - DNA, 18 bp, synthetic construct
  source 1..18: mol_type = other DNA
ctggaatgctgtgtttct

SEQ ID NO: 716 - DNA, 18 bp, synthetic construct
  source 1..18: mol_type = other DNA
gctcccattggctactta

SEQ ID NO: 717 - DNA, 18 bp, synthetic construct
  source 1..18: mol_type = other DNA
gcctacctcagtttccat

SEQ ID NO: 718 - DNA, 18 bp, synthetic construct
  source 1..18: mol_type = other DNA
ccgggtagcgctcagact

SEQ ID NO: 719 - DNA, 18 bp, synthetic construct
  source 1..18: mol_type = other DNA
gcagcagcaaccgggtag

SEQ ID NO: 720 - DNA, 18 bp, synthetic construct
  source 1..18: mol_type = other DNA
cggtccttgggcagcagc

SEQ ID NO: 721 - DNA, 18 bp, synthetic construct
  source 1..18: mol_type = other DNA
tccgactccgcggtcctt

SEQ ID NO: 722 - DNA, 18 bp, synthetic construct
  source 1..18: mol_type = other DNA
tctgcctgcgtccgactc

SEQ ID NO: 723 - DNA, 18 bp, synthetic construct
  source 1..18: mol_type = other DNA
gtccacatggtctgcctg

SEQ ID NO: 724 - DNA, 18 bp, synthetic construct
  source 1..18: mol_type = other DNA
gctcaccagggtccacat

SEQ ID NO: 725 - DNA, 18 bp, synthetic construct
  source 1..18: mol_type = other DNA
ccatctgggcaccgcgtt

SEQ ID NO: 726 - DNA, 18 bp, synthetic construct
  source 1..18: mol_type = other DNA
tgtagctggctcctccgg

SEQ ID NO: 727 - DNA, 18 bp, synthetic construct
  source 1..18: mol_type = other DNA
atgcctgctcagtgttgt

SEQ ID NO: 728 - DNA, 18 bp, synthetic construct
  source 1..18: mol_type = other DNA
caggagtggccggcagag

SEQ ID NO: 729 - DNA, 18 bp, synthetic construct
  source 1..18: mol_type = other DNA
cggcctctgggaaggggc

SEQ ID NO: 730 - DNA, 18 bp, synthetic construct
  source 1..18: mol_type = other DNA
actgcagtggaagccccg

SEQ ID NO: 731 - DNA, 18 bp, synthetic construct
  source 1..18: mol_type = other DNA
gcacccacggagttgtta

SEQ ID NO: 732 - DNA, 18 bp, synthetic construct
  source 1..18: mol_type = other DNA
agcacgtggagaagtccg

SEQ ID NO: 733 - DNA, 18 bp, synthetic construct
  source 1..18: mol_type = other DNA
ggaagcctggggcatggg

SEQ ID NO: 734 - DNA, 18 bp, synthetic construct
  source 1..18: mol_type = other DNA
aggtcgcagaaggcaccg

SEQ ID NO: 735 - DNA, 18 bp, synthetic construct
  source 1..18: mol_type = other DNA
tctttgccagggggtggg

SEQ ID NO: 736 - DNA, 18 bp, synthetic construct
  source 1..18: mol_type = other DNA
gaccgagctggacaaggc

SEQ ID NO: 737 - DNA, 18 bp, synthetic construct
  source 1..18: mol_type = other DNA
tcacagcaggtagaacca

SEQ ID NO: 738 - DNA, 18 bp, synthetic construct
  source 1..18: mol_type = other DNA
agcaggtggcgttgggca

SEQ ID NO: 739 - DNA, 18 bp, synthetic construct
  source 1..18: mol_type = other DNA
gatcaggtcacacacagt

SEQ ID NO: 740 - DNA, 18 bp, synthetic construct
  source 1..18: mol_type = other DNA
ttagtgaggaggtccgtg

SEQ ID NO: 741 - DNA, 18 bp, synthetic construct
  source 1..18: mol_type = other DNA
agctcacctccatgtcac

SEQ ID NO: 742 - DNA, 18 bp, synthetic construct
  source 1..18: mol_type = other DNA
gcagccccaggcccccga

SEQ ID NO: 743 - DNA, 18 bp, synthetic construct
  source 1..18: mol_type = other DNA
ggacagcagtgtatgtgg

SEQ ID NO: 744 - DNA, 18 bp, synthetic construct
  source 1..18: mol_type = other DNA
ggggcccctgttcacagg

SEQ ID NO: 745 - DNA, 18 bp, synthetic construct
  source 1..18: mol_type = other DNA
gtctggcaggctgaggtg

SEQ ID NO: 746 - DNA, 18 bp, synthetic construct
  source 1..18: mol_type = other DNA
ggacagctgctgacatta

SEQ ID NO: 747 - DNA, 18 bp, synthetic construct
  source 1..18: mol_type = other DNA
gacagcagccccactccc

SEQ ID NO: 748 - DNA, 18 bp, synthetic construct
  source 1..18: mol_type = other DNA
ctgggggcagcagtgctg

SEQ ID NO: 749 - DNA, 18 bp, synthetic construct
  source 1..18: mol_type = other DNA
acgatctcgcttcctcgc

SEQ ID NO: 750 - DNA, 18 bp, synthetic construct
  source 1..18: mol_type = other DNA
tggggtgggataaggaag

SEQ ID NO: 751 - DNA, 18 bp, synthetic construct
  source 1..18: mol_type = other DNA
gcaggtctgccccaccgg

SEQ ID NO: 752 - DNA, 18 bp, synthetic construct
  source 1..18: mol_type = other DNA
acagcatggggcaactgg

SEQ ID NO: 753 - DNA, 18 bp, synthetic construct
  source 1..18: mol_type = other DNA
cgttgcaggtgtagccag

SEQ ID NO: 754 - DNA, 18 bp, synthetic construct
  source 1..18: mol_type = other DNA
ggtggcaggctgggcaga

SEQ ID NO: 755 - DNA, 18 bp, synthetic construct
  source 1..18: mol_type = other DNA
tccccacactccacgtcc

SEQ ID NO: 756 - DNA, 18 bp, synthetic construct
  source 1..18: mol_type = other DNA
cctgtcggttgtctcggc

SEQ ID NO: 757 - DNA, 18 bp, synthetic construct
  source 1..18: mol_type = other DNA
gcgccgatcagcacaaca

SEQ ID NO: 758 - DNA, 18 bp, synthetic construct
  source 1..18: mol_type = other DNA
aaacacttggtacccctg

SEQ ID NO: 759 - DNA, 18 bp, synthetic construct
  source 1..18: mol_type = other DNA
tcaaggctgggtccctca

SEQ ID NO: 760 - DNA, 18 bp, synthetic construct
  source 1..18: mol_type = other DNA
actgtccctcacagcagc

SEQ ID NO: 761 - DNA, 18 bp, synthetic construct
  source 1..18: mol_type = other DNA
agtcttcagtactgtccc

SEQ ID NO: 762 - DNA, 18 bp, synthetic construct
  source 1..18: mol_type = other DNA
agggctgcagagtcttca

SEQ ID NO: 763 - DNA, 18 bp, synthetic construct
  source 1..18: mol_type = other DNA
tggggtcccgagggctgc

SEQ ID NO: 764 - DNA, 18 bp, synthetic construct
  source 1..18: mol_type = other DNA
accctccgagtggggtcc

SEQ ID NO: 765 - DNA, 18 bp, synthetic construct
  source 1..18: mol_type = other DNA
agcagagggcaccctccg

SEQ ID NO: 766 - DNA, 18 bp, synthetic construct
  source 1..18: mol_type = other DNA
gggaggcctgagcagagg

SEQ ID NO: 767 - DNA, 18 bp, synthetic construct
  source 1..18: mol_type = other DNA
ggaggtgctagggaggcc

SEQ ID NO: 768 - DNA, 18 bp, synthetic construct
  source 1..18: mol_type = other DNA
ttggttagggggaggtgc

SEQ ID NO: 769 - DNA, 18 bp, synthetic construct
  source 1..18: mol_type = other DNA
cagggagaatttggttag

SEQ ID NO: 770 - DNA, 18 bp, synthetic construct
  source 1..18: mol_type = other DNA
gaatggggtccagggaga

SEQ ID NO: 771 - DNA, 18 bp, synthetic construct
  source 1..18: mol_type = other DNA
ggggagctcagaatgggg

SEQ ID NO: 772 - DNA, 18 bp, synthetic construct
  source 1..18: mol_type = other DNA
ccatggtgatggggagct

SEQ ID NO: 773 - DNA, 18 bp, synthetic construct
  source 1..18: mol_type = other DNA
gccccacctcccatggtg

SEQ ID NO: 774 - DNA, 18 bp, synthetic construct
  source 1..18: mol_type = other DNA
ttagattgaggccccacc

SEQ ID NO: 775 - DNA, 18 bp, synthetic construct
  source 1..18: mol_type = other DNA
agggaaggccttagattg

SEQ ID NO: 776 - DNA, 18 bp, synthetic construct
  source 1..18: mol_type = other DNA
cccttctgacagggaagg

SEQ ID NO: 777 - DNA, 18 bp, synthetic construct
  source 1..18: mol_type = other DNA
tgccacaacccccttctg

SEQ ID NO: 778 - DNA, 18 bp, synthetic construct
  source 1..18: mol_type = other DNA
atgtggcttttgccacaa

SEQ ID NO: 779 - DNA, 18 bp, synthetic construct
  source 1..18: mol_type = other DNA
gcagcttgtaatgtggct

SEQ ID NO: 780 - DNA, 18 bp, synthetic construct
  source 1..18: mol_type = other DNA
ggaggggatggcagcttg

SEQ ID NO: 781 - DNA, 18 bp, synthetic construct
  source 1..18: mol_type = other DNA
actgaaacggggagggga

SEQ ID NO: 782 - DNA, 18 bp, synthetic construct
  source 1..18: mol_type = other DNA
cacagggtccactgaaac

SEQ ID NO: 783 - DNA, 18 bp, synthetic construct
  source 1..18: mol_type = other DNA
agcacctggccacagggt

SEQ ID NO: 784 - DNA, 18 bp, synthetic construct
  source 1..18: mol_type = other DNA
gatagggaaaagcacctg

SEQ ID NO: 785 - DNA, 18 bp, synthetic construct
  source 1..18: mol_type = other DNA
cacccctgtggataggga

SEQ ID NO: 786 - DNA, 18 bp, synthetic construct
  source 1..18: mol_type = other DNA
cacacacaaacacccctg

SEQ ID NO: 787 - DNA, 18 bp, synthetic construct
  source 1..18: mol_type = other DNA
cacacgcgcacacacaca

SEQ ID NO: 788 - DNA, 18 bp, synthetic construct
  source 1..18: mol_type = other DNA
tattgaaacgcacacgcg

SEQ ID NO: 789 - DNA, 18 bp, synthetic construct
  source 1..18: mol_type = other DNA
gtacaaactttattgaaa

SEQ ID NO: 790 - DNA, 18 bp, synthetic construct
  source 1..18: mol_type = other DNA
ttaagaaagtgtacaaac

SEQ ID NO: 791 - DNA, 18 bp, synthetic construct
  source 1..18: mol_type = other DNA
ttttttttttttaagaaa

SEQ ID NO: 792 - DNA, 18 bp, synthetic construct
  source 1..18: mol_type = other DNA
accctgtcttcacagcag

SEQ ID NO: 793 - DNA, 18 bp, synthetic construct
  source 1..18: mol_type = other DNA
agtgcaccctgtcttcac

SEQ ID NO: 794 - DNA, 18 bp, synthetic construct
  source 1..18: mol_type = other DNA
caggaagcctgagaaaaa

SEQ ID NO: 795 - DNA, 18 bp, synthetic construct
  source 1..18: mol_type = other DNA
gtgtgaaccaggtcgcag

SEQ ID NO: 796 - DNA, 18 bp, synthetic construct
  source 1..18: mol_type = other DNA
cagaaggcaccgtgcgga

SEQ ID NO: 797 - DNA, 18 bp, synthetic construct
  source 1..18: mol_type = other DNA
cgggtgtgaaccaggtcg

SEQ ID NO: 798 - DNA, 18 bp, synthetic construct
  source 1..18: mol_type = other DNA
acagcaggaagcctgaga

SEQ ID NO: 799 - DNA, 18 bp, synthetic construct
  source 1..18: mol_type = other DNA
tcttcacagcaggaagcc

SEQ ID NO: 800 - DNA, 18 bp, synthetic construct
  source 1..18: mol_type = other DNA
tgtgatgcagcgggtgtg

SEQ ID NO: 801 - DNA, 18 bp, synthetic construct
  source 1..18: mol_type = other DNA
tgcagcgggtgtgaacca

SEQ ID NO: 802 - DNA, 18 bp, synthetic construct
  source 1..18: mol_type = other DNA
acagcagtgcaccctgtc

SEQ ID NO: 803 - DNA, 18 bp, synthetic construct
  source 1..18: mol_type = other DNA
caccgtgcggacagcagt

SEQ ID NO: 804 - DNA, 18 bp, synthetic construct
  source 1..18: mol_type = other DNA
cacagcaggaagcctgag

SEQ ID NO: 805 - DNA, 18 bp, synthetic construct
  source 1..18: mol_type = other DNA
ctgtcttcacagcaggaa

SEQ ID NO: 806 - DNA, 18 bp, synthetic construct
  source 1..18: mol_type = other DNA
gtgatgcagcgggtgtga

SEQ ID NO: 807 - DNA, 18 bp, synthetic construct
  source 1..18: mol_type = other DNA
accgtgcggacagcagtg

SEQ ID NO: 808 - DNA, 18 bp, synthetic construct
  source 1..18: mol_type = other DNA
tcacagcaggaagcctga

SEQ ID NO: 809 - DNA, 18 bp, synthetic construct
  source 1..18: mol_type = other DNA
gaaggcaccgtgcggaca

SEQ ID NO: 810 - DNA, 18 bp, synthetic construct
  source 1..18: mol_type = other DNA
ccctgtcttcacagcagg

SEQ ID NO: 811 - DNA, 18 bp, synthetic construct
  source 1..18: mol_type = other DNA
tgtcttcacagcaggaag

SEQ ID NO: 812 - DNA, 18 bp, synthetic construct
  source 1..18: mol_type = other DNA
atgcagcgggtgtgaacc

SEQ ID NO: 813 - DNA, 18 bp, synthetic construct
  source 1..18: mol_type = other DNA
ggtgtgatgcagcgggtg

SEQ ID NO: 814 - DNA, 18 bp, synthetic construct
  source 1..18: mol_type = other DNA
ccaggtcgcagaaggcac

SEQ ID NO: 815 - DNA, 18 bp, synthetic construct
  source 1..18: mol_type = other DNA
accaggtcgcagaaggca

SEQ ID NO: 816 - DNA, 18 bp, synthetic construct
  source 1..18: mol_type = other DNA
gtgggtgtgatgcagcgg

SEQ ID NO: 817 - DNA, 18 bp, synthetic construct
  source 1..18: mol_type = other DNA
gtcttcacagcaggaagc

SEQ ID NO: 818 - DNA, 18 bp, synthetic construct
  source 1..18: mol_type = other DNA
cgtgcggacagcagtgca

SEQ ID NO: 819 - DNA, 18 bp, synthetic construct
  source 1..18: mol_type = other DNA
caccctgtcttcacagca

SEQ ID NO: 820 - DNA, 18 bp, synthetic construct
  source 1..18: mol_type = other DNA
cgcagaaggcaccgtgcg

SEQ ID NO: 821 - DNA, 18 bp, synthetic construct
  source 1..18: mol_type = other DNA
cgtgggtgtgatgcagcg

SEQ ID NO: 822 - DNA, 18 bp, synthetic construct
  source 1..18: mol_type = other DNA
agcgggtgtgaaccaggt

SEQ ID NO: 823 - DNA, 18 bp, synthetic construct
  source 1..18: mol_type = other DNA
cagtgcaccctgtcttca

SEQ ID NO: 824 - DNA, 18 bp, synthetic construct
  source 1..18: mol_type = other DNA
tcgcagaaggcaccgtgc

SEQ ID NO: 825 - DNA, 18 bp, synthetic construct
  source 1..18: mol_type = other DNA
ggcaccgtgcggacagca

SEQ ID NO: 826 - DNA, 18 bp, synthetic construct
  source 1..18: mol_type = other DNA
cagcaggaagcctgagaa

SEQ ID NO: 827 - DNA, 18 bp, synthetic construct
  source 1..18: mol_type = other DNA
aaccaggtcgcagaaggc

SEQ ID NO: 828 - DNA, 18 bp, synthetic construct
  source 1..18: mol_type = other DNA
gatgcagcgggtgtgaac

SEQ ID NO: 829 - DNA, 18 bp, synthetic construct
  source 1..18: mol_type = other DNA
gcagcgggtgtgaaccag

SEQ ID NO: 830 - DNA, 18 bp, synthetic construct
  source 1..18: mol_type = other DNA
gcgggtgtgaaccaggtc

SEQ ID NO: 831 - DNA, 18 bp, synthetic construct
  source 1..18: mol_type = other DNA
ttcacagcaggaagcctg

SEQ ID NO: 832 - DNA, 18 bp, synthetic construct
  source 1..18: mol_type = other DNA
gcaccgtgcggacagcag

SEQ ID NO: 833 - DNA, 18 bp, synthetic construct
  source 1..18: mol_type = other DNA
ggacagcagtgcaccctg

SEQ ID NO: 834 - DNA, 18 bp, synthetic construct
  source 1..18: mol_type = other DNA
gcagtgcaccctgtcttc

SEQ ID NO: 835 - DNA, 18 bp, synthetic construct
  source 1..18: mol_type = other DNA
aaggcaccgtgcggacag

SEQ ID NO: 836 - DNA, 18 bp, synthetic construct
  source 1..18: mol_type = other DNA
gcaggaagcctgagaaaa

SEQ ID NO: 837 - DNA, 18 bp, synthetic construct
  source 1..18: mol_type = other DNA
tgcggacagcagtgcacc

SEQ ID NO: 838 - DNA, 18 bp, synthetic construct
  source 1..18: mol_type = other DNA
gtgcaccctgtcttcaca

SEQ ID NO: 839 - DNA, 18 bp, synthetic construct
  source 1..18: mol_type = other DNA
gacagcagtgcaccctgt

SEQ ID NO: 840 - DNA, 18 bp, synthetic construct
  source 1..18: mol_type = other DNA
ggtcgcagaaggcaccgt

SEQ ID NO: 841 - DNA, 18 bp, synthetic construct
  source 1..18: mol_type = other DNA
tgaaccaggtcgcagaag

SEQ ID NO: 842 - DNA, 18 bp, synthetic construct
  source 1..18: mol_type = other DNA
gggtgtgaaccaggtcgc

SEQ ID NO: 843 - DNA, 18 bp, synthetic construct
  source 1..18: mol_type = other DNA
agaaggcaccgtgcggac

SEQ ID NO: 844 - DNA, 18 bp, synthetic construct
  source 1..18: mol_type = other DNA
tgggtgtgatgcagcggg

SEQ ID NO: 845 - DNA, 18 bp, synthetic construct
  source 1..18: mol_type = other DNA
tgtgaaccaggtcgcaga

SEQ ID NO: 846 - DNA, 18 bp, synthetic construct
  source 1..18: mol_type = other DNA
cagcagtgcaccctgtct

SEQ ID NO: 847 - DNA, 18 bp, synthetic construct
  source 1..18: mol_type = other DNA
gtgtgatgcagcgggtgt

SEQ ID NO: 848 - DNA, 18 bp, synthetic construct
  source 1..18: mol_type = other DNA
caggtcgcagaaggcacc

SEQ ID NO: 849 - DNA, 18 bp, synthetic construct
  source 1..18: mol_type = other DNA
gcggacagcagtgcaccc

SEQ ID NO: 850 - DNA, 18 bp, synthetic construct
  source 1..18: mol_type = other DNA
gtgcggacagcagtgcac

SEQ ID NO: 851 - DNA, 18 bp, synthetic construct
  source 1..18: mol_type = other DNA
gtgaaccaggtcgcagaa

SEQ ID NO: 852 - DNA, 18 bp, synthetic construct
  source 1..18: mol_type = other DNA
gtcgcagaaggcaccgtg

SEQ ID NO: 853 - DNA, 18 bp, synthetic construct
  source 1..18: mol_type = other DNA
agcaggaagcctgagaaa

SEQ ID NO: 854 - DNA, 18 bp, synthetic construct
  source 1..18: mol_type = other DNA
gcaccctgtcttcacagc

SEQ ID NO: 855 - DNA, 18 bp, synthetic construct
  source 1..18: mol_type = other DNA
  modified_base 1: mod_base = OTHER; note = 2'-MOE guanosine 3'-phosphorothioate
  modified_base 2: mod_base = OTHER; note = 2'-MOE thymidine 3'-phosphodiester
  modified_base 3..4: mod_base = OTHER; note = 2'-MOE 5-methylcytidine 3'-phosphorothioate
  modified_base 5: mod_base = OTHER; note = 2'-MOE adenosine 3'-phosphorothioate
  modified_base 6..8: mod_base = OTHER; note = 2'-MOE guanosine 3'-phosphorothioate
  modified_base 9: mod_base = OTHER; note = 2'-MOE adenosine 3'-phosphorothioate
  modified_base 10: mod_base = OTHER; note = 2'-MOE guanosine 3'-phosphodiester
  modified_base 11..12: mod_base = OTHER; note = 2'-MOE adenosine 3'-phosphorothioate
  modified_base 13..15: mod_base = OTHER; note = 2'-MOE thymidine 3'-phosphorothioate
  modified_base 16..17: mod_base = OTHER; note = 2'-MOE guanosine 3'-phosphorothioate
  modified_base 18: mod_base = OTHER; note = 2'-MOE thymidine
gtccagggagaatttggt

SEQ ID NO: 856 - DNA, 18 bp, synthetic construct
  source 1..18: mol_type = other DNA
  modified_base 1: mod_base = OTHER; note = 2'-MOE guanosine 3'-phosphorothioate
  modified_base 2: mod_base = OTHER; note = 2'-MOE thymidine 3'-phosphodiester
  modified_base 3..4: mod_base = OTHER; note = 2'-MOE guanosine 3'-phosphorothioate
  modified_base 5: mod_base = OTHER; note = 2'-MOE adenosine 3'-phosphorothioate
  modified_base 6: mod_base = OTHER; note = 2'-MOE thymidine 3'-phosphorothioate
  modified_base 7: mod_base = OTHER; note = 2'-MOE adenosine 3'-phosphorothioate
  modified_base 8..9: mod_base = OTHER; note = 2'-MOE guanosine 3'-phosphorothioate
  modified_base 10: mod_base = OTHER; note = 2'-MOE guanosine 3'-phosphodiester
  modified_base 11..14: mod_base = OTHER; note = 2'-MOE adenosine 3'-phosphorothioate
  modified_base 15: mod_base = OTHER; note = 2'-MOE guanosine 3'-phosphorothioate
  modified_base 16: mod_base = OTHER; note = 2'-MOE 5-methylcytidine 3'-phosphorothioate
  modified_base 17: mod_base = OTHER; note = 2'-MOE adenosine 3'-phosphorothioate
  modified_base 18: mod_base = OTHER; note = 2'-MOE 5-methylcytidine
gtggatagggaaaagcac

SEQ ID NO: 857 - DNA, 18 bp, synthetic construct
  source 1..18: mol_type = other DNA
  modified_base 1: mod_base = OTHER; note = 2'-MOE guanosine 3'-phosphorothioate
  modified_base 2: mod_base = OTHER; note = 2'-MOE guanosine 3'-phosphodiester
  modified_base 3: mod_base = OTHER; note = 2'-MOE guanosine 3'-phosphorothioate
  modified_base 4: mod_base = OTHER; note = 2'-MOE thymidine 3'-phosphorothioate
  modified_base 5..6: mod_base = OTHER; note = 2'-MOE 5-methylcytidine 3'-phosphorothioate
  modified_base 7: mod_base = OTHER; note = 2'-MOE adenosine 3'-phosphorothioate
  modified_base 8: mod_base = OTHER; note = 2'-MOE 5-methylcytidine 3'-phosphorothioate
  modified_base 9: mod_base = OTHER; note = 2'-MOE thymidine 3'-phosphorothioate
  modified_base 10: mod_base = OTHER; note = 2'-MOE guanosine 3'-phosphodiester
  modified_base 11..13: mod_base = OTHER; note = 2'-MOE adenosine 3'-phosphorothioate
  modified_base 14: mod_base = OTHER; note = 2'-MOE 5-methylcytidine 3'-phosphorothioate
  modified_base 15..17: mod_base = OTHER; note = 2'-MOE guanosine 3'-phosphorothioate
  modified_base 18: mod_base = OTHER; note = 2'-MOE guanosine
gggtccactgaaacgggg

SEQ ID NO: 858 - DNA, 18 bp, synthetic construct
  source 1..18: mol_type = other DNA
  modified_base 1: mod_base = OTHER; note = 2'-MOE guanosine 3'-phosphorothioate
  modified_base 2: mod_base = OTHER; note = 2'-MOE guanosine 3'-phosphodiester
  modified_base 3: mod_base = OTHER; note = 2'-MOE adenosine 3'-phosphorothioate
  modified_base 4: mod_base = OTHER; note = 2'-MOE thymidine 3'-phosphorothioate
  modified_base 5: mod_base = OTHER; note = 2'-MOE adenosine 3'-phosphorothioate
  modified_base 6..8: mod_base = OTHER; note = 2'-MOE guanosine 3'-phosphorothioate
  modified_base 9: mod_base = OTHER; note = 2'-MOE adenosine 3'-phosphorothioate
  modified_base 10: mod_base = OTHER; note = 2'-MOE adenosine 3'-phosphodiester
  modified_base 11..12: mod_base = OTHER; note = 2'-MOE adenosine 3'-phosphorothioate
  modified_base 13: mod_base = OTHER; note = 2'-MOE guanosine 3'-phosphorothioate
  modified_base 14: mod_base = OTHER; note = 2'-MOE 5-methylcytidine 3'-phosphorothioate
  modified_base 15: mod_base = OTHER; note = 2'-MOE adenosine 3'-phosphorothioate
  modified_base 16..17: mod_base = OTHER; note = 2'-MOE 5-methylcytidine 3'-phosphorothioate
  modified_base 18: mod_base = OTHER; note = 2'-MOE thymidine
ggatagggaaaagcacct

SEQ ID NO: 859 - DNA, 18 bp, synthetic construct
  source 1..18: mol_type = other DNA
  modified_base 1: mod_base = OTHER; note = 2'-MOE guanosine 3'-phosphorothioate
  modified_base 2: mod_base = OTHER; note = 2'-MOE guanosine 3'-phosphodiester
  modified_base 3: mod_base = OTHER; note = 2'-MOE thymidine 3'-phosphorothioate
  modified_base 4..5: mod_base = OTHER; note = 2'-MOE 5-methylcytidine 3'-phosphorothioate
  modified_base 6: mod_base = OTHER; note = 2'-MOE adenosine 3'-phosphorothioate
  modified_base 7..9: mod_base = OTHER; note = 2'-MOE guanosine 3'-phosphorothioate
  modified_base 10: mod_base = OTHER; note = 2'-MOE adenosine 3'-phosphodiester
  modified_base 11: mod_base = OTHER; note = 2'-MOE guanosine 3'-phosphorothioate
  modified_base 12..13: mod_base = OTHER; note = 2'-MOE adenosine 3'-phosphorothioate
  modified_base 14..16: mod_base = OTHER; note = 2'-MOE thymidine 3'-phosphorothioate
  modified_base 17: mod_base = OTHER; note = 2'-MOE guanosine 3'-phosphorothioate
  modified_base 18: mod_base = OTHER; note = 2'-MOE guanosine
ggtccagggagaatttgg

SEQ ID NO: 860 - DNA, 18 bp, synthetic construct
  source 1..18: mol_type = other DNA
  modified_base 1: mod_base = OTHER; note = 2'-MOE adenosine 3'-phosphorothioate
  modified_base 2: mod_base = OTHER; note = 2'-MOE adenosine 3'-phosphodiester
  modified_base 3: mod_base = OTHER; note = 2'-MOE adenosine 3'-phosphorothioate
  modified_base 4: mod_base = OTHER; note = 2'-MOE 5-methylcytidine 3'-phosphorothioate
  modified_base 5..8: mod_base = OTHER; note = 2'-MOE guanosine 3'-phosphorothioate
  modified_base 9: mod_base = OTHER; note = 2'-MOE adenosine 3'-phosphorothioate
  modified_base 10: mod_base = OTHER; note = 2'-MOE guanosine 3'-phosphodiester
  modified_base 11..13: mod_base = OTHER; note = 2'-MOE guanosine 3'-phosphorothioate
  modified_base 14: mod_base = OTHER; note = 2'-MOE adenosine 3'-phosphorothioate
  modified_base 15: mod_base = OTHER; note = 2'-MOE thymidine 3'-phosphorothioate
  modified_base 16..17: mod_base = OTHER; note = 2'-MOE guanosine 3'-phosphorothioate
  modified_base 18: mod_base = OTHER; note = 2'-MOE 5-methylcytidine
aaacggggaggggatggc

SEQ ID NO: 861 - DNA, 18 bp, synthetic construct
  source 1..18: mol_type = other DNA
  modified_base 1: mod_base = OTHER; note = 2'-MOE guanosine 3'-phosphorothioate
  modified_base 2: mod_base = OTHER; note = 2'-MOE adenosine 3'-phosphodiester
  modified_base 3..4: mod_base = OTHER; note = 2'-MOE adenosine 3'-phosphorothioate
  modified_base 5: mod_base = OTHER; note = 2'-MOE 5-methylcytidine 3'-phosphorothioate
  modified_base 6..9: mod_base = OTHER; note = 2'-MOE guanosine 3'-phosphorothioate
  modified_base 10: mod_base = OTHER; note = 2'-MOE adenosine 3'-phosphodiester
  modified_base 11..14: mod_base = OTHER; note = 2'-MOE guanosine 3'-phosphorothioate
  modified_base 15: mod_base = OTHER; note = 2'-MOE adenosine 3'-phosphorothioate
  modified_base 16: mod_base = OTHER; note = 2'-MOE thymidine 3'-phosphorothioate
  modified_base 17: mod_base = OTHER; note = 2'-MOE guanosine 3'-phosphorothioate
  modified_base 18: mod_base = OTHER; note = 2'-MOE guanosine
gaaacggggaggggatgg

SEQ ID NO: 862 - DNA, 18 bp, synthetic construct
  source 1..18: mol_type = other DNA
  modified_base 1: mod_base = OTHER; note = 2'-MOE thymidine 3'-phosphorothioate
  modified_base 2: mod_base = OTHER; note = 2'-MOE guanosine 3'-phosphodiester
  modified_base 3..5: mod_base = OTHER; note = 2'-MOE adenosine 3'-phosphorothioate
  modified_base 6: mod_base = OTHER; note = 2'-MOE 5-methylcytidine 3'-phosphorothioate
  modified_base 7..9: mod_base = OTHER; note = 2'-MOE guanosine 3'-phosphorothioate
  modified_base 10: mod_base = OTHER; note = 2'-MOE guanosine 3'-phosphodiester
  modified_base 11: mod_base = OTHER; note = 2'-MOE adenosine 3'-phosphorothioate
  modified_base 12..15: mod_base = OTHER; note = 2'-MOE guanosine 3'-phosphorothioate
  modified_base 16: mod_base = OTHER; note = 2'-MOE adenosine 3'-phosphorothioate
  modified_base 17: mod_base = OTHER; note = 2'-MOE thymidine 3'-phosphorothioate
  modified_base 18: mod_base = OTHER; note = 2'-MOE guanosine
tgaaacggggaggggatg

SEQ ID NO: 863 - DNA, 18 bp, synthetic construct
  source 1..18: mol_type = other DNA
  modified_base 1: mod_base = OTHER; note = 2'-MOE 5-methylcytidine 3'-phosphorothioate
  modified_base 2: mod_base = OTHER; note = 2'-MOE thymidine 3'-phosphodiester
  modified_base 3: mod_base = OTHER; note = 2'-MOE guanosine 3'-phosphorothioate
  modified_base 4..6: mod_base = OTHER; note = 2'-MOE adenosine 3'-phosphorothioate
  modified_base 7: mod_base = OTHER; note = 2'-MOE 5-methylcytidine 3'-phosphorothioate
  modified_base 8..9: mod_base = OTHER; note = 2'-MOE guanosine 3'-phosphorothioate
  modified_base 10: mod_base = OTHER; note = 2'-MOE guanosine 3'-phosphodiester
  modified_base 11: mod_base = OTHER; note = 2'-MOE guanosine 3'-phosphorothioate
  modified_base 12: mod_base = OTHER; note = 2'-MOE adenosine 3'-phosphorothioate
  modified_base 13..16: mod_base = OTHER; note = 2'-MOE guanosine 3'-phosphorothioate
  modified_base 17: mod_base = OTHER; note = 2'-MOE adenosine 3'-phosphorothioate
  modified_base 18: mod_base = OTHER; note = 2'-MOE thymidine
ctgaaacggggaggggat

SEQ ID NO: 864 - DNA, 18 bp, synthetic construct
  source 1..18: mol_type = other DNA
  modified_base 1: mod_base = OTHER; note = 2'-MOE adenosine 3'-phosphorothioate
  modified_base 2: mod_base = OTHER; note = 2'-MOE 5-methylcytidine 3'-phosphodiester
  modified_base 3: mod_base = OTHER; note = 2'-MOE thymidine 3'-phosphorothioate
  modified_base 4: mod_base = OTHER; note = 2'-MOE guanosine 3'-phosphorothioate
  modified_base 5..7: mod_base = OTHER; note = 2'-MOE adenosine 3'-phosphorothioate
  modified_base 8: mod_base = OTHER; note = 2'-MOE 5-methylcytidine 3'-phosphorothioate
  modified_base 9: mod_base = OTHER; note = 2'-MOE guanosine 3'-phosphorothioate
  modified_base 10: mod_base = OTHER; note = 2'-MOE guanosine 3'-phosphodiester
  modified_base 11..12: mod_base = OTHER; note = 2'-MOE guanosine 3'-phosphorothioate
  modified_base 13: mod_base = OTHER; note = 2'-MOE adenosine 3'-phosphorothioate
  modified_base 14..17: mod_base = OTHER; note = 2'-MOE guanosine 3'-phosphorothioate
  modified_base 18: mod_base = OTHER; note = 2'-MOE adenosine
actgaaacggggagggga

SEQ ID NO: 865 - DNA, 18 bp, synthetic construct
  source 1..18: mol_type = other DNA
  modified_base 1: mod_base = OTHER; note = 2'-MOE 5-methylcytidine 3'-phosphorothioate
  modified_base 2: mod_base = OTHER; note = 2'-MOE adenosine 3'-phosphodiester
  modified_base 3: mod_base = OTHER; note = 2'-MOE 5-methylcytidine 3'-phosphorothioate
  modified_base 4: mod_base = OTHER; note = 2'-MOE thymidine 3'-phosphorothioate
  modified_base 5: mod_base = OTHER; note = 2'-MOE guanosine 3'-phosphorothioate
  modified_base 6..8: mod_base = OTHER; note = 2'-MOE adenosine 3'-phosphorothioate
  modified_base 9: mod_base = OTHER; note = 2'-MOE 5-methylcytidine 3'-phosphorothioate
  modified_base 10: mod_base = OTHER; note = 2'-MOE guanosine 3'-phosphodiester
  modified_base 11..13: mod_base = OTHER; note = 2'-MOE guanosine 3'-phosphorothioate
  modified_base 14: mod_base = OTHER; note = 2'-MOE adenosine 3'-phosphorothioate
  modified_base 15..17: mod_base = OTHER; note = 2'-MOE guanosine 3'-phosphorothioate
  modified_base 18: mod_base = OTHER; note = 2'-MOE guanosine
cactgaaacggggagggg

SEQ ID NO: 866 - DNA, 18 bp, synthetic construct
  source 1..18: mol_type = other DNA
  modified_base 1: mod_base = OTHER; note = 2'-MOE guanosine 3'-phosphorothioate
  modified_base 2: mod_base = OTHER; note = 2'-MOE guanosine 3'-phosphodiester
  modified_base 3: mod_base = OTHER; note = 2'-MOE thymidine 3'-phosphorothioate
  modified_base 4..5: mod_base = OTHER; note = 2'-MOE 5-methylcytidine 3'-phosphorothioate
  modified_base 6: mod_base = OTHER; note = 2'-MOE adenosine 3'-phosphorothioate
  modified_base 7: mod_base = OTHER; note = 2'-MOE 5-methylcytidine 3'-phosphorothioate
  modified_base 8: mod_base = OTHER; note = 2'-MOE thymidine 3'-phosphorothioate
  modified_base 9: mod_base = OTHER; note = 2'-MOE guanosine 3'-phosphorothioate
  modified_base 10: mod_base = OTHER; note = 2'-MOE adenosine 3'-phosphodiester
  modified_base 11..12: mod_base = OTHER; note = 2'-MOE adenosine 3'-phosphorothioate
  modified_base 13: mod_base = OTHER; note = 2'-MOE 5-methylcytidine 3'-phosphorothioate
  modified_base 14..17: mod_base = OTHER; note = 2'-MOE guanosine 3'-phosphorothioate
  modified_base 18: mod_base = OTHER; note = 2'-MOE adenosine
ggtccactgaaacgggga

(19) United States
(12) Patent Application Publication
(10) Pub. No.: US 2025/0109396 A1
(43) Pub. Date: Apr. 3, 2025

(54) COMPOUNDS AND METHODS FOR MODULATING PROGRANULIN EXPRESSION

(71) Applicants: IONIS PHARMACEUTICALS, INC., Carlsbad, CA (US); SAINT LOUIS UNIVERSITY, St. Louis, MO (US)

(72) Inventors: Huynh-Hoa BUI, Carlsbad, CA (US); Paymaan JAFAR-NEJAD, Carlsbad, CA (US); Andrew Tuan Duc NGUYEN, Webster Groves, MO (US)

(21) Appl. No.: 18/710,928

(22) PCT Filed: Nov. 18, 2022
(86) PCT No.: PCT/US2022/080113

§ 371 Date: May 16, 2024

Related U.S. Application Data
(60) Provisional application No. 63/281,057, filed on Nov. 18, 2021.

(51) Int. Cl. C12N 15/113

(52) U.S. Cl. CPC C12N 15/113; C12N 2310/11; C12N 2310/315; C12N 2310/3341; C12N 2310/351

(57) ABSTRACT

Provided are oligomeric compounds, methods, and pharmaceutical compositions for modulating expression of progranulin RNA, or modulating expression of progranulin protein in a cell or subject. Such compounds, methods, and pharmaceutical compositions are useful to ameliorate at least one symptom or hallmark of a neurological disease or disorder. Such neurological diseases or disorders include those associated with insufficient expression of progranulin, including frontotemporal dementia, frontotemporal lobar degeneration, Alzheimer's disease, amyotrophic lateral sclerosis and neuronal ceroid lipofuscinosis. Such symptoms or hallmarks include deterioration in behavior and personality, language impairment, disturbances or alterations in muscle or motor functions, memory loss, cognitive dysfunction, tremor, seizures, or dizziness.

### STATEMENT OF GOVERNMENT SUPPORT

This invention was made with government support under AG064069 awarded by National Institute of Health/National Institute on Aging; and UL 1TR002345 awarded by National Institute of Health/National Center for Advancing Translational Sciences. The government has certain rights in the invention.

### SEQUENCE LISTING

The present application is being filed along with a Sequence Listing in electronic format. The Sequence Listing is provided as a file entitled BIOL0452WOSEQ.xml, created on Nov. 17, 2022, which is 839 KB in size. The information in the electronic format of the sequence listing is incorporated herein by reference in its entirety.

### FIELD

Provided are oligomeric compounds, methods, and pharmaceutical compositions for modulating expression of progranulin RNA, or modulating expression of progranulin protein in a cell or subject. Such compounds, methods, and pharmaceutical compositions are useful to ameliorate at least one symptom or hallmark of a neurological disease or disorder. Such neurological diseases or disorders include those associated with insufficient expression of progranulin, including frontotemporal dementia, frontotemporal lobar degeneration, Alzheimer's disease, amyotrophic lateral sclerosis, and neuronal ceroid lipofuscinosis. Such symptoms or hallmarks include deterioration in behavior and personality, language impairment, disturbances or alterations in muscle or motor functions, memory loss, cognitive dysfunction, tremor, seizures, or dizziness.

### BACKGROUND

The human gene GRN encodes human progranulin protein. Mutations in GRN lead to neurological diseases and disorders, including frontotemporal dementia (FTD), frontotemporal lobar degeneration (FTLD) neuronal ceroid lipofuscinosis (NCL), lysosomal storage diseases, Alzheimer's disease (AD), and amyotrophic lateral sclerosis (ALS). These diseases are associated with GRN and/or progranulin haploinsufficiency. FTD refers to a group of disorders caused by progressive nerve cell loss in the brain's frontal lobes or temporal lobes. Nerve cell damage caused by FTD leads to loss of function in the frontal lobes or temporal lobes, and is associated with deterioration in behavior and personality, language impairment, disturbances or alterations in muscle or motor functions, memory loss, cognitive dysfunction, tremor, seizures, or dizziness. FTD is associated with TDP-43 proteinopathies.

Currently there remains a need for therapies to treat neurological diseases and disorders associated with an insufficient amount of progranulin. It is therefore an object herein to provide oligomeric compounds, methods, and pharmaceutical compositions for the treatment of such diseases or disorders.

### BRIEF DESCRIPTION OF THE DRAWINGS

FIG. 1A, FIG. 1B, FIG. 1C, FIG. 1D, and FIG. 1E show human progranulin levels in H4 cell lysates or conditioned media following treatment with modified oligonucleotides. FIG. 1A shows the results of an ELISA assay in cell lysates, following treatment with (from left to right): control scrambled ASO, water, Compounds 1212637, 1212638, 1212639, 1212641, 1212642, 1212646, 1212647, 1212648, 1212660, 1212661, 1212664, 1212665, 1212667, 1212671, 1212672, 1212673, 1212674, 1212675, 1212676, 1212684. FIG. 1B is a Western blot of H4 cell lysates, following treatment of the cells with (from left to right) water, control scrambled ASO, Compounds 1212637, 1212638, 1212639, 1212640, 1212641, 1212642, 1212646, 1212647, 1212672, 1212674 1212675, 1212684. FIG. 1C is a Western blot of H4 cell lysates, following treatment of the cells with (from left to right) water, control scrambled ASO, Compounds 1212646, 1212647, 1212660, 1212661, 1212664, 1212665, 1212667, 1212672, 1212674. FIG. 1D is a Western blot of H4 cell lysates following treatment of the cells with (from left to right) water, control scrambled ASO, Compound 1212676. FIG. 1E is a Western blot of H4 cell lysates (top panel) or conditioned media (bottom panel), following treatment of the cells with (from left to right) water, control scrambled ASO, Compounds 1212637, 1212638, 1212639, 1212640, 1212641, 1212642.

FIG. 2A, FIG. 2B, FIG. 2C, FIG. 2D, FIG. 2E, and FIG. 2F are dose response curves of progranulin levels, presented as percent of GRN level in untreated cells as measured by ELISA, following treatment of H4 cells with either control scrambled ASO or Compounds 1212637, 1212638, 1212641, 1212646, 1212647, 1212672, respectively.

FIG. 3 is a Western blot of iPSC-derived neuron cell lysates, following treatment with control scrambled ASO or Compounds 1212638, 1212641, 1212646, 1212647, and 1212672, at the indicated concentrations (μM).

FIG. 4A and FIG. 4B show the results of ELISA assays of progranulin levels (ng/mg protein) in the cortex of male (FIG. 4A) and female (FIG. 4B) following administration of modified oligonucleotides. GRN levels in treated transgenic mice homozygous for human GRN are compared to GRN levels in saline treated transgenic GRN mice and to GRN levels in untreated non-transgenic mice. FIG. 4A (left to right): untreated non-transgenic mice, transgenic GRN mice: saline, control scrambled ASO, Compounds 1557990, 1557993, 1212647, 1557987. FIG. 4B (left to right) transgenic GRN mice: saline, control scrambled ASO, Compounds 1212640, 1557993, 1557994, 1212647, 1557987. FIG. 4C is a Western blot of progranulin in the cortex, thalamus, or hippocampus of untreated non-transgenic mice, or in transgenic GRN mice treated with saline control or Compound 1557993.

### SUMMARY

Provided herein are compounds, methods, and pharmaceutical compositions for modulating the amount of progranulin RNA and/or modulating the amount of progranulin protein in a cell or a subject. In certain embodiments, compounds useful for modulating the amount of progranulin RNA and/or progranulin protein are oligomeric compounds. In certain embodiments, oligomeric compounds increase the amount of progranulin RNA in a cell. In certain embodiments, oligomeric compounds increase the amount of progranulin protein in a cell. In certain embodiments, the oligomeric compound comprises a modified oligonucleotide. In certain embodiments, the subject has a neurological disease or disorder. In certain embodiments, the subject has a neurological disease or disorder associated with an insufficient amount of progranulin protein. In certain embodiments, the subject has FTD. In certain embodiments the subject has FTLD. In certain embodiments, the subject has NCL. In certain embodiments, the subject has a TDP-43 proteinopathy. In certain embodiments, the subject has a lysosomal storage disorder. In some embodiments, the subject has Alzheimer's disease (AD). In certain embodiments, the subject has amyotrophic lateral sclerosis (ALS).

Also provided are methods useful for ameliorating at least one symptom or hallmark of a neurological disease or disorder. In certain embodiments, the neurological disease or disorder is associated with an insufficient amount of progranulin protein. In certain embodiments, the neurological disease or disorder is FTD. In certain embodiments, the neurological disease or disorder is FTLD. In certain embodiments, the neurological disease or disorder is NCL. In certain embodiments, the neurological disease or disorder is a TDP-43 proteinopathy. In certain embodiments, the disease or disorder is a lysosomal storage disorder. In certain embodiments, the disease or disorder is ALS. In certain embodiments, the disease or disorder is AD. In certain embodiments, symptoms or hallmarks include deterioration in behavior and personality, language impairment, disturbances or alterations in muscle or motor functions, memory loss, cognitive dysfunction, tremor, seizures, or dizziness.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, and GenBank, ENSEMBL, and NCBI reference sequence records are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′-O(CH₂)₂OCH₃ group in place of the 2′—OH group of a ribosyl sugar moiety. A “2′-MOE sugar moiety” or a “2′-O-methoxyethyl sugar moiety” or a “2′-MOE modified sugar moiety” means a sugar moiety with a 2′-O(CH₂)₂OCH₃ group in place of the 2′—OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “2′-NMA” means a —O—CH₂—C(═O)—NH—CH₃ group in place of the 2′—OH group of a ribosyl sugar moiety. A “2′-NMA sugar moiety” or a “2′-NMA modified sugar moiety” is a sugar moiety with a 2′-O—CH₂—C(═O)—NH—CH₃ group in place of the 2′—OH group of a ribosyl sugar moiety. Unless otherwise indicated, a 2′-NMA sugar moiety is in the β-D configuration. “NMA” means O—N-methyl acetamide.

As used herein, “2′-NMA nucleoside” means a nucleoside comprising a 2′-NMA sugar moiety.

As used herein, “2′-OMe” means a 2′-OCH₃ group in place of the 2′—OH group of a ribosyl sugar moiety. A “2′-OMe sugar moiety” or a “2′-OMe modified sugar moiety” is a sugar moiety with a 2′-OCH₃ group in place of the 2′—OH group of a ribosyl sugar moiety. Unless otherwise indicated, a 2′-OMe sugar moiety is in the β-D configuration. “OMe” means O-methyl.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position.

A 5-methyl cytosine is a modified nucleobase.

As used herein, “administering” means providing a pharmaceutical agent to a subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark, or the delayed onset or slowing of progression in the severity or frequency of a symptom or hallmark. In certain embodiments, the symptom or hallmark is deterioration in behavior and personality, language impairment, disturbances or alterations in muscle or motor functions, memory loss, cognitive dysfunction, tremor, seizures, or dizziness.

As used herein, “antisense activity” means any detectable and/or measurable change attributable to the hybridization of an antisense compound to its target nucleic acid. In certain embodiments, antisense activity is an increase in the amount or expression of a target nucleic acid or protein encoded by such target nucleic acid compared to amount or expression of the target nucleic acid or target protein in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group. An “antisense compound” may also be an oligomeric duplex capable of achieving at least one antisense activity.

As used herein, “antisense oligonucleotide” means an oligonucleotide, including the oligonucleotide portion of an antisense compound, that is capable of hybridizing to a target nucleic acid and is capable of at least one antisense activity.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl moiety is a ribosyl moiety. In certain embodiments, the bicyclic sugar moiety does not comprise a furanosyl moiety.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) of cerebrospinal fluid and is biocompatible with CSF.

As used herein, “cEt” means a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. A “cEt sugar moiety” is a bicyclic sugar moiety with a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. “cEt” means constrained ethyl.

As used herein, “cEt nucleoside” means a nucleoside comprising a cEt sugar moiety.

As used herein, “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are modified oligonucleotides. In certain embodiments, the molecules are oligomeric compounds comprising modified oligonucleotides. In certain embodiments, the chiral center is at the phosphorous atom of a phosphorothioate internucleoside linkage. As used herein, “chirally controlled” in reference to an internucleoside linkage means chirality at that linkage is enriched for a particular stereochemical configuration.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G), and hypoxanthine (of inosine (I)) and cytosine (C), 5-methyl cytosine (^{m}C), uracil (U), or adenine (A). Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or target nucleic acid at each nucleobase of the shorter of the two oligonucleotides, or at each nucleoside if the oligonucleotides are the same length.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a covalently bound group of atoms that modifies one or more pharmacological properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “hotspot region” is a range of nucleobases on a target nucleic acid that is amenable to an oligomeric compound-mediated increase in the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “inosine” (I) or 9-β-D-Ribosylhypoxanthine means a nucleoside that contains a hypoxanthine nucleobase.

As used herein, “internucleoside linkage” is the covalent linkage between adjacent nucleosides in an oligonucleotide. As used herein, “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “modified nucleoside” means a nucleoside comprising a modified nucleobase and/or a modified sugar moiety.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.” As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutical composition” means a mixture of substances suitable for administering to a subject. For example, a pharmaceutical composition may comprise an oligomeric compound and a sterile aqueous solution.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension, and lozenges for the oral ingestion by a subject. In certain embodiments, a pharmaceutically acceptable diluent is sterile water, sterile saline, sterile buffer solution, or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “RNA” means an RNA transcript and includes pre-mRNA and mature mRNA unless otherwise specified.

As used herein “progranulin RNA” means an RNA transcript encoded by the progranulin gene, GRN, and includes pre-mRNA and mRNA unless otherwise specified. Progranulin nucleic acid and GRN nucleic acid are used interchangeably and refer to nucleic acid that encodes the progranulin protein. As used herein, “progranulin nucleic acid” and “GRN nucleic acid” are interchangeable and include progranulin DNA and progranulin RNA unless otherwise specified.

As used herein, “steric-blocking agent” means an antisense agent that acts, at least in part, due to directly binding to a target nucleic acid, thus blocking the interaction of the target nucleic acid with other nucleic acids or proteins.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, a stereorandom chiral center is a stereorandom phosphorothioate internucleoside linkage.

As used herein, “subject” means a human or non-human animal.

As used herein, “sugar moiety” means an unmodified sugar moiety or a modified sugar moiety. As used herein, “unmodified sugar moiety” means a 2′-OH(H) β-D ribosyl sugar moiety, as found in RNA (an “unmodified RNA sugar moiety”), or a 2′-H(H) β-D deoxyribosyl sugar moiety, as found in DNA (an “unmodified DNA sugar moiety”).

Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, ““sugar surrogate”” means a modified sugar moiety having other than a furanosyl moiety that can link a nucleobase to another group, such as an internucleoside linkage, conjugate group, or terminal group in an oligonucleotide, but which is not a furanosyl sugar moiety or a bicyclic sugar moiety. Modified nucleosides comprising sugar surrogates can be incorporated into one or more positions within an oligonucleotide and such oligonucleotides are capable of hybridizing to complementary oligomeric compounds or target nucleic acids. Examples of sugar surrogates include GNA (glycol nucleic acid), FHNA (fluoro hexitol nucleic acid), morpholino, and other structures described herein and known in the art. As used herein, “standard in vitro assay” means the assay described in Example 1 and reasonable variations thereof.

As used herein, “symptom or hallmark” means any physical feature or test result that indicates the existence or extent of a disease or disorder. In certain embodiments, a symptom is apparent to a subject or to a medical professional examining or testing the subject. In certain embodiments, a hallmark is apparent upon invasive diagnostic testing, including, but not limited to, post-mortem tests. In certain embodiments, a hallmark is apparent on a brain MRI scan.

As used herein, “target nucleic acid” and “target RNA” mean a nucleic acid that an antisense oligomeric or an oligomeric compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom or hallmark of a disease or disorder.

As used herein, “treating” means improving a subject's disease or disorder by administering an oligomeric compound described herein. In certain embodiments, treating a subject improves a symptom or hallmark relative to the same symptom in the absence of the treatment. In certain embodiments, treatment reduces in the severity or frequency of a symptom or hallmark, or delays the onset of a symptom or hallmark, slows the progression of a symptom or hallmark, or slows the severity or frequency of a symptom or hallmark.

### CERTAIN EMBODIMENTS

The present disclosure provides the following non-limiting numbered embodiments:

Embodiment 1. An oligomeric compound comprising a modified oligonucleotide consisting of 12 to 50 linked nucleosides, wherein the nucleobase sequence of the modified oligonucleotide is at least 80% complementary to an equal length portion of a progranulin nucleic acid, and wherein the modified oligonucleotide has at least one modification selected from a modified sugar moiety and a modified internucleoside linkage.
Embodiment 2. The oligomeric compound of embodiment 1, wherein the progranulin nucleic acid has the nucleobase sequence of SEQ ID NO: 1 or SEQ ID NO: 2.
Embodiment 3. An oligomeric compound comprising a modified oligonucleotide consisting of 18, 19, or 20 linked nucleosides, wherein the nucleobase sequence of the modified oligonucleotide comprises at least 16, at least 17, or 18 contiguous nucleobases of any of the nucleobase sequences of SEQ ID NOs:12-854, and wherein the modified oligonucleotide comprises at least one modification selected from a modified sugar moiety and a modified internucleoside linkage.
Embodiment 4. The oligomeric compound of any of embodiments 1-3, wherein the nucleobase sequence of the modified oligonucleotide comprises at least 16, at least 17, or 18 contiguous nucleobases of any of the nucleobase sequences of SEQ ID NOs: 34, 268, 345, 424, 501, 579, 657, 735, 800, 801, 806, 812, 813, 816, 821, 828, 844, and 847.
Embodiment 5. An oligomeric compound comprising a modified oligonucleotide consisting of 18 linked nucleosides, wherein the nucleobase sequence of the modified oligonucleotide consists of the nucleobase sequence of any of SEQ ID NOs: 12-854, and wherein the modified oligonucleotide comprises at least one modification selected from a modified sugar moiety and a modified internucleoside linkage.
Embodiment 6. The oligomeric compound of embodiment 5, wherein the nucleobase sequence of the modified oligonucleotide consists of 18 contiguous nucleobases of any of the nucleobase sequences of SEQ ID NOs: 34, 268, 345, 424, 501, 579, 657, 735, 800, 801, 806, 812, 813, 816, 821, 828, 844, and 847.
Embodiment 7. An oligomeric compound comprising a modified oligonucleotide consisting of 12 to 50 linked nucleosides, wherein the nucleobase sequence of the modified oligonucleotide comprises at least 8, at least 9, at least 10, at least 11, at least 12, at least 13, at least 14, at least 15, at least 16, at least 17, or 18 contiguous nucleobases complementary to an equal length portion within nucleobases 8497-8552 of SEQ ID NO: 1.
Embodiment 8. The oligomeric compound of embodiment 7, wherein the nucleobase sequence of the modified oligonucleotide consists of the nucleobase sequence of any of SEQ ID NOs: 34, 268, 345, 424, 501, 579, 657, 735, 800, 801, 806, 812, 813, 816, 821, 828, 844, and 847.
Embodiment 9. The oligomeric compound of any of embodiments 1-8, wherein the nucleobase sequence of the modified oligonucleotide is at least 85%, at least 90%, at least 95%, or is 100% complementary to an equal length portion within the nucleobase sequence of any of SEQ ID NO: 1 or SEQ ID NO: 2 when measured across the entire nucleobase sequence of the modified oligonucleotide.
Embodiment 10. The oligomeric compound of any of embodiments 1-9, wherein at least one nucleoside of the modified oligonucleotide comprises a modified sugar moiety.
Embodiment 11. The oligomeric compound of embodiment 10, wherein the modified sugar moiety comprises a bicyclic sugar moiety.
Embodiment 12. The oligomeric compound of embodiment 11, wherein the bicyclic sugar moiety comprises a 4′-2′ bridge selected from —CH₂—O— and —CH(CH₃)—O—.
Embodiment 13. The oligomeric compound of any of embodiments 10-12, wherein the modified sugar moiety comprises a non-bicyclic modified sugar moiety.
Embodiment 14. The oligomeric compound of embodiment 13, wherein the non-bicyclic modified sugar moiety is a 2′-MOE modified sugar moiety, a 2′-NMA modified sugar moiety, a 2′-OMe modified sugar moiety, or a 2′-F modified sugar moiety.
Embodiment 15. The oligomeric compound of embodiment 10, wherein the modified sugar moiety is a sugar surrogate.
Embodiment 16. The oligomeric compound of embodiment 15, wherein the sugar surrogate is a morpholino, modified morpholino, PNA, THP, or F-HNA.
Embodiment 17. The oligomeric compound of embodiment 10, wherein each nucleoside of the modified oligonucleotide comprises a modified sugar moiety.
Embodiment 18. The oligomeric compound of embodiment 17, wherein each modified sugar moiety is a 2′-MOE modified sugar moiety, a 2′-NMA modified sugar moiety, a 2′-OMe modified sugar moiety, or a 2′-F modified sugar moiety.
Embodiment 19. The oligomeric compound of embodiment 17, wherein each modified sugar moiety is a 2′-MOE modified sugar moiety.
Embodiment 20. The oligomeric compound of embodiment 17, wherein each modified sugar moiety is a sugar surrogate.
Embodiment 21. The oligomeric compound of embodiment 20, wherein each modified sugar moiety is a morpholino, modified morpholino, PNA, THP, or F-HNA.
Embodiment 22. The oligomeric compound of any of embodiments 1-21, wherein the modified oligonucleotide comprises at least one modified internucleoside linkage.
Embodiment 23. The oligomeric compound of embodiment 22, wherein the at least one modified internucleoside linkage is a phosphorothioate internucleoside linkage.
Embodiment 24. The oligomeric compound of embodiment 22, wherein each internucleoside linkage of the modified oligonucleotide is a modified internucleoside linkage.
Embodiment 25. The oligomeric compound of embodiment 22, wherein each internucleoside linkage is a phosphorothioate internucleoside linkage.
Embodiment 26. The oligomeric compound of any of embodiments 1-23, wherein at least one internucleoside linkage of the modified oligonucleotide is a phosphodiester internucleoside linkage.
Embodiment 27. The oligomeric compound of any of embodiments 22-23 or 26, wherein each internucleoside linkage of the modified oligonucleotide is independently selected from a phosphodiester internucleoside linkage and a phosphorothioate internucleoside linkage.
Embodiment 28. The oligomeric compound of any of embodiments 22-24 or 26-27, wherein at least 10, at least 11, at least 12, at least 13, at least 14, at least 15, at least 16, or at least 17 internucleoside linkages of the modified oligonucleotide are phosphorothioate internucleoside linkages.
Embodiment 29. The oligomeric compound of any of embodiments 1-23, wherein the modified oligonucleotide consists of 18 linked nucleosides and has an internucleoside linkage motif selected from sssssssssssssssss and sosssssssosssssss.
Embodiment 30. The oligomeric compound of embodiment 19, wherein the modified oligonucleotide consists of 18 linked nucleosides, wherein the internucleotide linkage motif of the modified oligonucleotide is sssssssssssssssss.
Embodiment 31. The oligomeric compound of embodiment 19, wherein the modified oligonucleotide consists of 18 linked nucleosides, wherein the internucleotide linkage motif of the modified oligonucleotide is sosssssssosssssss.
Embodiment 32. The oligomeric compound of any of embodiments 1-31, wherein the modified oligonucleotide comprises at least one modified nucleobase.
Embodiment 33. The oligomeric compound of embodiment 32, wherein the modified nucleobase is a 5-methyl cytosine or hypoxanthine.
Embodiment 34. The oligomeric compound of embodiment 33, wherein each cytosine is a 5-methyl cytosine.
Embodiment 35. An oligomeric compound comprising a modified oligonucleotide according to the following chemical notation 5′ to 3′):

(SEQ ID NO: 855)
GₑₛTₑₒ^{m}Cₑₛ^{m}CₑₛAₑₛGₑₛGₑₛGₑₛAₑₛGₑₒAₑₛAₑₛTₑₛTₑₛTₑₛGₑₛGₑₛTₑ;
(SEQ ID NO: 856)
GₑₛTₑₒGₑₛGₑₛAₑₛTₑₛAₑₛGₑₛGₑₛGₑₒAₑₛAₑₛAₑₛAₑₛGₑₛ^{m}CₑₛAₑₛ^{m}Cₑ;
(SEQ ID NO: 857)
GₑₛGₑₒGₑₛTₑₛ^{m}Cₑₛ^{m}CₑₛAₑₛ^{m}CₑₛTₑₛGₑₒAₑₛAₑₛAₑₛ^{m}CₑₛGₑₛGₑₛGₑₛGₑ;
(SEQ ID NO: 858)
GₑₛGₑₒAₑₛTₑₛAₑₛGₑₛGₑₛGₑₛAₑₛAₑₒAₑₛAₑₛGₑₛ^{m}CₑₛAₑₛ^{m}Cₑₛ^{m}CₑₛTₑ;
(SEQ ID NO: 859)
GₑₛGₑₒTₑₛ^{m}Cₑₛ^{m}CₑₛAₑₛGₑₛGₑₛGₑₛAₑₒGₑₛAₑₛAₑₛTₑₛTₑₛTₑₛGₑₛGₑ;
(SEQ ID NO: 860)
AₑₛAₑₒAₑₛ^{m}CₑₛGₑₛGₑₛGₑₛGₑₛAₑₛGₑₒGₑₛGₑₛGₑₛAₑₛTₑₛGₑₛGₑₛ^{m}Cₑ;
(SEQ ID NO: 861)
GₑₛAₑₒAₑₛAₑₛ^{m}CₑₛGₑₛGₑₛGₑₛGₑₛAₑₒGₑₛGₑₛGₑₛGₑₛAₑₛTₑₛGₑₛGₑ;
(SEQ ID NO: 862)
TₑₛGₑₒAₑₛAₑₛAₑₛ^{m}CₑₛGₑₛGₑₛGₑₛGₑₒAₑₛGₑₛGₑₛGₑₛGₑₛAₑₛTₑₛGₑ;
(SEQ ID NO: 863)
^{m}CₑₛTₑₒGₑₛAₑₛAₑₛAₑₛ^{m}CₑₛGₑₛGₑₛGₑₒGₑₛAₑₛGₑₛGₑₛGₑₛGₑₛAₑₛTₑ;
(SEQ ID NO: 864)
Aₑₛ^{m}CₑₒTₑₛGₑₛAₑₛAₑₛAₑₛ^{m}CₑₛGₑₛGₑₒGₑₛGₑₛAₑₛGₑₛGₑₛGₑₛGₑₛAₑ;
(SEQ ID NO: 865)
^{m}CₑₛAₑₒ^{m}CₑₛTₑₛGₑₛAₑₛAₑₛAₑₛ^{m}CₑₛGₑₒGₑₛGₑₛGₑₛAₑₛGₑₛGₑₛGₑₛGₑ;
or
(SEQ ID NO: 866)
GₑₛGₑₒTₑₛ^{m}Cₑₛ^{m}CₑₛAₑₛ^{m}CₑₛTₑₛGₑₛAₑₒAₑₛAₑₛ^{m}CₑₛGₑₛGₑₛGₑₛGₑₛAₑ);

wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.
   Embodiment 36. The oligomeric compound of any of embodiments 1-35, wherein the modified oligonucleotide is a pharmaceutically acceptable salt.
   Embodiment 37. The oligomeric compound of embodiment 36, wherein the modified oligonucleotide is a pharmaceutically acceptable salt comprising one or more cations selected from sodium, potassium, calcium, and magnesium.
   Embodiment 38. The oligomeric compound of any of embodiments 1-37, consisting of the modified oligonucleotide.
   Embodiment 39. The oligomeric compound of any of embodiments 1-37, wherein the oligomeric compound comprises a conjugate group.
   Embodiment 40. The oligomeric compound of embodiment 39, wherein the conjugate group comprises a conjugate linker and a conjugate moiety.
   Embodiment 41. The oligomeric compound of embodiment 40, wherein the conjugate linker consists of a single bond.
   Embodiment 42. The oligomeric compound of embodiment 40 or embodiment 41, wherein the conjugate linker is cleavable.
   Embodiment 43. The oligomeric compound of embodiment 40 or embodiment 41, wherein the conjugate linker comprises 1-3 linker nucleosides.
   Embodiment 44. The oligomeric compound of any of embodiments 40-42, wherein the conjugate linker does not comprise any linker nucleosides.
   Embodiment 45. The oligomeric compound of any of embodiments 39-44, wherein the conjugate group is attached to the 5′-end of the modified oligonucleotide.
   Embodiment 46. The oligomeric compound of any of embodiments 39-44, wherein the conjugate group is attached to the 3′-end of the modified oligonucleotide.
   Embodiment 47. The oligomeric compound of any of embodiments 39-44, wherein the oligomeric compound comprises a terminal group.
   Embodiment 48. The oligomeric compound of embodiment 47, wherein the terminal group is an abasic sugar moiety.
   Embodiment 49. The oligomeric compound of any of embodiments 1-48, wherein the oligomeric compound is a single-stranded oligomeric compound.
   Embodiment 50. An oligomeric compound comprising a modified oligonucleotide according to the following chemical notation: Aₑₛ^{m}CₑₒTₑₛGₑₛAₑₛAₑₛAₑₛ^{m}CₑₛGₑₛGₑₒGₑₛGₑₛAₑₛGₑₛGₑₛGₑₛGₑₛAₑ (SEQ ID NO 864), wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.
   Embodiment 51. An oligomeric compound comprising a modified oligonucleotide according to the following chemical notation: ^{m}CₑₛAₑₒ^{m}CₑₛTₑₛGₑₛAₑₛAₑₛAₑₛ^{m}CₑₛGₑₒGₑₛGₑₛGₑₛAₑₛGₑₛGₑₛGₑₛGₑ (SEQ ID NO 865), wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.
   Embodiment 52. A modified oligonucleotide according to the following chemical structure:

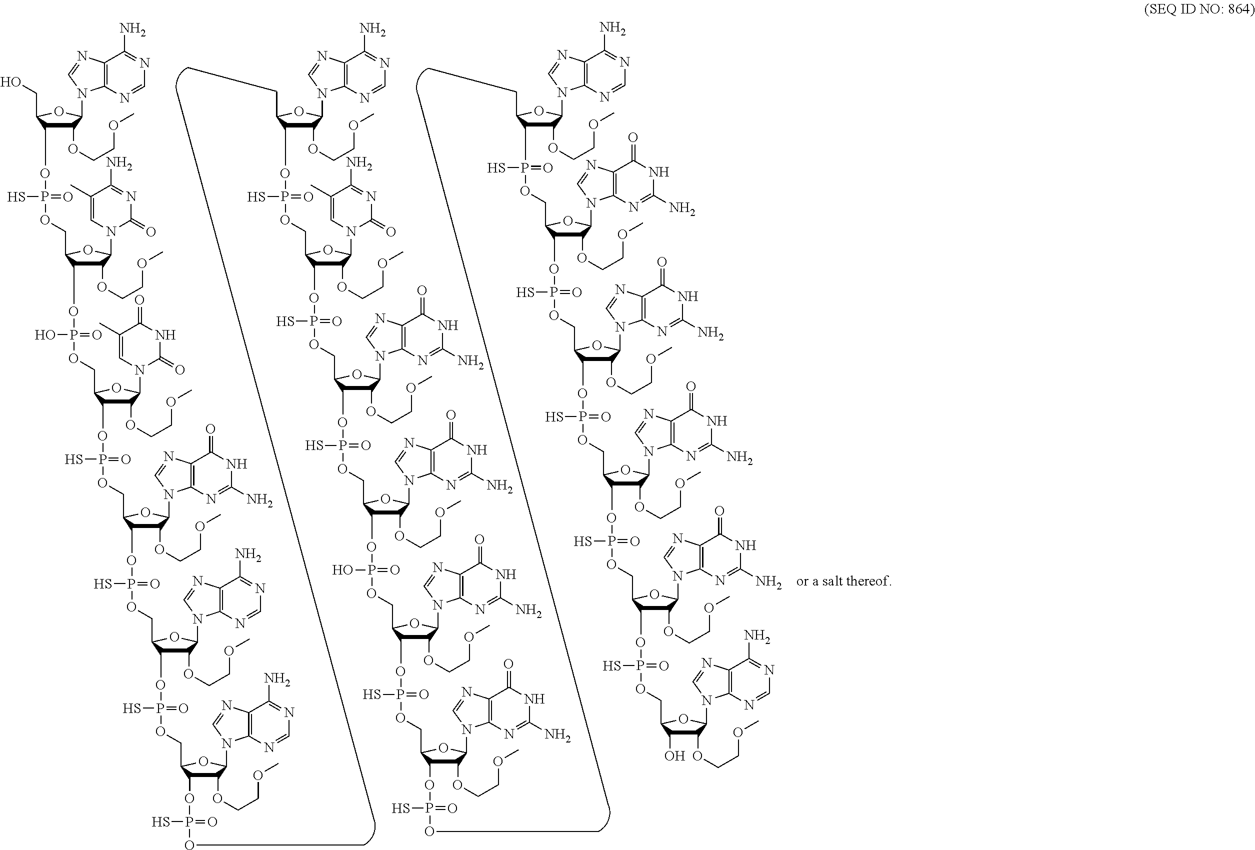

Embodiment 53. The modified oligonucleotide of embodiment 52, which is the sodium salt or the potassium salt.
Embodiment 54. A modified oligonucleotide according to the following chemical structure:

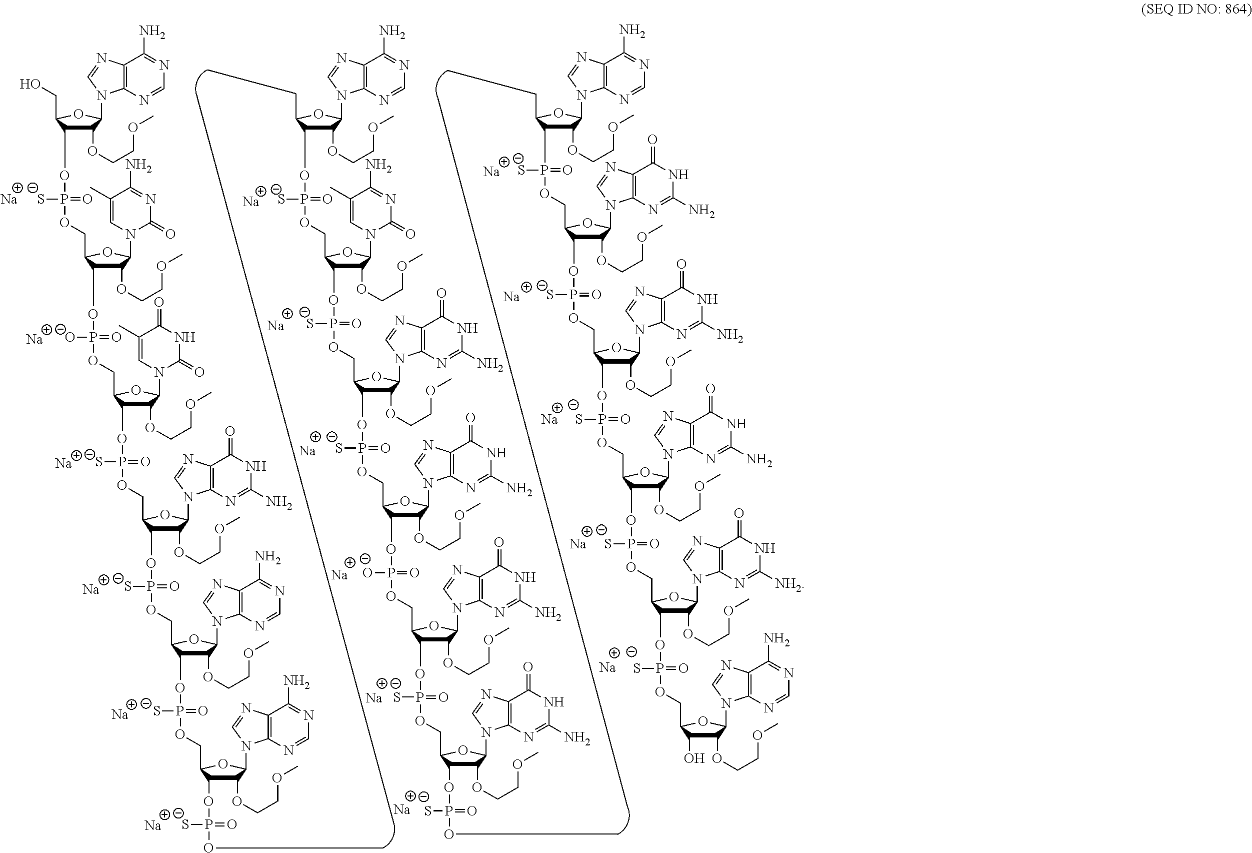

Embodiment 55. A modified oligonucleotide according to the following chemical structure:

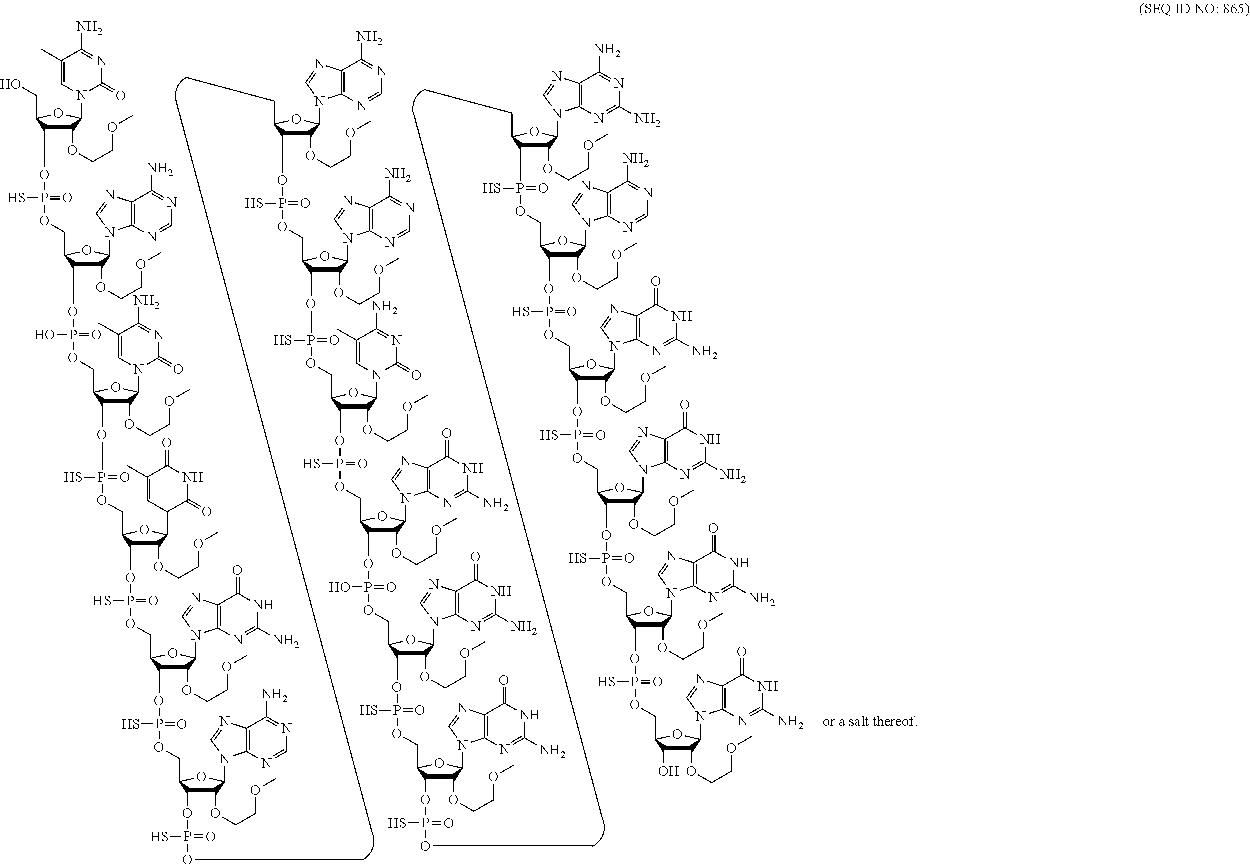

Embodiment 56. The modified oligonucleotide of embodiment 55, which is the sodium salt or the potassium salt.
Embodiment 57. A modified oligonucleotide according to the following chemical structure:

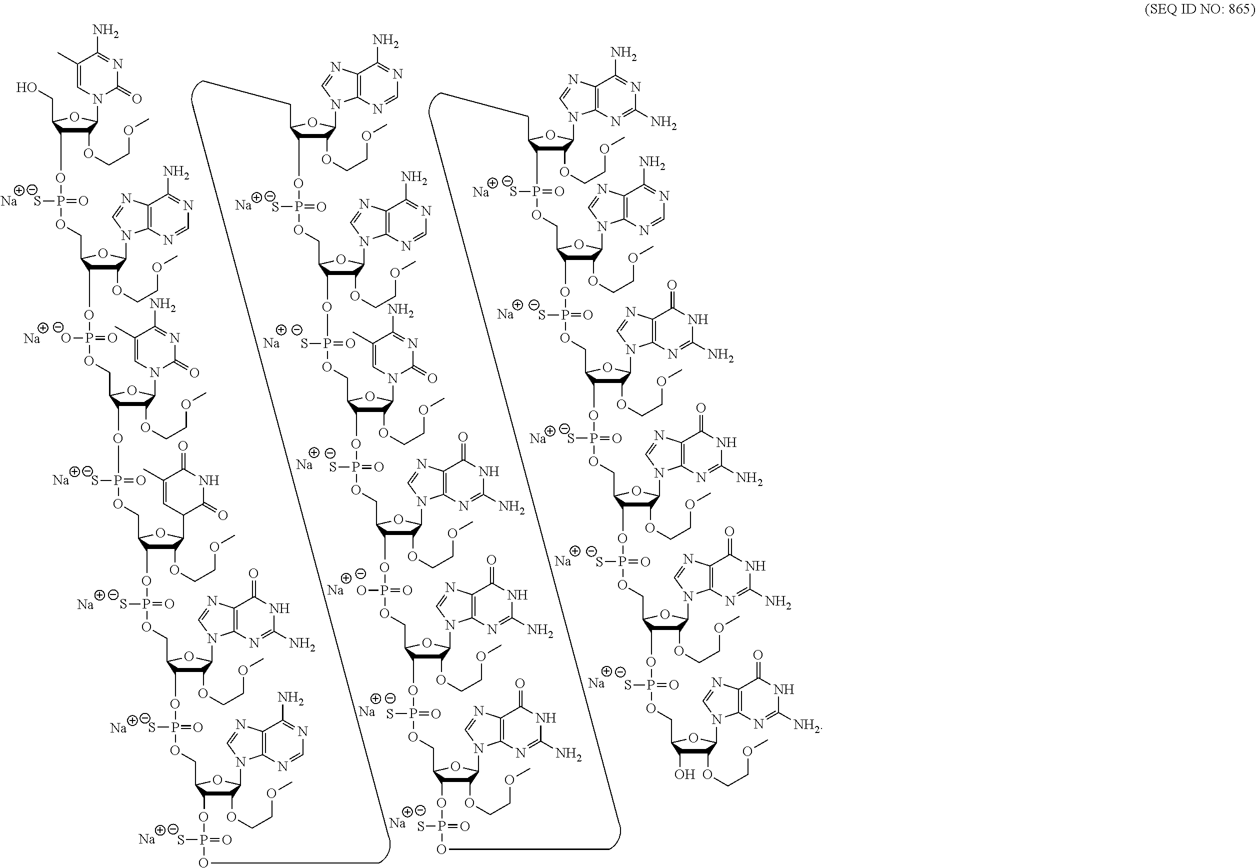

Embodiment 58. A chirally enriched population of oligomeric compounds of any of embodiments 1-51, or a chirally enriched population of modified oligonucleotides ofany of embodiments 52-57, wherein the population is enriched for oligomeric compounds comprising at least one particular phosphorothioate internucleoside linkage having a particular stereochemical configuration.
Embodiment 59. The chirally enriched population of embodiment 58, wherein the population is enriched for modified oligonucleotides comprising at least one particular phosphorothioate internucleoside linkage having the (Sp) configuration.
Embodiment 60. The chirally enriched population of embodiment 58, wherein the population is enriched for modified oligonucleotides comprising at least one particular phosphorothioate internucleoside linkage having the (Rp) configuration.
Embodiment 61. The chirally enriched population of embodiment 58, wherein the population is enriched for modified oligonucleotides having a particular, independently selected stereochemical configuration at each phosphorothioate internucleoside linkage.
Embodiment 62. The chirally enriched population of embodiment 61, wherein the population is enriched for modified oligonucleotides having the (Sp) configuration at each phosphorothioate internucleoside linkage or for modified oligonucleotides having the (Rp) configuration at each phosphorothioate internucleoside linkage.
Embodiment 63. The chirally enriched population of embodiment 61, wherein the population is enriched for modified oligonucleotides having the (Rp) configuration at one particular phosphorothioate internucleoside linkage and the (Sp) configuration at each of the remaining phosphorothioate internucleoside linkages.
Embodiment 64. The chirally enriched population of embodiment 61, wherein the population is enriched for modified oligonucleotides having at least 3 contiguous phosphorothioate internucleoside linkages in the Sp, Sp, and Rp configurations, in the 5′ to 3′ direction.
Embodiment 65. A population of oligomeric compounds of any of embodiments 1-51, or a population of modified oligonucleotides of any of embodiments 52-57, wherein all of the phosphorothioate internucleoside linkages of the modified oligonucleotide are stereorandom.
Embodiment 66. An antisense agent comprising an antisense compound, wherein the antisense compound is the oligomeric compound of any of embodiments 1-51.
Embodiment 67. The antisense agent of embodiment 66, wherein the antisense agent comprises a conjugate group, wherein the conjugate group comprises a cell-targeting moiety.
Embodiment 68. A pharmaceutical composition comprising an oligomeric compound of any of embodiments 1-51, a modified oligonucleotide of any of embodiments 52-57, a population of any of embodiments 58-65, or an antisense agent of embodiment 66 or embodiment 67, and a pharmaceutically acceptable diluent.
Embodiment 69. The pharmaceutical composition of embodiment 68, wherein the pharmaceutically acceptable diluent is artificial CSF (aCSF) or phosphate-buffered saline (PBS).
Embodiment 70. The pharmaceutical composition of embodiment 69, wherein the pharmaceutical composition consists essentially of the oligomeric compound of any of embodiments 1-51, the modified oligonucleotide of any of embodiments 52-57, the population of any of embodiments 58-65, the or the antisense agent of embodiment 66 or embodiment 67, and aCSF.
Embodiment 71. The pharmaceutical composition of embodiment 69, wherein the pharmaceutical composition consists essentially of the oligomeric compound of any of embodiments 1-51, the modified oligonucleotide of any of embodiments 52-57, the population of any of embodiments 58-65, or the antisense agent of embodiment 66 or embodiment 67, and PBS.
Embodiment 72. A method comprising administering to a subject an oligomeric compound of any of embodiments 1-51, a modified oligonucleotide of any of embodiments 52-57, a population of any of embodiments 58-65, an antisense agent of embodiment 66 or embodiment 67, or a pharmaceutical composition of any of embodiments 68-71.
Embodiment 73. A method of treating a disease or disorder associated with an insufficient expression of progranulin, comprising administering to a subject having or at risk for developing a disease or disorder associated with insufficient expression of progranulin a therapeutically effective amount of an oligomeric compound of any of embodiments 1-51, a modified oligonucleotide of any of embodiments 52-57, a population of any of embodiments 58-65, an antisense agent of embodiment 66 or embodiment 67, or a pharmaceutical composition of any of embodiments 68-71, thereby treating the disease or disorder associated with an insufficient expression of progranulin.
Embodiment 74. The method of embodiment 73, wherein the disease or disorder associated with an insufficient expression of progranulin is a neurological disease or disorder.
Embodiment 75. The method of embodiment 73 or embodiment 74, wherein the disease or disorder associated with an insufficient expression of progranulin is a lysosomal storage disorder or a TDP-43 proteinopathy.
Embodiment 76. The method of any of embodiments 73-75, wherein the disease or disorder associated with insufficient expression of progranulin is frontotemporal dementia (FTD), frontotemporal lobar degeneration (FTLD), neuronal ceroid lipofuscinosis (NCL), Alzheimer's disease (AD), or amyotrophic lateral sclerosis (ALS).
Embodiment 77. The method of any of embodiments 73-77, wherein at least one symptom or hallmark of the disease or disorder associated with insufficient expression of progranulin is ameliorated.
Embodiment 78. The method of embodiment 77, wherein the at least one symptom or hallmark is deterioration in behavior and personality, language impairment, alterations in muscle or motor functions, memory loss, cognitive dysfunction, tremor, seizures, or dizziness.
Embodiment 79. The method of embodiment 77 or embodiment 78, wherein administration of the oligomeric compound of any of embodiments 1-51, the modified oligonucleotide of any of embodiments 52-57, the population of any of embodiments 58-65, the antisense agent of embodiment 66 or embodiment 67, or the pharmaceutical composition of any of embodiments 68-71 improves behavior or personality, slows deterioration in behavior or personality, improves language ability, slows deterioration of language ability, improves muscle or motor function, slows deterioration in muscle or motor function, improves memory, slows deterioration in memory, improves cognitive function, slows deterioration of cognitive function, reduces tremors, reduces seizures, or reduces dizziness.
Embodiment 80. The method of any of embodiments 73-79, wherein the oligomeric compound of any of embodiments 1-51, the modified oligonucleotide of any of embodiments 52-57, the population of any of embodiments 58-65, the antisense agent of embodiment 66 or embodiment 67, or the pharmaceutical composition of any of embodiments 68-71 is administered to the central nervous system or systemically.
Embodiment 81. The method of any of embodiments 73-79, wherein the oligomeric compound of any of embodiments 1-51, the modified oligonucleotide of any of embodiments 52-57, the population of any of embodiments 58-65, the antisense agent of embodiment 66 or embodiment 67, or the pharmaceutical composition of any of embodiments 68-71 is administered any of intrathecally, systemically, subcutaneously, or intramuscularly.
Embodiment 82. The method of any of embodiments 73-81, wherein the subject is a human.
Embodiment 83. A method of increasing progranulin RNA or one or more splice variants of said progranulin RNA in a cell, comprising contacting the cell with an oligomeric compound of any of embodiments 1-51, a modified oligonucleotide of any of embodiments 52-57, a population of any of embodiments 58-65, or an antisense agent of embodiment 66 or embodiment 67.
Embodiment 84. A method of increasing progranulin protein in a cell, comprising contacting the cell with an oligomeric compound of any of embodiments 1-51, a modified oligonucleotide of any of embodiments 52-57, a population of any of embodiments 58-65, or an antisense agent of embodiment 66 or embodiment 67.
Embodiment 85. The method of embodiment 83 or embodiment 84, wherein the cell is a neuron.
Embodiment 86. The method of any of embodiments 83-85, wherein the cell is a human cell.
Embodiment 87. Use of an oligomeric compound of any of embodiments 1-51, a modified oligonucleotide of any of embodiments 52-57, a population of any of embodiments 58-65, an antisense agent of embodiment 66 or embodiment 67, or a pharmaceutical composition of any of embodiments 68-71 for treating a disease or disorder associated with an insufficient expression of progranulin.
Embodiment 88. Use of an oligomeric compound of any of embodiments 1-51, a modified oligonucleotide of any of embodiments 52-57, a population of any of embodiments 58-65, an antisense agent of embodiment 66 or embodiment 67, or a pharmaceutical composition of any of embodiments 68-71 in the manufacture of a medicament for treating a disease or disorder associated with an insufficient expression of progranulin.
Embodiment 89. The use of embodiment 87 or embodiment 88, wherein the disease or disorder associated with an insufficient expression of progranulin is a neurological disease or disorder.
Embodiment 90. The use of embodiment 87 or embodiment 88, wherein the disease or disorder associated with an insufficient expression of progranulin is a lysosomal storage disorder or a TDP-43 proteinopathy.
Embodiment 91. The use of any of embodiments 87-90, wherein the disease or disorder associated with insufficient expression of progranulin is frontotemporal dementia (FTD), frontotemporal lobar degeneration (FTLD), neuronal ceroid lipofuscinosis (NCL), Alzheimer's disease (AD), or amyotrophic lateral sclerosis (ALS).

### Certain Compositions

### 1. Compound No. 1557993

In certain embodiments, Compound No. 1557993 is characterized as a modified oligonucleotide having a nucleobase sequence (from 5′ to 3′) of ACTGAAACGGGGAGGGGA (SEQ ID NO 781), wherein each nucleoside comprises a 2′-MOE sugar moiety, wherein the internucleoside linkages between nucleosides 2 to 3, and 10 to 11 are phosphodiester internucleoside linkages, the internucleoside linkages between nucleosides 1 to 2, 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, and 17 to 18 are phosphorothioate internucleoside linkages, and wherein each cytosine is a 5-methyl cytosine.

In certain embodiments, Compound No. 1557993 is represented by the following chemical notation:

(SEQ ID NO 864)
Aₑₛ^{m}CₑₒTₑₛGₑₛAₑₛAₑₛAₑₛ^{m}CₑₛGₑₛGₑₒGₑₛGₑₛAₑₛGₑₛGₑₛGₑₛGₑₛAₑ,

wherein
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

In certain embodiments Compound No. 1557993 is represented by the following chemical structure:

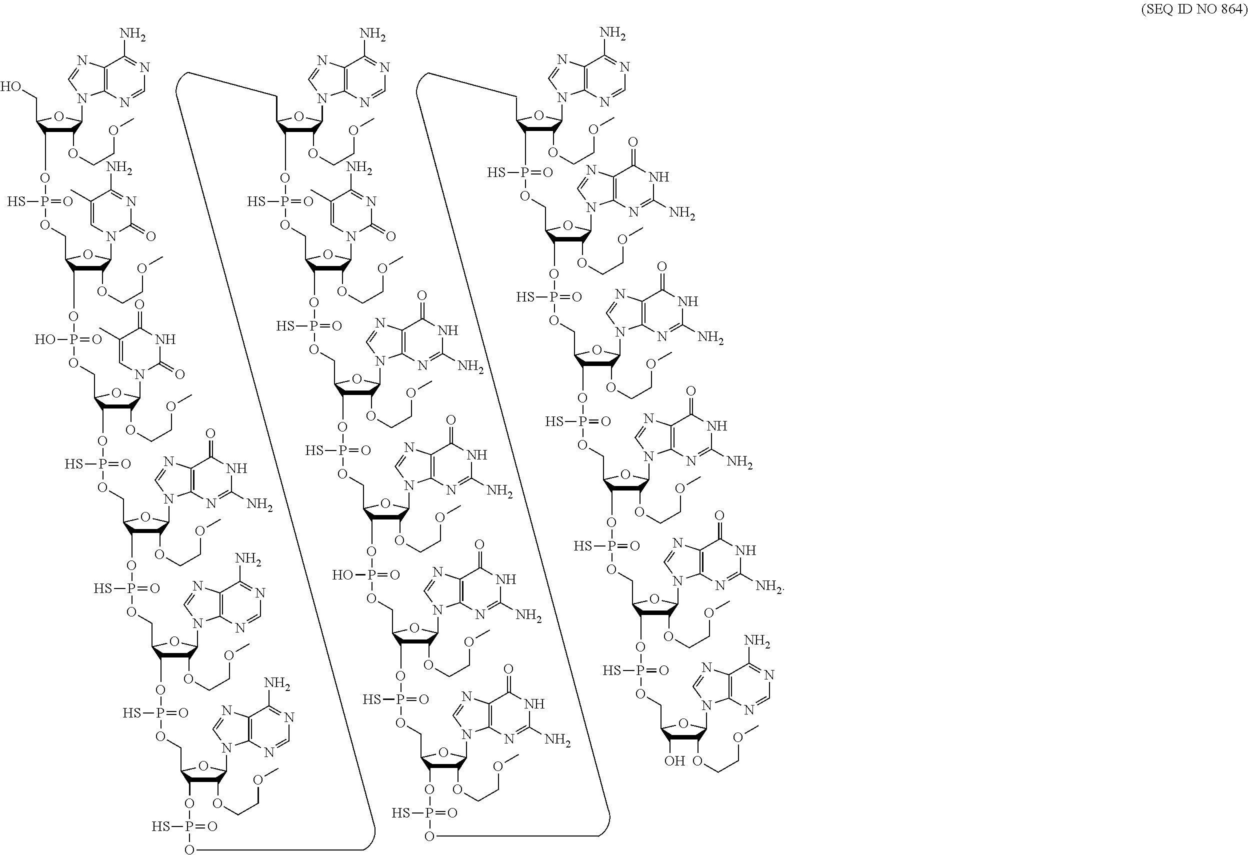

### Structure 1. Compound No. 1557993

In certain embodiments, an oligomeric compound comprises the sodium salt or the potassium salt of the modified oligonucleotide represented by Structure 1.

In certain embodiments the sodium salt of Compound No. 1557993 is represented by the following chemical structure:

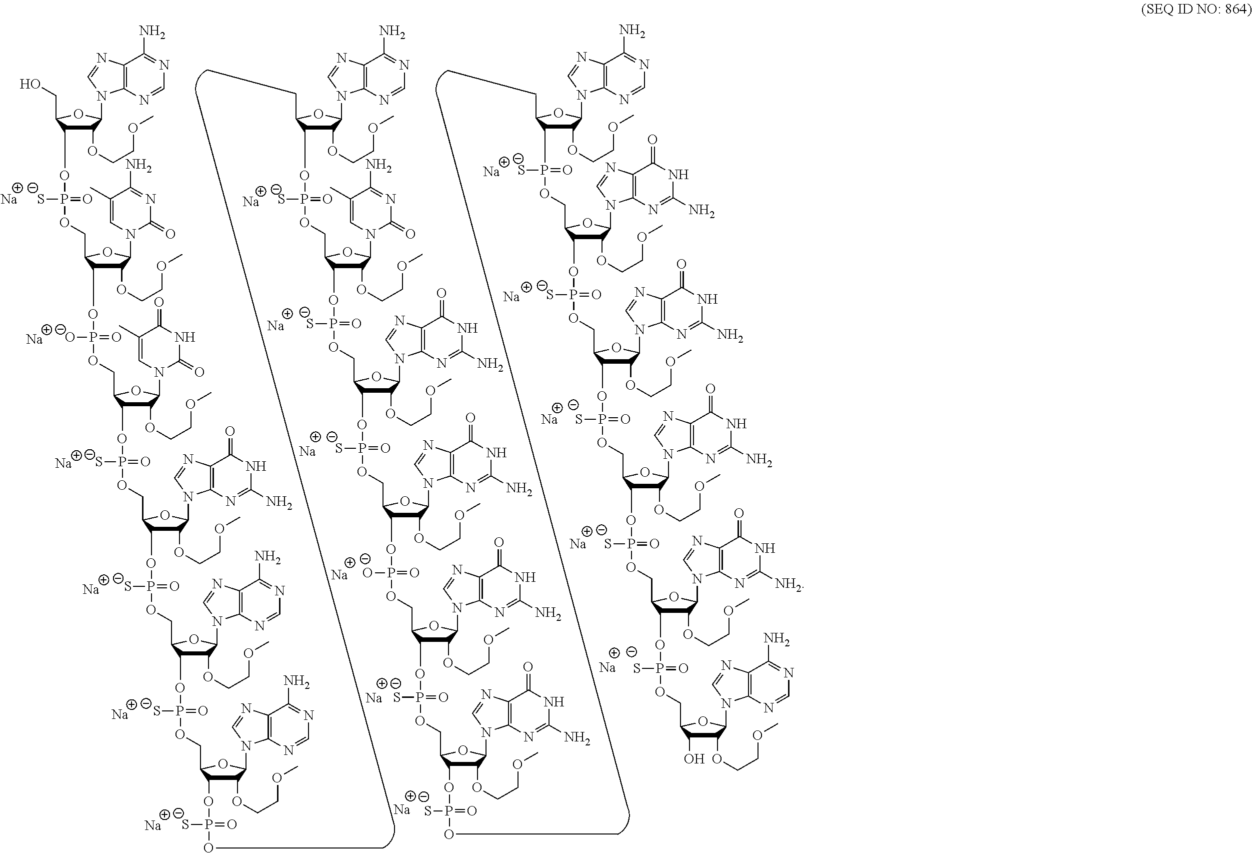

### Structure 2. The Sodium Salt of Compound No. 1557993

### 2. Compound No. 1557994

In certain embodiments, Compound No. 1557994 is characterized as a modified oligonucleotide having a sequence (from 5′ to 3′) of CACTGAAACGGGGAGGGG (SEQ ID NO 80), wherein each nucleoside comprises a 2′-MOE sugar moiety, wherein the internucleoside linkages between nucleosides 2 to 3, and 10 to 11 are phosphodiester internucleoside linkages, the internucleoside linkages between nucleosides 1 to 2, 3 to 4, 4 to 5, 5 to 6, 6 to 7, 7 to 8, 8 to 9, 9 to 10, 11 to 12, 12 to 13, 13 to 14, 14 to 15, 15 to 16, 16 to 17, and 17 to 18 are phosphorothioate internucleoside linkages, and wherein each cytosine is a 5-methyl cytosine.

In certain embodiments, Compound No. 1557994 is represented by the following chemical notation:

(SEQ ID NO 865)
^{m}CₑₛAₑₒ^{m}CₑₛTₑₛGₑₛAₑₛAₑₛAₑₛ^{m}CₑₛGₑₒGₑₛGₑₛGₑₛAₑₛGₑₛGₑₛGₑₛGₑ,

A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

In certain embodiments Compound No. 1557994 is represented by the following chemical structure:

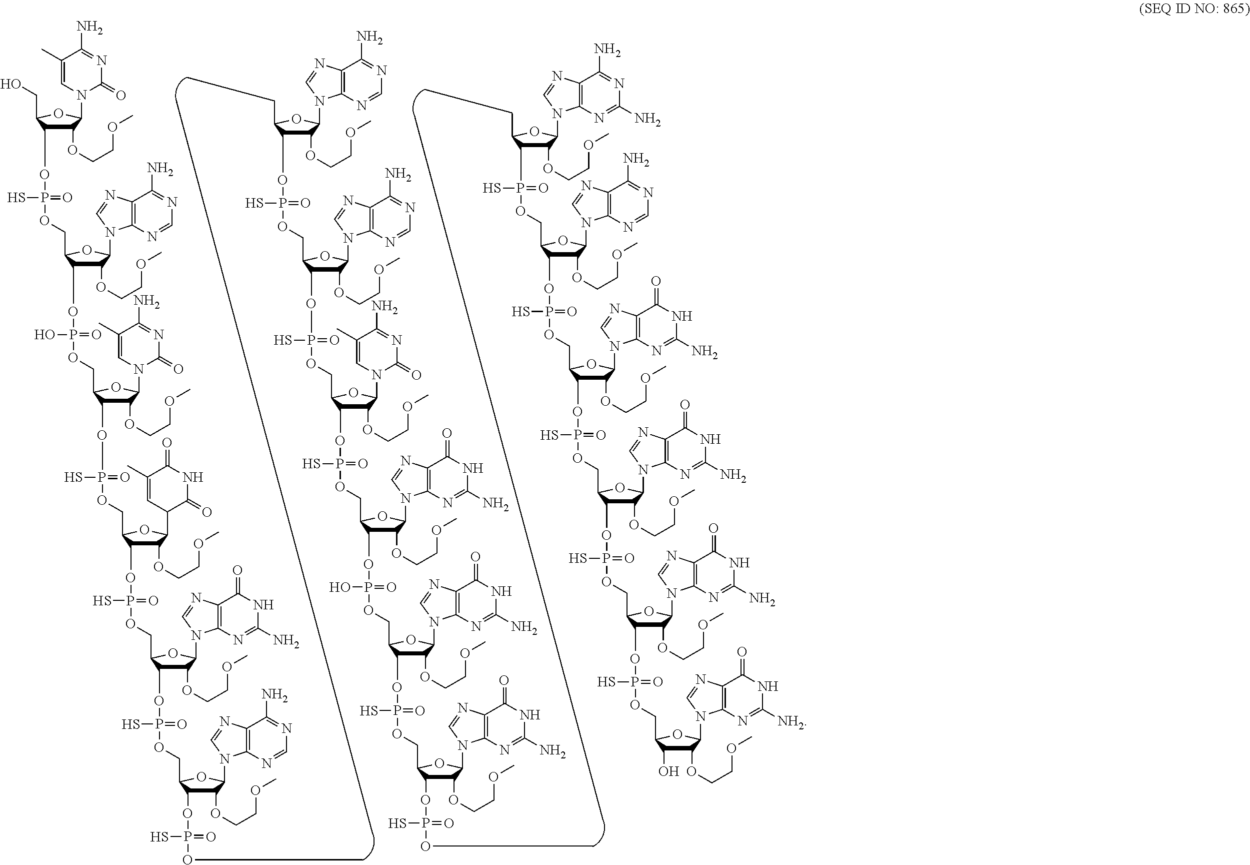

### Structure 3. Compound No. 1557994

In certain embodiments, an oligomeric compound comprises the sodium salt or the potassium salt of the modified oligonucleotide represented by Structure 3.

In certain embodiments the sodium salt of Compound No. 1557994 is represented by the following chemical structure:

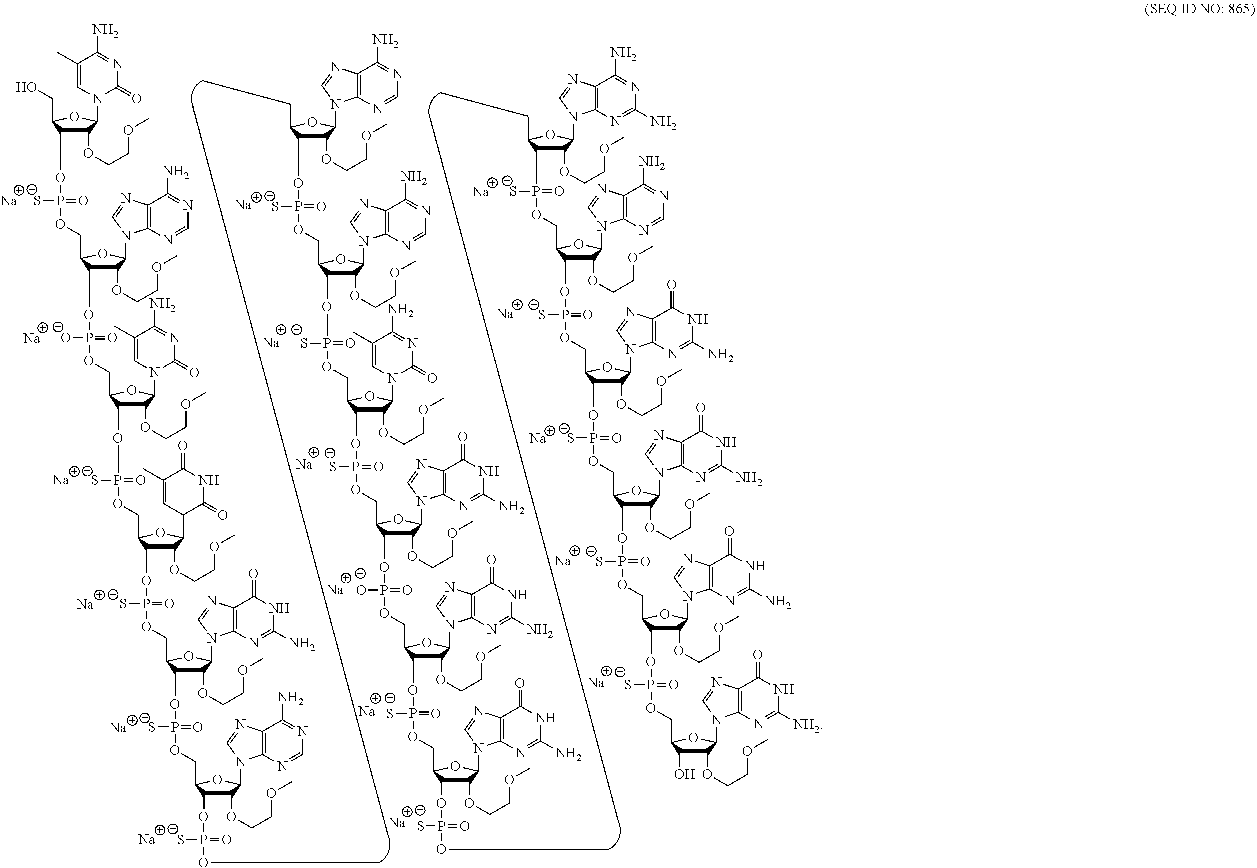

Structure 4. The sodium salt of Compound No. 1557994

### Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into modified oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 3′, 4′, and/or 5′ positions. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE” or “O-methoxyethyl”), and 2′-O—N-alkyl acetamide, e.g., 2′-O—N-methyl acetamide (“NMA”), 2′-O—N-dimethyl acetamide, 2′-O—N-ethyl acetamide, or 2′-O—N-propyl acetamide. For example, see U.S. Pat. No. 6,147,200, Prakash et al., 2003, Org. Lett., 5, 403-6.

A “2′-O—N-methyl acetamide nucleoside” or “2′-NMA nucleoside” is shown below:

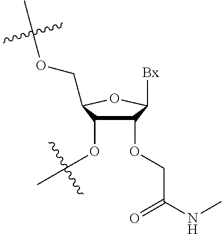

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)ON(CH₃)₂ (DMAOE), O(CH₂)₂O(CH₂)₂N(CH₃)₂ (DMAEOE), and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl, e.g., for example, OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, and OCH₂C(═O)—N(H)CH₃.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include but are not limited to: 4′-CH₂-2′, 4′—(CH₂)₂-2′, 4′—(CH₂)₃-2′, 4′—CH₂—O-2′ (“LNA”), 4′-CH₂—S-2′, 4′—(CH₂)₂—O-2′ (“ENA”), 4′-CH(CH₃)—O-2′ (referred to as “constrained ethyl” or “cEt” when in the S configuration), 4′-CH₂—O—CH₂-2′, 4′—CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′—C(RₐR_{b})—O—N(R)-2′, 4′—CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙO—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727. In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

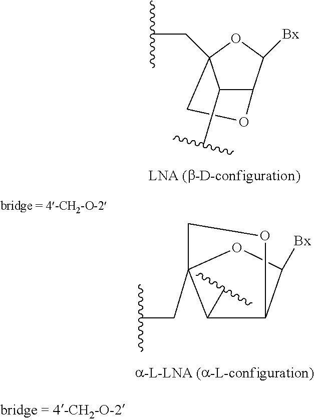

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“NINA”) (see, e.g., Leumann, CJ. Bioorg. & Med. Chem. 2002, 10, 841-854), fluoro HNA:

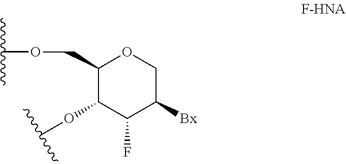

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

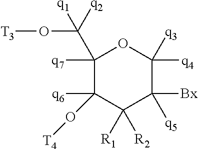

wherein, independently, for each of the modified THP nucleosides:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q are each H. In certain embodiments, at least one of q₁, q₂, q₃, q₄, q₅, q₆ and q is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

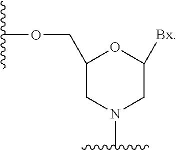

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside. Examples of modified nucleobases include 5-methyl cytosine.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyl adenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphodiesters, which contain a phosphodiester bond, P(O₂)═O, (also referred to as unmodified or naturally occurring linkages); phosphotriesters; methylphosphonates; methoxypropylphosphonates (“MOP”); phosphoramidates; phosphorothioates (P(O₂)═S); and phosphorodithioates (HS—P═S). Representative non-phosphorus containing internucleoside linking groups include but are not limited to methylenemethylimino (—CH₂—N(CH₃)—O—CH₂—); thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of modified oligonucleotides can be generated using synthetic methods known in the art, e.g., methods described in Oka et al., JACS, 2003, 125, 8307, Wan et al. Nuc. Acid. Res., 2014, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

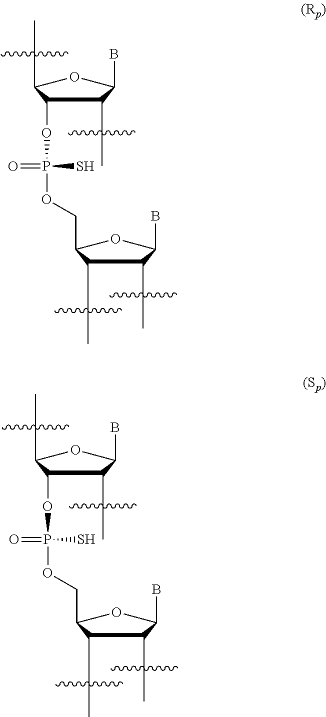

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (see for example, Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide, or portion thereof, in a defined pattern or sugar motif. In certain instances, such sugar motifs include but are not limited to any of the sugar modifications discussed herein.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE modified sugar moiety, a 2′-NMA modified sugar moiety, a 2′-OMe modified sugar moiety, and a 2′-F modified sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE modified sugar moiety, a 2′-NMA modified sugar moiety, a 2′-OMe modified sugar moiety, and a 2′-F modified sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, a bicyclic sugar moiety, or a sugar surrogate. In certain embodiments, the 2′-substituted sugar moiety is selected from a 2′-MOE modified sugar moiety, a 2′-NMA modified sugar moiety, a 2′-OMe modified sugar moiety, and a 2′-F modified sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE modified sugar moiety, a 2′-NMA modified sugar moiety, a 2′-OMe modified sugar moiety, and a 2′-F modified sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

### 2. Certain Nucleobase Motifs

In certain embodiments, oligonucleotides comprise modified and/or unmodified nucleobases arranged along the oligonucleotide, or portion thereof, in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

### 3. Certain Internucleoside Linkage Motifs

In certain embodiments, oligonucleotides comprise modified and/or unmodified internucleoside linkages arranged along the oligonucleotide, or portion thereof, in a defined pattern or motif. In certain embodiments, each internucleoside linking group is a phosphodiester internucleoside linkage. In certain embodiments, each internucleoside linking group of a modified oligonucleotide is a phosphorothioate internucleoside linkage. In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphodiester internucleoside linkages. In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphorothioate internucleoside linkages. In certain embodiments, modified oligonucleotides comprise at least 1, at least 2, at least 3, at least 4, or at least 5 phosphodiester internucleoside linkages and the remainder of the internucleoside linkages are phosphorothioate internucleoside linkages. In certain embodiments, modified oligonucleotides have an internucleoside linkage motif of (5′ to 3′): sssssssssssssssss, wherein each “s” represents a phosphorothioate internucleoside linkage. In certain embodiments, modified oligonucleotides have an internucleoside linkage motif of (5′ to 3′): sosssssssosssssss wherein each “s” represents a phosphorothioate internucleoside linkage and each “o” represents a phosphodiester internucleoside linkage.

### C. Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a particular sugar motif may be modified or unmodified and may or may not follow the modification pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C17 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

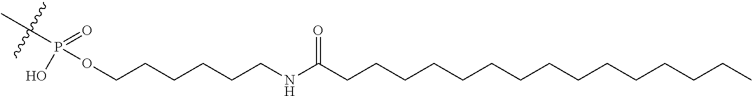 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are subunits making up a conjugate linker. In certain embodiments, the conjugate linker comprises a chain structure, such as a hydrocarbyl chain, or an oligomer of repeating units such as ethylene glycol, nucleosides, or amino acid units. In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to compounds, such as the oligonucleotides provided herein. In general, a bifunctional linking moiety comprises at least two functional groups. One of the functional groups is selected to react with a particular site on a compound and the other is selected to react with a conjugate moiety. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 1-3 linker nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxyribonucleoside that is attached to either the 3′ or 5-terminal nucleoside of an oligonucleotide by a phosphate internucleoside linkage and covalently attached to the remainder of the conjugate linker or conjugate moiety by a phosphodiester or phosphorothioate internucleoside linkage. In certain such embodiments, the cleavable moiety is 2′-deoxyadenosine.

### 1. Cell-Targeting Moieties

In certain embodiments, a conjugate group comprises a cell-targeting moiety. In certain embodiments, a conjugate group has the general formula:

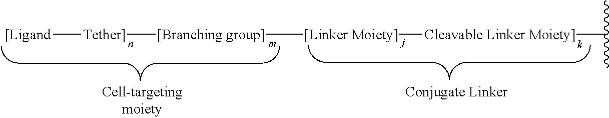

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

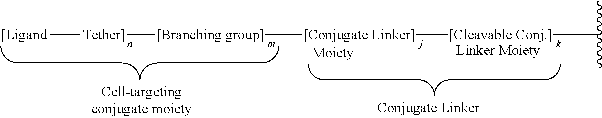

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphanates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### C. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### D. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce, modulate, or increase the amount or activity of a target nucleic acid by 25% or more in the standard in vitro assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, provided herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target nucleic acid results in exon inclusion. In certain embodiments, hybridization of an antisense compound to a target nucleic acid results in an increase in the amount or activity of a target nucleic acid. In certain embodiments, hybridization of an antisense compound complementary to a target nucleic acid results in alteration of splicing, leading to the inclusion or the exclusion of an exon in the mRNA.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### III. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target nucleic acid is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

A. Complementarity/Mismatches to the Target Nucleic Acid It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6 7, 8, 9, 10, 11, 12, 13, 14, 15, 16, 17, 18, 19, or 20 nucleobases in length.

In certain embodiments, oligonucleotides comprise one or more mismatched nucleobases relative to the target nucleic acid. In certain embodiments, the mismatch is at position 1, 2, 3, 4, 5, 6, 7, 8, 9, 10, 11, 12, 13, 14, 15, 16, 17, 18, 19, or 20 from the 5′-end of the oligonucleotide.

### B. Progranulin

In certain embodiments, oligomeric compounds comprise or consist of a modified oligonucleotide that is complementary to a target nucleic acid encoding progranulin, or a portion thereof. In certain embodiments, the target nucleic acid has the sequence set forth in SEQ ID NO: 1 (GENBANK Accession No. NC_000017.11 truncated from nucleotides 44342001 to 44356000), to SEQ ID NO: 2 (GENBANK Accession No. NM_002087.3), or to both. In certain embodiments, oligomeric compounds comprise or consist of a modified oligonucleotide consisting of 18, 19, or 20 linked nucleosides, wherein the nucleobase sequence of the modified oligonucleotide comprises at least 16, at least 17, or 18 contiguous nucleobases of any of the nucleobase sequences of SEQ ID NOs:12-854.

In certain embodiments, contacting a cell with the oligomeric compound modulates the amount of progranulin RNA in a cell. In certain embodiments, contacting a cell with the oligomeric compound increases the amount of progranulin RNA in a cell. In certain embodiments, contacting a cell with the oligomeric compound modulates the amount of progranulin protein in a cell. In certain embodiments, contacting a cell with the oligomeric compound increases the amount of progranulin protein in a cell. In certain embodiments, the oligomeric compound consists of a modified oligonucleotide.

In certain embodiments, contacting a cell in a subject with the oligomeric compound ameliorates one or more symptom or hallmark of a neurological disease or disorder. In certain embodiments, the neurological disease or disorder is FTD. In certain embodiments the neurological disease or disorder is FTLD. In certain embodiments, the neurological disease or disorder is NCL. In certain embodiments, the neurological disease or disorder is a TDP-43 proteinopathy. In certain embodiments, the disease or disorder is a lysosomal storage disorder. In some embodiments, the disease or disorder is ALS. In certain embodiments, the symptom or hallmark is any of deterioration in behavior and personality, language impairment, disturbances or alterations in muscle or motor functions, memory loss, cognitive dysfunction, tremor, seizures, or dizziness.

In certain embodiments, the oligomeric compound is capable of increasing progranulin RNA in vitro by at least 0.5 fold, at least 1 fold, at least 2 fold, or at least 3 fold when tested according to the standard in vitro assay.

### C. Certain Target Nucleic Acids in Certain Tissues

In certain embodiments, oligomeric compounds comprise or consist of a modified oligonucleotide comprising a portion that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include brain tissues, such as, cortex, hypothalamus, hippocampus, cerebellum, and coronal brain tissue.

### IV. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade artificial cerebrospinal fluid.

In certain embodiments, a pharmaceutical composition comprises a modified oligonucleotide and PBS. In certain embodiments, a pharmaceutical composition consists of a modified oligonucleotide and PBS. In certain embodiments, a pharmaceutical composition consists essentially of a modified oligonucleotide and PBS. In certain embodiments, the PBS is pharmaceutical grade PBS.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to a modified oligonucleotide, wherein the conjugate group is cleaved by endogenous nucleases within the body. In certain embodiments, prodrugs comprise one or more conjugate group attached to a modified oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), intraneural, perineural, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salt thereof” or “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na⁺ ions. However, the mass of the protons are nevertheless counted toward the weight of the dose, and the mass of the Na⁺ ions are not counted toward the weight of the dose. Thus, for example, a dose, or dosage unit, of 10 mg of Compound No. 1557993, equals the number of fully protonated molecules that weighs 10 mg. This would be equivalent to 10.51 mg of solvent-free, sodium acetate-free, anhydrous sodiated Compound No. 1557993. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium.

However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### V. Certain Hotspot Regions

In certain embodiments, nucleobases 8,497-8,552 of SEQ ID NO: 1 comprise a hotspot region. In certain embodiments, modified oligonucleotides are complementary to a portion within nucleobases 8,497-8,552 of SEQ ID NO: 1. In certain embodiments, modified oligonucleotides are 18 nucleobases in length. In certain embodiments, each nucleoside of the modified oligonucleotide comprises a 2′-MOE sugar moiety. In certain embodiments, all of the internucleoside linkages of the modified oligonucleotides are phosphorothioate internucleoside linkages. In certain embodiments, the modified oligonucleotide has an internucleoside linkage motif of (from 5′ to 3′): sosssssssosssssss.

The nucleobase sequences of SEQ ID NOs: 34, 268, 345, 424, 501, 579, 657, 735, 800, 801, 806, 812, 813, 816, 821, 828, 844, and 847 are complementary to a portion of nucleobases 8,497-8,552 of SEQ ID NO: 1.

The nucleobase sequence of Compound Nos.: 1212177, 1212178, 1212179, 1212180, 1212181, 1212182, 1212183, 1212184, 1366757, 1366758, 1366763, 1366769, 1366770, 1366773, 1366778, 1366785, 1366801, and 1366804 are complementary to a portion within nucleobases 8,497-8,552 of SEQ ID NO: 1.

In certain embodiments, modified oligonucleotides complementary to a portion within nucleobases 8,497-8,552 of SEQ ID NO: 1 achieve at least 100% expression of progranulin RNA in the standard in vitro assay. In certain embodiments, modified oligonucleotides complementary to a portion within nucleobases 8,497-8,552 of SEQ ID NO: 1 achieve an average of 147% expression of progranulin RNA in the standard in vitro assay.

### NONLIMITING DISCLOSURE AND INCORPORATION BY REFERENCE

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar moiety (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position. Finally, for clarity, unless otherwise indicated, the phrase “nucleobase sequence of SEQ ID NO: X”, refers only to the sequence of nucleobases in that SEQ ID NO: X, independent of any sugar modifications or internucleoside linkage modifications also described in such SEQ ID NO: X.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or $ such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate internucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or 3H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Examples

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Effect of Uniform MOE Modified Oligonucleotides with Uniform Phosphorothioate Internucleoside Linkages on Human Progranulin In Vitro, Single Dose

Modified oligonucleotides complementary to a human progranulin nucleic acid were designed and tested for their effect on progranulin RNA in vitro. The modified oligonucleotides were tested in a series of experiments that had the same culture conditions.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (GENBANK Accession No. NC_000017.11 truncated from nucleotides 44342001 to 44356000), to SEQ ID NO: 2 (GENBANK Accession No. NM_002087.3), or to both. ‘N/A’ indicates that the modified oligonucleotide is not 100% complementary to that particular target nucleic acid sequence.

The modified oligonucleotides in the table below are 18 nucleosides in length, the sugar motif for the modified oligonucleotides is (from 5′ to 3′): eeeeeeeeeeeeeeeeee; wherein “e” represents a 2′-MOE sugar moiety. The internucleoside linkage motif for the modified oligonucleotides is (from 5′ to 3′): sssssssssssssssss; wherein each “s” represents a phosphorothioate internucleoside linkage. Each cytosine residue is a 5-methyl cytosine.

Cultured A-431 cells were treated with modified oligonucleotide at a concentration of 6,000 nM by free uptake at a density of 10,000 cells per well. After a treatment period of approximately 48 hours, RNA was isolated from the cells and progranulin RNA levels were measured by quantitative real-time PCR. Human primer probe set RTS42426 (forward sequence AGGACTAACAGGGCAGTGG, designated herein as SEQ ID NO: 3; reverse sequence CAGCAGCCATACTTCCCA, designated herein as SEQ ID NO: 4; probe sequence TTGTCCAGCTCGGTCATGTGTCC designated herein as SEQ ID NO: 5) was used to measure upregulation of progranulin RNA. Progranulin RNA levels were normalized to total RNA content, as measured by RIBOGREEN®. Results are presented as percent progranulin RNA, relative to the amount in untreated control cells (% UTC). Each separate experiment described in this example is identified by an Assay Identification letter in the table column labeled “AID”.

The values marked with a “T” indicate that the RNAi compound is complementary to the amplicon region of the primer probe set. Values marked as “N.D.” are not defined. Each separate experiment described in this example is identified by an Assay Identification letter in the table column labeled “AID”.

TABLE 1
Uniform MOE modified oligonucleotides with uniform
PS linkages complementary to human progranulin

| | SEQ | SEQ | SEQ | SEQ | | | | |
|---|---|---|---|---|---|---|---|---|
| | ID | ID | ID | ID | | | | |
| | NO: | NO: | NO: | NO: | | | | |
| | 1 | 1 | 2 | 2 | | | | SEQ |
| Compound | Start | Stop | Start | Stop | | progranulin | | ID |
| No. | Site | Site | Site | Site | Sequence (5′ to 3′) | (% UTC) | AID | NO |
| 1211964 | 3086 | 3103 | 1 | 18 | ATCATGTGATTGGAGAAT | 98 | A | 12 |
| 1211974 | 3126 | 3143 | 41 | 58 | TCCTCCCTGCTTCCTCTC | 78 | A | 13 |
| 1211984 | 3166 | 3183 | 81 | 98 | CAGCCTGGAATGCTGTGT | 76 | A | 14 |
| 1211994 | 3206 | 3223 | 121 | 138 | ACCCGCTCCCATTGGCTA | 77 | A | 15 |
| 1212004 | 3246 | 3263 | 161 | 178 | ACCCGCCTACCTCAGTTT | 76 | A | 16 |
| 1212014 | 3283 | 3300 | 198 | 215 | ACCGGGTAGCGCTCAGAC | 101 | A | 17 |
| 1212024 | 3293 | 3310 | 208 | 225 | GGCAGCAGCAACCGGGTA | 54 | A | 18 |
| 1212034 | 3303 | 3320 | 218 | 235 | GCGGTCCTTGGGCAGCAG | 117 | A | 19 |
| 1212044 | 3313 | 3330 | 228 | 245 | GTCCGACTCCGCGGTCCT | 105 | A | 20 |
| 1212054 | N/A | N/A | 238 | 255 | GTCTGCCTGCGTCCGACT | 94 | A | 21 |
| 1212064 | 7156 | 7173 | 248 | 265 | GGTCCACATGGTCTGCCT | 88 | A | 22 |
| 1212074 | 7170 | 7187 | 262 | 279 | ACCCAGCTCACCAGGGTC | 99 | A | 23 |
| 1212084 | 7220 | 7237 | 312 | 329 | ACTGACCATCTGGGCACC | 54 | A | 24 |
| 1212094 | 7270 | 7287 | 362 | 379 | GCAGCTGTAGCTGGCTCC | 80 | A | 25 |
| 1212104 | 7443 | 7460 | 412 | 429 | CCCAGATGCCTGCTCAGT | 105 | A | 26 |
| 1212114 | 7493 | 7510 | 462 | 479 | AGATGCAGGAGTGGCCGG | 95 | A | 27 |
| 1212124 | N/A | N/A | 512 | 529 | TGCCACGGCCTCTGGGAA | 103 | A | 28 |
| 1212134 | 7708 | 7725 | 562 | 579 | TCTGCACTGCAGTGGAAG | 90 | A | 29 |
| 1212144 | 8234 | 8251 | 612 | 629 | GGATGGCACCCACGGAGT | 94 | A | 30 |
| 1212154 | 8284 | 8301 | 662 | 679 | AACACAGCACGTGGAGAA | 91 | A | 31 |
| 1212164 | N/A | N/A | 712 | 729 | CAGCAGGAAGCCTGGGGC | 92 | A | 32 |
| 1212174 | 8485 | 8502 | 762 | 779 | GAACCAGGTCGCAGAAGG | 97 | A | 33 |
| 1212184 | 8535 | 8552 | 812 | 829 | GAGCTTCTTTGCCAGGGG | 126 | A | 34 |
| 1212194 | 8698 | 8715 | 862 | 879 | CACATGACCGAGCTGGAC | 13† | A | 35 |
| 1212204 | 8748 | 8765 | 912 | 929 | GCAGCTCACAGCAGGTAG | 11† | A | 36 |
| 1212214 | N/A | N/A | 962 | 979 | GGAGCAGCAGGTGGCGTT | 94 | A | 37 |
| 1212224 | 9084 | 9101 | 1012 | 1029 | CTCTGGATCAGGTCACAC | 112 | A | 38 |
| 1212234 | 9134 | 9151 | 1062 | 1079 | GCAGCTTAGTGAGGAGGT | 89 | A | 39 |
| 1212244 | 9383 | 9400 | 1112 | 1129 | TGGGCAGCTCACCTCCAT | 40 | A | 40 |
| 1212254 | 9433 | 9450 | 1162 | 1179 | GGGCAGCAGCCCCAGGCC | 69 | A | 41 |
| 1212264 | 9572 | 9589 | 1212 | 1229 | CCGCGGGACAGCAGTGTA | N.D. | A | 42 |
| 1212274 | 9622 | 9639 | 1262 | 1279 | CTGGTGGGGCCCCTGTTC | 94 | A | 43 |
| 1212284 | 9672 | 9689 | 1312 | 1329 | TGTGGGTCTGGCAGGCTG | 79 | A | 44 |
| 1212294 | 9722 | 9739 | 1362 | 1379 | AGGAGGGACAGCTGCTGA | 87 | A | 45 |
| 1212304 | 9772 | 9789 | 1412 | 1429 | GATTGGACAGCAGCCCCA | 56 | A | 46 |
| 1212314 | 10041 | 10058 | 1462 | 1479 | TAGCCCTGGGGGCAGCAG | 111 | A | 47 |
| 1212324 | 10091 | 10108 | 1512 | 1529 | CAGCCACGATCTCGCTTC | 74 | A | 48 |
| 1212334 | 10141 | 10158 | 1562 | 1579 | GTCTCTGGGGTGGGATAA | 56 | A | 49 |
| 1212344 | 10191 | 10208 | 1612 | 1629 | GGGCAGCAGGTCTGCCCC | 97 | A | 50 |
| 1212354 | N/A | N/A | 1662 | 1679 | AGCACACAGCATGGGGCA | 80 | A | 51 |
| 1212364 | 10383 | 10400 | 1712 | 1729 | CTTCACGTTGCAGGTGTA | 106 | A | 52 |
| 1212374 | 10433 | 10450 | 1762 | 1779 | AGGAAGGTGGCAGGCTGG | 131 | A | 53 |
| 1212384 | 10483 | 10500 | 1812 | 1829 | GTCCTTCCCCACACTCCA | 88 | A | 54 |
| 1212393 | 10533 | 10550 | 1862 | 1879 | CCAGCCCTGTCGGTTGTC | 86 | A | 55 |
| 1212402 | 10672 | 10689 | 1912 | 1929 | CAGTGGCGCCGATCAGCA | 90 | A | 56 |
| 1212412 | 10722 | 10739 | 1962 | 1979 | TGCGCAAACACTTGGTAC | 110 | A | 57 |
| 1212422 | 10772 | 10789 | 2012 | 2029 | CTGTCTCAAGGCTGGGTC | 69 | A | 58 |
| 1212432 | 10790 | 10807 | 2030 | 2047 | TACTGTCCCTCACAGCAG | N.D. | A | 59 |
| 1212442 | 10800 | 10817 | 2040 | 2057 | GAGTCTTCAGTACTGTCC | 92 | A | 60 |
| 1212452 | 10810 | 10827 | 2050 | 2067 | GAGGGCTGCAGAGTCTTC | 101 | A | 61 |
| 1212462 | 10820 | 10837 | 2060 | 2077 | GTGGGGTCCCGAGGGCTG | 80 | A | 62 |
| 1212472 | 10830 | 10847 | 2070 | 2087 | CACCCTCCGAGTGGGGTC | 124 | A | 63 |
| 1212482 | 10840 | 10857 | 2080 | 2097 | GAGCAGAGGGCACCCTCC | 94 | A | 64 |
| 1212492 | 10850 | 10867 | 2090 | 2107 | AGGGAGGCCTGAGCAGAG | 91 | A | 65 |
| 1212502 | 10860 | 10877 | 2100 | 2117 | GGGAGGTGCTAGGGAGGC | 62 | A | 66 |
| 1212512 | 10870 | 10887 | 2110 | 2127 | TTTGGTTAGGGGGAGGTG | 107 | A | 67 |
| 1212522 | 10880 | 10897 | 2120 | 2137 | CCAGGGAGAATTTGGTTA | 96 | A | 68 |
| 1212532 | 10890 | 10907 | 2130 | 2147 | AGAATGGGGTCCAGGGAG | 63 | A | 69 |
| 1212542 | 10900 | 10917 | 2140 | 2157 | TGGGGAGCTCAGAATGGG | 120 | A | 70 |
| 1212552 | 10910 | 10927 | 2150 | 2167 | CCCATGGTGATGGGGAGC | 68 | A | 71 |
| 1212562 | 10920 | 10937 | 2160 | 2177 | GGCCCCACCTCCCATGGT | 88 | A | 72 |
| 1212572 | 10930 | 10947 | 2170 | 2187 | CTTAGATTGAGGCCCCAC | 87 | A | 73 |
| 1212582 | 10940 | 10957 | 2180 | 2197 | CAGGGAAGGCCTTAGATT | 114 | A | 74 |
| 1212592 | 10950 | 10967 | 2190 | 2207 | CCCCTTCTGACAGGGAAG | 59 | A | 75 |
| 1212602 | 10960 | 10977 | 2200 | 2217 | TTGCCACAACCCCCTTCT | 68 | A | 76 |
| 1212612 | 10970 | 10987 | 2210 | 2227 | AATGTGGCTTTTGCCACA | 85 | A | 77 |
| 1212622 | 10980 | 10997 | 2220 | 2237 | GGCAGCTTGTAATGTGGC | 86 | A | 78 |
| 1212632 | 10990 | 11007 | 2230 | 2247 | GGGAGGGGATGGCAGCTT | 138 | A | 79 |
| 1212642 | 11000 | 11017 | 2240 | 2257 | CACTGAAACGGGGAGGGG | 84 | A | 80 |
| 1212652 | 11010 | 11027 | 2250 | 2267 | CCACAGGGTCCACTGAAA | 98 | A | 81 |
| 1212662 | 11020 | 11037 | 2260 | 2277 | AAGCACCTGGCCACAGGG | 128 | A | 82 |
| 1212672 | 11030 | 11047 | 2270 | 2287 | GGATAGGGAAAAGCACCT | 82 | A | 83 |
| 1212682 | 11040 | 11057 | 2280 | 2297 | ACACCCCTGTGGATAGGG | 86 | A | 84 |
| 1212692 | 11050 | 11067 | 2290 | 2307 | ACACACACAAACACCCCT | 83 | A | 85 |
| 1212702 | 11060 | 11077 | 2300 | 2317 | GCACACGCGCACACACAC | 92 | A | 86 |
| 1212712 | 11070 | 11087 | 2310 | 2327 | TTATTGAAACGCACACGC | 94 | A | 87 |
| 1212722 | 11080 | 11097 | 2320 | 2337 | TGTACAAACTTTATTGAA | 76 | A | 88 |
| 1212732 | N/A | N/A | 2330 | 2347 | TTTAAGAAAGTGTACAAA | 62 | A | 89 |
| 1211965 | 3090 | 3107 | 5 | 22 | AGGGATCATGTGATTGGA | 85 | B | 90 |
| 1211975 | 3130 | 3147 | 45 | 62 | ACTCTCCTCCCTGCTTCC | 79 | B | 91 |
| 1211985 | 3170 | 3187 | 85 | 102 | GGGCCAGCCTGGAATGCT | 91 | B | 92 |
| 1211995 | 3210 | 3227 | 125 | 142 | GGCTACCCGCTCCCATTG | 77 | B | 93 |
| 1212005 | 3250 | 3267 | 165 | 182 | GATGACCCGCCTACCTCA | 84 | B | 94 |
| 1212015 | 3284 | 3301 | 199 | 216 | AACCGGGTAGCGCTCAGA | 75 | B | 95 |
| 1212025 | 3294 | 3311 | 209 | 226 | GGGCAGCAGCAACCGGGT | 57 | B | 96 |
| 1212035 | 3304 | 3321 | 219 | 236 | CGCGGTCCTTGGGCAGCA | 193 | B | 97 |
| 1212045 | 3314 | 3331 | 229 | 246 | CGTCCGACTCCGCGGTCC | 87 | B | 98 |
| 1212055 | N/A | N/A | 239 | 256 | GGTCTGCCTGCGTCCGAC | 75 | B | 99 |
| 1212065 | 7157 | 7174 | 249 | 266 | GGGTCCACATGGTCTGCC | 100 | B | 100 |
| 1212075 | 7175 | 7192 | 267 | 284 | AGGCCACCCAGCTCACCA | 88 | B | 101 |
| 1212085 | 7225 | 7242 | 317 | 334 | GCAGAACTGACCATCTGG | 91 | B | 102 |
| 1212095 | 7275 | 7292 | 367 | 384 | CGGCAGCAGCTGTAGCTG | 66 | B | 103 |
| 1212105 | 7448 | 7465 | 417 | 434 | GGCCACCCAGATGCCTGC | 101 | B | 104 |
| 1212115 | 7498 | 7515 | 467 | 484 | GGTAAAGATGCAGGAGTG | 82 | B | 105 |
| 1212125 | N/A | N/A | 517 | 534 | CCGCATGCCACGGCCTCT | 89 | B | 106 |
| 1212135 | 7713 | 7730 | 567 | 584 | GCCCGTCTGCACTGCAGT | 140 | B | 107 |
| 1212145 | 8239 | 8256 | 617 | 634 | GCACTGGATGGCACCCAC | 108 | B | 108 |
| 1212155 | 8289 | 8306 | 667 | 684 | ACCATAACACAGCACGTG | 88 | B | 109 |
| 1212165 | 8440 | 8457 | 717 | 734 | CTTCACAGCAGGAAGCCT | 126 | B | 110 |
| 1212175 | 8490 | 8507 | 767 | 784 | GGTGTGAACCAGGTCGCA | 116 | B | 111 |
| 1212185 | 8540 | 8557 | 817 | 834 | GCAGGGAGCTTCTTTGCC | 85 | B | 112 |
| 1212195 | 8703 | 8720 | 867 | 884 | CCGGACACATGACCGAGC | 30† | B | 113 |
| 1212205 | 8753 | 8770 | 917 | 934 | ACTGGGCAGCTCACAGCA | 7† | B | 114 |
| 1212215 | 9039 | 9056 | 967 | 984 | TGATCGGAGCAGCAGGTG | 114 | B | 115 |
| 1212225 | 9089 | 9106 | 1017 | 1034 | ACTTACTCTGGATCAGGT | 98 | B | 116 |
| 1212235 | 9139 | 9156 | 1067 | 1084 | CGCAGGCAGCTTAGTGAG | 96 | B | 117 |
| 1212245 | 9388 | 9405 | 1117 | 1134 | CCATCTGGGCAGCTCACC | 120 | B | 118 |
| 1212255 | 9438 | 9455 | 1167 | 1184 | TAAAAGGGCAGCAGCCCC | 85 | B | 119 |
| 1212265 | 9577 | 9594 | 1217 | 1234 | AAACCCCGCGGGACAGCA | 94 | B | 120 |
| 1212275 | 9627 | 9644 | 1267 | 1284 | GGCACCTGGTGGGGCCCC | 63 | B | 121 |
| 1212285 | 9677 | 9694 | 1317 | 1334 | AGGCTTGTGGGTCTGGCA | 111 | B | 122 |
| 1212295 | 9727 | 9744 | 1367 | 1384 | ATCGGAGGAGGGACAGCT | 81 | B | 123 |
| 1212305 | 9777 | 9794 | 1417 | 1434 | TCTGGGATTGGACAGCAG | 114 | B | 124 |
| 1212315 | 10046 | 10063 | 1467 | 1484 | ACGTGTAGCCCTGGGGGC | 104 | B | 125 |
| 1212325 | 10096 | 10113 | 1517 | 1534 | CAGTCCAGCCACGATCTC | 106 | B | 126 |
| 1212335 | 10146 | 10163 | 1567 | 1584 | CCGATGTCTCTGGGGTGG | 87 | B | 127 |
| 1212345 | 10196 | 10213 | 1617 | 1634 | GGCTCGGGCAGCAGGTCT | 163 | B | 128 |
| 1212355 | N/A | N/A | 1667 | 1684 | CTCGCAGCACACAGCATG | 82 | B | 129 |
| 1212365 | 10388 | 10405 | 1717 | 1734 | CGAGCCTTCACGTTGCAG | 54 | B | 130 |
| 1212375 | 10438 | 10455 | 1767 | 1784 | GGGCCAGGAAGGTGGCAG | 73 | B | 131 |
| 1212385 | 10488 | 10505 | 1817 | 1834 | GAAGTGTCCTTCCCCACA | 81 | B | 132 |
| 1212394 | 10538 | 10555 | 1867 | 1884 | CAGGCCCAGCCCTGTCGG | 96 | B | 133 |
| 1212403 | 10677 | 10694 | 1917 | 1934 | GACAGCAGTGGCGCCGAT | 80 | B | 134 |
| 1212413 | 10727 | 10744 | 1967 | 1984 | CTCCCTGCGCAAACACTT | 66 | B | 135 |
| 1212423 | 10777 | 10794 | 2017 | 2034 | AGCAGCTGTCTCAAGGCT | 69 | B | 136 |
| 1212433 | 10791 | 10808 | 2031 | 2048 | GTACTGTCCCTCACAGCA | 89 | B | 137 |
| 1212443 | 10801 | 10818 | 2041 | 2058 | AGAGTCTTCAGTACTGTC | 123 | B | 138 |
| 1212453 | 10811 | 10828 | 2051 | 2068 | CGAGGGCTGCAGAGTCTT | 73 | B | 139 |
| 1212463 | 10821 | 10838 | 2061 | 2078 | AGTGGGGTCCCGAGGGCT | 100 | B | 140 |
| 1212473 | 10831 | 10848 | 2071 | 2088 | GCACCCTCCGAGTGGGGT | 69 | B | 141 |
| 1212483 | 10841 | 10858 | 2081 | 2098 | TGAGCAGAGGGCACCCTC | 112 | B | 142 |
| 1212493 | 10851 | 10868 | 2091 | 2108 | TAGGGAGGCCTGAGCAGA | 100 | B | 143 |
| 1212503 | 10861 | 10878 | 2101 | 2118 | GGGGAGGTGCTAGGGAGG | 93 | B | 144 |
| 1212513 | 10871 | 10888 | 2111 | 2128 | ATTTGGTTAGGGGGAGGT | 58 | B | 145 |
| 1212523 | 10881 | 10898 | 2121 | 2138 | TCCAGGGAGAATTTGGTT | 108 | B | 146 |
| 1212533 | 10891 | 10908 | 2131 | 2148 | CAGAATGGGGTCCAGGGA | 100 | B | 147 |
| 1212543 | 10901 | 10918 | 2141 | 2158 | ATGGGGAGCTCAGAATGG | 73 | B | 148 |
| 1212553 | 10911 | 10928 | 2151 | 2168 | TCCCATGGTGATGGGGAG | 93 | B | 149 |
| 1212563 | 10921 | 10938 | 2161 | 2178 | AGGCCCCACCTCCCATGG | 104 | B | 150 |
| 1212573 | 10931 | 10948 | 2171 | 2188 | CCTTAGATTGAGGCCCCA | 65 | B | 151 |
| 1212583 | 10941 | 10958 | 2181 | 2198 | ACAGGGAAGGCCTTAGAT | 78 | B | 152 |
| 1212593 | 10951 | 10968 | 2191 | 2208 | CCCCCTTCTGACAGGGAA | 67 | B | 153 |
| 1212603 | 10961 | 10978 | 2201 | 2218 | TTTGCCACAACCCCCTTC | 98 | B | 154 |
| 1212613 | 10971 | 10988 | 2211 | 2228 | TAATGTGGCTTTTGCCAC | 94 | B | 155 |
| 1212623 | 10981 | 10998 | 2221 | 2238 | TGGCAGCTTGTAATGTGG | 79 | B | 156 |
| 1212633 | 10991 | 11008 | 2231 | 2248 | GGGGAGGGGATGGCAGCT | 149 | B | 157 |
| 1212643 | 11001 | 11018 | 2241 | 2258 | CCACTGAAACGGGGAGGG | 65 | B | 158 |
| 1212653 | 11011 | 11028 | 2251 | 2268 | GCCACAGGGTCCACTGAA | 110 | B | 159 |
| 1212663 | 11021 | 11038 | 2261 | 2278 | AAAGCACCTGGCCACAGG | 101 | B | 160 |
| 1212673 | 11031 | 11048 | 2271 | 2288 | TGGATAGGGAAAAGCACC | 79 | B | 161 |
| 1212683 | 11041 | 11058 | 2281 | 2298 | AACACCCCTGTGGATAGG | 84 | B | 162 |
| 1212693 | 11051 | 11068 | 2291 | 2308 | CACACACACAAACACCCC | 73 | B | 163 |
| 1212703 | 11061 | 11078 | 2301 | 2318 | CGCACACGCGCACACACA | 83 | B | 164 |
| 1212713 | 11071 | 11088 | 2311 | 2328 | TTTATTGAAACGCACACG | 97 | B | 165 |
| 1212723 | 11081 | 11098 | 2321 | 2338 | GTGTACAAACTTTATTGA | 89 | B | 166 |
| 1212733 | N/A | N/A | 2331 | 2348 | TTTTAAGAAAGTGTACAA | 68 | B | 167 |
| 1211966 | 3094 | 3111 | 9 | 26 | TTCTAGGGATCATGTGAT | 83 | C | 168 |
| 1211976 | 3134 | 3151 | 49 | 66 | AATCACTCTCCTCCCTGC | 92 | C | 169 |
| 1211986 | 3174 | 3191 | 89 | 106 | GGTGGGGCCAGCCTGGAA | 81 | C | 170 |
| 1211996 | 3214 | 3231 | 129 | 146 | TCAGGGCTACCCGCTCCC | 73 | C | 171 |
| 1212006 | 3254 | 3271 | 169 | 186 | GCGCGATGACCCGCCTAC | 87 | C | 172 |
| 1212016 | 3285 | 3302 | 200 | 217 | CAACCGGGTAGCGCTCAG | 75 | C | 173 |
| 1212026 | 3295 | 3312 | 210 | 227 | TGGGCAGCAGCAACCGGG | 38 | C | 174 |
| 1212036 | 3305 | 3322 | 220 | 237 | CCGCGGTCCTTGGGCAGC | 151 | C | 175 |
| 1212046 | 3315 | 3332 | 230 | 247 | GCGTCCGACTCCGCGGTC | 84 | C | 176 |
| 1212056 | N/A | N/A | 240 | 257 | TGGTCTGCCTGCGTCCGA | 63 | C | 177 |
| 1212066 | 7158 | 7175 | 250 | 267 | AGGGTCCACATGGTCTGC | 55 | C | 178 |
| 1212076 | 7180 | 7197 | 272 | 289 | TGTTAAGGCCACCCAGCT | 98 | C | 179 |
| 1212086 | 7230 | 7247 | 322 | 339 | ACAGGGCAGAACTGACCA | 114 | C | 180 |
| 1212096 | 7280 | 7297 | 372 | 389 | GGGGACGGCAGCAGCTGT | 79 | C | 181 |
| 1212106 | 7453 | 7470 | 422 | 439 | GCAGGGGCCACCCAGATG | 103 | C | 182 |
| 1212116 | 7503 | 7520 | 472 | 489 | GAGACGGTAAAGATGCAG | 85 | C | 183 |
| 1212126 | 7668 | 7685 | 522 | 539 | CATCCCCGCATGCCACGG | 83 | C | 184 |
| 1212136 | 7718 | 7735 | 572 | 589 | GGATCGCCCGTCTGCACT | 91 | C | 185 |
| 1212146 | 8244 | 8261 | 622 | 639 | TCAGGGCACTGGATGGCA | 102 | C | 186 |
| 1212156 | 8294 | 8311 | 672 | 689 | CATCGACCATAACACAGC | 78 | C | 187 |
| 1212166 | 8445 | 8462 | 722 | 739 | CCTGTCTTCACAGCAGGA | 73 | C | 188 |
| 1212176 | 8495 | 8512 | 772 | 789 | CAGCGGGTGTGAACCAGG | 61 | C | 189 |
| 1212186 | 8545 | 8562 | 822 | 839 | TCTGGGCAGGGAGCTTCT | 135† | C | 190 |
| 1212196 | 8708 | 8725 | 872 | 889 | TGCGTCCGGACACATGAC | 87† | C | 191 |
| 1212206 | 8758 | 8775 | 922 | 939 | TTCCCACTGGGCAGCTCA | 32† | C | 192 |
| 1212216 | 9044 | 9061 | 972 | 989 | GCAGGTGATCGGAGCAGC | 92 | C | 193 |
| 1212226 | 9094 | 9111 | 1022 | 1039 | GAGGCACTTACTCTGGAT | 89 | C | 194 |
| 1212236 | 9144 | 9161 | 1072 | 1089 | GTGTGCGCAGGCAGCTTA | 92 | C | 195 |
| 1212246 | 9393 | 9410 | 1122 | 1139 | TATAGCCATCTGGGCAGC | 143 | C | 196 |
| 1212256 | 9443 | 9460 | 1172 | 1189 | CTGGGTAAAAGGGCAGCA | 171 | C | 197 |
| 1212266 | 9582 | 9599 | 1222 | 1239 | CACGTAAACCCCGCGGGA | 101 | C | 198 |
| 1212276 | 9632 | 9649 | 1272 | 1289 | TCCAGGGCACCTGGTGGG | 116 | C | 199 |
| 1212286 | 9682 | 9699 | 1322 | 1339 | CTTCAAGGCTTGTGGGTC | 110 | C | 200 |
| 1212296 | 9732 | 9749 | 1372 | 1389 | CAGGTATCGGAGGAGGGA | 99 | C | 201 |
| 1212306 | N/A | N/A | 1422 | 1439 | CAGCCTCTGGGATTGGAC | 105 | C | 202 |
| 1212316 | 10051 | 10068 | 1472 | 1489 | TACACACGTGTAGCCCTG | 108 | C | 203 |
| 1212326 | 10101 | 10118 | 1522 | 1539 | TTCTCCAGTCCAGCCACG | 67 | C | 204 |
| 1212336 | 10151 | 10168 | 1572 | 1589 | CACAGCCGATGTCTCTGG | 85 | C | 205 |
| 1212346 | 10201 | 10218 | 1622 | 1639 | ACCCAGGCTCGGGCAGCA | 108 | C | 206 |
| 1212356 | 10343 | 10360 | 1672 | 1689 | CGATCCTCGCAGCACACA | 103 | C | 207 |
| 1212366 | 10393 | 10410 | 1722 | 1739 | AGGATCGAGCCTTCACGT | 123 | C | 208 |
| 1212376 | 10443 | 10460 | 1772 | 1789 | GCTACGGGCCAGGAAGGT | 106 | C | 209 |
| 1212386 | 10493 | 10510 | 1822 | 1839 | TGGCAGAAGTGTCCTTCC | 96 | C | 210 |
| 1212395 | 10543 | 10560 | 1872 | 1889 | GACAGCAGGCCCAGCCCT | 91 | C | 211 |
| 1212404 | 10682 | 10699 | 1922 | 1939 | AGCAGGACAGCAGTGGCG | 82 | C | 212 |
| 1212414 | 10732 | 10749 | 1972 | 1989 | GGGGCCTCCCTGCGCAAA | 98 | C | 213 |
| 1212424 | 10782 | 10799 | 2022 | 2039 | CTCACAGCAGCTGTCTCA | 86 | C | 214 |
| 1212434 | 10792 | 10809 | 2032 | 2049 | AGTACTGTCCCTCACAGC | 102 | C | 215 |
| 1212444 | 10802 | 10819 | 2042 | 2059 | CAGAGTCTTCAGTACTGT | 74 | C | 216 |
| 1212454 | 10812 | 10829 | 2052 | 2069 | CCGAGGGCTGCAGAGTCT | 106 | C | 217 |
| 1212464 | 10822 | 10839 | 2062 | 2079 | GAGTGGGGTCCCGAGGGC | 92 | C | 218 |
| 1212474 | 10832 | 10849 | 2072 | 2089 | GGCACCCTCCGAGTGGGG | 108 | C | 219 |
| 1212484 | 10842 | 10859 | 2082 | 2099 | CTGAGCAGAGGGCACCCT | 72 | C | 220 |
| 1212494 | 10852 | 10869 | 2092 | 2109 | CTAGGGAGGCCTGAGCAG | 80 | C | 221 |
| 1212504 | 10862 | 10879 | 2102 | 2119 | GGGGGAGGTGCTAGGGAG | 112 | C | 222 |
| 1212514 | 10872 | 10889 | 2112 | 2129 | AATTTGGTTAGGGGGAGG | 73 | C | 223 |
| 1212524 | 10882 | 10899 | 2122 | 2139 | GTCCAGGGAGAATTTGGT | 69 | C | 224 |
| 1212534 | 10892 | 10909 | 2132 | 2149 | TCAGAATGGGGTCCAGGG | 63 | C | 225 |
| 1212544 | 10902 | 10919 | 2142 | 2159 | GATGGGGAGCTCAGAATG | 70 | C | 226 |
| 1212554 | 10912 | 10929 | 2152 | 2169 | CTCCCATGGTGATGGGGA | 92 | C | 227 |
| 1212564 | 10922 | 10939 | 2162 | 2179 | GAGGCCCCACCTCCCATG | 69 | C | 228 |
| 1212574 | 10932 | 10949 | 2172 | 2189 | GCCTTAGATTGAGGCCCC | 114 | C | 229 |
| 1212584 | 10942 | 10959 | 2182 | 2199 | GACAGGGAAGGCCTTAGA | 110 | C | 230 |
| 1212594 | 10952 | 10969 | 2192 | 2209 | ACCCCCTTCTGACAGGGA | 91 | C | 231 |
| 1212604 | 10962 | 10979 | 2202 | 2219 | TTTTGCCACAACCCCCTT | 73 | C | 232 |
| 1212614 | 10972 | 10989 | 2212 | 2229 | GTAATGTGGCTTTTGCCA | 126 | C | 233 |
| 1212624 | 10982 | 10999 | 2222 | 2239 | ATGGCAGCTTGTAATGTG | 88 | C | 234 |
| 1212634 | 10992 | 11009 | 2232 | 2249 | CGGGGAGGGGATGGCAGC | 95 | C | 235 |
| 1212644 | 11002 | 11019 | 2242 | 2259 | TCCACTGAAACGGGGAGG | 88 | C | 236 |
| 1212654 | 11012 | 11029 | 2252 | 2269 | GGCCACAGGGTCCACTGA | 81 | C | 237 |
| 1212664 | 11022 | 11039 | 2262 | 2279 | AAAAGCACCTGGCCACAG | 93 | C | 238 |
| 1212674 | 11032 | 11049 | 2272 | 2289 | GTGGATAGGGAAAAGCAC | 113 | C | 239 |
| 1212684 | 11042 | 11059 | 2282 | 2299 | AAACACCCCTGTGGATAG | 84 | C | 240 |
| 1212694 | 11052 | 11069 | 2292 | 2309 | GCACACACACAAACACCC | 97 | C | 241 |
| 1212704 | 11062 | 11079 | 2302 | 2319 | ACGCACACGCGCACACAC | 75 | C | 242 |
| 1212714 | 11072 | 11089 | 2312 | 2329 | CTTTATTGAAACGCACAC | 74 | C | 243 |
| 1212724 | 11082 | 11099 | 2322 | 2339 | AGTGTACAAACTTTATTG | 87 | C | 244 |
| 1212734 | N/A | N/A | 2332 | 2349 | TTTTTAAGAAAGTGTACA | 79 | C | 245 |
| 887390 | 10548 | 10565 | 1877 | 1894 | GTAGGGACAGCAGGCCCA | 85 | D | 246 |
| 1211967 | 3098 | 3115 | 13 | 30 | CCATTTCTAGGGATCATG | 80 | D | 247 |
| 1211977 | 3138 | 3155 | 53 | 70 | CTCAAATCACTCTCCTCC | 76 | D | 248 |
| 1211987 | 3178 | 3195 | 93 | 110 | TAGAGGTGGGGCCAGCCT | 97 | D | 249 |
| 1211997 | 3218 | 3235 | 133 | 150 | GGGATCAGGGCTACCCGC | 99 | D | 250 |
| 1212007 | 3258 | 3275 | 173 | 190 | CCCAGCGCGATGACCCGC | 108 | D | 251 |
| 1212017 | 3286 | 3303 | 201 | 218 | GCAACCGGGTAGCGCTCA | 72 | D | 252 |
| 1212027 | 3296 | 3313 | 211 | 228 | TTGGGCAGCAGCAACCGG | 42 | D | 253 |
| 1212037 | 3306 | 3323 | 221 | 238 | TCCGCGGTCCTTGGGCAG | 114 | D | 254 |
| 1212047 | 3316 | 3333 | 231 | 248 | TGCGTCCGACTCCGCGGT | 87 | D | 255 |
| 1212057 | N/A | N/A | 241 | 258 | ATGGTCTGCCTGCGTCCG | 76 | D | 256 |
| 1212067 | 7159 | 7176 | 251 | 268 | CAGGGTCCACATGGTCTG | 73 | D | 257 |
| 1212077 | 7185 | 7202 | 277 | 294 | CCTGCTGTTAAGGCCACC | 55 | D | 258 |
| 1212087 | 7235 | 7252 | 327 | 344 | AGGCCACAGGGCAGAACT | 86 | D | 259 |
| 1212097 | 7285 | 7302 | 377 | 394 | CAGAAGGGGACGGCAGCA | 80 | D | 260 |
| 1212107 | 7458 | 7475 | 427 | 444 | ACCTGGCAGGGGCCACCC | 102 | D | 261 |
| 1212117 | 7508 | 7525 | 477 | 494 | TCCCTGAGACGGTAAAGA | 102 | D | 262 |
| 1212127 | 7673 | 7690 | 527 | 544 | ATGGCCATCCCCGCATGC | 92 | D | 263 |
| 1212137 | 7723 | 7740 | 577 | 594 | AAGCAGGATCGCCCGTCT | 120 | D | 264 |
| 1212147 | 8249 | 8266 | 627 | 644 | GACTATCAGGGCACTGGA | 62 | D | 265 |
| 1212157 | 8299 | 8316 | 677 | 694 | GGAGCCATCGACCATAAC | 114 | D | 266 |
| 1212167 | 8450 | 8467 | 727 | 744 | TGCACCCTGTCTTCACAG | 101 | D | 267 |
| 1212177 | 8500 | 8517 | 777 | 794 | TGATGCAGCGGGTGTGAA | 88 | D | 268 |
| 1212187 | 8550 | 8567 | 827 | 844 | AGTCCTCTGGGCAGGGAG | 114† | D | 269 |
| 1212197 | 8713 | 8730 | 877 | 894 | GACCGTGCGTCCGGACAC | 4† | D | 270 |
| 1212207 | 8763 | 8780 | 927 | 944 | CATACTTCCCACTGGGCA | 69† | D | 271 |
| 1212217 | 9049 | 9066 | 977 | 994 | GCAGTGCAGGTGATCGGA | 84 | D | 272 |
| 1212227 | 9099 | 9116 | 1027 | 1044 | TTGGAGAGGCACTTACTC | 74 | D | 273 |
| 1212237 | N/A | N/A | 1077 | 1094 | CCACTGTGTGCGCAGGCA | 97 | D | 274 |
| 1212247 | 9398 | 9415 | 1127 | 1144 | GCAGGTATAGCCATCTGG | 100 | D | 275 |
| 1212257 | N/A | N/A | 1177 | 1194 | ACAGCCTGGGTAAAAGGG | 163 | D | 276 |
| 1212267 | 9587 | 9604 | 1227 | 1244 | TGTCACACGTAAACCCCG | 73 | D | 277 |
| 1212277 | 9637 | 9654 | 1277 | 1294 | CTCCATCCAGGGCACCTG | 80 | D | 278 |
| 1212287 | 9687 | 9704 | 1327 | 1344 | TCTCTCTTCAAGGCTTGT | 84 | D | 279 |
| 1212297 | 9737 | 9754 | 1377 | 1394 | GGCAGCAGGTATCGGAGG | 110 | D | 280 |
| 1212307 | N/A | N/A | 1427 | 1444 | GCAGACAGCCTCTGGGAT | 81 | D | 281 |
| 1212317 | 10056 | 10073 | 1477 | 1494 | TCAGCTACACACGTGTAG | 104 | D | 282 |
| 1212327 | 10106 | 10123 | 1527 | 1544 | GCATCTTCTCCAGTCCAG | 113 | D | 283 |
| 1212337 | 10156 | 10173 | 1577 | 1594 | CTGGTCACAGCCGATGTC | 70 | D | 284 |
| 1212347 | 10206 | 10223 | 1627 | 1644 | CTCCCACCCAGGCTCGGG | 93 | D | 285 |
| 1212357 | 10348 | 10365 | 1677 | 1694 | GCTGGCGATCCTCGCAGC | 85 | D | 286 |
| 1212367 | 10398 | 10415 | 1727 | 1744 | CTCGCAGGATCGAGCCTT | 71 | D | 287 |
| 1212377 | 10448 | 10465 | 1777 | 1794 | TGAGGGCTACGGGCCAGG | 86 | D | 288 |
| 1212387 | 10498 | 10515 | 1827 | 1844 | TATCATGGCAGAAGTGTC | 71 | D | 289 |
| 1212405 | 10687 | 10704 | 1927 | 1944 | AAGCCAGCAGGACAGCAG | 119 | D | 290 |
| 1212415 | 10737 | 10754 | 1977 | 1994 | AGCGCGGGGCCTCCCTGC | 96 | D | 291 |
| 1212425 | 10783 | 10800 | 2023 | 2040 | CCTCACAGCAGCTGTCTC | 74 | D | 292 |
| 1212435 | 10793 | 10810 | 2033 | 2050 | CAGTACTGTCCCTCACAG | 94 | D | 293 |
| 1212445 | 10803 | 10820 | 2043 | 2060 | GCAGAGTCTTCAGTACTG | 97 | D | 294 |
| 1212455 | 10813 | 10830 | 2053 | 2070 | CCCGAGGGCTGCAGAGTC | 74 | D | 295 |
| 1212465 | 10823 | 10840 | 2063 | 2080 | CGAGTGGGGTCCCGAGGG | 79 | D | 296 |
| 1212475 | 10833 | 10850 | 2073 | 2090 | GGGCACCCTCCGAGTGGG | 78 | D | 297 |
| 1212485 | 10843 | 10860 | 2083 | 2100 | CCTGAGCAGAGGGCACCC | 88 | D | 298 |
| 1212495 | 10853 | 10870 | 2093 | 2110 | GCTAGGGAGGCCTGAGCA | 76 | D | 299 |
| 1212505 | 10863 | 10880 | 2103 | 2120 | AGGGGGAGGTGCTAGGGA | 153 | D | 300 |
| 1212515 | 10873 | 10890 | 2113 | 2130 | GAATTTGGTTAGGGGGAG | 97 | D | 301 |
| 1212525 | 10883 | 10900 | 2123 | 2140 | GGTCCAGGGAGAATTTGG | 112 | D | 302 |
| 1212535 | 10893 | 10910 | 2133 | 2150 | CTCAGAATGGGGTCCAGG | 108 | D | 303 |
| 1212545 | 10903 | 10920 | 2143 | 2160 | TGATGGGGAGCTCAGAAT | 80 | D | 304 |
| 1212555 | 10913 | 10930 | 2153 | 2170 | CCTCCCATGGTGATGGGG | 105 | D | 305 |
| 1212565 | 10923 | 10940 | 2163 | 2180 | TGAGGCCCCACCTCCCAT | 78 | D | 306 |
| 1212575 | 10933 | 10950 | 2173 | 2190 | GGCCTTAGATTGAGGCCC | 62 | D | 307 |
| 1212585 | 10943 | 10960 | 2183 | 2200 | TGACAGGGAAGGCCTTAG | 89 | D | 308 |
| 1212595 | 10953 | 10970 | 2193 | 2210 | AACCCCCTTCTGACAGGG | 89 | D | 309 |
| 1212605 | 10963 | 10980 | 2203 | 2220 | CTTTTGCCACAACCCCCT | 101 | D | 310 |
| 1212615 | 10973 | 10990 | 2213 | 2230 | TGTAATGTGGCTTTTGCC | 110 | D | 311 |
| 1212625 | 10983 | 11000 | 2223 | 2240 | GATGGCAGCTTGTAATGT | 135 | D | 312 |
| 1212635 | 10993 | 11010 | 2233 | 2250 | ACGGGGAGGGGATGGCAG | 135 | D | 313 |
| 1212645 | 11003 | 11020 | 2243 | 2260 | GTCCACTGAAACGGGGAG | 103 | D | 314 |
| 1212655 | 11013 | 11030 | 2253 | 2270 | TGGCCACAGGGTCCACTG | 83 | D | 315 |
| 1212665 | 11023 | 11040 | 2263 | 2280 | GAAAAGCACCTGGCCACA | 123 | D | 316 |
| 1212675 | 11033 | 11050 | 2273 | 2290 | TGTGGATAGGGAAAAGCA | 115 | D | 317 |
| 1212685 | 11043 | 11060 | 2283 | 2300 | CAAACACCCCTGTGGATA | 88 | D | 318 |
| 1212695 | 11053 | 11070 | 2293 | 2310 | CGCACACACACAAACACC | 74 | D | 319 |
| 1212705 | 11063 | 11080 | 2303 | 2320 | AACGCACACGCGCACACA | 82 | D | 320 |
| 1212715 | 11073 | 11090 | 2313 | 2330 | ACTTTATTGAAACGCACA | 49 | D | 321 |
| 1212725 | 11083 | 11100 | 2323 | 2340 | AAGTGTACAAACTTTATT | 80 | D | 322 |
| 1212735 | N/A | N/A | 2333 | 2350 | TTTTTTAAGAAAGTGTAC | 84 | D | 323 |
| 1211968 | 3102 | 3119 | 17 | 34 | CACCCCATTTCTAGGGAT | 94 | E | 324 |
| 1211978 | 3142 | 3159 | 57 | 74 | TCTACTCAAATCACTCTC | 119 | E | 325 |
| 1211988 | 3182 | 3199 | 97 | 114 | AATATAGAGGTGGGGCCA | 103 | E | 326 |
| 1211998 | 3222 | 3239 | 137 | 154 | GCCAGGGATCAGGGCTAC | 100 | E | 327 |
| 1212008 | 3262 | 3279 | 177 | 194 | AGACCCCAGCGCGATGAC | 116 | E | 328 |
| 1212018 | 3287 | 3304 | 202 | 219 | AGCAACCGGGTAGCGCTC | 99 | E | 329 |
| 1212028 | 3297 | 3314 | 212 | 229 | CTTGGGCAGCAGCAACCG | 71 | E | 330 |
| 1212038 | 3307 | 3324 | 222 | 239 | CTCCGCGGTCCTTGGGCA | 96 | E | 331 |
| 1212048 | 3317 | 3334 | 232 | 249 | CTGCGTCCGACTCCGCGG | 96 | E | 332 |
| 1212058 | N/A | N/A | 242 | 259 | CATGGTCTGCCTGCGTCC | 107 | E | 333 |
| 1212068 | 7160 | 7177 | 252 | 269 | CCAGGGTCCACATGGTCT | 81 | E | 334 |
| 1212078 | 7190 | 7207 | 282 | 299 | CCAGCCCTGCTGTTAAGG | 100 | E | 335 |
| 1212088 | 7240 | 7257 | 332 | 349 | GCAGCAGGCCACAGGGCA | 133 | E | 336 |
| 1212098 | N/A | N/A | 382 | 399 | TTGTCCAGAAGGGGACGG | 120 | E | 337 |
| 1212108 | 7463 | 7480 | 432 | 449 | CATCAACCTGGCAGGGGC | 99 | E | 338 |
| 1212118 | 7513 | 7530 | 482 | 499 | GGAAGTCCCTGAGACGGT | 87 | E | 339 |
| 1212128 | 7678 | 7695 | 532 | 549 | CAGTGATGGCCATCCCCG | 130 | E | 340 |
| 1212138 | 7728 | 7745 | 582 | 599 | TTTGGAAGCAGGATCGCC | 112 | E | 341 |
| 1212148 | 8254 | 8271 | 632 | 649 | GAACTGACTATCAGGGCA | 90 | E | 342 |
| 1212158 | 8304 | 8321 | 682 | 699 | CCCCAGGAGCCATCGACC | 100 | E | 343 |
| 1212168 | 8455 | 8472 | 732 | 749 | AGCAGTGCACCCTGTCTT | 97 | E | 344 |
| 1212178 | 8505 | 8522 | 782 | 799 | GGGTGTGATGCAGCGGGT | 104 | E | 345 |
| 1212188 | 8555 | 8572 | 832 | 849 | CTGTTAGTCCTCTGGGCA | 42† | E | 346 |
| 1212198 | 8718 | 8735 | 882 | 899 | ACCGGGACCGTGCGTCCG | 27† | E | 347 |
| 1212208 | 8768 | 8785 | 932 | 949 | GCAGCCATACTTCCCACT | 15† | E | 348 |
| 1212218 | 9054 | 9071 | 982 | 999 | GGGCAGCAGTGCAGGTGA | 73 | E | 349 |
| 1212228 | 9104 | 9121 | 1032 | 1049 | TCTCCTTGGAGAGGCACT | 140 | E | 350 |
| 1212238 | N/A | N/A | 1082 | 1099 | ATCCCCCACTGTGTGCGC | 122 | E | 351 |
| 1212248 | 9403 | 9420 | 1132 | 1149 | CGGCAGCAGGTATAGCCA | 144 | E | 352 |
| 1212258 | N/A | N/A | 1182 | 1199 | AGCACACAGCCTGGGTAA | 94 | E | 353 |
| 1212268 | 9592 | 9609 | 1232 | 1249 | CTGCGTGTCACACGTAAA | 88 | E | 354 |
| 1212278 | 9642 | 9659 | 1282 | 1299 | GCCTTCTCCATCCAGGGC | 97 | E | 355 |
| 1212288 | 9692 | 9709 | 1332 | 1349 | GGACATCTCTCTTCAAGG | 90 | E | 356 |
| 1212298 | 9742 | 9759 | 1382 | 1399 | GAGTTGGCAGCAGGTATC | 158 | E | 357 |
| 1212308 | N/A | N/A | 1432 | 1449 | GAGCAGCAGACAGCCTCT | 104 | E | 358 |
| 1212318 | 10061 | 10078 | 1482 | 1499 | GCCCCTCAGCTACACACG | 88 | E | 359 |
| 1212328 | 10111 | 10128 | 1532 | 1549 | GGCAGGCATCTTCTCCAG | 87 | E | 360 |
| 1212338 | 10161 | 10178 | 1582 | 1599 | GTGTGCTGGTCACAGCCG | 110 | E | 361 |
| 1212348 | 10211 | 10228 | 1632 | 1649 | CCCAGCTCCCACCCAGGC | 107 | E | 362 |
| 1212358 | 10353 | 10370 | 1682 | 1699 | GCAGTGCTGGCGATCCTC | 82 | E | 363 |
| 1212368 | 10403 | 10420 | 1732 | 1749 | TCCTTCTCGCAGGATCGA | 93 | E | 364 |
| 1212378 | 10453 | 10470 | 1782 | 1799 | CCACGTGAGGGCTACGGG | 115 | E | 365 |
| 1212388 | 10503 | 10520 | 1832 | 1849 | CTGGTTATCATGGCAGAA | 85 | E | 366 |
| 1212396 | 10553 | 10570 | 1882 | 1899 | TGGCGGTAGGGACAGCAG | 130 | E | 367 |
| 1212406 | 10692 | 10709 | 1932 | 1949 | AGCGGAAGCCAGCAGGAC | 93 | E | 368 |
| 1212416 | 10742 | 10759 | 1982 | 1999 | GTCCCAGCGCGGGGCCTC | 113 | E | 369 |
| 1212426 | 10784 | 10801 | 2024 | 2041 | CCCTCACAGCAGCTGTCT | 113 | E | 370 |
| 1212436 | 10794 | 10811 | 2034 | 2051 | TCAGTACTGTCCCTCACA | 96 | E | 371 |
| 1212446 | 10804 | 10821 | 2044 | 2061 | TGCAGAGTCTTCAGTACT | 112 | E | 372 |
| 1212456 | 10814 | 10831 | 2054 | 2071 | TCCCGAGGGCTGCAGAGT | 104 | E | 373 |
| 1212466 | 10824 | 10841 | 2064 | 2081 | CCGAGTGGGGTCCCGAGG | 75 | E | 374 |
| 1212476 | 10834 | 10851 | 2074 | 2091 | AGGGCACCCTCCGAGTGG | 104 | E | 375 |
| 1212486 | 10844 | 10861 | 2084 | 2101 | GCCTGAGCAGAGGGCACC | 122 | E | 376 |
| 1212496 | 10854 | 10871 | 2094 | 2111 | TGCTAGGGAGGCCTGAGC | 86 | E | 377 |
| 1212506 | 10864 | 10881 | 2104 | 2121 | TAGGGGGAGGTGCTAGGG | 139 | E | 378 |
| 1212516 | 10874 | 10891 | 2114 | 2131 | AGAATTTGGTTAGGGGGA | 117 | E | 379 |
| 1212526 | 10884 | 10901 | 2124 | 2141 | GGGTCCAGGGAGAATTTG | 90 | E | 380 |
| 1212536 | 10894 | 10911 | 2134 | 2151 | GCTCAGAATGGGGTCCAG | 110 | E | 381 |
| 1212546 | 10904 | 10921 | 2144 | 2161 | GTGATGGGGAGCTCAGAA | 88 | E | 382 |
| 1212556 | 10914 | 10931 | 2154 | 2171 | ACCTCCCATGGTGATGGG | 83 | E | 383 |
| 1212566 | 10924 | 10941 | 2164 | 2181 | TTGAGGCCCCACCTCCCA | 101 | E | 384 |
| 1212576 | 10934 | 10951 | 2174 | 2191 | AGGCCTTAGATTGAGGCC | 75 | E | 385 |
| 1212586 | 10944 | 10961 | 2184 | 2201 | CTGACAGGGAAGGCCTTA | 98 | E | 386 |
| 1212596 | 10954 | 10971 | 2194 | 2211 | CAACCCCCTTCTGACAGG | 93 | E | 387 |
| 1212606 | 10964 | 10981 | 2204 | 2221 | GCTTTTGCCACAACCCCC | 104 | E | 388 |
| 1212616 | 10974 | 10991 | 2214 | 2231 | TTGTAATGTGGCTTTTGC | 98 | E | 389 |
| 1212626 | 10984 | 11001 | 2224 | 2241 | GGATGGCAGCTTGTAATG | 135 | E | 390 |
| 1212636 | 10994 | 11011 | 2234 | 2251 | AACGGGGAGGGGATGGCA | 85 | E | 391 |
| 1212646 | 11004 | 11021 | 2244 | 2261 | GGTCCACTGAAACGGGGA | 124 | E | 392 |
| 1212656 | 11014 | 11031 | 2254 | 2271 | CTGGCCACAGGGTCCACT | 84 | E | 393 |
| 1212666 | 11024 | 11041 | 2264 | 2281 | GGAAAAGCACCTGGCCAC | 110 | E | 394 |
| 1212676 | 11034 | 11051 | 2274 | 2291 | CTGTGGATAGGGAAAAGC | 96 | E | 395 |
| 1212686 | 11044 | 11061 | 2284 | 2301 | ACAAACACCCCTGTGGAT | 81 | E | 396 |
| 1212696 | 11054 | 11071 | 2294 | 2311 | GCGCACACACACAAACAC | 108 | E | 397 |
| 1212706 | 11064 | 11081 | 2304 | 2321 | AAACGCACACGCGCACAC | 109 | E | 398 |
| 1212716 | 11074 | 11091 | 2314 | 2331 | AACTTTATTGAAACGCAC | 107 | E | 399 |
| 1212726 | 11084 | 11101 | 2324 | 2341 | AAAGTGTACAAACTTTAT | 112 | E | 400 |
| 1212736 | N/A | N/A | 2334 | 2351 | TTTTTTTAAGAAAGTGTA | 94 | E | 401 |
| 887369 | 10508 | 10525 | 1837 | 1854 | CAGGTCTGGTTATCATGG | 98 | F | 402 |
| 1211969 | 3106 | 3123 | 21 | 38 | CCCACACCCCATTTCTAG | 115 | F | 403 |
| 1211979 | 3146 | 3163 | 61 | 78 | CTTTTCTACTCAAATCAC | 94 | F | 404 |
| 1211989 | 3186 | 3203 | 101 | 118 | TATCAATATAGAGGTGGG | 110 | F | 405 |
| 1211999 | 3226 | 3243 | 141 | 158 | ATTGGCCAGGGATCAGGG | 105 | F | 406 |
| 1212009 | 3266 | 3283 | 181 | 198 | CTACAGACCCCAGCGCGA | 98 | F | 407 |
| 1212019 | 3288 | 3305 | 203 | 220 | CAGCAACCGGGTAGCGCT | 106 | F | 408 |
| 1212029 | 3298 | 3315 | 213 | 230 | CCTTGGGCAGCAGCAACC | 75 | F | 409 |
| 1212039 | 3308 | 3325 | 223 | 240 | ACTCCGCGGTCCTTGGGC | 108 | F | 410 |
| 1212049 | 3318 | 3335 | 233 | 250 | CCTGCGTCCGACTCCGCG | 102 | F | 411 |
| 1212059 | N/A | N/A | 243 | 260 | ACATGGTCTGCCTGCGTC | 97 | F | 412 |
| 1212069 | 7161 | 7178 | 253 | 270 | ACCAGGGTCCACATGGTC | 95 | F | 413 |
| 1212079 | 7195 | 7212 | 287 | 304 | AGCCACCAGCCCTGCTGT | 98 | F | 414 |
| 1212089 | 7245 | 7262 | 337 | 354 | TCCAGGCAGCAGGCCACA | 112 | F | 415 |
| 1212099 | N/A | N/A | 387 | 404 | GCCATTTGTCCAGAAGGG | 109 | F | 416 |
| 1212109 | 7468 | 7485 | 437 | 454 | GTGGGCATCAACCTGGCA | 99 | F | 417 |
| 1212119 | 7518 | 7535 | 487 | 504 | CAACTGGAAGTCCCTGAG | 90 | F | 418 |
| 1212129 | 7683 | 7700 | 537 | 554 | GGCAGCAGTGATGGCCAT | 132 | F | 419 |
| 1212139 | 7733 | 7750 | 587 | 604 | TGATCTTTGGAAGCAGGA | 93 | F | 420 |
| 1212149 | 8259 | 8276 | 637 | 654 | CATTCGAACTGACTATCA | 99 | F | 421 |
| 1212159 | 8309 | 8326 | 687 | 704 | AGCACCCCCAGGAGCCAT | 84 | F | 422 |
| 1212169 | 8460 | 8477 | 737 | 754 | CGGACAGCAGTGCACCCT | 129 | F | 423 |
| 1212179 | 8510 | 8527 | 787 | 804 | CCCGTGGGTGTGATGCAG | 238 | F | 424 |
| 1212189 | 8560 | 8577 | 837 | 854 | CTGCCCTGTTAGTCCTCT | 8† | F | 425 |
| 1212199 | 8723 | 8740 | 887 | 904 | AGGGCACCGGGACCGTGC | 8† | F | 426 |
| 1212209 | 8773 | 8790 | 937 | 954 | GGGCAGCAGCCATACTTC | 22† | F | 427 |
| 1212219 | 9059 | 9076 | 987 | 1004 | CTTGGGGGCAGCAGTGCA | 151 | F | 428 |
| 1212229 | 9109 | 9126 | 1037 | 1054 | AGCGTTCTCCTTGGAGAG | 115 | F | 429 |
| 1212239 | 9358 | 9375 | 1087 | 1104 | TTCACATCCCCCACTGTG | 91 | F | 430 |
| 1212249 | 9408 | 9425 | 1137 | 1154 | GTAGACGGCAGCAGGTAT | 135 | F | 431 |
| 1212259 | N/A | N/A | 1187 | 1204 | CTCACAGCACACAGCCTG | 85 | F | 432 |
| 1212269 | 9597 | 9614 | 1237 | 1254 | CCCTTCTGCGTGTCACAC | 113 | F | 433 |
| 1212279 | 9647 | 9664 | 1287 | 1304 | CTGGGGCCTTCTCCATCC | 110 | F | 434 |
| 1212289 | 9697 | 9714 | 1337 | 1354 | ACAGGGGACATCTCTCTT | 92 | F | 435 |
| 1212299 | 9747 | 9764 | 1387 | 1404 | GACGTGAGTTGGCAGCAG | 116 | F | 436 |
| 1212309 | 10016 | 10033 | 1437 | 1454 | GGTCCGAGCAGCAGACAG | 105 | F | 437 |
| 1212319 | 10066 | 10083 | 1487 | 1504 | ACACTGCCCCTCAGCTAC | 96 | F | 438 |
| 1212329 | 10116 | 10133 | 1537 | 1554 | CGGCGGGCAGGCATCTTC | 91 | F | 439 |
| 1212339 | 10166 | 10183 | 1587 | 1604 | AGCTGGTGTGCTGGTCAC | 103 | F | 440 |
| 1212349 | 10216 | 10233 | 1637 | 1654 | GCAGGCCCAGCTCCCACC | 97 | F | 441 |
| 1212359 | 10358 | 10375 | 1687 | 1704 | GGGCAGCAGTGCTGGCGA | 133 | F | 442 |
| 1212369 | 10408 | 10425 | 1737 | 1754 | CCACTTCCTTCTCGCAGG | 97 | F | 443 |
| 1212379 | 10458 | 10475 | 1787 | 1804 | CACACCCACGTGAGGGCT | 100 | F | 444 |
| 1212397 | N/A | N/A | 1887 | 1904 | CGCCCTGGCGGTAGGGAC | 74 | F | 445 |
| 1212407 | 10697 | 10714 | 1937 | 1954 | TGCGCAGCGGAAGCCAGC | 114 | F | 446 |
| 1212417 | 10747 | 10764 | 1987 | 2004 | GGGGCGTCCCAGCGCGGG | 99 | F | 447 |
| 1212427 | 10785 | 10802 | 2025 | 2042 | TCCCTCACAGCAGCTGTC | 105 | F | 448 |
| 1212437 | 10795 | 10812 | 2035 | 2052 | TTCAGTACTGTCCCTCAC | 111 | F | 449 |
| 1212447 | 10805 | 10822 | 2045 | 2062 | CTGCAGAGTCTTCAGTAC | 113 | F | 450 |
| 1212457 | 10815 | 10832 | 2055 | 2072 | GTCCCGAGGGCTGCAGAG | 126 | F | 451 |
| 1212467 | 10825 | 10842 | 2065 | 2082 | TCCGAGTGGGGTCCCGAG | 113 | F | 452 |
| 1212477 | 10835 | 10852 | 2075 | 2092 | GAGGGCACCCTCCGAGTG | 112 | F | 453 |
| 1212487 | 10845 | 10862 | 2085 | 2102 | GGCCTGAGCAGAGGGCAC | 97 | F | 454 |
| 1212497 | 10855 | 10872 | 2095 | 2112 | GTGCTAGGGAGGCCTGAG | 103 | F | 455 |
| 1212507 | 10865 | 10882 | 2105 | 2122 | TTAGGGGGAGGTGCTAGG | 139 | F | 456 |
| 1212517 | 10875 | 10892 | 2115 | 2132 | GAGAATTTGGTTAGGGGG | 99 | F | 457 |
| 1212527 | 10885 | 10902 | 2125 | 2142 | GGGGTCCAGGGAGAATTT | 97 | F | 458 |
| 1212537 | 10895 | 10912 | 2135 | 2152 | AGCTCAGAATGGGGTCCA | 84 | F | 459 |
| 1212547 | 10905 | 10922 | 2145 | 2162 | GGTGATGGGGAGCTCAGA | 86 | F | 460 |
| 1212557 | 10915 | 10932 | 2155 | 2172 | CACCTCCCATGGTGATGG | 114 | F | 461 |
| 1212567 | 10925 | 10942 | 2165 | 2182 | ATTGAGGCCCCACCTCCC | 96 | F | 462 |
| 1212577 | 10935 | 10952 | 2175 | 2192 | AAGGCCTTAGATTGAGGC | 97 | F | 463 |
| 1212587 | 10945 | 10962 | 2185 | 2202 | TCTGACAGGGAAGGCCTT | 84 | F | 464 |
| 1212597 | 10955 | 10972 | 2195 | 2212 | ACAACCCCCTTCTGACAG | 90 | | 465 |
| 1212607 | 10965 | 10982 | 2205 | 2222 | GGCTTTTGCCACAACCCC | 102 | F | 466 |
| 1212617 | 10975 | 10992 | 2215 | 2232 | CTTGTAATGTGGCTTTTG | 113 | F | 467 |
| 1212627 | 10985 | 11002 | 2225 | 2242 | GGGATGGCAGCTTGTAAT | 128 | F | 468 |
| 1212637 | 10995 | 11012 | 2235 | 2252 | AAACGGGGAGGGGATGGC | 106 | F | 469 |
| 1212647 | 11005 | 11022 | 2245 | 2262 | GGGTCCACTGAAACGGGG | 131 | F | 470 |
| 1212657 | 11015 | 11032 | 2255 | 2272 | CCTGGCCACAGGGTCCAC | 114 | F | 471 |
| 1212667 | 11025 | 11042 | 2265 | 2282 | GGGAAAAGCACCTGGCCA | 87 | F | 472 |
| 1212677 | 11035 | 11052 | 2275 | 2292 | CCTGTGGATAGGGAAAAG | 108 | F | 473 |
| 1212687 | 11045 | 11062 | 2285 | 2302 | CACAAACACCCCTGTGGA | 95 | F | 474 |
| 1212697 | 11055 | 11072 | 2295 | 2312 | CGCGCACACACACAAACA | 117 | F | 475 |
| 1212707 | 11065 | 11082 | 2305 | 2322 | GAAACGCACACGCGCACA | 87 | F | 476 |
| 1212717 | 11075 | 11092 | 2315 | 2332 | AAACTTTATTGAAACGCA | 77 | F | 477 |
| 1212727 | 11085 | 11102 | 2325 | 2342 | GAAAGTGTACAAACTTTA | 90 | F | 478 |
| 1212737 | N/A | N/A | 2335 | 2352 | TTTTTTTTAAGAAAGTGT | 111 | F | 479 |
| 1211970 | 3110 | 3127 | 25 | 42 | TCGCCCCACACCCCATTT | 78 | G | 480 |
| 1211980 | 3150 | 3167 | 65 | 82 | GTTTCTTTTCTACTCAAA | 106 | G | 481 |
| 1211990 | 3190 | 3207 | 105 | 122 | TACTTATCAATATAGAGG | 105 | G | 482 |
| 1212000 | 3230 | 3247 | 145 | 162 | TTCCATTGGCCAGGGATC | 196 | G | 483 |
| 1212010 | 3270 | 3287 | 185 | 202 | CAGACTACAGACCCCAGC | 104 | G | 484 |
| 1212020 | 3289 | 3306 | 204 | 221 | GCAGCAACCGGGTAGCGC | 96 | G | 485 |
| 1212030 | 3299 | 3316 | 214 | 231 | TCCTTGGGCAGCAGCAAC | 78 | G | 486 |
| 1212040 | 3309 | 3326 | 224 | 241 | GACTCCGCGGTCCTTGGG | 112 | G | 487 |
| 1212050 | N/A | N/A | 234 | 251 | GCCTGCGTCCGACTCCGC | 129 | G | 488 |
| 1212060 | N/A | N/A | 244 | 261 | CACATGGTCTGCCTGCGT | 102 | G | 489 |
| 1212070 | 7162 | 7179 | 254 | 271 | CACCAGGGTCCACATGGT | 87 | G | 490 |
| 1212080 | 7200 | 7217 | 292 | 309 | GTTCCAGCCACCAGCCCT | 261 | G | 491 |
| 1212090 | 7250 | 7267 | 342 | 359 | CGGGGTCCAGGCAGCAGG | 123 | G | 492 |
| 1212100 | N/A | N/A | 392 | 409 | TGTGGGCCATTTGTCCAG | 85 | G | 493 |
| 1212110 | 7473 | 7490 | 442 | 459 | GAGCAGTGGGCATCAACC | 124 | G | 494 |
| 1212120 | 7523 | 7540 | 492 | 509 | GGCAGCAACTGGAAGTCC | 94 | G | 495 |
| 1212130 | 7688 | 7705 | 542 | 559 | CCGTGGGCAGCAGTGATG | 133 | G | 496 |
| 1212140 | N/A | N/A | 592 | 609 | TTACCTGATCTTTGGAAG | 122 | G | 497 |
| 1212150 | 8264 | 8281 | 642 | 659 | CCGGGCATTCGAACTGAC | 81 | G | 498 |
| 1212160 | 8314 | 8331 | 692 | 709 | GGGGCAGCACCCCCAGGA | 114 | G | 499 |
| 1212170 | 8465 | 8482 | 742 | 759 | CCGTGCGGACAGCAGTGC | 100 | G | 500 |
| 1212180 | 8515 | 8532 | 792 | 809 | GGGTGCCCGTGGGTGTGA | 294 | G | 501 |
| 1212190 | N/A | N/A | 842 | 859 | GGCCACTGCCCTGTTAGT | 83+ | G | 502 |
| 1212200 | 8728 | 8745 | 892 | 909 | CCATCAGGGCACCGGGAC | 7+ | G | 503 |
| 1212210 | 8778 | 8795 | 942 | 959 | GCATTGGGCAGCAGCCAT | 35+ | G | 504 |
| 1212220 | 9064 | 9081 | 992 | 1009 | AGTGTCTTGGGGGCAGCA | 123 | G | 505 |
| 1212230 | 9114 | 9131 | 1042 | 1059 | GTGGTAGCGTTCTCCTTG | 90 | G | 506 |
| 1212240 | 9363 | 9380 | 1092 | 1109 | CACATTTCACATCCCCCA | 117 | G | 507 |
| 1212250 | 9413 | 9430 | 1142 | 1159 | CGACTGTAGACGGCAGCA | 107 | G | 508 |
| 1212260 | 9552 | 9569 | 1192 | 1209 | TGGTCCTCACAGCACACA | 99 | G | 509 |
| 1212270 | 9602 | 9619 | 1242 | 1259 | AGGTACCCTTCTGCGTGT | 91 | G | 510 |
| 1212280 | 9652 | 9669 | 1292 | 1309 | GTGAGCTGGGGCCTTCTC | 86 | G | 511 |
| 1212290 | 9702 | 9719 | 1342 | 1359 | TTATCACAGGGGACATCT | 82 | G | 512 |
| 1212300 | 9752 | 9769 | 1392 | 1409 | CCCCAGACGTGAGTTGGC | 104 | G | 513 |
| 1212310 | 10021 | 10038 | 1442 | 1459 | CTGGTGGTCCGAGCAGCA | 102 | G | 514 |
| 1212320 | 10071 | 10088 | 1492 | 1509 | CGCTGACACTGCCCCTCA | 97 | G | 515 |
| 1212330 | 10121 | 10138 | 1542 | 1559 | AAGCCCGGCGGGCAGGCA | 78 | G | 516 |
| 1212340 | 10171 | 10188 | 1592 | 1609 | CGGGCAGCTGGTGTGCTG | 97 | G | 517 |
| 1212350 | 10221 | 10238 | 1642 | 1659 | TGGCAGCAGGCCCAGCTC | 113 | G | 518 |
| 1212360 | 10363 | 10380 | 1692 | 1709 | CAGCCGGGCAGCAGTGCT | 106 | G | 519 |
| 1212370 | 10413 | 10430 | 1742 | 1759 | AGAGACCACTTCCTTCTC | 91 | G | 520 |
| 1212380 | 10463 | 10480 | 1792 | 1809 | TCCTTCACACCCACGTGA | 92 | G | 521 |
| 1212389 | 10513 | 10530 | 1842 | 1859 | GGCAGCAGGTCTGGTTAT | 78 | G | 522 |
| 1212398 | N/A | N/A | 1892 | 1909 | ACAGACGCCCTGGCGGTA | 105 | G | 523 |
| 1212408 | 10702 | 10719 | 1942 | 1959 | CTGGCTGCGCAGCGGAAG | 102 | G | 524 |
| 1212418 | 10752 | 10769 | 1992 | 2009 | TCAAAGGGGCGTCCCAGC | 87 | G | 525 |
| 1212428 | 10786 | 10803 | 2026 | 2043 | GTCCCTCACAGCAGCTGT | 76 | G | 526 |
| 1212438 | 10796 | 10813 | 2036 | 2053 | CTTCAGTACTGTCCCTCA | 100 | G | 527 |
| 1212448 | 10806 | 10823 | 2046 | 2063 | GCTGCAGAGTCTTCAGTA | 87 | G | 528 |
| 1212458 | 10816 | 10833 | 2056 | 2073 | GGTCCCGAGGGCTGCAGA | 97 | G | 529 |
| 1212468 | 10826 | 10843 | 2066 | 2083 | CTCCGAGTGGGGTCCCGA | 78 | G | 530 |
| 1212478 | 10836 | 10853 | 2076 | 2093 | AGAGGGCACCCTCCGAGT | 132 | G | 531 |
| 1212488 | 10846 | 10863 | 2086 | 2103 | AGGCCTGAGCAGAGGGCA | 81 | G | 532 |
| 1212498 | 10856 | 10873 | 2096 | 2113 | GGTGCTAGGGAGGCCTGA | 102 | G | 533 |
| 1212508 | 10866 | 10883 | 2106 | 2123 | GTTAGGGGGAGGTGCTAG | 140 | G | 534 |
| 1212518 | 10876 | 10893 | 2116 | 2133 | GGAGAATTTGGTTAGGGG | 106 | G | 535 |
| 1212528 | 10886 | 10903 | 2126 | 2143 | TGGGGTCCAGGGAGAATT | 85 | G | 536 |
| 1212538 | 10896 | 10913 | 2136 | 2153 | GAGCTCAGAATGGGGTCC | 96 | G | 537 |
| 1212548 | 10906 | 10923 | 2146 | 2163 | TGGTGATGGGGAGCTCAG | 109 | G | 538 |
| 1212558 | 10916 | 10933 | 2156 | 2173 | CCACCTCCCATGGTGATG | 123 | G | 539 |
| 1212568 | 10926 | 10943 | 2166 | 2183 | GATTGAGGCCCCACCTCC | 100 | G | 540 |
| 1212578 | 10936 | 10953 | 2176 | 2193 | GAAGGCCTTAGATTGAGG | 76 | G | 541 |
| 1212588 | 10946 | 10963 | 2186 | 2203 | TTCTGACAGGGAAGGCCT | 104 | G | 542 |
| 1212598 | 10956 | 10973 | 2196 | 2213 | CACAACCCCCTTCTGACA | 98 | G | 543 |
| 1212608 | 10966 | 10983 | 2206 | 2223 | TGGCTTTTGCCACAACCC | 100 | G | 544 |
| 1212618 | 10976 | 10993 | 2216 | 2233 | GCTTGTAATGTGGCTTTT | 68 | G | 545 |
| 1212628 | 10986 | 11003 | 2226 | 2243 | GGGGATGGCAGCTTGTAA | 145 | G | 546 |
| 1212638 | 10996 | 11013 | 2236 | 2253 | GAAACGGGGAGGGGATGG | 92 | G | 547 |
| 1212648 | 11006 | 11023 | 2246 | 2263 | AGGGTCCACTGAAACGGG | 110 | G | 548 |
| 1212658 | 11016 | 11033 | 2256 | 2273 | ACCTGGCCACAGGGTCCA | 163 | G | 549 |
| 1212668 | 11026 | 11043 | 2266 | 2283 | AGGGAAAAGCACCTGGCC | 102 | G | 550 |
| 1212678 | 11036 | 11053 | 2276 | 2293 | CCCTGTGGATAGGGAAAA | 127 | G | 551 |
| 1212688 | 11046 | 11063 | 2286 | 2303 | ACACAAACACCCCTGTGG | 106 | G | 552 |
| 1212698 | 11056 | 11073 | 2296 | 2313 | ACGCGCACACACACAAAC | 115 | G | 553 |
| 1212708 | 11066 | 11083 | 2306 | 2323 | TGAAACGCACACGCGCAC | 81 | G | 554 |
| 1212718 | 11076 | 11093 | 2316 | 2333 | CAAACTTTATTGAAACGC | 100 | G | 555 |
| 1212728 | 11086 | 11103 | 2326 | 2343 | AGAAAGTGTACAAACTTT | 111 | G | 556 |
| 1212738 | N/A | N/A | 2336 | 2353 | TTTTTTTTTAAGAAAGTG | 100 | G | 557 |
| 1211971 | 3114 | 3131 | 29 | 46 | CCTCTCGCCCCACACCCC | 65 | H | 558 |
| 1211981 | 3154 | 3171 | 69 | 86 | CTGTGTTTCTTTTCTACT | 90 | H | 559 |
| 1211991 | 3194 | 3211 | 109 | 126 | TGGCTACTTATCAATATA | 95 | H | 560 |
| 1212001 | 3234 | 3251 | 149 | 166 | CAGTTTCCATTGGCCAGG | 104 | H | 561 |
| 1212011 | 3274 | 3291 | 189 | 206 | CGCTCAGACTACAGACCC | 100 | H | 562 |
| 1212021 | 3290 | 3307 | 205 | 222 | AGCAGCAACCGGGTAGCG | 82 | H | 563 |
| 1212031 | 3300 | 3317 | 215 | 232 | GTCCTTGGGCAGCAGCAA | 73 | H | 564 |
| 1212041 | 3310 | 3327 | 225 | 242 | CGACTCCGCGGTCCTTGG | 57 | H | 565 |
| 1212051 | N/A | N/A | 235 | 252 | TGCCTGCGTCCGACTCCG | 86 | H | 566 |
| 1212061 | N/A | N/A | 245 | 262 | CCACATGGTCTGCCTGCG | 101 | H | 567 |
| 1212071 | 7163 | 7180 | 255 | 272 | TCACCAGGGTCCACATGG | 151 | H | 568 |
| 1212081 | 7205 | 7222 | 297 | 314 | ACCGCGTTCCAGCCACCA | 103 | H | 569 |
| 1212091 | 7255 | 7272 | 347 | 364 | TCCTCCGGGGTCCAGGCA | 81 | H | 570 |
| 1212101 | 7428 | 7445 | 397 | 414 | AGTGTTGTGGGCCATTTG | 77 | H | 571 |
| 1212111 | 7478 | 7495 | 447 | 464 | CGGCAGAGCAGTGGGCAT | 115 | H | 572 |
| 1212121 | 7528 | 7545 | 497 | 514 | GAAGGGGCAGCAACTGGA | 115 | H | 573 |
| 1212131 | 7693 | 7710 | 547 | 564 | AAGCCCCGTGGGCAGCAG | 298 | H | 574 |
| 1212141 | N/A | N/A | 597 | 614 | AGTTGTTACCTGATCTTT | 103 | H | 575 |
| 1212151 | 8269 | 8286 | 647 | 664 | GAAGTCCGGGCATTCGAA | 68 | H | 576 |
| 1212161 | 8319 | 8336 | 697 | 714 | GGCATGGGGCAGCACCCC | 143 | H | 577 |
| 1212171 | 8470 | 8487 | 747 | 764 | AGGCACCGTGCGGACAGC | 78 | H | 578 |
| 1212181 | 8520 | 8537 | 797 | 814 | GGGGTGGGTGCCCGTGGG | 228 | H | 579 |
| 1212191 | N/A | N/A | 847 | 864 | GACAAGGCCACTGCCCTG | 77† | H | 580 |
| 1212201 | 8733 | 8750 | 897 | 914 | TAGAACCATCAGGGCACC | 13† | H | 581 |
| 1212211 | 8783 | 8800 | 947 | 964 | GTTGGGCATTGGGCAGCA | 361+ | H | 582 |
| 1212221 | 9069 | 9086 | 997 | 1014 | CACACAGTGTCTTGGGGG | 73 | H | 583 |
| 1212231 | 9119 | 9136 | 1047 | 1064 | GGTCCGTGGTAGCGTTCT | 102 | H | 584 |
| 1212241 | 9368 | 9385 | 1097 | 1114 | CATGTCACATTTCACATC | 114 | H | 585 |
| 1212251 | 9418 | 9435 | 1147 | 1164 | GCCCCCGACTGTAGACGG | 104 | H | 586 |
| 1212261 | 9557 | 9574 | 1197 | 1214 | GTATGTGGTCCTCACAGC | 114 | H | 587 |
| 1212271 | 9607 | 9624 | 1247 | 1264 | TTCACAGGTACCCTTCTG | 62 | H | 588 |
| 1212281 | 9657 | 9674 | 1297 | 1314 | CTGAGGTGAGCTGGGGCC | 76 | H | 589 |
| 1212291 | 9707 | 9724 | 1347 | 1364 | TGACATTATCACAGGGGA | 73 | H | 590 |
| 1212301 | 9757 | 9774 | 1397 | 1414 | CCACTCCCCAGACGTGAG | 91 | H | 591 |
| 1212311 | 10026 | 10043 | 1447 | 1464 | CAGTGCTGGTGGTCCGAG | 77 | H | 592 |
| 1212321 | 10076 | 10093 | 1497 | 1514 | TTCCTCGCTGACACTGCC | 94 | H | 593 |
| 1212331 | 10126 | 10143 | 1547 | 1564 | TAAGGAAGCCCGGCGGGC | 85 | H | 594 |
| 1212341 | 10176 | 10193 | 1597 | 1614 | CCCACCGGGCAGCTGGTG | 78 | H | 595 |
| 1212351 | 10226 | 10243 | 1647 | 1664 | GCAACTGGCAGCAGGCCC | 121 | H | 596 |
| 1212361 | 10368 | 10385 | 1697 | 1714 | GTAGCCAGCCGGGCAGCA | 94 | H | 597 |
| 1212371 | 10418 | 10435 | 1747 | 1764 | TGGGCAGAGACCACTTCC | 98 | H | 598 |
| 1212381 | 10468 | 10485 | 1797 | 1814 | CCACGTCCTTCACACCCA | 84 | H | 599 |
| 1212390 | 10518 | 10535 | 1847 | 1864 | GTCTCGGCAGCAGGTCTG | 141 | H | 600 |
| 1212399 | 10657 | 10674 | 1897 | 1914 | GCACAACAGACGCCCTGG | 73 | H | 601 |
| 1212409 | 10707 | 10724 | 1947 | 1964 | TACCCCTGGCTGCGCAGC | 77 | H | 602 |
| 1212419 | 10757 | 10774 | 1997 | 2014 | GTCCCTCAAAGGGGCGTC | 83 | H | 603 |
| 1212429 | 10787 | 10804 | 2027 | 2044 | TGTCCCTCACAGCAGCTG | 76 | H | 604 |
| 1212439 | 10797 | 10814 | 2037 | 2054 | TCTTCAGTACTGTCCCTC | 76 | H | 605 |
| 1212449 | 10807 | 10824 | 2047 | 2064 | GGCTGCAGAGTCTTCAGT | 95 | H | 606 |
| 1212459 | 10817 | 10834 | 2057 | 2074 | GGGTCCCGAGGGCTGCAG | 24 | H | 607 |
| 1212469 | 10827 | 10844 | 2067 | 2084 | CCTCCGAGTGGGGTCCCG | 92 | H | 608 |
| 1212479 | 10837 | 10854 | 2077 | 2094 | CAGAGGGCACCCTCCGAG | 61 | H | 609 |
| 1212489 | 10847 | 10864 | 2087 | 2104 | GAGGCCTGAGCAGAGGGC | 72 | H | 610 |
| 1212499 | 10857 | 10874 | 2097 | 2114 | AGGTGCTAGGGAGGCCTG | 69 | H | 611 |
| 1212509 | 10867 | 10884 | 2107 | 2124 | GGTTAGGGGGAGGTGCTA | 206 | H | 612 |
| 1212519 | 10877 | 10894 | 2117 | 2134 | GGGAGAATTTGGTTAGGG | 57 | H | 613 |
| 1212529 | 10887 | 10904 | 2127 | 2144 | ATGGGGTCCAGGGAGAAT | 73 | H | 614 |
| 1212539 | 10897 | 10914 | 2137 | 2154 | GGAGCTCAGAATGGGGTC | 90 | H | 615 |
| 1212549 | 10907 | 10924 | 2147 | 2164 | ATGGTGATGGGGAGCTCA | 85 | H | 616 |
| 1212559 | 10917 | 10934 | 2157 | 2174 | CCCACCTCCCATGGTGAT | 105 | H | 617 |
| 1212569 | 10927 | 10944 | 2167 | 2184 | AGATTGAGGCCCCACCTC | 56 | H | 618 |
| 1212579 | 10937 | 10954 | 2177 | 2194 | GGAAGGCCTTAGATTGAG | 104 | H | 619 |
| 1212589 | 10947 | 10964 | 2187 | 2204 | CTTCTGACAGGGAAGGCC | 91 | H | 620 |
| 1212599 | 10957 | 10974 | 2197 | 2214 | CCACAACCCCCTTCTGAC | 70 | H | 621 |
| 1212609 | 10967 | 10984 | 2207 | 2224 | GTGGCTTTTGCCACAACC | 88 | H | 622 |
| 1212619 | 10977 | 10994 | 2217 | 2234 | AGCTTGTAATGTGGCTTT | 82 | H | 623 |
| 1212629 | 10987 | 11004 | 2227 | 2244 | AGGGGATGGCAGCTTGTA | 222 | H | 624 |
| 1212639 | 10997 | 11014 | 2237 | 2254 | TGAAACGGGGAGGGGATG | 79 | H | 625 |
| 1212649 | 11007 | 11024 | 2247 | 2264 | CAGGGTCCACTGAAACGG | 74 | H | 626 |
| 1212659 | 11017 | 11034 | 2257 | 2274 | CACCTGGCCACAGGGTCC | 55 | H | 627 |
| 1212669 | 11027 | 11044 | 2267 | 2284 | TAGGGAAAAGCACCTGGC | 74 | H | 628 |
| 1212679 | 11037 | 11054 | 2277 | 2294 | CCCCTGTGGATAGGGAAA | 167 | H | 629 |
| 1212689 | 11047 | 11064 | 2287 | 2304 | CACACAAACACCCCTGTG | 89 | H | 630 |
| 1212699 | 11057 | 11074 | 2297 | 2314 | CACGCGCACACACACAAA | 99 | H | 631 |
| 1212709 | 11067 | 11084 | 2307 | 2324 | TTGAAACGCACACGCGCA | 105 | H | 632 |
| 1212719 | 11077 | 11094 | 2317 | 2334 | ACAAACTTTATTGAAACG | 87 | H | 633 |
| 1212729 | 11087 | 11104 | 2327 | 2344 | AAGAAAGTGTACAAACTT | 84 | H | 634 |
| 1212739 | N/A | N/A | 2337 | 2354 | TTTTTTTTTTAAGAAAGT | 72 | H | 635 |
| 1211972 | 3118 | 3135 | 33 | 50 | GCTTCCTCTCGCCCCACA | 85 | I | 636 |
| 1211982 | 3158 | 3175 | 73 | 90 | AATGCTGTGTTTCTTTTC | 86 | I | 637 |
| 1211992 | 3198 | 3215 | 113 | 130 | CCATTGGCTACTTATCAA | 81 | I | 638 |
| 1212002 | 3238 | 3255 | 153 | 170 | ACCTCAGTTTCCATTGGC | 89 | I | 639 |
| 1212012 | 3278 | 3295 | 193 | 210 | GTAGCGCTCAGACTACAG | 93 | I | 640 |
| 1212022 | 3291 | 3308 | 206 | 223 | CAGCAGCAACCGGGTAGC | 82 | I | 641 |
| 1212032 | 3301 | 3318 | 216 | 233 | GGTCCTTGGGCAGCAGCA | 69 | I | 642 |
| 1212042 | 3311 | 3328 | 226 | 243 | CCGACTCCGCGGTCCTTG | 95 | I | 643 |
| 1212052 | N/A | N/A | 236 | 253 | CTGCCTGCGTCCGACTCC | 87 | I | 644 |
| 1212062 | 7154 | 7171 | 246 | 263 | TCCACATGGTCTGCCTGC | 90 | I | 645 |
| 1212072 | 7164 | 7181 | 256 | 273 | CTCACCAGGGTCCACATG | 117 | I | 646 |
| 1212082 | 7210 | 7227 | 302 | 319 | TGGGCACCGCGTTCCAGC | 86 | I | 647 |
| 1212092 | 7260 | 7277 | 352 | 369 | CTGGCTCCTCCGGGGTCC | 80 | I | 648 |
| 1212102 | 7433 | 7450 | 402 | 419 | TGCTCAGTGTTGTGGGCC | 107 | I | 649 |
| 1212112 | 7483 | 7500 | 452 | 469 | GTGGCCGGCAGAGCAGTG | 106 | I | 650 |
| 1212122 | 7533 | 7550 | 502 | 519 | TCTGGGAAGGGGCAGCAA | 187 | I | 651 |
| 1212132 | 7698 | 7715 | 552 | 569 | AGTGGAAGCCCCGTGGGC | 108 | I | 652 |
| 1212142 | N/A | N/A | 602 | 619 | CACGGAGTTGTTACCTGA | 86 | I | 653 |
| 1212152 | 8274 | 8291 | 652 | 669 | GTGGAGAAGTCCGGGCAT | 90 | I | 654 |
| 1212162 | 8324 | 8341 | 702 | 719 | CCTGGGGCATGGGGCAGC | 220 | I | 655 |
| 1212172 | 8475 | 8492 | 752 | 769 | GCAGAAGGCACCGTGCGG | 121 | I | 656 |
| 1212182 | 8525 | 8542 | 802 | 819 | GCCAGGGGGGGGTGCCC | 172 | I | 657 |
| 1212192 | 8688 | 8705 | 852 | 869 | AGCTGGACAAGGCCACTG | 27† | I | 658 |
| 1212202 | 8738 | 8755 | 902 | 919 | GCAGGTAGAACCATCAGG | 10† | I | 659 |
| 1212212 | N/A | N/A | 952 | 969 | GTGGCGTTGGGCATTGGG | 391 | I | 660 |
| 1212222 | 9074 | 9091 | 1002 | 1019 | GGTCACACACAGTGTCTT | 90 | I | 661 |
| 1212232 | 9124 | 9141 | 1052 | 1069 | GAGGAGGTCCGTGGTAGC | 118 | I | 662 |
| 1212242 | 9373 | 9390 | 1102 | 1119 | ACCTCCATGTCACATTTC | 87 | I | 663 |
| 1212252 | 9423 | 9440 | 1152 | 1169 | CCCAGGCCCCCGACTGTA | 97 | I | 664 |
| 1212262 | 9562 | 9579 | 1202 | 1219 | GCAGTGTATGTGGTCCTC | 111 | I | 665 |
| 1212272 | 9612 | 9629 | 1252 | 1269 | CCCTGTTCACAGGTACCC | 119 | I | 666 |
| 1212282 | 9662 | 9679 | 1302 | 1319 | GCAGGCTGAGGTGAGCTG | 239 | I | 667 |
| 1212292 | 9712 | 9729 | 1352 | 1369 | GCTGCTGACATTATCACA | 117 | I | 668 |
| 1212302 | 9762 | 9779 | 1402 | 1419 | CAGCCCCACTCCCCAGAC | 90 | I | 669 |
| 1212312 | 10031 | 10048 | 1452 | 1469 | GGCAGCAGTGCTGGTGGT | 122 | I | 670 |
| 1212322 | 10081 | 10098 | 1502 | 1519 | CTCGCTTCCTCGCTGACA | 77 | I | 671 |
| 1212332 | 10131 | 10148 | 1552 | 1569 | TGGGATAAGGAAGCCCGG | 85 | I | 672 |
| 1212342 | 10181 | 10198 | 1602 | 1619 | TCTGCCCCACCGGGCAGC | 103 | I | 673 |
| 1212352 | 10231 | 10248 | 1652 | 1669 | ATGGGGCAACTGGCAGCA | 153 | I | 674 |
| 1212362 | 10373 | 10390 | 1702 | 1719 | CAGGTGTAGCCAGCCGGG | 80 | I | 675 |
| 1212372 | 10423 | 10440 | 1752 | 1769 | CAGGCTGGGCAGAGACCA | 83 | I | 676 |
| 1212382 | 10473 | 10490 | 1802 | 1819 | ACACTCCACGTCCTTCAC | 86 | I | 677 |
| 1212391 | 10523 | 10540 | 1852 | 1869 | CGGTTGTCTCGGCAGCAG | 92 | I | 678 |
| 1212400 | 10662 | 10679 | 1902 | 1919 | GATCAGCACAACAGACGC | 85 | I | 679 |
| 1212410 | 10712 | 10729 | 1952 | 1969 | CTTGGTACCCCTGGCTGC | 123 | I | 680 |
| 1212420 | 10762 | 10779 | 2002 | 2019 | GCTGGGTCCCTCAAAGGG | 88 | I | 681 |
| 1212430 | 10788 | 10805 | 2028 | 2045 | CTGTCCCTCACAGCAGCT | 79 | I | 682 |
| 1212440 | 10798 | 10815 | 2038 | 2055 | GTCTTCAGTACTGTCCCT | 109 | I | 683 |
| 1212450 | 10808 | 10825 | 2048 | 2065 | GGGCTGCAGAGTCTTCAG | 81 | I | 684 |
| 1212460 | 10818 | 10835 | 2058 | 2075 | GGGGTCCCGAGGGCTGCA | 89 | I | 685 |
| 1212470 | 10828 | 10845 | 2068 | 2085 | CCCTCCGAGTGGGGTCCC | 72 | I | 686 |
| 1212480 | 10838 | 10855 | 2078 | 2095 | GCAGAGGGCACCCTCCGA | 87 | I | 687 |
| 1212490 | 10848 | 10865 | 2088 | 2105 | GGAGGCCTGAGCAGAGGG | 88 | I | 688 |
| 1212500 | 10858 | 10875 | 2098 | 2115 | GAGGTGCTAGGGAGGCCT | 94 | I | 689 |
| 1212510 | 10868 | 10885 | 2108 | 2125 | TGGTTAGGGGGAGGTGCT | 75 | I | 690 |
| 1212520 | 10878 | 10895 | 2118 | 2135 | AGGGAGAATTTGGTTAGG | 73 | I | 691 |
| 1212530 | 10888 | 10905 | 2128 | 2145 | AATGGGGTCCAGGGAGAA | 158 | I | 692 |
| 1212540 | 10898 | 10915 | 2138 | 2155 | GGGAGCTCAGAATGGGGT | 92 | I | 693 |
| 1212550 | 10908 | 10925 | 2148 | 2165 | CATGGTGATGGGGAGCTC | 87 | I | 694 |
| 1212560 | 10918 | 10935 | 2158 | 2175 | CCCCACCTCCCATGGTGA | 115 | I | 695 |
| 1212570 | 10928 | 10945 | 2168 | 2185 | TAGATTGAGGCCCCACCT | 85 | I | 696 |
| 1212580 | 10938 | 10955 | 2178 | 2195 | GGGAAGGCCTTAGATTGA | 68 | I | 697 |
| 1212590 | 10948 | 10965 | 2188 | 2205 | CCTTCTGACAGGGAAGGC | 105 | I | 698 |
| 1212600 | 10958 | 10975 | 2198 | 2215 | GCCACAACCCCCTTCTGA | 88 | I | 699 |
| 1212610 | 10968 | 10985 | 2208 | 2225 | TGTGGCTTTTGCCACAAC | 102 | I | 700 |
| 1212620 | 10978 | 10995 | 2218 | 2235 | CAGCTTGTAATGTGGCTT | 84 | I | 701 |
| 1212630 | 10988 | 11005 | 2228 | 2245 | GAGGGGATGGCAGCTTGT | 153 | I | 702 |
| 1212640 | 10998 | 11015 | 2238 | 2255 | CTGAAACGGGGAGGGGAT | 87 | I | 703 |
| 1212650 | 11008 | 11025 | 2248 | 2265 | ACAGGGTCCACTGAAACG | 96 | I | 704 |
| 1212660 | 11018 | 11035 | 2258 | 2275 | GCACCTGGCCACAGGGTC | 89 | I | 705 |
| 1212670 | 11028 | 11045 | 2268 | 2285 | ATAGGGAAAAGCACCTGG | 93 | I | 706 |
| 1212680 | 11038 | 11055 | 2278 | 2295 | ACCCCTGTGGATAGGGAA | 90 | I | 707 |
| 1212690 | 11048 | 11065 | 2288 | 2305 | ACACACAAACACCCCTGT | 94 | I | 708 |
| 1212700 | 11058 | 11075 | 2298 | 2315 | ACACGCGCACACACACAA | 143 | I | 709 |
| 1212710 | 11068 | 11085 | 2308 | 2325 | ATTGAAACGCACACGCGC | 100 | I | 710 |
| 1212720 | 11078 | 11095 | 2318 | 2335 | TACAAACTTTATTGAAAC | 93 | I | 711 |
| 1212730 | 11088 | 11105 | 2328 | 2345 | TAAGAAAGTGTACAAACT | 80 | I | 712 |
| 1212740 | N/A | N/A | 2338 | 2355 | TTTTTTTTTTTAAGAAAG | 81 | I | 713 |
| 1211973 | 3122 | 3139 | 37 | 54 | CCCTGCTTCCTCTCGCCC | 53 | J | 714 |
| 1211983 | 3162 | 3179 | 77 | 94 | CTGGAATGCTGTGTTTCT | 132 | J | 715 |
| 1211993 | 3202 | 3219 | 117 | 134 | GCTCCCATTGGCTACTTA | 170 | J | 716 |
| 1212003 | 3242 | 3259 | 157 | 174 | GCCTACCTCAGTTTCCAT | 81 | J | 717 |
| 1212013 | 3282 | 3299 | 197 | 214 | CCGGGTAGCGCTCAGACT | 100 | J | 718 |
| 1212023 | 3292 | 3309 | 207 | 224 | GCAGCAGCAACCGGGTAG | 111 | J | 719 |
| 1212033 | 3302 | 3319 | 217 | 234 | CGGTCCTTGGGCAGCAGC | 101 | J | 720 |
| 1212043 | 3312 | 3329 | 227 | 244 | TCCGACTCCGCGGTCCTT | 109 | J | 721 |
| 1212053 | N/A | N/A | 237 | 254 | TCTGCCTGCGTCCGACTC | 85 | J | 722 |
| 1212063 | 7155 | 7172 | 247 | 264 | GTCCACATGGTCTGCCTG | 86 | J | 723 |
| 1212073 | 7165 | 7182 | 257 | 274 | GCTCACCAGGGTCCACAT | 84 | J | 724 |
| 1212083 | 7215 | 7232 | 307 | 324 | CCATCTGGGCACCGCGTT | 46 | J | 725 |
| 1212093 | 7265 | 7282 | 357 | 374 | TGTAGCTGGCTCCTCCGG | 90 | J | 726 |
| 1212103 | 7438 | 7455 | 407 | 424 | ATGCCTGCTCAGTGTTGT | 83 | J | 727 |
| 1212113 | 7488 | 7505 | 457 | 474 | CAGGAGTGGCCGGCAGAG | 82 | J | 728 |
| 1212123 | N/A | N/A | 507 | 524 | CGGCCTCTGGGAAGGGGC | 93 | J | 729 |
| 1212133 | 7703 | 7720 | 557 | 574 | ACTGCAGTGGAAGCCCCG | 122 | J | 730 |
| 1212143 | 8229 | 8246 | 607 | 624 | GCACCCACGGAGTTGTTA | 75 | J | 731 |
| 1212153 | 8279 | 8296 | 657 | 674 | AGCACGTGGAGAAGTCCG | 93 | J | 732 |
| 1212163 | N/A | N/A | 707 | 724 | GGAAGCCTGGGGCATGGG | 100 | J | 733 |
| 1212173 | 8480 | 8497 | 757 | 774 | AGGTCGCAGAAGGCACCG | 85 | J | 734 |
| 1212183 | 8530 | 8547 | 807 | 824 | TCTTTGCCAGGGGGTGGG | 153 | J | 735 |
| 1212193 | 8693 | 8710 | 857 | 874 | GACCGAGCTGGACAAGGC | 10† | J | 736 |
| 1212203 | 8743 | 8760 | 907 | 924 | TCACAGCAGGTAGAACCA | 18† | J | 737 |
| 1212213 | N/A | N/A | 957 | 974 | AGCAGGTGGCGTTGGGCA | 108 | J | 738 |
| 1212223 | 9079 | 9096 | 1007 | 1024 | GATCAGGTCACACACAGT | 79 | J | 739 |
| 1212233 | 9129 | 9146 | 1057 | 1074 | TTAGTGAGGAGGTCCGTG | 83 | J | 740 |
| 1212243 | 9378 | 9395 | 1107 | 1124 | AGCTCACCTCCATGTCAC | 108 | J | 741 |
| 1212253 | 9428 | 9445 | 1157 | 1174 | GCAGCCCCAGGCCCCCGA | 83 | J | 742 |
| 1212263 | 9567 | 9584 | 1207 | 1224 | GGACAGCAGTGTATGTGG | 93 | J | 743 |
| 1212273 | 9617 | 9634 | 1257 | 1274 | GGGGCCCCTGTTCACAGG | 71 | J | 744 |
| 1212283 | 9667 | 9684 | 1307 | 1324 | GTCTGGCAGGCTGAGGTG | 98 | J | 745 |
| 1212293 | 9717 | 9734 | 1357 | 1374 | GGACAGCTGCTGACATTA | 102 | J | 746 |
| 1212303 | 9767 | 9784 | 1407 | 1424 | GACAGCAGCCCCACTCCC | 62 | J | 747 |
| 1212313 | 10036 | 10053 | 1457 | 1474 | CTGGGGGCAGCAGTGCTG | 158 | J | 748 |
| 1212323 | 10086 | 10103 | 1507 | 1524 | ACGATCTCGCTTCCTCGC | 110 | J | 749 |
| 1212333 | 10136 | 10153 | 1557 | 1574 | TGGGGTGGGATAAGGAAG | 107 | J | 750 |
| 1212343 | 10186 | 10203 | 1607 | 1624 | GCAGGTCTGCCCCACCGG | 83 | J | 751 |
| 1212353 | N/A | N/A | 1657 | 1674 | ACAGCATGGGGCAACTGG | 82 | J | 752 |
| 1212363 | 10378 | 10395 | 1707 | 1724 | CGTTGCAGGTGTAGCCAG | 93 | J | 753 |
| 1212373 | 10428 | 10445 | 1757 | 1774 | GGTGGCAGGCTGGGCAGA | 215 | J | 754 |
| 1212383 | 10478 | 10495 | 1807 | 1824 | TCCCCACACTCCACGTCC | 83 | J | 755 |
| 1212392 | 10528 | 10545 | 1857 | 1874 | CCTGTCGGTTGTCTCGGC | 101 | J | 756 |
| 1212401 | 10667 | 10684 | 1907 | 1924 | GCGCCGATCAGCACAACA | 86 | J | 757 |
| 1212411 | 10717 | 10734 | 1957 | 1974 | AAACACTTGGTACCCCTG | 94 | J | 758 |
| 1212421 | 10767 | 10784 | 2007 | 2024 | TCAAGGCTGGGTCCCTCA | 86 | J | 759 |
| 1212431 | 10789 | 10806 | 2029 | 2046 | ACTGTCCCTCACAGCAGC | 82 | J | 760 |
| 1212441 | 10799 | 10816 | 2039 | 2056 | AGTCTTCAGTACTGTCCC | 97 | J | 761 |
| 1212451 | 10809 | 10826 | 2049 | 2066 | AGGGCTGCAGAGTCTTCA | 88 | J | 762 |
| 1212461 | 10819 | 10836 | 2059 | 2076 | TGGGGTCCCGAGGGCTGC | 90 | J | 763 |
| 1212471 | 10829 | 10846 | 2069 | 2086 | ACCCTCCGAGTGGGGTCC | 78 | J | 764 |
| 1212481 | 10839 | 10856 | 2079 | 2096 | AGCAGAGGGCACCCTCCG | 97 | J | 765 |
| 1212491 | 10849 | 10866 | 2089 | 2106 | GGGAGGCCTGAGCAGAGG | 75 | J | 766 |
| 1212501 | 10859 | 10876 | 2099 | 2116 | GGAGGTGCTAGGGAGGCC | 64 | J | 767 |
| 1212511 | 10869 | 10886 | 2109 | 2126 | TTGGTTAGGGGGAGGTGC | 102 | J | 768 |
| 1212521 | 10879 | 10896 | 2119 | 2136 | CAGGGAGAATTTGGTTAG | 85 | J | 769 |
| 1212531 | 10889 | 10906 | 2129 | 2146 | GAATGGGGTCCAGGGAGA | 71 | J | 770 |
| 1212541 | 10899 | 10916 | 2139 | 2156 | GGGGAGCTCAGAATGGGG | 102 | J | 771 |
| 1212551 | 10909 | 10926 | 2149 | 2166 | CCATGGTGATGGGGAGCT | 72 | J | 772 |
| 1212561 | 10919 | 10936 | 2159 | 2176 | GCCCCACCTCCCATGGTG | 92 | J | 773 |
| 1212571 | 10929 | 10946 | 2169 | 2186 | TTAGATTGAGGCCCCACC | 89 | J | 774 |
| 1212581 | 10939 | 10956 | 2179 | 2196 | AGGGAAGGCCTTAGATTG | 95 | J | 775 |
| 1212591 | 10949 | 10966 | 2189 | 2206 | CCCTTCTGACAGGGAAGG | 84 | J | 776 |
| 1212601 | 10959 | 10976 | 2199 | 2216 | TGCCACAACCCCCTTCTG | 80 | J | 777 |
| 1212611 | 10969 | 10986 | 2209 | 2226 | ATGTGGCTTTTGCCACAA | 144 | J | 778 |
| 1212621 | 10979 | 10996 | 2219 | 2236 | GCAGCTTGTAATGTGGCT | 82 | J | 779 |
| 1212631 | 10989 | 11006 | 2229 | 2246 | GGAGGGGATGGCAGCTTG | 89 | J | 780 |
| 1212641 | 10999 | 11016 | 2239 | 2256 | ACTGAAACGGGGAGGGGA | 97 | J | 781 |
| 1212651 | 11009 | 11026 | 2249 | 2266 | CACAGGGTCCACTGAAAC | 76 | J | 782 |
| 1212661 | 11019 | 11036 | 2259 | 2276 | AGCACCTGGCCACAGGGT | 88 | J | 783 |
| 1212671 | 11029 | 11046 | 2269 | 2286 | GATAGGGAAAAGCACCTG | 86 | J | 784 |
| 1212681 | 11039 | 11056 | 2279 | 2296 | CACCCCTGTGGATAGGGA | 95 | J | 785 |
| 1212691 | 11049 | 11066 | 2289 | 2306 | CACACACAAACACCCCTG | 78 | J | 786 |
| 1212701 | 11059 | 11076 | 2299 | 2316 | CACACGCGCACACACACA | 145 | J | 787 |
| 1212711 | 11069 | 11086 | 2309 | 2326 | TATTGAAACGCACACGCG | 86 | J | 788 |
| 1212721 | 11079 | 11096 | 2319 | 2336 | GTACAAACTTTATTGAAA | 142 | J | 789 |
| 1212731 | 11089 | 11106 | 2329 | 2346 | TTAAGAAAGTGTACAAAC | 99 | J | 790 |
| 1212741 | N/A | N/A | 2339 | 2356 | TTTTTTTTTTTTAAGAAA | 95 | J | 791 |

### Example 2: Effect of Uniform MOE Modified Oligonucleotides with Uniform Phosphorothioate Internucleoside Linkages on Human Progranulin In Vitro, Single Dose

Modified oligonucleotides complementary to a human progranulin nucleic acid were designed and tested for their effect on progranulin RNA in vitro. The modified oligonucleotides were tested in a series of experiments that had the same culture conditions.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (GENBANK Accession No. NC_000017.11 truncated from nucleotides 44342001 to 44356000), to SEQ ID NO: 2 (GENBANK Accession No. NM_002087.3), or to both. ‘N/A’ indicates that the modified oligonucleotide is not 100% complementary to that particular target nucleic acid sequence.

The modified oligonucleotides in the table below are 18 nucleosides in length, wherein the sugar motif for the modified oligonucleotides is (from 5′ to 3′): eeeeeeeeeeeeeeeeee; wherein “e” represents a 2′-MOE sugar moiety. The internucleoside linkage motif for the modified oligonucleotides is (from 5′ to 3′): sssssssssssssssss; wherein each “s” represents a phosphorothioate internucleoside linkage. Each cytosine residue is a 5-methyl cytosine.

Cultured A-431 cells were treated with modified oligonucleotide at a concentration of 4,000 nM by free uptake at a density of 10,000 cells per well. After a treatment period of approximately 48 hours, RNA was isolated from the cells and progranulin RNA levels were measured by quantitative real-time PCR. Human primer probe set RTS42426 (described herein above) was used to measure upregulation of progranulin RNA. Progranulin RNA levels were normalized to total RNA content, as measured by RIBOGREEN®. Results are presented as percent progranulin RNA, relative to the amount in untreated control cells (% UTC).

TABLE 2
Uniform MOE modified oligonucleotides with uniform PS linkages
complementary to human progranulin

| | SEQ | SEQ | SEQ | SEQ | | | | |
|---|---|---|---|---|---|---|---|---|
| | ID | ID | ID | ID | | | | |
| | NO: 1 | NO: 1 | NO: 2 | NO: 2 | | | | SEQ |
| Compound | Start | Stop | Start | Stop | | progranulin | | ID |
| No. | Site | Site | Site | Site | Sequence (5′ to 3′) | (% UTC) | AID | NO |
| 1212165 | 8440 | 8457 | 717 | 734 | CTTCACAGCAGGAAGCCT | 109 | K | 110 |
| 1212166 | 8445 | 8462 | 722 | 739 | CCTGTCTTCACAGCAGGA | 101 | K | 188 |
| 1212167 | 8450 | 8467 | 727 | 744 | TGCACCCTGTCTTCACAG | 135 | K | 267 |
| 1212168 | 8455 | 8472 | 732 | 749 | AGCAGTGCACCCTGTCTT | 91 | K | 344 |
| 1212169 | 8460 | 8477 | 737 | 754 | CGGACAGCAGTGCACCCT | 119 | K | 423 |
| 1212170 | 8465 | 8482 | 742 | 759 | CCGTGCGGACAGCAGTGC | 88 | K | 500 |
| 1212171 | 8470 | 8487 | 747 | 764 | AGGCACCGTGCGGACAGC | 66 | K | 578 |
| 1212172 | 8475 | 8492 | 752 | 769 | GCAGAAGGCACCGTGCGG | 96 | K | 656 |
| 1212173 | 8480 | 8497 | 757 | 774 | AGGTCGCAGAAGGCACCG | 79 | K | 734 |
| 1212174 | 8485 | 8502 | 762 | 779 | GAACCAGGTCGCAGAAGG | 100 | K | 33 |
| 1212175 | 8490 | 8507 | 767 | 784 | GGTGTGAACCAGGTCGCA | 106 | K | 111 |
| 1212176 | 8495 | 8512 | 772 | 789 | CAGCGGGTGTGAACCAGG | 74 | K | 189 |
| 1212177 | 8500 | 8517 | 777 | 794 | TGATGCAGCGGGTGTGAA | 131 | K | 268 |
| 1212178 | 8505 | 8522 | 782 | 799 | GGGTGTGATGCAGCGGGT | 115 | K | 345 |
| 1366749 | 8447 | 8464 | 724 | 741 | ACCCTGTCTTCACAGCAG | 112 | K | 792 |
| 1366750 | 8452 | 8469 | 729 | 746 | AGTGCACCCTGTCTTCAC | 90 | K | 793 |
| 1366751 | 8432 | 8449 | N/A | N/A | CAGGAAGCCTGAGAAAAA | 75 | K | 794 |
| 1366752 | 8489 | 8506 | 766 | 783 | GTGTGAACCAGGTCGCAG | 91 | K | 795 |
| 1366753 | 8474 | 8491 | 751 | 768 | CAGAAGGCACCGTGCGGA | 81 | K | 796 |
| 1366754 | 8492 | 8509 | 769 | 786 | CGGGTGTGAACCAGGTCG | 84 | K | 797 |
| 1366755 | 8436 | 8453 | N/A | N/A | ACAGCAGGAAGCCTGAGA | 107 | K | 798 |
| 1366756 | 8441 | 8458 | 718 | 735 | TCTTCACAGCAGGAAGCC | 100 | K | 799 |
| 1366757 | 8502 | 8519 | 779 | 796 | TGTGATGCAGCGGGTGTG | 109 | K | 800 |
| 1366758 | 8497 | 8514 | 774 | 791 | TGCAGCGGGTGTGAACCA | 122 | K | 801 |
| 1366759 | 8457 | 8474 | 734 | 751 | ACAGCAGTGCACCCTGTC | 81 | K | 802 |
| 1366760 | 8467 | 8484 | 744 | 761 | CACCGTGCGGACAGCAGT | 106 | K | 803 |
| 1366761 | 8437 | 8454 | N/A | N/A | CACAGCAGGAAGCCTGAG | 66 | K | 804 |
| 1366762 | 8444 | 8461 | 721 | 738 | CTGTCTTCACAGCAGGAA | 96 | K | 805 |
| 1366763 | 8501 | 8518 | 778 | 795 | GTGATGCAGCGGGTGTGA | 151 | K | 806 |
| 1366764 | 8466 | 8483 | 743 | 760 | ACCGTGCGGACAGCAGTG | 74 | K | 807 |
| 1366765 | 8438 | 8455 | N/A | N/A | TCACAGCAGGAAGCCTGA | 81 | K | 808 |
| 1366766 | 8472 | 8489 | 749 | 766 | GAAGGCACCGTGCGGACA | 78 | K | 809 |
| 1366767 | 8446 | 8463 | 723 | 740 | CCCTGTCTTCACAGCAGG | 71 | K | 810 |
| 1366768 | 8443 | 8460 | 720 | 737 | TGTCTTCACAGCAGGAAG | 79 | K | 811 |
| 1366769 | 8498 | 8515 | 775 | 792 | ATGCAGCGGGTGTGAACC | 118 | K | 812 |
| 1366770 | 8504 | 8521 | 781 | 798 | GGTGTGATGCAGCGGGTG | 114 | K | 813 |
| 1366771 | 8482 | 8499 | 759 | 776 | CCAGGTCGCAGAAGGCAC | 87 | K | 814 |
| 1366772 | 8483 | 8500 | 760 | 777 | ACCAGGTCGCAGAAGGCA | 118 | K | 815 |
| 1366773 | 8507 | 8524 | 784 | 801 | GTGGGTGTGATGCAGCGG | 103 | K | 816 |
| 1366774 | 8442 | 8459 | 719 | 736 | GTCTTCACAGCAGGAAGC | 87 | K | 817 |
| 1366775 | 8464 | 8481 | 741 | 758 | CGTGCGGACAGCAGTGCA | 97 | K | 818 |
| 1366776 | 8448 | 8465 | 725 | 742 | CACCCTGTCTTCACAGCA | 82 | K | 819 |
| 1366777 | 8476 | 8493 | 753 | 770 | CGCAGAAGGCACCGTGCG | 102 | K | 820 |
| 1366778 | 8508 | 8525 | 785 | 802 | CGTGGGTGTGATGCAGCG | 100 | K | 821 |
| 1366779 | 8494 | 8511 | 771 | 788 | AGCGGGTGTGAACCAGGT | 85 | K | 822 |
| 1366780 | 8453 | 8470 | 730 | 747 | CAGTGCACCCTGTCTTCA | 170 | K | 823 |
| 1366781 | 8477 | 8494 | 754 | 771 | TCGCAGAAGGCACCGTGC | 87 | K | 824 |
| 1366782 | 8469 | 8486 | 746 | 763 | GGCACCGTGCGGACAGCA | 56 | K | 825 |
| 1366783 | 8435 | 8452 | N/A | N/A | CAGCAGGAAGCCTGAGAA | 132 | K | 826 |
| 1366784 | 8484 | 8501 | 761 | 778 | AACCAGGTCGCAGAAGGC | 81 | K | 827 |
| 1366785 | 8499 | 8516 | 776 | 793 | GATGCAGCGGGTGTGAAC | 132 | K | 828 |
| 1366786 | 8496 | 8513 | 773 | 790 | GCAGCGGGTGTGAACCAG | 76 | K | 829 |
| 1366787 | 8493 | 8510 | 770 | 787 | GCGGGTGTGAACCAGGTC | 95 | K | 830 |
| 1366788 | 8439 | 8456 | 716 | 733 | TTCACAGCAGGAAGCCTG | 93 | K | 831 |
| 1366789 | 8468 | 8485 | 745 | 762 | GCACCGTGCGGACAGCAG | 95 | K | 832 |
| 1366790 | 8459 | 8476 | 736 | 753 | GGACAGCAGTGCACCCTG | 153 | K | 833 |
| 1366791 | 8454 | 8471 | 731 | 748 | GCAGTGCACCCTGTCTTC | 94 | K | 834 |
| 1366792 | 8471 | 8488 | 748 | 765 | AAGGCACCGTGCGGACAG | 65 | K | 835 |
| 1366793 | 8433 | 8450 | N/A | N/A | GCAGGAAGCCTGAGAAAA | 87 | K | 836 |
| 1366794 | 8462 | 8479 | 739 | 756 | TGCGGACAGCAGTGCACC | 119 | K | 837 |
| 1366795 | 8451 | 8468 | 728 | 745 | GTGCACCCTGTCTTCACA | 88 | K | 838 |
| 1366796 | 8458 | 8475 | 735 | 752 | GACAGCAGTGCACCCTGT | 90 | K | 839 |
| 1366797 | 8479 | 8496 | 756 | 773 | GGTCGCAGAAGGCACCGT | 97 | K | 840 |
| 1366798 | 8486 | 8503 | 763 | 780 | TGAACCAGGTCGCAGAAG | 108 | K | 841 |
| 1366799 | 8491 | 8508 | 768 | 785 | GGGTGTGAACCAGGTCGC | 105 | K | 842 |
| 1366800 | 8473 | 8490 | 750 | 767 | AGAAGGCACCGTGCGGAC | 69 | K | 843 |
| 1366801 | 8506 | 8523 | 783 | 800 | TGGGTGTGATGCAGCGGG | 129 | K | 844 |
| 1366802 | 8488 | 8505 | 765 | 782 | TGTGAACCAGGTCGCAGA | 182 | K | 845 |
| 1366803 | 8456 | 8473 | 733 | 750 | CAGCAGTGCACCCTGTCT | 118 | K | 846 |
| 1366804 | 8503 | 8520 | 780 | 797 | GTGTGATGCAGCGGGTGT | 116 | K | 847 |
| 1366805 | 8481 | 8498 | 758 | 775 | CAGGTCGCAGAAGGCACC | 112 | K | 848 |
| 1366806 | 8461 | 8478 | 738 | 755 | GCGGACAGCAGTGCACCC | 92 | K | 849 |
| 1366807 | 8463 | 8480 | 740 | 757 | GTGCGGACAGCAGTGCAC | 81 | K | 850 |
| 1366808 | 8487 | 8504 | 764 | 781 | GTGAACCAGGTCGCAGAA | 124 | K | 851 |
| 1366809 | 8478 | 8495 | 755 | 772 | GTCGCAGAAGGCACCGTG | 114 | K | 852 |
| 1366810 | 8434 | 8451 | N/A | N/A | AGCAGGAAGCCTGAGAAA | 97 | K | 853 |
| 1366811 | 8449 | 8466 | 726 | 743 | GCACCCTGTCTTCACAGC | 82 | K | 854 |

### Example 3: Effect of Modified Oligonucleotides on Human Progranulin In Vitro, Multiple Doses

Modified oligonucleotides selected from the example above were tested in A-431 cells. The modified oligonucleotides were tested in a series of experiments using the same culture conditions. The results for each experiment are presented in separate tables shown below.

Cultured A-431 cells were plated at a density of 10,000 cells per well and were treated by free uptake at various doses, as specified in the tables below. After a treatment period of approximately 48 hours, total RNA was isolated from the cells and progranulin RNA levels were measured by quantitative real-time PCR. Human progranulin primer probe set RTS42426 (described herein above) was used to measure progranulin RNA. Progranulin RNA levels were normalized to total RNA content, as measured by RIBOGREEN®. Results are presented in the tables below as percent progranulin RNA, relative to the amount in untreated control cells (% UTC). Fold increase in the amount of progranulin RNA at 20 μM relative to untreated control cells was calculated and is presented in the tables below.

TABLE 3
Dose-dependent increase in human progranulin
RNA by modified oligonucleotides

| Compound | progranulin RNA (% UTC) | | | | Fold Increase |
|---|---|---|---|---|---|
| No. | 313 nM | 1250 nM | 5000 nM | 20000 nM | @ 20 μM |
| 1212035 | 175 | 221 | 271 | 334 | 3.34 |
| 1212135 | 122 | 123 | 131 | 146 | 1.46 |
| 1212165 | 144 | 147 | 154 | 156 | 1.56 |
| 1212175 | 133 | 127 | 155 | 166 | 1.66 |
| 1212184 | 135 | 141 | 144 | 189 | 1.89 |
| 1212245 | 131 | 147 | 141 | 137 | 1.37 |
| 1212246 | 184 | 185 | 216 | 254 | 2.54 |
| 1212264 | 111 | 115 | 133 | 127 | 1.27 |
| 1212345 | 137 | 173 | 200 | 250 | 2.50 |
| 1212366 | 104 | 104 | 112 | 122 | 1.22 |
| 1212373 | 289 | 312 | 328 | 284 | 2.84 |
| 1212374 | 120 | 126 | 131 | 194 | 1.94 |
| 1212432 | 104 | 98 | 107 | 131 | 1.31 |
| 1212472 | 117 | 138 | 181 | 210 | 2.10 |
| 1212542 | 130 | 143 | 190 | 248 | 2.48 |
| 1212614 | 174 | 167 | 171 | 261 | 2.61 |
| 1212632 | 122 | 122 | 143 | 166 | 1.66 |
| 1212633 | 121 | 135 | 161 | 182 | 1.82 |
| 1212662 | 110 | 104 | 97 | 111 | 1.11 |

TABLE 4
Dose-dependent increase in human progranulin RNA
by modified oligonucleotides

| | | | | | Fold |
|---|---|---|---|---|---|
| Compound | progranulin RNA (% UTC) | | | | Increase @ |
| No. | 313 nM | 1250 nM | 5000 nM | 20000 nM | 20 μM |
| 1212036 | 107 | 231 | 267 | 337 | 3.37 |
| 1212088 | 104 | 110 | 129 | 128 | 1.28 |
| 1212128 | 153 | 143 | 155 | 152 | 1.52 |
| 1212169 | 189 | 162 | 166 | 210 | 2.10 |
| 1212228 | 167 | 194 | 196 | 208 | 2.08 |
| 1212248 | 123 | 136 | 150 | 157 | 1.57 |
| 1212256 | 171 | 211 | 249 | 278 | 2.78 |
| 1212257 | 115 | 120 | 150 | 145 | 1.45 |
| 1212298 | 138 | 136 | 146 | 163 | 1.63 |
| 1212373 | 293 | 315 | 339 | 307 | 3.07 |
| 1212396 | 119 | 118 | 120 | 196 | 1.96 |
| 1212505 | 122 | 144 | 157 | 178 | 1.78 |
| 1212506 | 152 | 177 | 205 | 239 | 2.39 |
| 1212625 | 101 | 106 | 127 | 126 | 1.26 |
| 1212626 | 110 | 121 | 136 | 152 | 1.52 |
| 1212627 | 143 | 152 | 148 | 131 | 1.31 |
| 1212635 | 115 | 103 | 103 | 186 | 1.86 |
| 1212647 | 184 | 162 | 153 | 197 | 1.97 |
| 1212665 | 104 | 103 | 107 | 118 | 1.18 |

TABLE 5
Dose-dependent increase in human progranulin
RNA by modified oligonucleotides

| | progranulin RNA (% UTC) | | | | Fold |
|---|---|---|---|---|---|
| Compound | 313 | 1250 | 5000 | | Increase |
| No. | nM | nM | nM | 20000 nM | @ 20 μM |
| 1212000 | 94 | 108 | 109 | 127 | 1.27 |
| 1212050 | 112 | 108 | 102 | 98 | 0.98 |
| 1212071 | 171 | 174 | 147 | 145 | 1.45 |
| 1212080 | 100 | 125 | 141 | 237 | 2.37 |
| 1212121 | 113 | 118 | 150 | 184 | 1.84 |
| 1212129 | 140 | 154 | 162 | 186 | 1.86 |
| 1212161 | 151 | 165 | 189 | 223 | 2.23 |
| 1212179 | 229 | 222 | 215 | 238 | 2.38 |
| 1212180 | 359 | 365 | 405 | 427 | 4.27 |
| 1212219 | 194 | 182 | 179 | 196 | 1.96 |
| 1212249 | 138 | 133 | 158 | 241 | 2.41 |
| 1212359 | 147 | 159 | 164 | 217 | 2.17 |
| 1212373 | 266 | 286 | 293 | 263 | 2.63 |
| 1212390 | 130 | 160 | 255 | 281 | 2.81 |
| 1212507 | 113 | 130 | 170 | 268 | 2.68 |
| 1212508 | 129 | 120 | 147 | 217 | 2.17 |
| 1212628 | 143 | 152 | 213 | 219 | 2.19 |
| 1212658 | 102 | 104 | 136 | 200 | 2.00 |
| 1212679 | 185 | 175 | 137 | 167 | 1.67 |

TABLE 6
Dose-dependent increase in human progranulin
RNA by modified oligonucleotides

| | | | | | Fold |
|---|---|---|---|---|---|
| Compound | progranulin RNA (% UTC) | | | | Increase @ |
| No. | 313 nM | 1250 nM | 5000 nM | 20000 nM | 20 μM |
| 1211983 | 162 | 205 | 177 | 194 | 1.94 |
| 1211993 | 200 | 179 | 162 | 183 | 1.83 |
| 1212122 | 137 | 171 | 206 | 209 | 2.09 |
| 1212131 | 146 | 173 | 280 | 352 | 3.52 |
| 1212181 | 231 | 243 | 174 | 179 | 1.79 |
| 1212182 | 264 | 285 | 265 | 270 | 2.70 |
| 1212183 | 224 | 240 | 246 | 293 | 2.93 |
| 1212282 | 92 | 117 | 116 | 154 | 1.54 |
| 1212313 | 120 | 166 | 158 | 157 | 1.57 |
| 1212352 | 145 | 157 | 188 | 217 | 2.17 |
| 1212373 | 274 | 275 | 252 | 244 | 2.44 |
| 1212509 | 144 | 173 | 239 | 336 | 3.36 |
| 1212530 | 70 | 105 | 118 | 132 | 1.32 |
| 1212611 | 100 | 108 | 135 | 207 | 2.07 |
| 1212629 | 125 | 146 | 177 | 233 | 2.33 |
| 1212630 | 106 | 116 | 106 | 140 | 1.40 |
| 1212700 | 132 | 185 | 207 | 282 | 2.82 |
| 1212701 | 157 | 212 | 235 | 297 | 2.97 |
| 1212721 | 135 | 201 | 255 | 350 | 3.50 |

TABLE 7
Dose-dependent increase in human progranulin
RNA by modified oligonucleotides

| | progranulin RNA (% UTC) | | | | Fold |
|---|---|---|---|---|---|
| Compound | 313 | 1250 | 5000 | | Increase |
| No. | nM | nM | nM | 20000 nM | @ 20 μM |
| 1212167 | 90 | 98 | 111 | 108 | 1.08 |
| 1212169 | 93 | 88 | 97 | 87 | 0.87 |
| 1212177 | 126 | 131 | 144 | 172 | 1.72 |
| 1212178 | 107 | 98 | 91 | 100 | 1.00 |
| 1366758 | 125 | 103 | 106 | 139 | 1.39 |
| 1366763 | 113 | 160 | 169 | 172 | 1.72 |
| 1366769 | 144 | 123 | 104 | 102 | 1.02 |
| 1366772 | 87 | 111 | 100 | 115 | 1.15 |
| 1366780 | 110 | 108 | 124 | 142 | 1.42 |
| 1366783 | 101 | 123 | 105 | 153 | 1.53 |
| 1366785 | 134 | 134 | 143 | 169 | 1.69 |
| 1366790 | 140 | 130 | 142 | 136 | 1.36 |
| 1366794 | 93 | 103 | 102 | 118 | 1.18 |
| 1366801 | 100 | 107 | 122 | 142 | 1.42 |
| 1366802 | 118 | 136 | 129 | 148 | 1.48 |
| 1366803 | 109 | 105 | 113 | 107 | 1.07 |
| 1366804 | 117 | 108 | 105 | 105 | 1.05 |
| 1366808 | 94 | 110 | 97 | 111 | 1.11 |

### Example 4: Design of Uniform MOE Modified Oligonucleotides with Mixed PS/PO Internucleoside Linkages Complementary to Human Progranulin RNA

Modified oligonucleotides complementary to a human progranulin RNA were designed as described in Table 8 below.

The modified oligonucleotides in the table below are 18 nucleosides in length, and the sugar motif for the modified oligonucleotides is (from 5′ to 3′): eeeeeeeeeeeeeeeeee; wherein “e” represents a 2′-MOE sugar moiety. The internucleoside linkage motif for the modified oligonucleotides is (from 5′ to 3′): sosssssssosssssss; wherein each “s” represents a phosphorothioate internucleoside linkage, and each “o” represents a phosphodiester internucleoside linkage.

Each cytosine residue is a 5-methyl cytosine. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (described herein above) to SEQ ID NO: 2 (described herein above), or to both. ‘N/A’ indicates that the modified oligonucleotide is not 100% complementary to that particular target nucleic acid sequence.

TABLE 8
Uniform 2′-MOE modified oligonucleotides with mixed PS/PO
linkages complementary to human GRN

| | SEQ ID | SEQ ID | SEQ ID | SEQ ID | | |
|---|---|---|---|---|---|---|
| | NO: 1 | NO: 1 | NO: 2 | NO: 2 | | SEQ |
| Compound | Start | Stop | Start | Stop | | ID |
| No. | Site | Site | Site | Site | Sequence (5′ to 3′) | NO |
| 1557984 | 10882 | 10899 | 2122 | 2139 | GTCCAGGGAGAATTTGGT | 224 |
| 1557988 | 10883 | 10900 | 2123 | 2140 | GGTCCAGGGAGAATTTGG | 302 |
| 1557989 | 10995 | 11012 | 2235 | 2252 | AAACGGGGAGGGGATGGC | 469 |
| 1557990 | 10996 | 11013 | 2236 | 2253 | GAAACGGGGAGGGGATGG | 547 |
| 1557991 | 10997 | 11014 | 2237 | 2254 | TGAAACGGGGAGGGGATG | 625 |
| 1557992 | 10998 | 11015 | 2238 | 2255 | CTGAAACGGGGAGGGGAT | 703 |
| 1557993 | 10999 | 11016 | 2239 | 2256 | ACTGAAACGGGGAGGGGA | 781 |
| 1557994 | 11000 | 11017 | 2240 | 2257 | CACTGAAACGGGGAGGGG | 80 |
| 1557995 | 11004 | 11021 | 2244 | 2261 | GGTCCACTGAAACGGGGA | 392 |
| 1557986 | 11005 | 11022 | 2245 | 2262 | GGGTCCACTGAAACGGGG | 470 |
| 1557987 | 11030 | 11047 | 2270 | 2287 | GGATAGGGAAAAGCACCT | 83 |
| 1557985 | 11032 | 11049 | 2272 | 2289 | GTGGATAGGGAAAAGCAC | 239 |

### Example 5: Effect of Modified Oligonucleotides Complementary to Human GRN in H4 Cells; Single Dose

Modified oligonucleotides selected from the examples above were tested for their effect on progranulin protein levels in vitro. Also tested was a control scrambled modified oligonucleotide (“control scrambled ASO”) (uniformly 2′MOE-modified, phosphorothioate backbone having a nucleobase sequence (from 5′ to 3′):

(SEQ ID NO: 10))
TTAGTTTAATCACGCTCG.

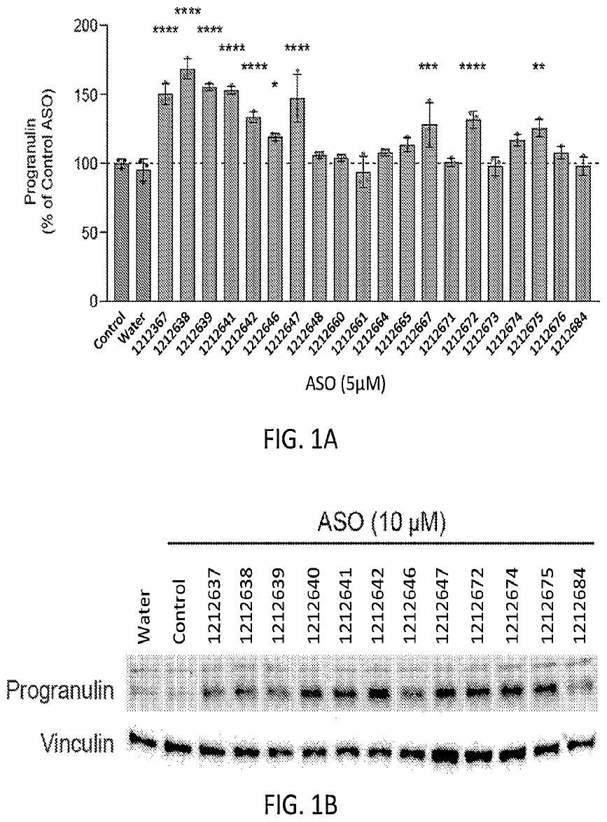

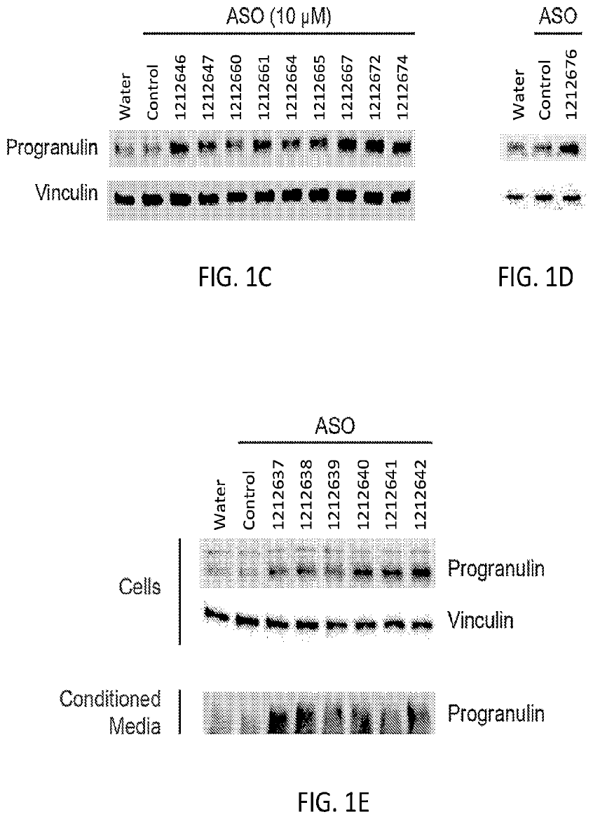

H4 human neuroglioma cells (ATCC HTB-148) were treated with modified oligonucleotide at a concentration of 5000 nM for 24 hours. Progranulin levels were measured in cell lysates by ELISA. Progranulin concentrations were determined in duplicate using 10-15 μl of lysates per well (typically 8-20 μg of total protein per well) using a sandwich ELISA assay (R&D Systems, DPGRN0). Data are presented in FIG. 1A as means±/−SD, * indicates p<0.05; ** indicates p<0.01, *** indicates p<0.001, **** indicates p<0.0002, as determined by one-way ANOVA with Dunnett post hoc test.

H4 human neuroglioma cells were treated with modified oligonucleotide at a concentration of 10 μM for 24 hours. Progranulin levels were measured in cell lysates by Western blot (FIG. 1B, FIG. 1C, and FIG. 1D). In a separate experiment, H4 cells were treated with 10 μM modified oligonucleotide for 24 hours; progranulin levels in the cell lysates, as well as secreted progranulin levels in the conditioned media were assessed by Western blot. Modified oligonucleotides that increased cellular progranulin levels (FIG. 1E top panel) also increased secreted progranulin levels in the media (FIG. 1E bottom panel).

Primary antibodies used for immunoblot analysis include: an anti-human progranulin linker 5 polyclonal antibody #614 that recognizes an epitope between amino acids 497-515 (Nguyen, A. D., et al., J. Biol. Chem. 288, 8627-8635 (2013)), an anti-human vinculin monoclonal antibody (loading control for protein level) (Cell Signaling Technology, 13901).

### Example 6: Effect of Modified Oligonucleotides Complementary to Human GRN In Vitro; Multiple Dose

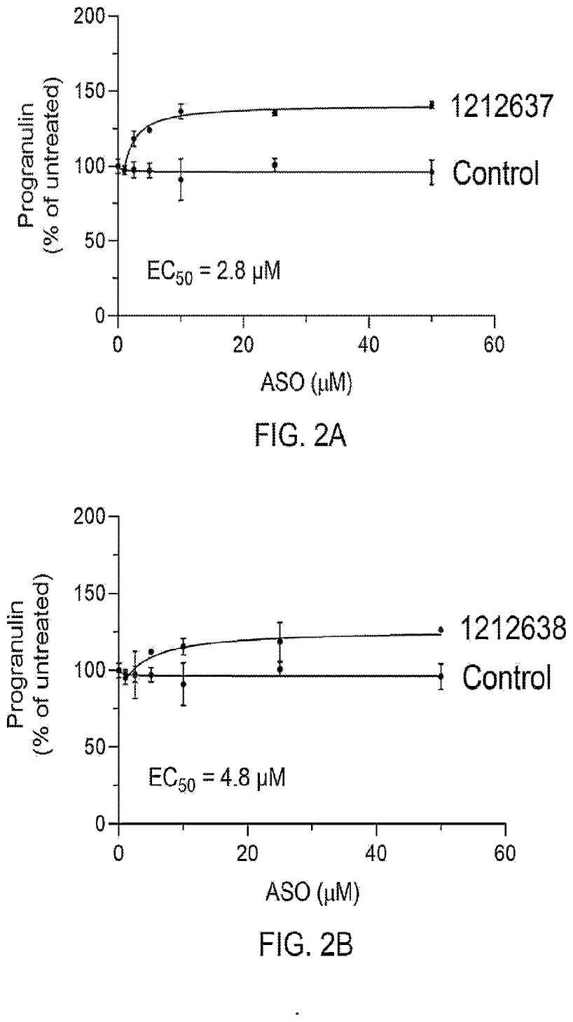

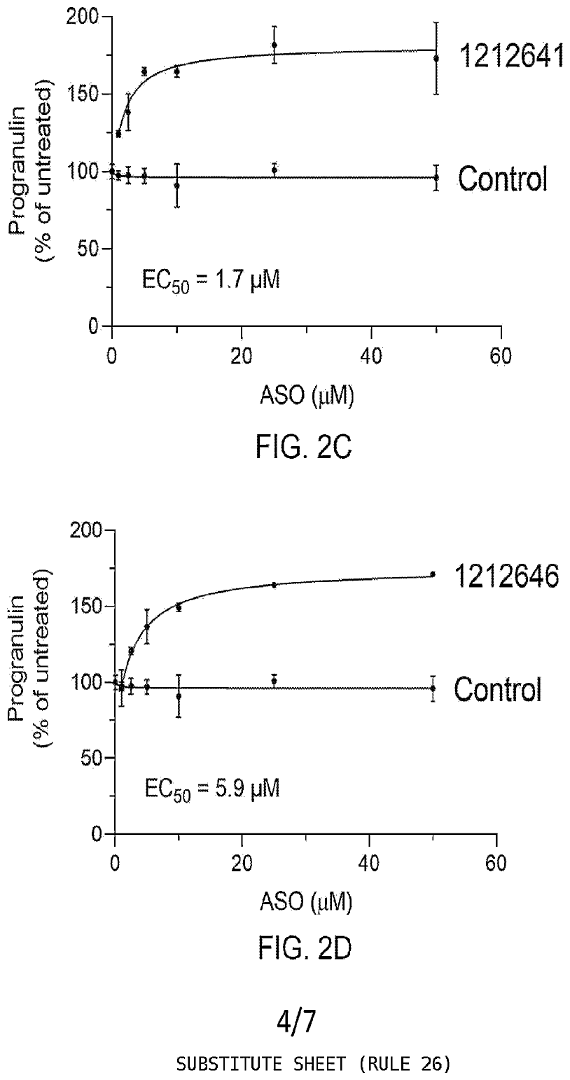

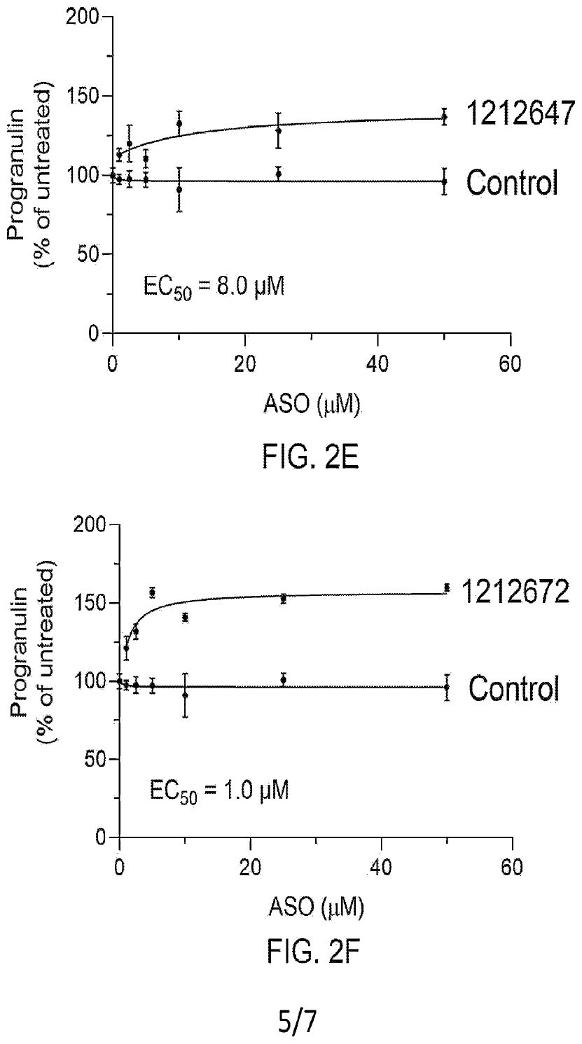

Modified oligonucleotides selected from the examples above, and the control scrambled ASO described above, were tested for their effect on progranulin protein levels in H4 cells. Cells were treated with modified oligonucleotide at the doses indicated in FIGS. 2A-2F for 24 hours. Progranulin levels were measured in cell lysates by ELISA, as in Example 5. Data are presented as means±SD, with EC₅₀ values as indicated.

### Example 7: Effect of Modified Oligonucleotides Complementary to Human GRN in iPSC-Derived Neurons

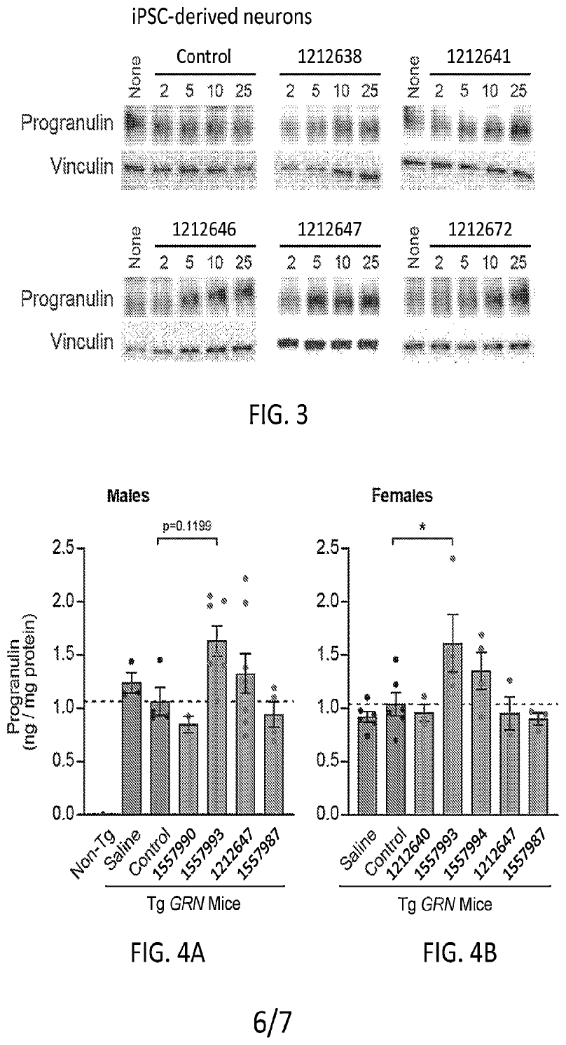

Modified oligonucleotides selected from the examples above, and the control scrambled ASO described above, were tested for their effect on progranulin protein levels in iPSC-derived neurons. Human iPSCs harboring doxycycline-responsive Neurogenin-2 (NGN2) expression for differentiation into i3 cortical neurons were prepared as described (Fernandopulle, M. S., et al., Curr. Protoc. Cell Biol. 79, e51 (2018)). After 2 weeks of differentiation, the i3 neurons were treated with modified oligonucleotide at the indicated concentrations (μM) for 3-4 days. Western blot analysis was conducted as in Example 5, with the data shown in FIG. 3.

### Example 8: Effect of Modified Oligonucleotides Complementary to Human GRN In Vivo

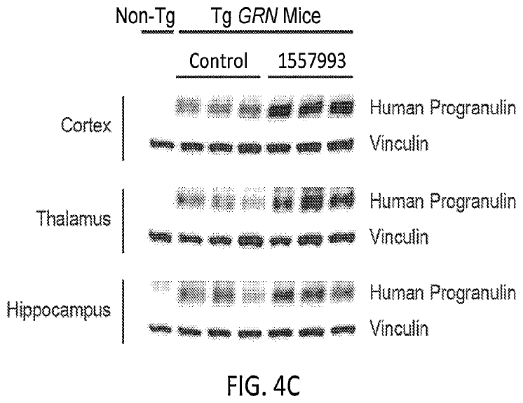

Homozygous knock in mice for human GRN were prepared from a heterozygous C₅₇BL/6J breeder pair (Petkau et al., Neurobiol. Dis. 153, 105314 (2021)). These transgenic GRN mice were treated with 500 μg modified oligonucleotide by bolus intracerebroventricular injection into the right lateral ventricle (Jafar-Nejad, P., et al., Nucleic Acids Res. 49, 657-673 (2021); Farr, S. A., et al., J. Alzheimers Dis. 40, 1005-1016 (2014)). Three weeks post administration, mice were sacrificed and brain tissues were collected for protein analysis. Human progranulin levels in the cortex of mice treated with the indicated modified oligonucleotide, the control scrambled ASO described above, and saline-treated mice as determined by ELISA (progranulin levels determined in duplicate), are shown in FIG. 4B and FIG. 4C. Human progranulin levels were increased by 53% and 55% in male and female mice, respectively, that were treated with Compound 1557993, compared to mice of the same sex that received the scrambled control ASO. Data are presented as means±SEM; *indicates p<0.05, as determined by one-way ANOVA with Dunnett post hoc test. Non-Tg=untreated non-transgenic mice, which are littermates of the transgenic mice and do not carry the human GRN alleles. A Western blot of human progranulin in cortex, thalamus, and hippocampus of male mice treated with control, or with Compound 1557993 (FIG. 4C) was conducted as in Example 5, and shows increased human progranulin in the cortex, thalamus, and hippocampus of male mice treated with the compound.

1. An oligomeric compound comprising a modified oligonucleotide consisting of 12 to 50 linked nucleosides, wherein the nucleobase sequence of the modified oligonucleotide is at least 80% complementary to an equal length portion of a progranulin nucleic acid, and wherein the modified oligonucleotide has at least one modification selected from a modified sugar moiety and a modified internucleoside linkage.

2. The oligomeric compound of claim 1, wherein the progranulin nucleic acid has the nucleobase sequence of SEQ ID NO: 1 or SEQ ID NO: 2.

3. An oligomeric compound comprising a modified oligonucleotide consisting of 18, 19, or 20 linked nucleosides, wherein the nucleobase sequence of the modified oligonucleotide comprises at least 16, at least 17, or 18 contiguous nucleobases of any of the nucleobase sequences of SEQ ID NOs:12-854, and wherein the modified oligonucleotide comprises at least one modification selected from a modified sugar moiety and a modified internucleoside linkage.

4. The oligomeric compound of any of claims 1-3, wherein the nucleobase sequence of the modified oligonucleotide comprises at least 16, at least 17, or 18 contiguous nucleobases of any of the nucleobase sequences of SEQ ID NOs: 34, 268, 345, 424, 501, 579, 657, 735, 800, 801, 806, 812, 813, 816, 821, 828, 844, and 847.

5. An oligomeric compound comprising a modified oligonucleotide consisting of 18 linked nucleosides, wherein the nucleobase sequence of the modified oligonucleotide consists of the nucleobase sequence of any of SEQ ID NOs: 12-854, and wherein the modified oligonucleotide comprises at least one modification selected from a modified sugar moiety and a modified internucleoside linkage.

6. The oligomeric compound of claim 5, wherein the nucleobase sequence of the modified oligonucleotide consists of 18 contiguous nucleobases of any of the nucleobase sequences of SEQ ID NOs: 34, 268, 345, 424, 501, 579, 657, 735, 800, 801, 806, 812, 813, 816, 821, 828, 844, and 847.

7. An oligomeric compound comprising a modified oligonucleotide consisting of 12 to 50 linked nucleosides, wherein the nucleobase sequence of the modified oligonucleotide comprises at least 8, at least 9, at least 10, at least 11, at least 12, at least 13, at least 14, at least 15, at least 16, at least 17, or 18 contiguous nucleobases complementary to an equal length portion within nucleobases 8497-8552 of SEQ ID NO: 1.

8. The oligomeric compound of claim 7, wherein the nucleobase sequence of the modified oligonucleotide consists of the nucleobase sequence of any of SEQ ID NOs: 34, 268, 345, 424, 501, 579, 657, 735, 800, 801, 806, 812, 813, 816, 821, 828, 844, and 847.

9. The oligomeric compound of any of claims 1-8, wherein the nucleobase sequence of the modified oligonucleotide is at least 85%, at least 90%, at least 95%, or is 100% complementary to an equal length portion within the nucleobase sequence of any of SEQ ID NO: 1 or SEQ ID NO: 2 when measured across the entire nucleobase sequence of the modified oligonucleotide.

10. The oligomeric compound of any of claims 1-9, wherein at least one nucleoside of the modified oligonucleotide comprises a modified sugar moiety.

11. The oligomeric compound of claim 10, wherein the modified sugar moiety comprises a bicyclic sugar moiety.

12. The oligomeric compound of claim 11, wherein the bicyclic sugar moiety comprises a 4′-2′ bridge selected from —CH₂—O— and —CH(CH₃)—O—.

13. The oligomeric compound of any of claims 10-12, wherein the modified sugar moiety comprises a non-bicyclic modified sugar moiety.

14. The oligomeric compound of claim 13, wherein the non-bicyclic modified sugar moiety is a 2′-MOE modified sugar moiety, a 2′-NMA modified sugar moiety, a 2′-OMe modified sugar moiety, or a 2′-F modified sugar moiety.

15. The oligomeric compound of claim 10, wherein the modified sugar moiety is a sugar surrogate.

16. The oligomeric compound of claim 15, wherein the sugar surrogate is a morpholino, modified morpholino, PNA, THP, or F-HNA.

17. The oligomeric compound of claim 10, wherein each nucleoside of the modified oligonucleotide comprises a modified sugar moiety.

18. The oligomeric compound of claim 17, wherein each modified sugar moiety is a 2′-MOE modified sugar moiety, a 2′-NMA modified sugar moiety, a 2′-OMe modified sugar moiety, or a 2′-F modified sugar moiety.

19. The oligomeric compound of claim 17, wherein each modified sugar moiety is a 2′-MOE modified sugar moiety.

20. The oligomeric compound of claim 17, wherein each modified sugar moiety is a sugar surrogate.

21. The oligomeric compound of claim 20, wherein each modified sugar moiety is a morpholino, modified morpholino, PNA, THP, or F-HNA.

22. The oligomeric compound of any of claims 1-21, wherein the modified oligonucleotide comprises at least one modified internucleoside linkage.

23. The oligomeric compound of claim 22, wherein the at least one modified internucleoside linkage is a phosphorothioate internucleoside linkage.

24. The oligomeric compound of claim 22, wherein each internucleoside linkage of the modified oligonucleotide is a modified internucleoside linkage.

25. The oligomeric compound of claim 22, wherein each internucleoside linkage is a phosphorothioate internucleoside linkage.

26. The oligomeric compound of any of claims 1-23, wherein at least one internucleoside linkage of the modified oligonucleotide is a phosphodiester internucleoside linkage.

27. The oligomeric compound of any of claims 22-23 or 26, wherein each internucleoside linkage of the modified oligonucleotide is independently selected from a phosphodiester internucleoside linkage and a phosphorothioate internucleoside linkage.

28. The oligomeric compound of any of claims 22-24 or 26-27, wherein at least 10, at least 11, at least 12, at least 13, at least 14, at least 15, at least 16, or at least 17 internucleoside linkages of the modified oligonucleotide are phosphorothioate internucleoside linkages.

29. The oligomeric compound of any of claims 1-23, wherein the modified oligonucleotide consists of 18 linked nucleosides and has an internucleoside linkage motif selected from sssssssssssssssss and sosssssssosssssss.

30. The oligomeric compound of claim 19, wherein the modified oligonucleotide consists of 18 linked nucleosides, wherein the internucleotide linkage motif of the modified oligonucleotide is sssssssssssssssss.

31. The oligomeric compound of claim 19, wherein the modified oligonucleotide consists of 18 linked nucleosides, wherein the internucleotide linkage motif of the modified oligonucleotide is sosssssssosssssss.

32. The oligomeric compound of any of claims 1-31, wherein the modified oligonucleotide comprises at least one modified nucleobase.

33. The oligomeric compound of claim 32, wherein the modified nucleobase is a 5-methyl cytosine or hypoxanthine.

34. The oligomeric compound of claim 33, wherein each cytosine is a 5-methyl cytosine.

35. An oligomeric compound comprising a modified oligonucleotide according to any one of the following chemical notations (5′ to 3′):
(SEQ ID NO: 855)
GₑₛTₑₒ^{m}Cₑₛ^{m}CₑₛAₑₛGₑₛGₑₛGₑₛAₑₛGₑₒAₑₛAₑₛTₑₛTₑₛTₑₛGₑₛGₑₛTₑ;
(SEQ ID NO: 856)
GₑₛTₑₒGₑₛGₑₛAₑₛTₑₛAₑₛGₑₛGₑₛGₑₒAₑₛAₑₛAₑₛAₑₛGₑₛ^{m}CₑₛAₑₛ^{m}Cₑ;
(SEQ ID NO: 857)
GₑₛGₑₒGₑₛTₑₛ^{m}Cₑₛ^{m}CₑₛAₑₛ^{m}CₑₛTₑₛGₑₒAₑₛAₑₛAₑₛ^{m}CₑₛGₑₛGₑₛGₑₛGₑ;
(SEQ ID NO: 858)
GₑₛGₑₒAₑₛTₑₛAₑₛGₑₛGₑₛGₑₛAₑₛAₑₒAₑₛAₑₛGₑₛ^{m}CₑₛAₑₛ^{m}Cₑₛ^{m}CₑₛTₑ;
(SEQ ID NO: 859)
GₑₛGₑₒTₑₛ^{m}Cₑₛ^{m}CₑₛAₑₛGₑₛGₑₛGₑₛAₑₒGₑₛAₑₛAₑₛTₑₛTₑₛTₑₛGₑₛGₑ;
(SEQ ID NO: 860)
AₑₛAₑₒAₑₛ^{m}CₑₛGₑₛGₑₛGₑₛGₑₛAₑₛGₑₒGₑₛGₑₛGₑₛAₑₛTₑₛGₑₛGₑₛ^{m}Cₑ;
(SEQ ID NO: 861)
GₑₛAₑₒAₑₛAₑₛ^{m}CₑₛGₑₛGₑₛGₑₛGₑₛAₑₒGₑₛGₑₛGₑₛGₑₛAₑₛTₑₛGₑₛGₑ;
(SEQ ID NO: 862)
TₑₛGₑₒAₑₛAₑₛAₑₛ^{m}CₑₛGₑₛGₑₛGₑₛGₑₒAₑₛGₑₛGₑₛGₑₛGₑₛAₑₛTₑₛGₑ;
(SEQ ID NO: 863)
^{m}CₑₛTₑₒGₑₛAₑₛAₑₛAₑₛ^{m}CₑₛGₑₛGₑₛGₑₒGₑₛAₑₛGₑₛGₑₛGₑₛGₑₛAₑₛTₑ;
(SEQ ID NO: 864)
Aₑₛ^{m}CₑₒTₑₛGₑₛAₑₛAₑₛAₑₛ^{m}CₑₛGₑₛGₑₒGₑₛGₑₛAₑₛGₑₛGₑₛGₑₛGₑₛAₑ;
(SEQ ID NO: 865)
^{m}CₑₛAₑₒ^{m}CₑₛTₑₛGₑₛAₑₛAₑₛAₑₛ^{m}CₑₛGₑₒGₑₛGₑₛGₑₛAₑₛGₑₛGₑₛGₑₛGₑ;
or
(SEQ ID NO: 866)
GₑₛGₑₒTₑₛ^{m}Cₑₛ^{m}CₑₛAₑₛ^{m}CₑₛTₑₛGₑₛAₑₒAₑₛAₑₛ^{m}CₑₛGₑₛGₑₛGₑₛGₑₛAₑ);
wherein:
wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

36. The oligomeric compound of any of claims 1-35, wherein the modified oligonucleotide is a pharmaceutically acceptable salt.

37. The oligomeric compound of claim 36, wherein the modified oligonucleotide is a pharmaceutically acceptable salt comprising one or more cations selected from sodium, potassium, calcium, and magnesium.

38. The oligomeric compound of any of claims 1-37, consisting of the modified oligonucleotide.

39. The oligomeric compound of any of claims 1-37, wherein the oligomeric compound comprises a conjugate group.

40. The oligomeric compound of claim 39, wherein the conjugate group comprises a conjugate linker and a conjugate moiety.

41. The oligomeric compound of claim 40, wherein the conjugate linker consists of a single bond.

42. The oligomeric compound of claim 40 or claim 41, wherein the conjugate linker is cleavable.

43. The oligomeric compound of claim 40 or claim 41, wherein the conjugate linker comprises 1-3 linker nucleosides.

44. The oligomeric compound of any of claims 40-42, wherein the conjugate linker does not comprise any linker nucleosides.

45. The oligomeric compound of any of claims 39-44, wherein the conjugate group is attached to the 5′-end of the modified oligonucleotide.

46. The oligomeric compound of any of claims 39-44, wherein the conjugate group is attached to the 3′-end of the modified oligonucleotide.

47. The oligomeric compound of any of claims 39-44, wherein the oligomeric compound comprises a terminal group.

48. The oligomeric compound of claim 47, wherein the terminal group is an abasic sugar moiety.

49. The oligomeric compound of any of claims 1-48, wherein the oligomeric compound is a single-stranded oligomeric compound.

50. An oligomeric compound comprising a modified oligonucleotide according to the following chemical notation:
(SEQ ID NO 864)
Aₑₛ^{m}CₑₒTₑₛGₑₛAₑₛAₑₛAₑₛ^{m}CₑₛGₑₛGₑₒGₑₛGₑₛAₑₛGₑₛGₑₛGₑₛGₑₛAₑ,
wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

51. An oligomeric compound comprising a modified oligonucleotide according to the following chemical notation:
(SEQ ID NO 865)
^{m}CₑₛAₑₒ^{m}CₑₛTₑₛGₑₛAₑₛAₑₛAₑₛ^{m}CₑₛGₑₒGₑₛGₑₛGₑₛAₑₛGₑₛGₑₛGₑₛGₑ,
wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

52. A modified oligonucleotide according to the following chemical structure:

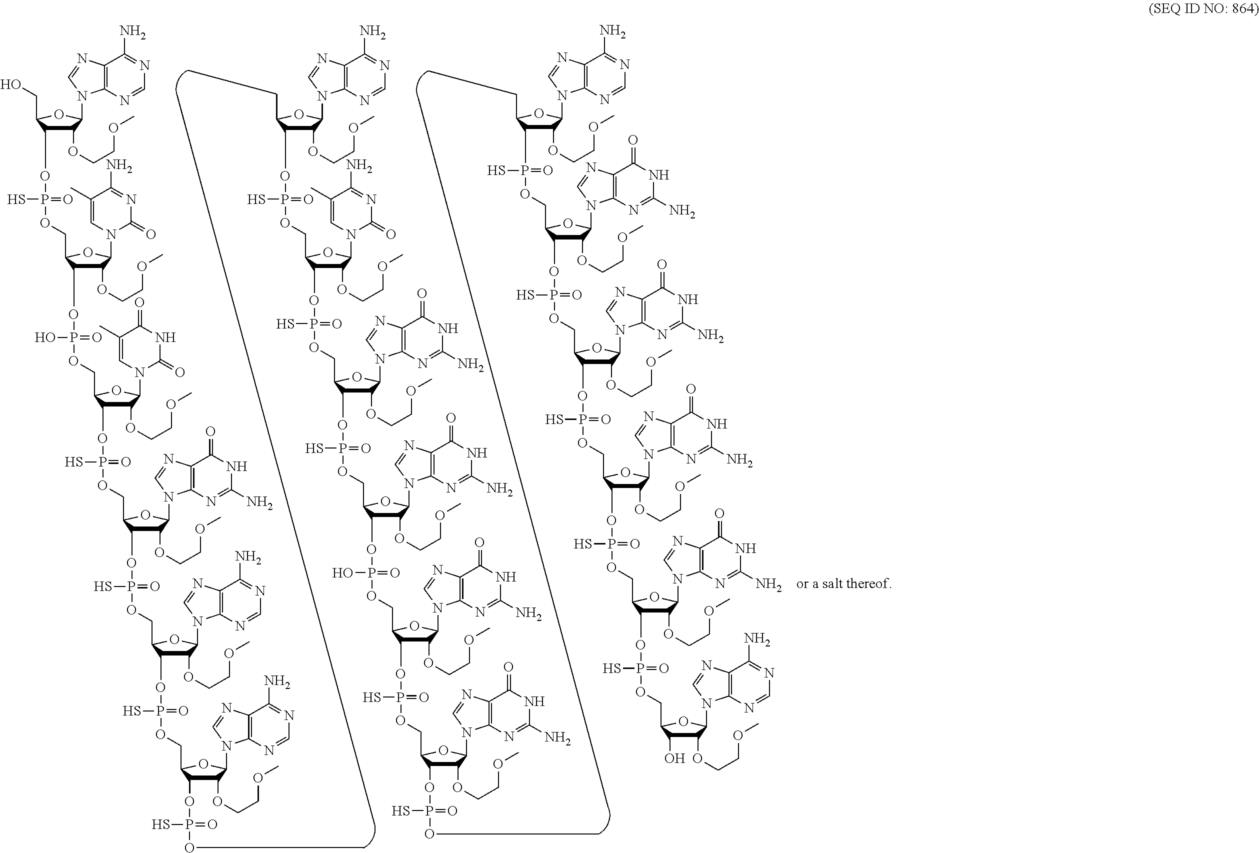

53. The modified oligonucleotide of claim 52, which is the sodium salt or the potassium salt.

54. A modified oligonucleotide according to the following chemical structure:

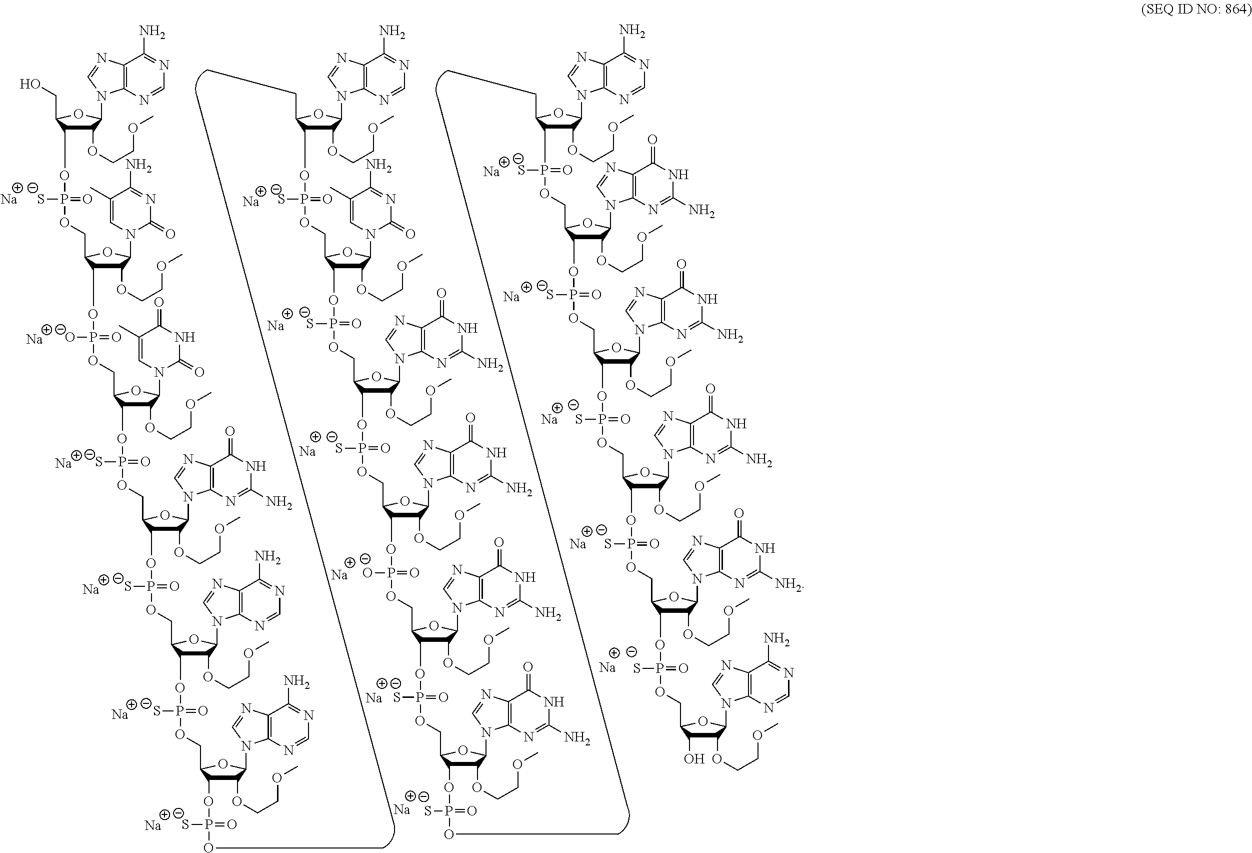

55. A modified oligonucleotide according to the following chemical structure:

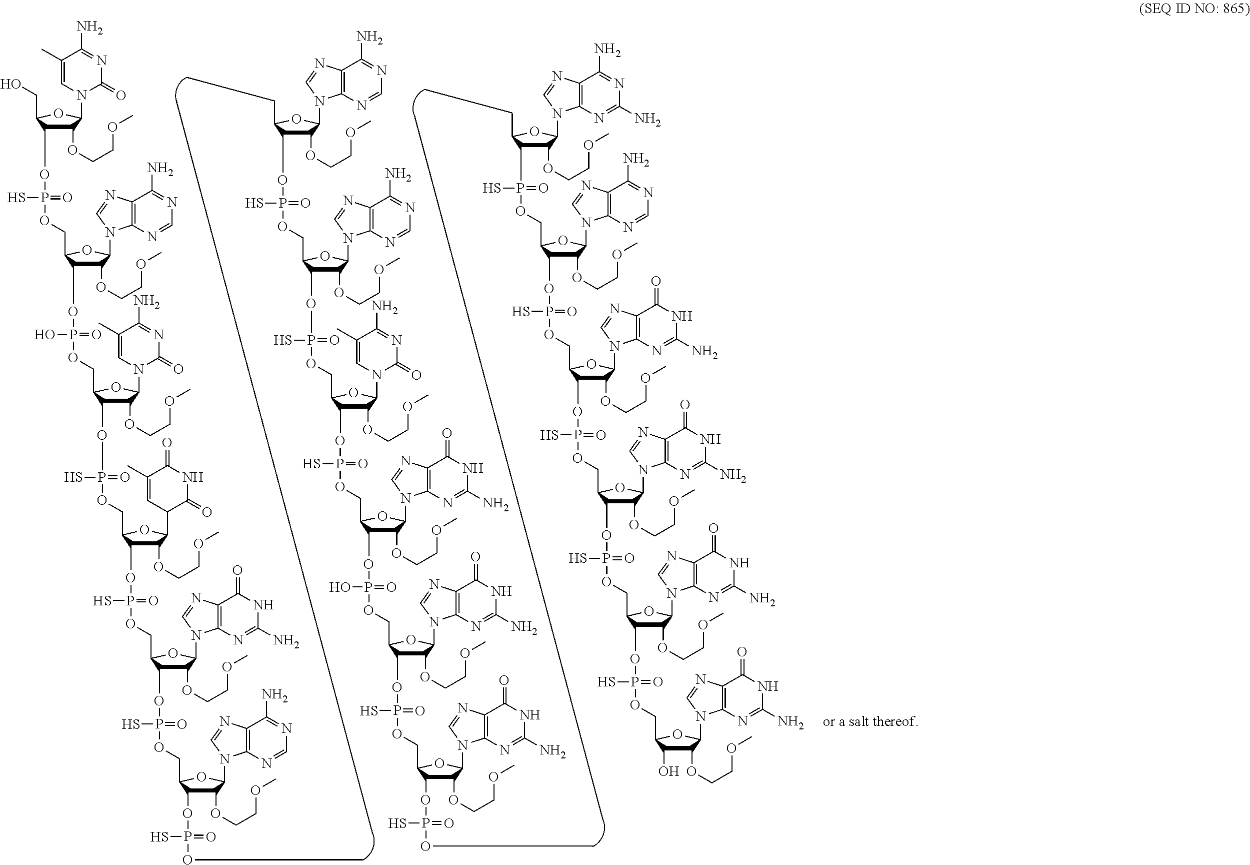

56. The modified oligonucleotide of claim 55, which is the sodium salt or the potassium salt.

57. A modified oligonucleotide according to the following chemical structure:

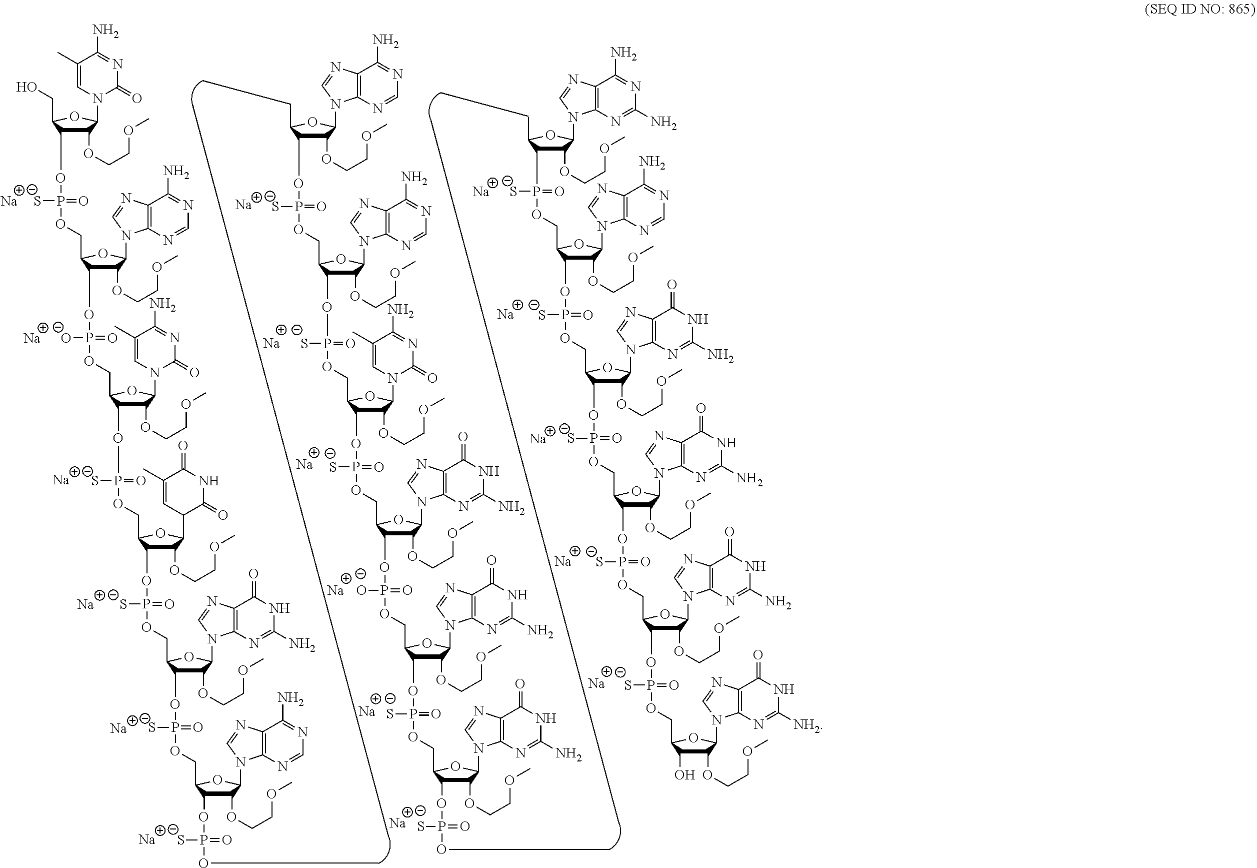

58. A chirally enriched population of oligomeric compounds of any of claims 1-51, or a chirally enriched population of modified oligonucleotides ofany of claims 52-57, wherein the population is enriched for oligomeric compounds comprising at least one particular phosphorothioate internucleoside linkage having a particular stereochemical configuration.

59. The chirally enriched population of claim 58, wherein the population is enriched for modified oligonucleotides comprising at least one particular phosphorothioate internucleoside linkage having the (Sp) configuration.

60. The chirally enriched population of claim 58, wherein the population is enriched for modified oligonucleotides comprising at least one particular phosphorothioate internucleoside linkage having the (Rp) configuration.

61. The chirally enriched population of claim 58, wherein the population is enriched for modified oligonucleotides having a particular, independently selected stereochemical configuration at each phosphorothioate internucleoside linkage.

62. The chirally enriched population of claim 61, wherein the population is enriched for modified oligonucleotides having the (Sp) configuration at each phosphorothioate internucleoside linkage or for modified oligonucleotides having the (Rp) configuration at each phosphorothioate internucleoside linkage.

63. The chirally enriched population of claim 61, wherein the population is enriched for modified oligonucleotides having the (Rp) configuration at one particular phosphorothioate internucleoside linkage and the (Sp) configuration at each of the remaining phosphorothioate internucleoside linkages.

64. The chirally enriched population of claim 61, wherein the population is enriched for modified oligonucleotides having at least 3 contiguous phosphorothioate internucleoside linkages in the Sp, Sp, and Rp configurations, in the 5′ to 3′ direction.

65. A population of oligomeric compounds of any of claims 1-51, or a population of modified oligonucleotides of any of claims 52-57, wherein all of the phosphorothioate internucleoside linkages of the modified oligonucleotide are stereorandom.

66. An antisense agent comprising an antisense compound, wherein the antisense compound is the oligomeric compound of any of claims 1-51.

67. The antisense agent of claim 66, wherein the antisense agent comprises a conjugate group, wherein the conjugate group comprises a cell-targeting moiety.

68. A pharmaceutical composition comprising an oligomeric compound of any of claims 1-51, a modified oligonucleotide of any of claims 52-57, a population of any of claims 58-65, or an antisense agent of claim 66 or claim 67, and a pharmaceutically acceptable diluent.

69. The pharmaceutical composition of claim 68, wherein the pharmaceutically acceptable diluent is artificial CSF (aCSF) or phosphate-buffered saline (PBS).

70. The pharmaceutical composition of claim 69, wherein the pharmaceutical composition consists essentially of the oligomeric compound of any of claims 1-51, the modified oligonucleotide of any of claims 52-57, the population of any of claims 58-65, the or the antisense agent of claim 66 or claim 67, and aCSF.

71. The pharmaceutical composition of claim 69, wherein the pharmaceutical composition consists essentially of the oligomeric compound of any of claims 1-51, the modified oligonucleotide of any of claims 52-57, the population of any of claims 58-65, or the antisense agent of claim 66 or claim 67, and PBS.

72. A method comprising administering to a subject an oligomeric compound of any of claims 1-51, a modified oligonucleotide of any of claims 52-57, a population of any of claims 58-65, an antisense agent of claim 66 or claim 67, or a pharmaceutical composition of any of claims 68-71.

73. A method of treating a disease or disorder associated with an insufficient expression of progranulin, comprising administering to a subject having or at risk for developing a disease or disorder associated with insufficient expression of progranulin a therapeutically effective amount of an oligomeric compound of any of claims 1-51, a modified oligonucleotide of any of claims 52-57, a population of any of claims 58-65, an antisense agent of claim 66 or claim 67, or a pharmaceutical composition of any of claims 68-71, thereby treating the disease or disorder associated with an insufficient expression of progranulin.

74. The method of claim 73, wherein the disease or disorder associated with an insufficient expression of progranulin is a neurological disease or disorder.

75. The method of claim 73 or claim 74, wherein the disease or disorder associated with an insufficient expression of progranulin is a lysosomal storage disorder or a TDP-43 proteinopathy.

76. The method of any of claims 73-75, wherein the disease or disorder associated with insufficient expression of progranulin is frontotemporal dementia (FTD), frontotemporal lobar degeneration (FTLD), neuronal ceroid lipofuscinosis (NCL), Alzheimer's disease (AD), or amyotrophic lateral sclerosis (ALS).

77. The method of any of claims 73-77, wherein at least one symptom or hallmark of the disease or disorder associated with insufficient expression of progranulin is ameliorated.

78. The method of claim 77, wherein the at least one symptom or hallmark is deterioration in behavior and personality, language impairment, alterations in muscle or motor functions, memory loss, cognitive dysfunction, tremor, seizures, or dizziness.

79. The method of claim 77 or claim 78, wherein administration of the oligomeric compound of any of claims 1-51, the modified oligonucleotide of any of claims 52-57, the population of any of claims 58-65, the antisense agent of claim 66 or claim 67, or the pharmaceutical composition of any of claims 68-71 improves behavior or personality, slows deterioration in behavior or personality, improves language ability, slows deterioration of language ability, improves muscle or motor function, slows deterioration in muscle or motor function, improves memory, slows deterioration in memory, improves cognitive function, slows deterioration of cognitive function, reduces tremors, reduces seizures, or reduces dizziness.

80. The method of any of claims 73-79, wherein the oligomeric compound of any of claims 1-51, the modified oligonucleotide of any of claims 52-57, the population of any of claims 58-65, the antisense agent of claim 66 or claim 67, or the pharmaceutical composition of any of claims 68-71 is administered to the central nervous system or systemically.

81. The method of any of claims 73-79, wherein the oligomeric compound of any of claims 1-51, the modified oligonucleotide of any of claims 52-57, the population of any of claims 58-65, the antisense agent of claim 66 or claim 67, or the pharmaceutical composition of any of claims 68-71 is administered any of intrathecally, systemically, subcutaneously, or intramuscularly.

82. The method of any of claims 73-81, wherein the subject is a human.

83. A method of increasing progranulin RNA or one or more splice variants of said progranulin RNA in a cell, comprising contacting the cell with an oligomeric compound of any of claims 1-51, a modified oligonucleotide of any of claims 52-57, a population of any of claims 58-65, or an antisense agent of claim 66 or claim 67.

84. A method of increasing progranulin protein in a cell, comprising contacting the cell with an oligomeric compound of any of claims 1-51, a modified oligonucleotide of any of claims 52-57, a population of any of claims 58-65, or an antisense agent of claim 66 or claim 67.

85. The method of claim 83 or claim 84, wherein the cell is a neuron.

86. The method of any of claims 83-85, wherein the cell is a human cell.

87. Use of an oligomeric compound of any of claims 1-51, a modified oligonucleotide of any of claims 52-57, a population of any of claims 58-65, an antisense agent of claim 66 or claim 67, or a pharmaceutical composition of any of claims 68-71 for treating a disease or disorder associated with an insufficient expression of progranulin.

88. Use of an oligomeric compound of any of claims 1-51, a modified oligonucleotide of any of claims 52-57, a population of any of claims 58-65, an antisense agent of claim 66 or claim 67, or a pharmaceutical composition of any of claims 68-71 in the manufacture of a medicament for treating a disease or disorder associated with an insufficient expression of progranulin.

89. The use of claim 87 or claim 88, wherein the disease or disorder associated with an insufficient expression of progranulin is a neurological disease or disorder.

90. The use of claim 87 or claim 88, wherein the disease or disorder associated with an insufficient expression of progranulin is a lysosomal storage disorder or a TDP-43 proteinopathy.

91. The use of any of claims 87-90, wherein the disease or disorder associated with insufficient expression of progranulin is frontotemporal dementia (FTD), frontotemporal lobar degeneration (FTLD), neuronal ceroid lipofuscinosis (NCL), Alzheimer's disease (AD), or amyotrophic lateral sclerosis (ALS).
